(19) United States
(12) Patent Application Publication
(10) Pub. No.: US 2025/0223324 A1
(43) Pub. Date: Jul. 10, 2025

(54) DIPHTHERIA-BASED INTRANEURAL DELIVERY VEHICLES AND PRODUCTION PROCESSES THEREOF

(71) Applicants: NEW YORK UNIVERSITY, New York, NY (US); WAKE FOREST UNIVERSITY HEALTH SCIENCES, Winston-Salem, NC (US)

(72) Inventors: Konstantin ICHTCHENKO, New York, NY (US); Cesar H. BENJUMEA, Brooklyn, NY (US); Patrick MCNUTT, Trinity, NC (US); Jonathan LIU, New York, NY (US)

(21) Appl. No.: 19/011,169

(22) Filed: Jan. 6, 2025

Related U.S. Application Data
(60) Provisional application No. 63/618,199, filed on Jan. 5, 2024.

(51) Int. Cl. C07K 14/34; A61K 38/00; A61P 25/28; C07K 1/107; C07K 14/33; C12P 21/02

(52) U.S. Cl. CPC C07K 14/34; A61P 25/28; C07K 1/1077; C07K 14/33; C12P 21/02; A61K 38/00; C07K 2319/035

(57) ABSTRACT

The present disclosure is directed to fusion proteins and propeptide fusions comprising a diphtheria toxin catalytic domain and translocation domain with a receptor binding domain from a Clostridial neurotoxin. The fusion protein has a therapeutic cargo positioned upstream of the catalytic domain. Also disclosed are therapeutic agents, treatment methods, propeptide fusions, isolated nucleic acid molecules, expression systems, host cells, and methods of expressing fusion proteins.

This application claims the benefit of U.S. Provisional Patent Application Ser. No. 63/618,199, filed Jan. 5, 2024, which is hereby incorporated by reference in its entirety.

This invention was made with government support under R01 AI093504 awarded by the National Institutes of Health. The government has certain rights in the invention.

### FIELD

The present disclosure relates to diphtheria toxin fusion protein delivery vehicles containing therapeutic cargo, propeptide fusions, methods of production, and methods of use.

### SEQUENCE LISTING STATEMENT

This application contains a computer readable Sequence Listing which has been submitted electronically in XML format and is hereby incorporated by reference in its entirety. The XML file was created on Jan. 3, 2025, is named 147462002531.xml and is 220,259 bytes in size.

### BACKGROUND

Treating botulinum neurotoxin (BoNT) intoxication presents significant challenges due to the mechanisms by which this toxin exerts its effect. BoNT causes flaccid paralysis by cleaving SNARE proteins essential for neurotransmitter release.

Current treatments, such as antitoxins, are limited in efficacy once the toxins have entered neurons.

The botulinum neurotoxin's heterodimer comprises three major functional domains: 1) the Light Chain Zn²⁺-metalloprotease domain (LC; Catalytic Domain); 2) the Heavy Chain C-terminal domain (HC_{C}; Receptor Binding Domain); and 3) the HC N-terminal domain (HC_{N}; Translocation domain), responsible for the passage of the LC through the endosomal membrane to the neuronal cytoplasm. BoNT LC accumulated in the neuronal cytoplasm proteolytically cleaves Soluble N-ethylmaleimide-sensitive factor Attachment protein REceptor (SNARE) proteins, preventing functional assembly of the tripartite complex of SNAP25/VAMP/Syntaxin required for synaptic transmission, and caused the flaccid paralysis characteristic of clinical botulism.

The current treatment for botulism is a post-exposure prophylaxis with equine-derived Heptavalent Botulinum AntiToxin (HBAT) combined with chronic ventilation and supportive care as needed. HBAT neutralizes toxin in the bloodstream but is ineffective once toxin has bound to or been internalized into neurons (Simpson L., “Identification of the Major Steps in Botulinum Toxin Action,” Annual Review of Pharmacology and Toxicology 44(1):167-193 (2004)). The stark limitations of current botulism treatments have necessitated a search for pharmacotherapies that accelerate symptomatic reversal.

A BoNT based biotherapeutic comprising a single domain antibody (sdAb; B8) cargo genetically fused to C1ad—a botulinum neurotoxin-based delivery vehicle was developed (B8C1ad). B8C1ad can enter neurons and protect SNARE proteins by inhibiting LC/A1 catalytic activity in situ. Post-symptomatic administration of B8C1ad produced antidotal rescue in mice, guinea pigs, and nonhuman primates after a lethal BoNT/A1 botulism challenge.

A critical limitation of B8C1ad has been the intrinsic latent toxicity of the delivery vehicle C1ad, which decreases the therapeutic window of B8C1ad (NO Adverse Events Level (NOAEL): 0.4 mg/kg, EC₅₀: 0.025 mg/kg, LD₅₀: 5 mg/kg). Although the available dose ranges have proven effective, the C1ad toxicity has limited the administration of larger therapeutic doses. Notably, the maximum therapeutic dose that has been administered corresponds to the NOAEL value. This dose also corresponds to the maximum observed therapeutic effect, a fact that leads to the hypothesis that delivery vehicles with improved safety profiles could be more effective.

Another important limitation of a C1ad botulinum neurotoxin-based delivery vehicle is its inability to translocate a large variety of protein cargos that do not share the same properties as the native botulinum toxin light chain metalloprotease, which is able to undergo globular melting during translocation through the endosomal pore followed by refolding/restoration of enzymatic activity after LC entry into neuronal cytosol. Multiple experiments have shown that the efficiency of the cargo delivery fused to N-terminus of metalloprotease-inactivated LC substantially decreases as the cargo increases in size and rigidity. Interestingly, a single domain antibody such as B8 is able to share, at least in part, the above-mentioned properties of BoNT light chain and has been shown to be active after translocation to the cytoplasm. However, protein cargos such as eGFP (27 kDa) and Halotag7 (33 kDa) seem to have a negative effect on translocation efficiency.

Thus, despite recent advances in targeted drug delivery, there is still a need to develop reliable systems capable of delivering large amounts of therapeutic cargo in a safe and effective manner into the cytoplasmic compartment of neurons to treat BoNT intoxication as well as other neuronal conditions and diseases. Currently available and described delivery vehicles have limitations related to size, rigidity, and/or structural integrity of the cargos that can be effectively delivered into the neuronal cytoplasm in an active form.

### SUMMARY

One aspect of the present disclosure relates to a fusion protein comprising a catalytic domain of a Diphtheria toxin (DT-C), wherein the catalytic domain (DT-C) comprises one or more mutations that inactivate the catalytic domain (DT-C), a translocation domain of a diphtheria toxin (DT-T), wherein the catalytic domain (DT-C) and the translocation domain (DT-T) are linked by a disulfide bond; and a receptor-binding domain (RBD) of a Clostridium neurotoxin protein positioned downstream of the translocation domain (DT-T), wherein the receptor-binding domain (RBD) possesses neuron-specific binding activity, and wherein the fusion protein is capable of delivering a cargo to neural cytoplasm of a cell.

Another aspect of the present disclosure relates to a propeptide fusion comprising a catalytic domain of a diphtheria toxin (DT-C), wherein the catalytic domain (DT-C) comprises one or more mutations that inactivate the catalytic domain (DT-C); a translocation domain of a diphtheria toxin (DT-T), wherein the catalytic domain (DT-C) and the translocation domain (DT-T) are linked by a disulfide bond; a first protease cleavage site between the catalytic domain (DT-C) and the translocation domain (DT-T); and a receptor-binding domain (RBD) of a Clostridium neurotoxin protein positioned downstream of the translocation domain (DT-T), wherein the receptor-binding domain (RBD) possesses neuron-specific binding activity, and wherein the fusion protein is capable of delivering a cargo to neural cytoplasm of a cell.

A further aspect of the present disclosure relates to a fusion protein produced by cleaving the propeptide fusion of the present disclosure at the first protease cleavage site, wherein the catalytic domain (DT-C) and the translocation domain (DT-T) are linked by a disulfide bond.

Another aspect of the present disclosure relates to an isolated nucleic acid molecule encoding the propeptide fusion of the present disclosure.

Further aspects of the present disclosure relate to an expression system comprising the nucleic acid molecule of the present disclosure in a heterologous vector and a host cell comprising the nucleic acid molecule of the present disclosure.

Yet another aspect of the present disclosure relates to a method of expressing a fusion protein. This method involves providing a nucleic acid construct comprising a nucleic acid molecule encoding the propeptide fusion of the present disclosure; a heterologous promoter operably linked to the nucleic acid molecule; and a 3′ regulatory region operably linked to the nucleic acid molecule. The nucleic acid construct is introduced into a host cell under conditions effective to express a propeptide of the fusion protein.

Another aspect of the present disclosure relates to a method of attaching a cargo polypeptide to a fusion protein. This method involves contacting (i) a cargo protein comprising a first member of a peptide fusion tag binding pair and (ii) a DTnd fusion protein as described herein comprising a second member of a peptide fusion tag binding pair with (iii) a biotinylated SnoopLigase to form a complex; capturing the complex on a streptavidin matrix to immobilize the complex; and eluting the cargo protein attached to the fusion protein.

Yet another aspect of the present disclosure relates to a therapeutic agent comprising the fusion protein of the present disclosure and a pharmaceutically acceptable carrier.

A further aspect of the present disclosure relates to a method for treating a subject for toxic effects of a neurotoxin. This method involves administering the therapeutic agent of the present disclosure to the subject under conditions effective to treat the subject for toxic effects of the neurotoxin.

Another aspect of the present disclosure relates to a method of treating a neurological condition. This method involves administering a fusion protein of the present disclosure to a subject under conditions effective to provide treatment to the subject.

Disclosed herein is the development of a novel diphtheria-based intraneural delivery vehicle, DTnd, designed to address the limitations of current botulinum neurotoxin (BoNT) therapeutic delivery systems. Traditional BoNT-based delivery vehicles, such as C1ad, have shown efficacy in delivering therapeutic cargos into neurons but are hindered by intrinsic toxicity and limited capacity to translocate larger or more rigid protein cargos. To overcome these challenges, DTnd has been engineered by inactivating the catalytic domain of diphtheria toxin (DT-C) and substituting its receptor-binding domain with that of a BoNT, thereby enhancing neuronal specificity and safety. This innovative approach aims to provide a more effective and safer method for delivering therapeutic agents into neuronal cytoplasm, potentially revolutionizing the treatment of botulism and other neurological conditions.

The present disclosure overcomes the disadvantages of prior approaches and satisfies the need for an advanced delivery system capable of transporting therapeutic agents directly into neuronal cells to neutralize these toxins effectively. The development of such delivery vehicles, like the diphtheria-based intraneural delivery vehicle (DTnd), aims to overcome these limitations by providing a safe and efficient means of delivering therapeutic agents into the neuronal cytoplasm, thereby enhancing the treatment of BoNT intoxication. Additionally, these delivery systems hold significant potential for treating other neuronal diseases, such as Alzheimer's disease, Parkinson's disease, and prion diseases, by enabling the targeted delivery of therapeutic cargos to specific intracellular targets within neurons, thus opening new avenues for the treatment of a variety of neurological conditions.

The present disclosure also provides methods to efficiently combine a cargo molecule with the DTnd delivery vehicle as described herein.

### BRIEF DESCRIPTION OF THE DRAWINGS

FIG. 1 is a schematic representation of the mechanism/cellular entry of Diphtheria Toxin (DT) and is taken from prior art (see FIG. 1 of Ladokhin “pH-Triggered Conformational Switching along the Membrane Insertion Pathway of the Diphtheria Toxin T-Domain,” Toxins (Basel) 5(8):1362-1380 (2013), which is hereby incorporated by reference in its entirety). FIG. 1 shows the endosomal pathway followed by the Diphtheria Toxin (DT) and the role of its three domains: receptor-binding (R) domain (also called DT-R domain herein), responsible for initiating endocytosis by binding to the heparin-binding EGF (epidermal growth factor)-like receptor; translocation (T)-domain (also called DT-T domain herein); and catalytic (C)-domain (also called DT-C domain herein), blocking protein synthesis via modification of elongation factor 2.

FIGS. 2A-D are schematic representations of various embodiments of proteins and fusion proteins. FIG. 2A is a schematic representation of the Botulinum Toxin (BoNT) depicting its three functional domains: the light chain (LC), translocation domain (TD) and receptor-binding domain (RBD), as well as the disulfide bond between LC and TD in the processed heterodimer. FIG. 2B is a schematic representation of the BoNT/Cl-based therapeutic B8C1ad (ad indicates atoxic derivative), depicting the mutations E₄₂₅>A, H₄₂₈>G and Y₅₇₀>A that inactivate the LC metalloprotease (LC/C1ad, described in Vazquez-Cintron et al., “Engineering Botulinum Neurotoxin C1 as a Molecular Vehicle for Intra-Neuronal Drug Delivery,” Sci Rep 7:42923 (2017), which is hereby incorporated by reference in its entirety), and the single domain antibody B8 cargo, genetically fused to inactivated LC metalloprotease. FIG. 2C is a schematic representation of the Diphtheria Toxin (DT), depicting its three functional domains: the catalytic domain (DT-C or ADP-R), the translocation domain (DT TD or DT-T) and receptor-binding domain (DT RBD or DT-R). The disulfide bond between two cysteine residues in DT-C and DT-T, as well as the furin protease cleavage site between DT-C and DT-T are also depicted. FIG. 2D is a schematic representation of one embodiment of the DT-based therapeutic B8DTnd described herein, depicting enzyme-inactivating mutations K₅₁>E and E₁₄₈>K with green arrows in the DT-C domain (ADP-Rnd where nd indicates non-toxic derivative) (the mutations are numbered according to Kimura et al., “Transgenic Mice Expressing a Fully Nontoxic Diphtheria Toxin Mutant, not CRM197 Mutant, Acquire Immune Tolerance against Diphtheria Toxin,” Journal of Biochemistry 142(1):105-112 (2007), which is hereby incorporated by reference in its entirety), and mutations K₁₂₅>S, Q₁₈₄>S, and R₁₇₃>A in the DT-C domain and, K227>S, Q₂₄₅>S, E₂₉₂>S and K₃₈₅>G with black arrows in the DT-T domain to suppress the immune response developed by human/animal-treated subjects as a consequence of repeated DT administration (Schmohl et al., “Mutagenic Deimmunization of Diphtheria Toxin for Use in Biologic Drug Development,” Toxins (Basel) 7(10):4067-4082 (2015), which is hereby incorporated by reference in its entirety), sdAb B8 (single domain antibody B8) as cargo fused to inactivated DT-C, and BoNT RBD replacing the native DT RBD.

FIG. 3 is a schematic representation of one embodiment of a DTnd intraneuronal delivery vehicle depicting the relative positioning of the functional domains and linkers that connect such domains.

FIG. 4 is a propeptide fusion sequence of one embodiment of an exemplary DTnd delivery vehicle (SEQ ID NO:89).

FIG. 5 is a photograph of a coomassie brilliant blue R250 stained SDS PAGE gel depicting each step of DTnd purification by tandem affinity chromatography. Lane 1: insoluble fraction obtained after Sf9 cell lysis and protein extraction. Lanes 2-8: steps of Ni²⁺-NTA affinity chromatography. Lane 2: loading material, supernatant of Sf9 extract containing DTnd. Lane 3: flow through. Lane 4: first wash with 15 mM imidazole buffer.; Lane 5: second wash with 15 mM imidazole buffer. Lane 6: first wash with 45 mM imidazole buffer. Lane 7: second wash with 45 mM imidazole buffer. Lane 8: eluate obtained with 250 mM imidazole buffer. Lanes 9-13: steps of StrepTactin affinity chromatography of eluate from Lane 8. Lane 9: flow through. Lanes 10 and 11: sequential washes with high (1 M NaCl, pH 8) salt buffer. Lane 12: wash with low salt buffer. Lane 13: eluate obtained with 5 mM D-desthiobiotin.

FIG. 6 is a propeptide fusion sequence of one embodiment of an exemplary therapeutic cargo molecule sdAb B8 (SEQ ID NO:91).

FIGS. 7A-B are photographs of coomassie brilliant blue R250 stained SDS PAGE gels analyzing in FIG. 7A each step of MBP-B8 purification by tandem affinity chromatography, and in FIG. 7B the subsequent size exclusion chromatography (SEC). FIG. 7A shows tandem affinity chromatography. Lane 1: insoluble fraction obtained after E. coli cell lysis and protein extraction. Lanes 2-6: steps of Ni²⁺-penta affinity chromatography. Lane 2: loading material, soluble fraction of E. coli extract containing MBP-B8. Lane 3: flow through. Lane 4: wash with high salt (1M NaCl) buffer. Lane 5: wash with 5 mM imidazole buffer. Lane 6: eluate obtained with 250 mM imidazole buffer. Lanes 7-10: steps of amylose affinity chromatography of the material shown in Lane 6. Lane 7: flow through. Lane 8: wash with high (1 M NaCl, pH 8) salt buffer. Lane 9: wash with low salt buffer. Lane 10: eluate obtained with 25 mM maltose. FIG. 7B shows SEC fractions obtained on HiLoad 26/600 Superdex 200pg column. Lane 1: Loading material (amylose resin eluate). Lanes 2-16: fractions collected from each peak (chromatography profile is not shown). Fractions 8-16 were collected, concentrated, dialyzed, and stored for further use.

FIGS. 8A-C are schematic illustrations of the principle of the SnoopLigase mechanism. The illustration is taken from FIG. 1 of Buldun et al., “SnoopLigase Catalyzes Peptide-Peptide Locking and Enables Solid-Phase Conjugate Isolation,” Journal of the American Chemical Society 140(8):3008-3018 (2018), which is hereby incorporated by reference in its entirety. FIG. 8A is a schematic illustration of domain splitting. The C-terminal domain of RrgA (Protein Data Bank 2WW8) was split into three parts and engineered such that the reactive Lys is located on SnoopTagJr (turquoise), the reactive Asn on DogTag (yellow), and the catalytic Glu on SnoopLigase (blue) (key residues highlighted in red). FIG. 8B shows the molecular basis for isopeptide bond formation in RrgA. Glu₈₀₃ promotes isopeptide bond formation between Lys₇₄₂ and Asn₈₅₄, eliminating ammonia. FIG. 8C shows a schematic of the use of SnoopLigase to direct peptide-peptide ligation (the isopeptide bond is represented in red).

FIG. 9 is an illustration of amino acid sequences of SnoopLigase and protein fusion tags. FIG. 9 shows amino acid sequences of precursor RrgA C-terminal domain and engineered partners. A sequence alignment of the C-terminal domain of RrgA and proteins/peptides derived from this domain is shown. Amino acid mutations shown in green contributed to higher activity/solubility/expression level of the proteins/peptide fusion tags. Amino acids in red constitute the reactive Lys residue in precursor and SnoopTagJr, the reactive Asn residue in precursor and DogTag, and the catalytic Glu residue in precursor and SnoopLigase.

FIG. 10 is a fusion sequence of Halotag7SnoopLigase with Avi tag (HalobtnSNL; SEQ ID NO:111).

FIG. 11 is a photograph showing biotinylated Halotag7-SnoopLigase (HalobtnSNL) expression and purification. A coomassie brilliant blue R250-stained SDS PAGE gel analyzing each step of biotinylated Halotag7-SnoopLigase purification by tandem affinity chromatography is shown. Lane 1: insoluble fraction obtained after E. coli cell lysis and protein extraction. Lanes 2-6: steps of Ni2+-penta affinity chromatography. Lane 2: loading material, soluble fraction of E. coli extract containing biotinylated Halotag7-SnoopLigase. Lane 3: flow through. Lane 4: wash with high salt (1M NaCl) buffer. Lane 5: wash with 5 mM imidazole buffer. Lane 6: eluate obtained with 250 mM imidazole buffer. Lanes 7-10: steps of StrepTactin chromatography of the eluate shown in Lane 6. Lane 7: flow through. Lane 8: wash with high (1 M NaCl, pH 8) salt buffer. Lane 9: wash with low salt buffer. Lane 10: eluate obtained with 5 mM D-desthiobiotin and 50 mM biotin.

FIGS. 12A-B are photographs of B8DTnd in vitro synthesis and purification. FIG. 12A is a coomassie-stained SDS PAGE gel showing biotinylated HaloTag-SnoopLigase-mediated isopeptide conjugation of the components “A” and “B”, removal of the enzyme from the reaction mixture and purification of the fusion protein. The reaction of isopeptide conjugation were assembled with all components and enzyme present in the aqueous phase. Lane 1: reaction mixture at the time 0. Lane 2: reaction mixture after 24 h incubation at 4° C. Lane 3: reaction mixture after addition of TEV protease at time 0 to the protein mixture shown in Lane 2. Lane 4: mixture after incubation of the reaction with TEV protease after 6h incubation at 28° C. Lane 5: supernatant after capturing biotinylated SnoopLigase in complex with the fusion protein by streptavidin matrix. Lane 6: streptavidin matrix wash with high salt (1M NaCl, 0.1% Tween, pH 8) buffer. Lane 7: streptavidin matrix wash with high salt (1M NaCl, pH 8) buffer. Lane 8: streptavidin matrix wash with 50 mM glycine-HCl, pH 3. Lanes 9-10: eluate from streptavidin matrix obtained with 50 mM glycine-HCl buffer, pH 2. FIG. 12B is a coomassie-stained SDS PAGE gel depicting different protein species involved in synthesis, processing, and purification of B8DTnd through SnoopLigase-mediated isopeptide fusion reaction. In the SnoopLigase-mediated reaction: HalobtnSNL (53 kDa), MBP-B8 (66 kDa), DTnd (100 kDa), MBP-B8DTnd (166 kDa). In the TEV-mediated cleavage reaction: B8DTnd (1l6 kDa), MBP-TEV (70 kDa), MBP (50 kDa), Halotag7 (33 kDa), btnSNL (17 kDa).

FIG. 13 is a propeptide fusion sequence of one embodiment of an exemplary therapeutic cargo molecule C1 (MBP-JSGC1; SEQ ID NO:93).

FIGS. 14A-C are schematic illustrations of exemplary fusion proteins. FIG. 14A illustrates an example of one embodiment of DTnd depicting its three functional domains: the inactivated catalytic domain (DT-C, or ADP-Rnd), translocation domain (TD), and receptor-binding domain (RBD), the disulfide bond between two cysteine residues in DT-C and DT-TD, the furin cleavage site between DT-C and DT-TD, the enzyme-inactivating mutations K₅₁>E and E₁₄₈>K in light arrows, the mutations K₁₂₅>S, R₁₇₃>A, Q₂₄₅>S, K₃₈₅>G, E₂₉₂>S, Q₁₈₄>S and K227>S to suppress the immune response in black arrows, and SnoopTagJr at the N-terminus for SnoopLigase-mediated isopeptide bond conjugation. FIG. 14B illustrates one embodiment of B8DTnd, depicting therapeutic cargo sdAb B8 as cargo fused to DTnd via an isopeptide bond between DogTag and SnoopTagJr. FIG. 14C illustrates one embodiment of C1DTnd, depicting therapeutic cargo sdAb JSG-C1 (JC1) as cargo fused to DTnd via an isopeptide bond between DogTag and SnoopTagJr.

FIGS. 15A-C are graphs of ligated B8DTnd efficacy studies. Shown are in vivo studies in mice evaluating the efficacy of enzymatically fused in vitro B8DTnd in preventing death and clinical toxicity after treatment with different doses 2 h after 2 LD₅₀ BoNT/A1 intoxication challenge. FIG. 15A is a graph of the Clinical Severity Score (CSS) over time with different dose treatment groups. FIG. 15B is a graph of survival statistics over time with different dose treatment groups. FIG. 15C is a graph of the 10-day survival rate of different dose treatment groups. VH: vehicle used as control (DTnd).

FIGS. 16A-C are graphs of ligated C1DTnd efficacy studies. Shown are in vivo studies in mice evaluating the efficacy of enzymatically fused in vitro C1DTnd in preventing death and clinical toxicity after treatment with different doses 2 h after 2 LD₅₀ BoNT/B1 intoxication challenge. FIG. 16A is a graph of the Clinical Severity Score (CSS) over time with different dose treatment groups. FIG. 16B is a graph of the survival statistics over time with different dose treatment groups. FIG. 16C is a graph of the 10-day survival rate of different dose treatment groups. VH: vehicle used as control (DTnd).

FIG. 17 is a photograph of furin-mediated cleavage of DTnd in vitro. Shown is a coomassie-stained SDS PAGE gel showing products of the cleavage reaction obtained after DTnd incubation with furin under different w/w DTnd:furin ratios in vitro. Lane 1: DTnd before cleavage under reducing conditions. Lanes 2-6: samples after 1 h incubation at 25° C. with DTnd:furin w/w ratios 234, 119, 80, 60, and 48 respectively, under reducing conditions. Lanes 7-11: samples after 3 h incubation at 25° C. with DTnd: furin w/w ratios 234, 119, 80, 60, and 48 respectively, under reducing conditions. Lanes 12-16: samples shown in Lanes 7-11 respectively under non-reducing conditions.

FIGS. 18A-F are schematic illustrations and graphs comparing a BoNT delivery vehicle with a DT delivery vehicle. FIG. 18A is a schematic illustration of a BoNT delivery vehicle with anti-BoNT/A antibody B8 cargo at the amino terminus. The LC/C1ad domain has an E₄₂₅>A, an H₄₂₈>G, and a Y₅₇₀>A mutation. A disulfide bond connects the light chain (LC) and the heavy chain (HC). The heavy chain includes the BoNT translocation domain and the BoNT receptor binding domain. Each of the B8LC, the HC/C1, the C1ad, and the B8C1ad vehicles are shown. FIG. 18B is a graph showing the median Clinical Severity Score (CSS) over time with different dose treatment groups 0-10 days after BoNT/A1 intoxication. FIG. 18C is a graph of the survival statistics over time of treatment with the maximum tolerated dose (MTD) after 2LD₅o BoNT/A1 challenge. FIG. 18D a schematic illustration of a DT delivery vehicle with anti-BoNT/A antibody B8 cargo at the amino terminus. The DT-C domain (ADP-Rnd) includes enzyme-inactivating mutations K₅₁>E and E₁₄₈>K and mutations K₁₂₅>S, R₁₇₃>A, and Q₁₈₄>S in the DT-C domain and, K₂₂₇>S, Q₂₄₅>S, E₂₉₂>S, and K₃₈₅>G in the DT-TD (DT-T domain), and BoNT RBD replacing the native DT RBD. A disulfide bond connects the DT-C(ADP-Rnd) domains and the DT-T domain. Each of the DTnd and B8DTnd delivery vehicles are shown. FIG. 18E is a graph showing toxic signs over time with different dose treatment groups 0-10 days after BoNT/A1 intoxication. FIG. 18F is a graph of the survival probability over time of treatment with different doses of B8DTnd after 2LD₅o BoNT/A1 challenge.

FIG. 19 is an illustration of the sequence of one embodiment of an engineered scFv of an anti-phosphorylated tau antibody (SEQ ID NO:98) that can be used as a cargo in the DTnd delivery vehicle.

FIG. 20 is an illustration of the sequence of one embodiment of an anti “beta sheet” scFv propeptide fusion (SEQ ID NO:97) that can be used as a cargo in the DTnd delivery vehicle.

FIG. 21 is a schematic illustration showing routes of administration for botulinum toxin and its use for targeting peripheral or central neural systems and is taken from prior art. The figure was produced by using free images taken from Servier Medical Art (smart.servier, accessed on 8 Sep. 2021), a service to medicine provided by Les Laboratoires Servier (www.servier, accessed on 8 Sep. 2021).

### DETAILED DESCRIPTION

The present disclosure is directed to fusion proteins and propeptide fusions capable of carrying therapeutic cargo to intracellular targets. In some embodiments, the fusion proteins and propeptide fusions include a diphtheria catalytic domain (DT-C) with inactivating mutations, a diphtheria translocation domain (DT-T), and a Clostridium neurotoxin receptor binding domain. In some embodiments, the fusion proteins and propeptide fusions include a therapeutic cargo.

Unless otherwise indicated, the definitions and embodiments described in this and other sections are intended to be applicable to all embodiments and aspects of the present disclosure herein described for which they are suitable as would be understood by a person of ordinary skill in the art.

Singular forms “a”, “an”, and “the” include plural references unless the context clearly dictates otherwise. Thus, for example, a reference to “a method” includes one or more methods, and/or steps of the type described herein and/or which will become apparent to a person of ordinary skill in the art upon reading this disclosure. In another example, reference to “a cell” includes both a single cell and a plurality of cells.

The term “about” includes being within a statistically meaningful range of a value. Such a range can be within an order of magnitude, such as within 10% or within 5% of a given value or range.

The term “and/or” as used herein means that the listed items are present, or used, individually or in combination. In effect, this term means that “at least one of” or “one or more” of the listed items is used or present.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers, and/or steps. The foregoing also applies to words having similar meanings such as the terms, “including”, “involving”, “having”, and their derivatives.

The terms “nucleic acid”, “nucleotide”, or “polynucleotide” sequence are used interchangeably, and refer to a polymeric compound comprised of covalently linked subunits called nucleotides. Nucleic acids include polyribonucleic acid (“RNA”) and polydeoxyribonucleic acid (“DNA”), both of which may be single-stranded or double-stranded. DNA includes, but is not limited to, cDNA, genomic DNA, plasmid DNA, synthetic DNA, and semi-synthetic DNA. DNA may be linear, circular, or supercoiled.

A “reference sequence” means a nucleic acid or amino acid used as a comparator for another nucleic acid or amino acid, respectively, when determining sequence identity. A reference sequence can be a wild-type sequence.

“Sequence identity,” “percent identity,” or “% identical” refers to the exactness of a match between a reference sequence and a sequence being compared to it when optimally aligned. For example, sequence alignments and percent identity calculations may be determined using a variety of comparison methods designed to detect homologous sequences including, but not limited to, the Multalin program (Corpet, “Multiple Sequence Alignment with Hierarchical Clustering,” Nucleic Acids Res. 16:10881-90 (1988), which is hereby incorporated by reference in its entirety) or the Megalign® program of the LASERGENE® bioinformatics computing suite (DNASTAR® Inc., Madison, Wis.). Sequences may also be aligned using algorithms known in the art including, but not limited to, CLUSTAL V algorithm or the BLASTN or BLAST 2 sequence programs.

### Diphtheria Toxin Domains and Botulinum Toxin Receptor Domains

One aspect of the present disclosure relates to a fusion protein comprising a catalytic domain of a diphtheria toxin (DT-C), wherein the catalytic domain (DT-C) comprises one or more mutations that inactivate the catalytic domain (DT-C), a translocation domain of a diphtheria toxin (DT-T), wherein the catalytic domain (DT-C) and the translocation domain (DT-T) are linked by a disulfide bond; and a receptor-binding domain (RBD) of a Clostridium neurotoxin protein positioned downstream of the translocation domain (DT-T), wherein the receptor-binding domain (RBD) possesses neuron-specific binding activity, and wherein the fusion protein is capable of delivering a cargo to neural cytoplasm of a cell.

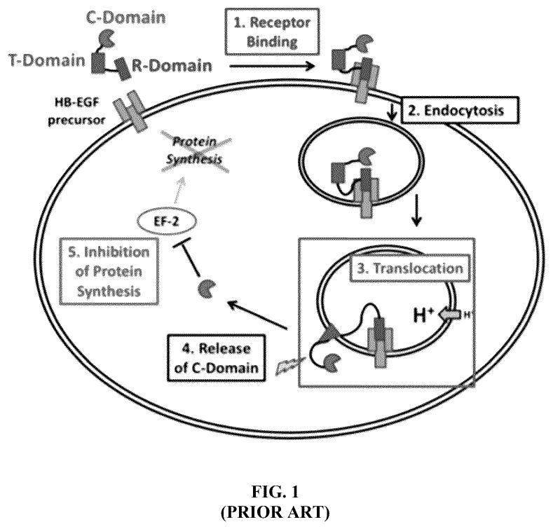

Diphtheria toxin (DT) is the main virulence factor of Corynebacterium diphtheriae. DT is a potent bacterial protein toxin composed of three functional domains: receptor-binding (DT-R), translocation (DT-T), and catalytic (DT-C). It exerts its pathogenic effects by delivering a lethal cargo to cells that express heparin-binding epidermal growth factor (HB-EGF) on their surface. Upon binding to the HB-EGF receptor via the DT-R domain, DT is internalized through endocytosis (see FIG. 1 and Ladokhin A., “pH-Triggered Conformational Switching along the Membrane Insertion Pathway of the Diphtheria Toxin T-Domain,” Toxins (Basel) 5(8):1362-1380 (2013), which is hereby incorporated by reference in its entirety). The acidic environment within the endosome triggers a conformational change in the DT-T domain, facilitating the translocation of the DT-C domain into the cytosol. Once inside the cytosol, the C domain is proteolytically cleaved and subsequently inhibits protein synthesis by ADP-ribosylating its intracellular target, elongation factor 2 (EF2), leading to the termination of protein synthesis cell death.

One embodiment of an exemplary wild-type diphtheria toxin propeptide sequence comprising the DT-C, DT-T, and DT-R domains is UniProt Accession No. P00588 DTX_CORBE, which is hereby incorporated by reference in its entirety. Wild type UniProt Accession No. P00588 diphtheria toxin from Corynebacterium diphtheriae has an amino acid sequence as set forth below (SEQ ID NO:1):

MLVRGYVVSRKLFASILIGALLGIGAPPSAHAGADDVVDSSKSFVMENF
SSYHGTKPGYVDSIQKGIQKPKSGTQGNYDDDWKGFYSTDNKYDAAGYS
VDNENPLSGKAGGVVKVTYPGLTKVLALKVDNAETIKKELGLSLTEPLM
EQVGTEEFIKRFGDGASRVVLSLPFAEGSSSVEYINNWEQAKALSVELE
INFETRGKRGQDAMYEYMAQACAGNRVRRSVGSSLSCINLDWDVIRDKT
KTKIESLKEHGPIKNKMSESPNKTVSEEKAKQYLEEFHQTALEHPELSE
LKTVTGTNPVFAGANYAAWAVNVAQVIDSETADNLEKTTAALSILPGIG
SVMGIADGAVHHNTEEIVAQSIALSSLMVAQAIPLVGELVDIGFAAYNF
VESIINLFQVVHNSYNRPAYSPGHKTQPFLHDGYAVSWNTVEDSIIRTG
FQGESGHDIKITAENTPLPIAGVLLPTIPGKLDVNKSKTHISVNGRKIR
MRCRAIDGDVTFCRPKSPVYVGNGVHANLHVAFHRSSSEKIHSNEISSD
SIGVLGYQKTVDHTKVNSKLSLFFEIKS

The propeptide sequence of DT includes a signal peptide of 32 amino acids at the N-terminus (indicated in bold text) that is removed upon secretion of the DT across the bacterial plasma membrane.

An exemplary embodiment of diphtheria toxin without the signal peptide is set forth as SEQ ID NO:2 below:

GADDVVDSSKSFVMENFSSYHGTKPGYVDSIQKGIQKPKSGTQGNYDDD
WKGFYSTDNKYDAAGYSVDNENPLSGKAGGVVKVTYPGLTKVLALKVDN
AETIKKELGLSLTEPLMEQVGTEEFIKRFGDGASRVVLSLPFAEGSSSV
EYINNWEQAKALSVELEINFETRGKRGQDAMYEYMAQACAGNRVRRSVG
SSLSCINLDWDVIRDKTKTKIESLKEHGPIKNKMSESPNKTVSEEKAKQ
YLEEFHQTALEHPELSELKTVTGTNPVFAGANYAAWAVNVAQVIDSETA
DNLEKTTAALSILPGIGSVMGIADGAVHHNTEEIVAQSIALSSLMVAQA
IPLVGELVDIGFAAYNFVESIINLFQVVHNSYNRPAYSPGHKTQPFLHD
GYAVSWNTVEDSIIRTGFQGESGHDIKITAENTPLPIAGVLLPTIPGKL
DVNKSKTHISVNGRKIRMRCRAIDGDVTFCRPKSPVYVGNGVHANLHVA
FHRSSSEKIHSNEISSDSIGVLGYQKTVDHTKVNSKLSLFFEIKS

The mature DT toxin has a disulfide between the DT-C domain and the DT-T domain. The cysteine residues that form the disulfide bond are highlighted in bold text in SEQ ID NO:2 (Cys₁₈₆ and Cys₂₀₁). Diphtheria toxin also includes a furin protease cleavage site (RVRR; SEQ ID NO:86; shown in bold italics in SEQ ID NO:2 above) located between the DT-C and DT-T domains. The disulfide bonds are broken after acidification of the endosome, and the DT-C domain is translocated into the cytoplasm through the DT-T domain, which forms a pore. The furin cleavage site in diphtheria toxin is cleaved after the toxin is internalized by a cell, specifically within the endosomal compartment.

The catalytic domain (DT-C) of wild type diphtheria toxin (SEQ ID NO:3) is set forth as shown below:

GADDVVDSSKSFVMENFSSYHGTKPGYVDSIQKGIQKPKSGTQGNYDDD
WKGFYSTDNKYDAAGYSVDNENPLSGKAGGVVKVTYPGLTKVLALKVDN
AETIKKELGLSLTEPLMEQVGTEEFIKRFGDGASRVVLSLPFAEGSSSV
EYINNWEQAKALSVELEINFETRGKRGQDAMYEYMAQACAGN

In some embodiments, the DT-C domain comprises inactivating mutations K₅₁>E and E₁₄₈>K. Inactivating mutations may be introduced into the DT-C domain to produce DT-Cnd at positions K₅₁>E and E₁₄₈>K numbered according to SEQ ID NO:2. In these and other embodiments, the mutations are represented by nomenclature which identifies the wild-type amino acid residue at a specific position (e.g., K₅₁) followed by the symbol “>” indicating a change from that amino acid to the amino acid immediately following the “>” symbol (e.g., >E). For example, the “K₅₁>E” nomenclature indicates a change from a lysine (K) residue at position 51 to a glutamic acid residue (E).

The DT-Cnd sequence with inactivating mutations at positions K₅₁>E and E₁₄₈>K is set forth as SEQ ID NO:4 below. The K₅₁>E and E₁₄₈>K mutations are highlighted in bold text in the following sequence (SEQ ID NO:4):

GADDVVDSSKSFVMENFSSYHGTKPGYVDSIQKGIQKPKSGTQGNYDDD
WEGFYSTDNKYDAAGYSVDNENPLSGKAGGVVKVTYPGLTKVLALKVDN
AETIKKELGLSLTEPLMEQVGTEEFIKRFGDGASRVVLSLPFAEGSSSV
KYINNWEQAKALSVELEINFETRGKRGQDAMYEYMAQACAGN

The terms “non-toxic derivative” or “nd” are used interchangeably herein and are used identify a protein or propeptide fusion that has a modified version of the diphtheria toxin catalytic domain that is devoid of some, most, or all ADP ribosylation catalytic activity. In some embodiments, the catalytic domain (DT-C) is devoid of all ADP ribosylation activity. ADP ribosylation activity may be measured by any assay known to those of skill in the art. For example, the ADP ribosylation activity of the DT-C can be measured indirectly by assessing its downstream effects, such as cytotoxicity in cells, using any appropriate in vitro or in vivo assay (see e.g., Kimura et al., “Transgenic Mice Expressing a Fully Nontoxic Diphtheria Toxin Mutant, not CRM197 Mutant, Acquire Immune Tolerance against Diphtheria Toxin,” J. Biochemistry 142(1)105-112 (2007), which is hereby incorporated by reference in its entirety). In some embodiments, the DT-C domain is truncated or present in a minimal sequence that allows its connection to the translocation domain (DT-T) via disulfide bridge. In some embodiments, the DT-C and DT-T domains are separated by a cleavable linker.

Additional mutations at K₁₂₅>S, R₁₇₃>A, and Q₁₈₄>S were introduced into the catalytic domain of DT-C along with the K₅₁>E and E₁₄₈>K inactivating mutations to produce SEQ ID NO:5 as follows:

GADDVVDSSKSFVMENFSSYHGTKPGYVDSIQKGIQKPKSGTQGN
YDDDWEGFYSTDNKYDAAGYSVDNENPLSGKAGGVVKVTYPGLTK
VLALKVDNAETIKKELGLSLTEPLMEQVGTEEFISRFGDGASRVV
LSLPFAEGSSSVKYINNWEQAKALSVELEINFETRGKAGQDAMYE
YMASACAGN

The positions of various K₅₁>E, K₁₂₅>S, E₁₄₈>K, R₁₇₃>A, and Q₁₈₄>S mutations are indicated in bold text in SEQ ID NO:5 above.

In some embodiments, the DT-C domain comprises an amino acid sequence that has at least 80%, 83%, 85%, 90%, 93%, 95%, 96%, 97%, 98%, 99%, or 100% sequence identity (or any number or range therein) to the amino acid sequence of DT-C of SEQ ID NO:3. In some embodiments, the DT-C domain comprises an amino acid sequence that has at least 80%, 83%, 85%, 90%, 93%, 95%, 96%, 97%, 98%, 99%, or 100% sequence identity (or any number or range therein) to the amino acid sequence of DT-C of SEQ ID NO:4. In some embodiments, the DT-C domain comprises an amino acid sequence that has at least 80%, 83%, 85%, 90%, 93%, 95%, 96%, 97%, 98%, 99%, or 100% sequence identity (or any number or range therein) to the amino acid sequence of DT-C of SEQ ID NO:5. In some embodiments, the catalytic domain (DT-C) of the fusion protein or the propeptide fusion of the disclosure comprises the amino acid sequence of SEQ ID NO:4 or a sequence having at least 95% sequence identity to SEQ ID NO:4. In some embodiments, the catalytic domain (DT-C) of the fusion protein or the propeptide fusion of the disclosure comprises the amino acid sequence of SEQ ID NO:5 or a sequence having at least 95% sequence identity to SEQ ID NO:5.

The sequence of the wild-type diphtheria toxin translocation domain (DT-T), which is responsible for the translocation of the DT-C domain to the cytosol is set forth as SEQ ID NO:6, as follows:

SVGSSLSCINLDWDVIRDKTKTKIESLKEHGPISNKMSESPNKTV
SEEKAKSYLEEFHQTALEHPELSELKTVTGTNPVFAGANYAAWAV
NVAQVIDSSTADNLEKTTAALSILPGIGSVMGIADGAVHHNTEEI
VAQSIALSSLMVAQAIPLVGELVDIGFAAYNFVESIINLFQVVHN
SYNR

In some embodiments, mutations are introduced into the fusion protein or propeptide fusion to suppress the immune response of the human/animal treatment subject caused by repeated use of the native DT sequence. In some embodiments, the catalytic domain (DT-C) and/or the translocation domain (DT-T) further comprise one or more mutations that suppress an immune response of a human subject administered the fusion protein. In some embodiments, an immune response to suppress the immune response of the human/animal treatment subject is caused by repeated use of the native DT sequence. In some embodiments, the one or more mutations that suppress an immune response of a human subject administered the fusion protein comprise one or more of K₁₂₅>S, R₁₇₃>A and Q₁₈₄>S of SEQ ID NO:5 and Q₂₄₅>S, E₂₉₂>S, and K₂₂₇>S in the DT-T domain of SEQ ID NO:7 or SEQ ID NO:8 and K₃₈₅>G in the linker sequence of SEQ ID NO:8. Additional mutations K₁₂₅>S, R₁₇₃>A, Q₁₈₄>S Q₂₄₅>S, E₂₉₂>S, K₂₂₇>S, and K₃₈₅>G (numbered according to SEQ ID NO:2) were introduced to suppress an immune response of a human subject administered the fusion protein. The genetic constructs, expression systems, and processing methods described herein are shown to produce a family of recombinant DT derivatives, with conformational and trafficking properties similar to the wild type DT toxins. The DT toxins described herein provide an increased safety margin while at the same time providing a decreased risk of immunogenic response as compared to other non-toxic derivatives.

The sequence of an exemplary DT-T domain comprising additional mutations to suppress an immune response is set forth as SEQ ID NO:7, as follows:

SVGSSLSCINLDWDVIRDKTKTKIESLKEHGPISNKMSESPNKTVSE
EKAKSYLEEFHQTALEHPELSELKTVTGTNPVFAGANYAAWAVNVAQ
VIDSSTADNLEKTTAALSILPGIGSVMGIADGAVHHNTEEIVAQSIA
LSSLMVAQAIPLVGELVDIGFAAYNFVESIINLFQVVHNSYNR

The locations of the various mutations are indicated in bold text in SEQ ID NO:7. In some embodiments, the one or more mutations that suppress an immune response of a human subject administered the fusion protein comprise one or more of Q₂₄₅>S, E₂₉₂>S, and K₂₂₇>S of SEQ ID NO:7.

The sequence of an exemplary DT-T domain comprising additional mutations to suppress an immune response including an additional linker sequence is set forth as SEQ ID NO:8, as follows:

SVGSSLSCINLDWDVIRDKTKTKIESLKEHGPISNKMSESPNKTV
SEEKAKSYLEEFHQTALEHPELSELKTVTGTNPVFAGANYAAWAV
NVAQVIDSSTADNLEKTTAALSILPGIGSVMGIADGAVHHNTEEI
VAQSIALSSLMVAQAIPLVGELVDIGFAAYNFVESIINLFQVVHN
SYNRPAYSPGHGTQPFL

The locations of the various mutations are indicated in bold text in SEQ ID NO:8. In some embodiments, the one or more mutations that suppress an immune response of a human subject administered the fusion protein comprise one or more of Q₂₄₅>S, K₃₈₅>G, E₂₉₂>S, and K₂₂₇>S of SEQ ID NO:8.

In some embodiments, the DT-T domain comprises an amino acid sequence that has at least 80%, 83%, 85%, 90%, 93%, 95%, 96%, 97%, 98%, 99%, or 100% sequence identity (or any number or range therein) to the amino acid sequence of DT-T of SEQ ID NO:6. In some embodiments, the DT-T domain comprises an amino acid sequence that has at least 80%, 83%, 85%, 90%, 93%, 95%, 96%, 97%, 98%, 99%, or 100% sequence identity (or any number or range therein) to the amino acid sequence of DT-T of SEQ ID NO:7. In some embodiments, the DT-T domain comprises an amino acid sequence that has at least 80%, 83%, 85%, 90%, 93%, 95%, 96%, 97%, 98%, 99%, or 100% sequence identity (or any number or range therein) to the amino acid sequence of DT-T of SEQ ID NO:8. In some embodiments, the translocation domain (DT-T) of the fusion protein or the propeptide fusion of the disclosure comprises the amino acid sequence of SEQ ID NO:6 or a sequence having at least 95% sequence identity to SEQ ID NO:6. In some embodiments, the translocation domain (DT-T) of the fusion protein or the propeptide fusion of the disclosure comprises the amino acid sequence of SEQ ID NO:7 or a sequence having at least 95% sequence identity to SEQ ID NO:7. In some embodiments, the translocation domain (DT-T) of the fusion protein or the propeptide fusion of the disclosure comprises the amino acid sequence of SEQ ID NO:8 or a sequence having at least 95% sequence identity to SEQ ID NO:8.

In some embodiments, the fusion protein or propeptide fusion comprises a Receptor-Binding Domain (RBD) of a Clostridium neurotoxin. The Clostridium neurotoxins are a family of structurally similar proteins that target the neuronal machinery for synaptic vesicle exocytosis. Produced by anaerobic bacteria of the Clostridium genus, botulinum neurotoxins and tetanus neurotoxins are the most poisonous substances known on a per-weight basis, with an LD₅₀ in the range of 0.5-2.5 ng/kg when administered by intravenous or intramuscular routes (National Institute of Occupational Safety and Healthy, “Registry of Toxic Effects of Chemical Substances (R-TECS),” Cincinnati, Ohio: National Institute of Occupational Safety and Health (1996), which is hereby incorporated by reference in its entirety). In some embodiments, the Clostridium species is a Clostridium botulinum species, a Clostridium butyricum species, a Clostridium baratii species, a Clostridium argentinense species, or a Clostridium tetani species.

Common structural features of the wild-type Clostridium botulinum neurotoxins are illustrated in U.S. Pat. No. 7,785,606 to Ichtchenko and Band, which is hereby incorporated by reference in its entirety. These structural features are illustrated using BoNT/A as an example but are generalized among all BoNT serotypes.

Botulinum neurotoxins are synthesized as single chain propeptides which are later activated by a specific proteolysis cleavage event, generating a dimer joined by a disulfide bond. The mature BoNT/A is composed of three functional domains of Mr ˜50,000, where the catalytic function responsible for toxicity is confined to the light chain (residues 1-437), the translocation activity is associated with the N-terminal half of the heavy chain (residues 448-872), and cell binding is associated with its C-terminal half (residues 873-1,295) (Johnson, “Clostridial Toxins as Therapeutic Agents: Benefits of Nature's Most Toxic Proteins,” Annu. Rev. Microbiol. 53:551-575 (1999); Montecucco et al., “Structure and Function of Tetanus and Botulinum Neurotoxins,” Q. Rev. Biophys. 28:423-472 (1995), which are hereby incorporated by reference in their entirety).

The Botulinum Neurotoxin A1 (BoNT/A1) receptor binding domain (RBD) specifically binds two receptors on the neuronal surface: a high affinity protein receptor, Synaptic Vesicle glycoprotein 2 (SV2), and low affinity lipid receptor, ganglioside (sialic acid containing glycosphingolipid). These receptors are the main entities responsible to conferring BoNT/A1 its superior neuronal cell target specificity.

In some embodiments, the Clostridium botulinum neurotoxin comprises serotype A1 (BoNT/A1). An exemplary wild-type BoNT/A1 propeptide sequence comprising the BoNT/A1-C, BoNT/A1-T and BoNT/A1-R domains is GenBank Accession No. CAL82360.1, which is hereby incorporated by reference in its entirety. Wild type BoNT/A1 has an amino acid sequence as set forth below (SEQ ID NO:9):

MPFVNKQFNYKDPVNGVDIAYIKIPNAGQMQPVKAFKIHNKIWVI
PERDTFTNPEEGDLNPPPEAKQVPVSYYDSTYLSTDNEKDNYLKG
VTKLFERIYSTDLGRMLLTSIVRGIPFWGGSTIDTELKVIDTNCI
NVIQPDGSYRSEELNLVIIGPSADIIQFECKSFGHEVLNLTRNGY
GSTQYIRFSPDFTFGFEESLEVDTNPLLGAGKFATDPAVTLAHEL
IHAGHRLYGIAINPNRVFKVNTNAYYEMSGLEVSFEELRTFGGHD
AKFIDSLQENEFRLYYYNKFKDIASTLNKAKSIVGTTASLQYMKN
VFKEKYLLSEDTSGKFSVDKLKFDKLYKMLTEIYTEDNFVKFFKV
LNRKTYLNFDKAVFKINIVPKVNYTIYDGFNLRNTNLAANFNGQN
TEINNMNFTKLKNFTGLFEFYKLLCVRGIITSKTKSLDKGYNKAL
NDLCIKVNNWDLFFSPSEDNFTNDLNKGEEITSDTNIEAAEENIS
LDLIQQYYLTFNFDNEPENISIENLSSDIIGQLELMPNIERFPNG
KKYELDKYTMFHYLRAQEFEHGKSRIALTNSVNEALLNPSRVYTF
FSSDYVKKVNKATEAAMFLGWVEQLVYDFTDETSEVSTTDKIADI
TIIIPYIGPALNIGNMLYKDDFVGALIFSGAVILLEFIPEIAIPV
LGTFALVSYIANKVLTVQTIDNALSKRNEKWDEVYKYIVTNWLAK
VNTQIDLIRKKMKEALENQAEATKAIINYQYNQYTEEEKNNINFN
IDDLSSKLNESINKAMININKFLNQCSVSYLMNSMIPYGVKRLED
FDASLKDALLKYIYDNRGTLIGQVDRLKDKVNNTLSTDIPFQLSK
YVDNQRLLSTFTEYIKNIINTSILNLRYESNHLIDLSRYASKINI
GSKVNFDPIDKNQIQLENLESSKIEVILKNAIVYNSMYENFSTSF
WIRIPKYENSISLNNEYTIINCMENNSGWKVSLNYGEIIWTLQDT
QEIKQRVVFKYSQMINISDYINRWIFVTITNNRLNNSKIYINGRL
IDQKPISNLGNIHASNNIMFKLDGCRDTHRYIWIKYFNLFDKELN
EKEIKDLYDNQSNSGILKDFWGDYLQYDKPYYMLNLYDPNKYVDV
NNVGIRGYMYLKGPRGSVMTTNIYLNSSLYRGTKFIIKKYASGNK
DNIVRNNDRVYINVVVKNKEYRLATNASQAGVEKILSALEIPDVG
NLSQVVVMKSKNDQGITNKCKMNLQDNNGNDIGFIGFHQFNNIAK
LVASNWYNRQIERSSRTLGCSWEFIPVDDGWGERPL

In some embodiments, the fusion protein or propeptide comprises a BoNT/A1 receptor binding domain (RBD) as set forth as SEQ ID NO: 10 below:

NIINTSILNLRYESNHLIDLSRYASKINIGSKVNFDPIDKNQIQL
FNLESSKIEVILKNAIVYNSMYENFSTSFWIRIPKYENSISLNNE
YTIINCMENNSGWKVSLNYGEIIWTLQDTQEIKQRVVFKYSQMIN
ISDYINRWIFVTITNNRLNNSKIYINGRLIDQKPISNLGNIHASN
NIMFKLDGCRDTHRYIWIKYFNLEDKELNEKEIKDLYDNQSNSGI
LKDFWGDYLQYDKPYYMLNLYDPNKYVDVNNVGIRGYMYLKGPRG
SVMTTNIYLNSSLYRGTKFIIKKYASGNKDNIVRNNDRVYINVVV
KNKEYRLATNASQAGVEKILSALEIPDVGNLSQVVVMKSKNDQGI
TNKCKMNLQDNNGNDIGFIGFHQFNNIAKLVASNWYNRQIERSSR
TLGCSWEFIPVDDGWGERPL

In some embodiments, the RBD domain comprises an amino acid sequence that has at least 80%, 83%, 85%, 90%, 93%, 95%, 96%, 97%, 98%, 99%, or 100% sequence identity (or any number or range therein) to the amino acid sequence of SEQ ID NO:10. In some embodiments, the receptor-binding domain (RBD) domain derived from the Clostridium botulinum neurotoxin protein A1 (BoNT/A1) specifically binds to Synaptic Vesicle glycoprotein 2 (SV2) and ganglioside receptors on a neuronal surface.

Receptor binding domains from other BoNT serotypes are suitable for use in the present disclosure. Exemplary sequences for various BoNT serotypes are provided in Table 1 infra. BoNT serotype sequences, the receptors that the various serotypes interact with, and the enzymatic targets and cleavage sites of the various serotypes are described in Peck et al., “Historical Perspectives and Guidelines for Botulinum Neurotoxin Subtype Nomenclature,” Toxins 9(1):38 (2017), which is hereby incorporated by reference in its entirety. BoNTs have a “double-receptor” binding mode: a high affinity protein receptor (e.g., SV2A, SV2B, SV2C, Syt-I and Syt-II), and low affinity lipid receptor comprising gangliosides (e.g. GT1b and GD1a, GD1b). Different serotypes show different affinities to a combination of these receptors. See also Dong et al., “Botulinum and Tetanus Neurotoxins.” Annual Review Biochemistry 88:811-837 (2018) and Chen et al., “Emerging Opportunities for Serotypes of Botulinum Neurotoxins,” Toxins 4(11):1196-1222 (2012) (e.g., Table 2), each of which is hereby incorporated by reference in its entirety.

In some embodiments, the receptor binding domain (RBD) is an RBD of a Clostridium neurotoxin BoNT/A1, BoNT/A2, BoNT/A3, BoNT/A4, BoNT/A5, BoNT/A6, BoNT/A7, BoNT/A8, BoNT/B1, BoNT/B2, BoNT/B3, BoNT/B4, BoNT/B5, BoNT/B6, BoNT/B7, BoNT/B8, BoNT/C1, BoNT/CD, BoNT/D, BoNT/DC, BoNT/E1, BoNT/E2, BoNT/E3, BoNT/E4, BoNT/E5, BoNT/E6, BoNT/E7, BoNT/E8, BoNT/E9, BoNT/E10, BoNT/E11, BoNT/E12, BoNT/F1, BoNT/F2, BoNT/F3, BoNT/F4, BoNT/F5, BoNT/F6, BoNT/F7, BoNT/F8, BoNT/G, BoNT/FA(H), BoNT/X, or TeNT of any one of SEQ ID NOs:9-53. The receptor binding domain sequences of these Clostridium neurotoxin sequence are provided in Table 1 infra.

In some embodiments, the RBD is a Clostridium tetani tetanus neurotoxin. The receptor binding domain sequences of tetanospasmin toxin is provided in Table 1 infra.

In some embodiments, the Clostridial receptor binding domain (RBD) has neuron-specific binding activity. The term “neuron-specific binding activity” refers to the selective affinity and interaction of these receptor binding domains with neuronal cells. This specificity is characterized by the ability of the receptor binding domains to recognize and bind to unique molecular structures, such as gangliosides and protein receptors, that are predominantly or exclusively expressed on the surface of neurons.

In some embodiments, the fusion protein comprises a cargo polypeptide upstream of the DT-C domain. Cargo molecules are described in more detail infra.

The terms “linker” and “spacer” are used interchangeably herein. In some embodiments, the various domains of the fusion protein, the propeptide fusion, or the cargo polypeptide are separated by an amino acid linker sequence. This and other amino acid linker (or spacer) sequences described herein may comprise 1, 2, 3, 4, 5, 6, 7, 8, 9, 10, 11, 12, 13, 14, 15, 16, 17, 18, 19, 20, 21-25, 26-30, 31-35, or 40, 45, or more, amino acid residues. The amino acid linker (or spacer) sequence may serve to preserve and protect the conformational independence of DT-C, the DT-T, the RBD, and the cargo to not interfere with their enzymatic or biological activity (such as receptor or antigen binding). In some embodiments, the fusion protein or the propeptide fusion comprise one or more amino acid linker sequences positioned between one or more of the cargo, the catalytic domain (DT-C), the translocation domain (DT-T), and the receptor-binding domain (RBD) domain.

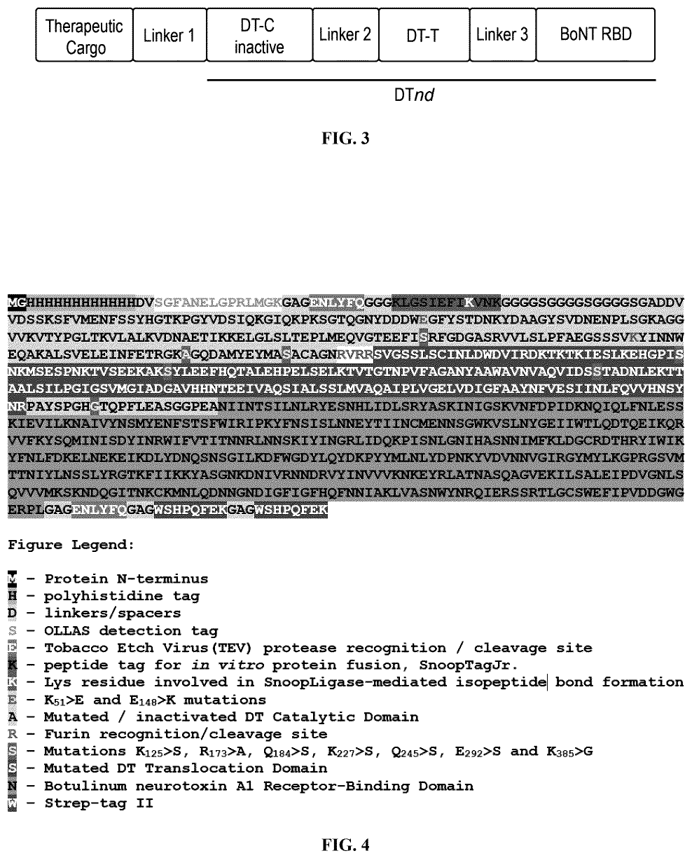

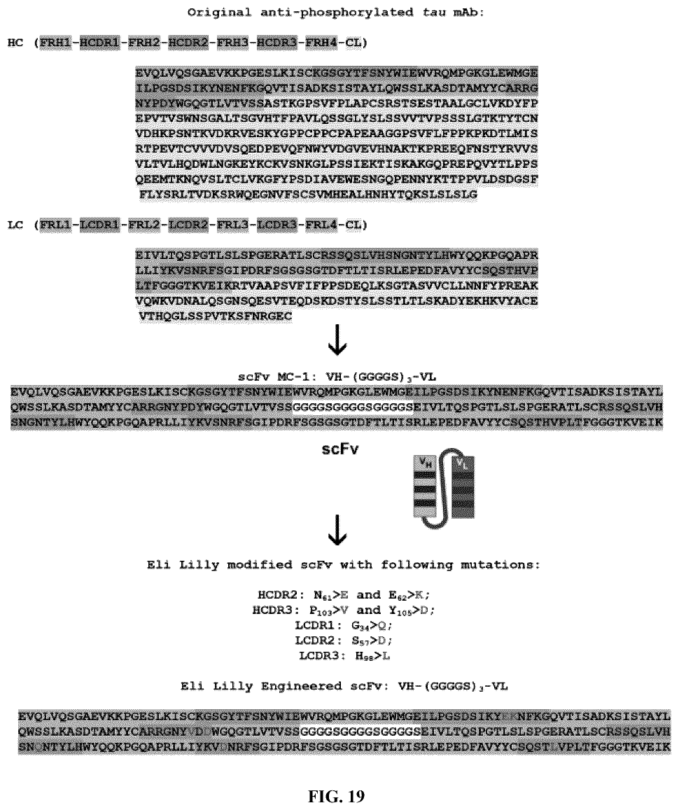

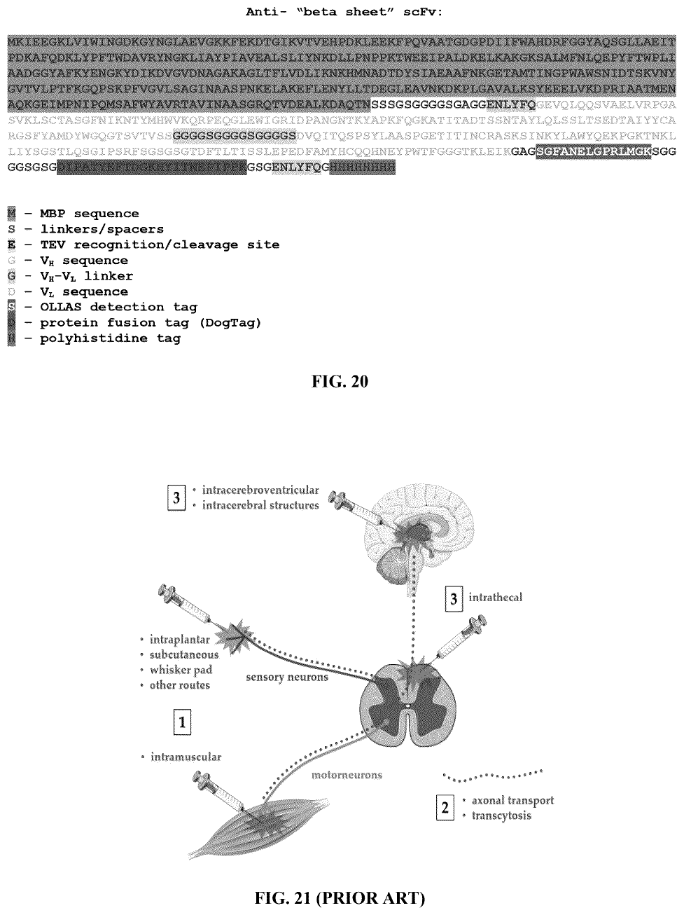

Exemplary amino acid linker sequences are shown below, without limitation. In some embodiments, the linker comprises the sequence G, SS, GAG, GGG, SSG, GSGGSG (SEQ ID NO:54), AAASGGSGGGGSGGGGSGP (SEQ ID NO:55), AAASGGSGGGGSGGGGS (SEQ ID NO:56), GGGGSGGGGSGGGGSGGGGS (SEQ ID NO:57), GGGGSGGGGSGGGGSGGGGSG (SEQ ID NO:58), SGSGGGGSGAG (SEQ ID NO:59), SGSNGGAGQSGAGEGGGGSGGGGSGGGGS (SEQ ID NO:60), T SGGGGSGGGGSGGGGSGGGGSGTSGSTGGGGSGGGGSGAG (SEQ ID NO:61), SSSGSGGGGSGAG (SEQ ID NO:62), GGGGSGGGGSGGGGS (SEQ ID NO:63), GGGGSGGGGS (SEQ ID NO:64), GGGGS (SEQ ID NO:65), GGGGGGGG (SEQ ID NO:66), GGGGGG (SEQ ID NO:67), GSAGSAAGSGEF (SEQ ID NO:68), VPGVGVPGVG (SEQ ID NO:69), PAYSPGHGTQPFLEASGGPEA (SEQ ID NO:70), SGGGGGSGGGGASG (SEQ ID NO:71), and ARGGASG (SEQ ID NO:72). In considering suitable sequences for linkers, it may be desirable to avoid creating any new restriction sites or other instabilities in the expression system. Suitable linkers may also be designed to keep the single chain antibody or other cargo moiety independent of the rest of the polypeptide structure to enable antigen binding. The positions and sequences of various specific linkers (also called spacers) are illustrated in FIGS. 3, 4, 6, 10, 13, 19, and 20 herein.

In some embodiments, the fusion protein, the propeptide fusion, and/or the cargo protein comprise one or more detection tags. A detection tag serves as a molecular marker that facilitates, e.g., the identification, tracking, and purification of the protein within various biological and experimental contexts. In some embodiments, the detection tag is capable of detecting delivery of the fusion protein or portion thereof to the neuronal cytoplasm. In some embodiments, the detection tag is an affinity purification tag used for purification of the fusion protein or propeptide, e.g., with affinity chromatography as described infra. In some embodiments, the detection tag may be a flag tag, a histidine tag (His-tag) HHHHHH (SEQ ID NO:73) or HHHHHHHHHHHH (SEQ ID NO:74), an OLLAS tag SGFANELGPRLMGK (SEQ ID NO:75), a STREP-tag II tag WSHPQFEK (SEQ ID NO:76), a hemagglutinin (HA) tag, a Myc tag, a V5 tag, a glutathione-S-transferase (GST) tag, a Maltose Binding Protein (MBP) tag MKIEEGKLVIWINGDKGYNGLAEVGKKFEKDTGIKVTVEHPDKLEEKFPQVAATGDGPDII FWAH DRFGGYAQSGLLAEITPDKAFQDKLYPFTWDAVRYNGKLIAYPIAVEALSLIYNKDLLPNPPKTW EEIPALDKELKAKGKSALMFNLQEPYFTWPLIAADGGYAFKYENGKYDIKDVGVDNAGAKAGLTF LVDLIKNKHMNADTDYSIAEAAFNKGETAMTINGPWAWSNIDTSKVNYGVTVLPTFKGQPSKPFV GVLSAGINAASPNKELAKEFLENYLLTDEGLEAVNKDKPLGAVALKSYEEELVKDPRIAATMENA QKGEIMPNIPQMSAFWYAVRTAVINAASGRQTVDEALKDAQTN (SEQ ID NO:77), a Green Fluorescent Protein (GFP) tag, a Myc-Pyruvate Kinase tag, a Vesicular Stomatitis Virus Glycoprotein (VSV-G) tag, a Halotag7 EIGTGFPFDPHYVEVLGERMHYVDVGPRDGTPVLFLHGNPTSSYVWRNIIPHVAPTHRCIAPDLI GMGKSDKPDLGYFFDDHVRFMDAFIEALGLEEVVLVIHDWGSALGFHWAKRNPERVKGIAFMEFI RPIPTWDEWPEFARETFQAFRTTDVGRKLIIDQNVFIEGTLPMGVVRPLTEVEMDHYREPFLNPV DREPLWRFPNELPIAGEPANIVALVEEYMDWLHQSPVPKLLFWGTPGVLIPPAEAARLAKSLPNC KAVDIGPGLNLLQEDNPDLIGSEIARWLSTLEI (SEQ ID NO:78), an Avi Tag GLNDI FEAQKIEWHE (SEQ ID NO:79), or other similar tag sequence otherwise known in the art. Exemplary affinity purification tags are His-tag, GST-tag, Flag-tag, MPB, HA tag STREP-tag II tag, VSV-G tag, Halotag7 tag, C-tag, and Avi Tag. In some embodiments, an affinity purification tag is an immobilization sequence.

In some embodiments, the fusion protein, the propeptide fusion, or the cargo protein comprise one or more protease cleavage site(s). Exemplary protease cleavage sites may include, without limitation, a thrombin cleavage site LVPRGS (SEQ ID NO:80), a Factor Xa cleavage site IEGR (SEQ ID NO:81) or IDGR (SEQ ID NO:82), an enterokinase cleavage site DDDDK (SEQ ID NO:83), a Tobacco Etch Virus (TEV) protease site ENLY FQX (SEQ ID NO:84) where X is G or S, a PreScission™ protease site LEVLPQGP (SEQ ID NO:85), and a furin protease cleavage site RVRR (SEQ ID NO:86).

The Tobacco Etch Virus (TEV) protease cleaves between the Gln and Gly or Ser residues of the amino acid sequence ENLYFQ (G/S) (SEQ ID NO:84).

In some embodiments, the protease cleavage site is a highly specific protease cleavage site that has three or more specific adjacent amino acid residues that are recognized by the highly specific protease to permit cleavage (e.g., an enterokinase protease cleavage site, a TEV protease cleavage site, a furin protease cleavage site). In contrast, a low-specificity protease cleavage site has two or less adjacent amino acid residues that are recognized by a protease to enable cleavage (e.g., a trypsin cleavage site). As can be appreciated by a person of ordinary skill in the art, selecting a particularly suitable highly specific protease can depend on the specific conditions under which cleavage is taking place. While one highly specific protease may be most effective under one set of conditions, another highly specific protease may be most effective under a different set of conditions.

In some embodiments, the fusion protein or the propeptide fusion comprises a peptide fusion tag for in vitro protein fusion. In some embodiments, the cargo comprises a peptide fusion tag for in vitro protein fusion. In some embodiments the peptide fusion tag is a SnoopTagJr tag or a DogTag tag. In some embodiments, the SnoopTagJr tag comprises KLGSIEFIKVNK (SEQ ID NO:87). In some embodiments, the DogTag comprises DIPATYEFTDGKHYITNEPIPPK (SEQ ID NO:88). SnoopLigase is an engineered protein ligase that facilitates the covalent bonding of two protein fragments through a peptide bond formation. The ligation reaction facilitated by SnoopLigase results in the formation of an isopeptide bond, a type of covalent bond formed between the side chains of amino acids. This enzyme operates by recognizing and binding to specific peptide fusion tags, termed SnoopTags, which are genetically encoded into the target proteins. Upon binding, SnoopLigase catalyzes the ligation reaction, resulting in a stable and precise protein assembly. SnoopTagJr and DogTag are recognized by SnoopLigase and used to direct peptide-peptide ligation (FIGS. 8A-C). In some embodiments, the peptide fusion tag is positioned upstream of the DT-C domain.

In some embodiments the fusion protein or the propeptide fusion comprises a protease cleavage site between the DT-C and the DT-D domain. In some embodiments the fusion protein or the propeptide fusion comprises a furin protease site between the DT-C and the DT-D domain. In some embodiments, the fusion protein or the propeptide fusion comprises a protease cleavage site upstream of the DT-C domain between one or more detection tags and the DT-C domain. In some embodiments, the fusion protein or the propeptide fusion comprises a protease cleavage site downstream of the RBD and between the RBD and one or more detection tags (see, e.g., FIG. 4).

In some embodiments, the cargo protein comprises a protease cleavage site between one or more detection tags and the therapeutic cargo. In some embodiments, the cargo protein comprises a protease cleavage site between the peptide fusion tag and one or more detection tags (see, e.g., FIG. 6).

Another aspect of the present disclosure relates to a propeptide fusion comprising a catalytic domain of a diphtheria toxin (DT-C), wherein the catalytic domain (DT-C) comprises one or more mutations that inactivate the catalytic domain (DT-C); a translocation domain of a diphtheria toxin (DT-T), wherein the catalytic domain (DT-C) and the translocation domain (DT-T) are linked by a disulfide bond; a first protease cleavage site between the catalytic domain (DT-C) and the translocation domain (DT-T); and a receptor-binding domain (RBD) of a Clostridium neurotoxin protein positioned downstream of the translocation domain (DT-T), wherein the receptor-binding domain (RBD) possesses neuron-specific binding activity, and wherein the fusion protein is capable of delivering a cargo to neural cytoplasm of a cell.

In some embodiments, an exemplary propeptide fusion (also called a DTnd delivery vehicle) is SEQ ID NO:89 as shown in FIG. 4 and is set forth below:

MGHHHHHHHHHHHHDVSGFANELGPRLMGKGAGENLYFQGGGKLG
SIEFIKVNKGGGGSGGGGSGGGGSGADDVVDSSKSFVMENFSSYH
GTKPGYVDSIQKGIQKPKSGTQGNYDDDWEGFYSTDNKYDAAGYS
VDNENPLSGKAGGVVKVTYPGLTKVLALKVDNAETIKKELGLSLT
EPLMEQVGTEEFISRFGDGASRVVLSLPFAEGSSSVKYINNWEQA
KALSVELEINFETRGKAGQDAMYEYMASACAGNRVRRSVGSSLSC
INLDWDVIRDKTKTKIESLKEHGPISNKMSESPNKTVSEEKAKSY
LEEFHQTALEHPELSELKTVTGTNPVFAGANYAAWAVNVAQVIDS
STADNLEKTTAALSILPGIGSVMGIADGAVHHNTEEIVAQSIALS
SLMVAQAIPLVGELVDIGFAAYNFVESIINLFQVVHNSYNRPAYS
PGHGTQPFLEASGGPEANIINTSILNLRYESNHLIDLSRYASKIN
IGSKVNFDPIDKNQIQLENLESSKIEVILKNAIVYNSMYENFSTS
FWIRIPKYFNSISLNNEYTIINCMENNSGWKVSLNYGEIIWTLQD
TQEIKQRVVFKYSQMINISDYINRWIFVTITNNRLNNSKIYINGR
LIDQKPISNLGNIHASNNIMFKLDGCRDTHRYIWIKYFNLFDKEL
NEKEIKDLYDNQSNSGILKDFWGDYLQYDKPYYMLNLYDPNKYVD
VNNVGIRGYMYLKGPRGSVMTTNIYLNSSLYRGTKFIIKKYASGN
KDNIVRNNDRVYINVVVKNKEYRLATNASQAGVEKILSALEIPDV
GNLSQVVVMKSKNDQGITNKCKMNLQDNNGNDIGFIGFHQFNNIA
KLVASNWYNRQIERSSRTLGCSWEFIPVDDGWGERPLGAGENLYF
QGAGWSHPQFEKGAGWSHPQFEK

In some embodiments, the propeptide fusion comprises a peptide fusion tag for in vitro protein fusion. In some embodiments, the peptide fusion tag is a DogTag or a SnoopTagJr. In some embodiments, the propeptide fusion comprises one or more detection tags. In some embodiments, the propeptide fusion comprises one or more amino acid linker sequences positioned between one or more of the catalytic domain (DT-C), the translocation domain (DT-T), and the receptor-binding domain (RBD) domain. In some embodiments, the propeptide fusion comprises one or more affinity purification tags. In some embodiments, the propeptide fusion comprises a first affinity purification tag positioned upstream of an N-terminal detection tag, a second protease cleavage site positioned between the first affinity purification tag and a peptide fusion tag for in vitro protein fusion, a second affinity purification tag located downstream of the receptor-binding domain (RBD), and a third protease cleavage site positioned between the receptor-binding domain and the second affinity purification tag. In some embodiments, the first, second, and third protease cleavage sites are independently selected from a furin recognition/cleavage site, an enterokinase cleavage site, and a TEV recognition/cleavage sequence.

In some embodiments, the DTnd delivery vehicle comprises in the following order: (i) a peptide fusion tag (such as, e.g., SnoopTagJr), (ii) a DT-C domain of any one of SEQ ID NOs:3-5, (iii) a protease cleavage site (such as, e.g., furin), (iv) a DT-T domain of any one of SEQ ID NOs:6-8, and (v) a BoNT RBD of any one of SEQ ID NOs:10-52 or a tetanus neurotoxin RBD of SEQ ID NO:53.

In some embodiments, the DTnd delivery vehicle comprises in the following order: (i) one or more detection tags (such as, e.g., a histidine tag and/or an OLLAS tag) (ii) a protease cleavage site (such as, e.g., TEV protease cleavage site), (iii) a peptide fusion tag (such as, e.g., SnoopTagJr), (iv) a DT-C domain of any one of SEQ ID NOs:3-5, (v) a protease cleavage site (such as, e.g., furin), (vi) a DT-T domain of any one of SEQ ID NOs:6-8, (vii) BoNT RBD of any one of SEQ ID NOs:10-52 or a tetanus neurotoxin RBD of SEQ ID NO:53, (viii) a protease cleavage site (such as, e.g., a TEV site), and (ix) one or more detection tags (such as, e.g., a Strep-tag).

In some embodiments, the propeptide fusion comprises an accelerated degradation domain positioned at an N- or C-terminus of the fusion protein. An accelerated degradation domain is a specialized protein sequence engineered to promote the rapid degradation of a fusion protein or target protein within a cellular environment. This domain functions by recruiting the cellular ubiquitin-proteasome system, which tags the protein for proteolysis, thereby reducing its half-life and ensuring its swift removal from the cell. The incorporation of an accelerated degradation domain can be particularly advantageous in experimental and therapeutic contexts where the timely downregulation of a protein is desired. For instance, it can be used to control the levels of a potentially toxic protein, regulate the timing of signaling pathways, or study the effects of transient protein expression. The accelerated degradation domain can be designed to respond to specific cellular signals or conditions, providing a versatile tool for precise temporal control over protein stability and function. An exemplary accelerated degradation domain is the F₃₉>V mutated FKBP protein (UniProt Accession No. P62942, which is hereby incorporated by reference in its entirety), which is referred to as a conditional Destabilization Tag Binding Protein (DTBP). See Nabet et al., “The dTAG System for Immediate and Target-Specific Protein Degradation,” Nature Chemical Biology 14:431-441 (2018), which is hereby incorporated by reference in its entirety. When fused with a target protein at the N- or C-terminus activated degradation of chimeric derivatives occurs upon addition of cell-permeable heterobifunctional degraders like DTAG-13.

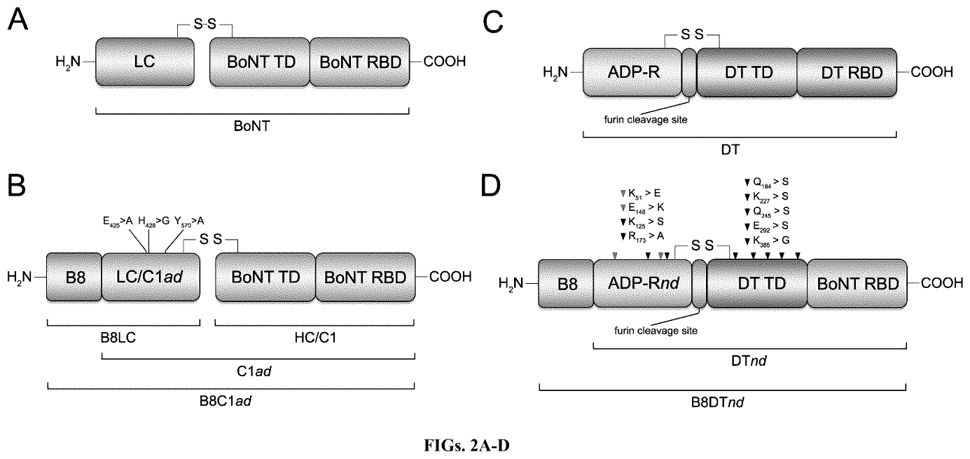

Altogether, the fusion protein represents a diphtheria-based intraneural delivery vehicle named DTnd (FIG. 2D). The DTnd vehicle is able to deliver to the neural cytoplasm the therapeutic cargo that is a C-terminally fused cargo sequence attached to the N-terminus of the DT inactivated catalytic (DT-C) domain. (FIG. 3). The protein cargo can be either genetically fused to the sequence of DTnd, followed by the expression of the entire protein in the system of choice, or it can be enzymatically fused to the delivery vehicle after production and purification of the delivery vehicle and cargo in separate expression platforms. The therapeutic molecule includes a therapeutic cargo fused to a delivery vehicle via linker 1, where the delivery vehicle comprises the inactivated DT-C domain fused via linker 2 to the DT-T domain, and the DT-T domain fused via linker 3 to a neuron-specific receptor-binding domain such as BoNT/A1 RBD (FIG. 3).

A non-limiting example of Linker 1 is a flexible protein sequence, a short (10-25 aa) peptide fusion tag that allow post-translational fusion of the therapeutic cargo with the inactivated DT-C, or a combination of both. A non-limiting example of Linker 2 is the native DT sequence that contains a furin cleavage site, a thrombin cleavage site or another proteolytically cleavable sequence. A non-limiting example of Linker 3 is the native DT sequence (FIG. 4). In some embodiments, the protein may include detection or purification tags in different locations of the sequence that may not affect the activity of the functional domains. Such detection or purification tags may be located at the N and C terminus of the whole sequence or both.

In some embodiments, isolated fusion proteins of the present disclosure are physiologically active. This physiological activity includes, but is not limited to, any one or more of toxin immunogenicity, trans- and intra-cellular trafficking, and cell recognition, which are properties of a wild-type Clostridial neurotoxin.

In some embodiments, the fusion protein is capable of delivering a cargo to neural cytoplasm of a cell. In some embodiments, the fusion protein comprises a single chain antibody positioned upstream of the light chain region and further includes a detection tag (DT)N-terminal to the single chain antibody, where the detection tag is capable of detecting delivery of the single chain antibody to neuronal cytoplasm. Suitable examples of detection tags are discussed infra. In some embodiments, the fusion protein does not contain any detection tags.

A further aspect of the present disclosure relates to a fusion protein produced by cleaving the propeptide fusion of the present disclosure at the first protease cleavage site, wherein the catalytic domain (DT-C) and the translocation domain (DT-T) are linked by a disulfide bond.

### Therapeutic Cargo

In some embodiments, the DTnd delivery vehicle comprises a cargo. The terms “cargo” and “therapeutic cargo” are used interchangeably herein. In some embodiments, a cargo is a molecule that may be used to treat a condition or disease. In some embodiments the fusion protein or the propeptide fusion comprise a cargo. In some embodiments, a cargo comprises a polypeptide, an RNA molecule, a DNA molecule, or a small molecule. In some embodiments, the cargo is a polypeptide.

In some embodiments, the cargo is positioned upstream of the catalytic domain (DT-C).

In some embodiments, the cargo is an antibody. Antibody-related molecules, domains, fragments, portions, etc., useful as cargo of the present disclosure include, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, C_{L} and CH_{I} domains; (ii) a F(ab′)₂ fragment, a bivalent fragment comprising two Fab fragments linked by a disulfide bridge at the hinge region; (iii) a Fd fragment consisting of the V_{H} and CH_{I} domains; (iv) a Fv fragment consisting of the V_{L} and V_{H} domains of a single arm of an antibody, (v) a dAb fragment (Ward et al., “Binding Activities of a Repertoire of Single Immunoglobulin Variable Domains Secreted From Escherichia coli,” Nature 341:544-46 (1989), which is hereby incorporated by reference in its entirety), which consists of a V_{H} domain; and (vi) an isolated complementary determining region (CDR). As such “antibody fragments” can comprise a portion of a full-length antibody, generally the antigen binding or variable region thereof. Examples of antibody fragments include Fab, Fab′, F(ab′)₂, and Fv fragments; diabodies; linear antibodies; single-chain antibody molecules; and multi-specific antibodies formed from antibody fragments.

In some embodiments, the antibody is a single domain antibody. As used herein, the term “single domain antibody”, or “sdAb” means an immunoglobulin single chain variable domain on a single polypeptide, which is capable of specifically binding to an epitope of an antigen without pairing with an additional variable immunoglobulin domain. One example of immunoglobulin single chain variable domains includes “VHH domains” (or simply “VHHs”) from camelids. Another example of immunoglobulin single variable domains includes “domain antibodies,” such as the immunoglobulin single variable domains VH and VL (VH domains and VL domains, when fused together in artificial constructs). In some embodiments, the cargo comprises a B8 single domain antibody, a JSG-C1 single domain antibody (JC1), or an anti-tau single domain antibody 2B8.

Methods of obtaining VHH domains binding to a specific antigen or epitope have been described earlier, e.g., in PCT Publication Nos. WO 2006/040153 and WO 2006/122786, which are hereby incorporated by reference in their entirety. As also described therein in detail, VHH domains derived from camelids can be “humanized” by replacing one or more amino acid residues in the amino acid sequence of the original VHH sequence by one or more of the amino acid residues that occur at the corresponding position(s) in a VH domain from a conventional 4-chain antibody from a human being. A humanized VHH domain can contain one or more fully human framework region sequences.

Single chain antibodies or fragments thereof can be produced from multi-chain antibodies (Sheets et al., “Efficient Construction of a Large Nonimmune Phage Antibody Library: The Production of High-Affinity Human Single-Chain Antibodies to Protein Antigens,” PNAS USA 95(11):6157-6162 (1998), which is hereby incorporated by reference in its entirety) or can be derived from species that naturally produce single chain antibodies, such as sharks and camelids (Dumoulin et al., “Single-Domain Antibody Fragments with High Conformational Stability,” Protein Science: A Publication of the Protein Society 11(3):500-515 (2002), which is hereby incorporated by reference in its entirety).

As used herein, the terms “single chain antibodies” or “single chain Fv (scFv)” may refer to an antibody fusion molecule of the two domains of the Fv fragment, V_{L} and V_{H}. Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv). See, e.g., Bird et al., “Single-Chain Antigen-Binding Proteins,” Science 242:423-26 (1988) and Huston et al., “Protein Engineering of Antibody Binding Sites: Recovery of Specific Activity in an Anti-Digoxin Single-Chain Fv Analogue Produced in Escherichia coli,” Proc. Natl. Acad. Sci. USA 85:5879-83 (1988), which are hereby incorporated by reference in their entirety. Such single chain antibodies are included by reference to the term “antibody” fragments and can be prepared by recombinant techniques or enzymatic or chemical cleavage of intact antibodies.

In some embodiments, the fusion protein or the propeptide fusion comprises a B8 sdAb. The B8 sdAb is a single chain VHH camelid antibody against BoNT/A. The amino acid sequence of B8 is set forth as SEQ ID NO:90, as follows:

QAHVQLQQSGGGLVQPGGSLRLSCAASGSIFSIYAMGWYRQAPGK
QRELVAAISSYGSTNYADSVKGRFTISRDNAKNTVYLQMNSLKPE
DTAVYYCNADIATMTAVGGFDYWGQGTQVTVSSAHHSEDPTSQS

In some embodiments, the cargo is expressed as a propeptide fusion comprising one or more purification tags, one or more protease cleavage sites, and/or a peptide fusion tag. In some embodiments, the peptide fusion tag is DogTag or Snooptag Jr. In some embodiments, the B8 cargo comprises the sequence as set forth in SEQ ID NO:91, as follows:

MKIEEGKLVIWINGDKGYNGLAEVGKKFEKDTGIKVTVEHPDKLE
EKFPQVAATGDGPDIIFWAHDRFGGYAQSGLLAEITPDKAFQDKL
YPFTWDAVRYNGKLIAYPIAVEALSLIYNKDLLPNPPKTWEEIPA
LDKELKAKGKSALMENLQEPYFTWPLIAADGGYAFKYENGKYDIK
DVGVDNAGAKAGLTELVDLIKNKHMNADTDYSIAEAAFNKGETAM
TINGPWAWSNIDTSKVNYGVTVLPTFKGQPSKPFVGVLSAGINAA
SPNKELAKEFLENYLLTDEGLEAVNKDKPLGAVALKSYEEELVKD
PRIAATMENAQKGEIMPNIPQMSAFWYAVRTAVINAASGRQTVDE
ALKDAQTNSSSGSGGGGSGAGENLYFQSGSNGGAGQSGAGEGGGG
SGGGGSGGGGSQAHVQLQQSGGGLVQPGGSLRLSCAASGSIFSIY
AMGWYRQAPGKQRELVAAISSYGSTNYADSVKGRFTISRDNAKNT
VYLQMNSLKPEDTAVYYCNADIATMTAVGGFDYWGQGTQVTVSSA
HHSEDPTSQSTSGGGGSGGGGSGGGGSGGGGSGTSGSTGGGGSGG
GGSGAGDIPATYEFTDGKHYITNEPIPPKGAGENLYFQGHHHHHH

In some embodiments, the fusion protein or the propeptide fusion comprises a JSG-C1 sdAb. In some embodiments, the cargo comprises a JSG-C1 sdAb. The JSG-C1 sdAb is a single chain VHH camelid antibody that binds and inhibits the catalytic activity of the Light Chain of the BoNT/B1 neurotoxin. The amino acid sequence of JSG-C1 is set forth as SEQ ID NO:92, as follows:

AGTSQVQLVESGGGLVQTGGSLRLSCAASGRTERRNTMGWFRQAP
GKVREFVAAISWSGDRTYCADSVKGRFTISRDNAKNTVDLLMNSL
KPEDTAIYYCAADGTASVENSYASADRNKYNYWGQGTQVTVSSGS
TA

In some embodiments, the JSG-C1 cargo is expressed as a propeptide fusion comprising one or more purification tags, one or more protease cleavage sites, and/or a peptide fusion tag. In some embodiments, the peptide fusion tag is DogTag or Snooptag Jr. In some embodiments, the cargo comprises the sequence as set forth in SEQ ID NO:93, as follows:

MKIEEGKLVIWINGDKGYNGLAEVGKKFEKDTGIKVTVEHPDKLE
EKFPQVAATGDGPDIIFWAHDRFGGYAQSGLLAEITPDKAFQDKL
YPFTWDAVRYNGKLIAYPIAVEALSLIYNKDLLPNPPKTWEEIPA
LDKELKAKGKSALMENLQEPYFTWPLIAADGGYAFKYENGKYDIK
DVGVDNAGAKAGLTELVDLIKNKHMNADTDYSIAEAAFNKGETAM
TINGPWAWSNIDTSKVNYGVTVLPTFKGQPSKPFVGVLSAGINAA
SPNKELAKEFLENYLLTDEGLEAVNKDKPLGAVALKSYEEELVKD
PRIAATMENAQKGEIMPNIPQMSAFWYAVRTAVINAASGRQTVDE
ALKDAQTNSSSGSGGGGSGAGENLYFQSGSNGGAGQSGAGEGGGG
SGGGGSGGGGSAGTSQVQLVESGGGLVQTGGSLRLSCAASGRTER
RNTMGWFRQAPGKVREFVAAISWSGDRTYCADSVKGRFTISRDNA
KNTVDLLMNSLKPEDTAIYYCAADGTASVENSYASADRNKYNYWG
QGTQVTVSSGSTATSGGGGSGGGGSGGGGSGGGGSGTSGSTGGGG
SGGGGSGAGDIPATYEFTDGKHYITNEPIPPKGAGENLYFQGHHH
HHH

Additional sdAb targeting BoNTs are described in Lam et al., “Probing the structure and function of the protease domain of botulinum neurotoxins using single-domain antibodies,” PLoS Pathogens 18.1 e1010169 (2022) and Tremblay et al., “Camelid VHH Antibodies that Neutralize Botulinum Neurotoxin Serotype E Intoxication or Protease Function” Toxins 12.10 611(2020), each of which is hereby incorporated by reference in its entirety.

In some embodiments, the fusion protein or the propeptide fusion comprises a 2B8 sdAb. The 2B8 sdAb is a single chain VHH camelid antibody against the pathological conformations of tau protein (see e.g., PCT Publication No. WO2019161384, which is hereby incorporated by reference in its entirety). The amino acid sequence of 2B8 is set forth as SEQ ID NO:94 below:

QVQLAESGGGLVQAGGSLRLSCVVSGRTESTSQMGWFRQPPGKER
ELVARISWRGKQHYADSVKGRFTISRDYAKNTVYLQMNGLKSEDT
AVYYCAADRRRTYLGQQHDYWGQGTLVTVSS

In some embodiments, the 2B8 cargo is expressed as a propeptide fusion comprising one or more purification tags, one or more protease cleavage sites, and/or a peptide fusion tag. In some embodiments, the peptide fusion tag is DogTag or Snooptag Jr. In some embodiments, the cargo comprises the sequence as set forth in SEQ ID NO:95, as follows:

MKIEEGKLVIWINGDKGYNGLAEVGKKFEKDTGIKVTVEHPDKLE
EKFPQVAATGDGPDIIFWAHDRFGGYAQSGLLAEITPDKAFQDKL
YPFTWDAVRYNGKLIAYPIAVEALSLIYNKDLLPNPPKTWEEIPA
LDKELKAKGKSALMENLQEPYFTWPLIAADGGYAFKYENGKYDIK
DVGVDNAGAKAGLTELVDLIKNKHMNADTDYSIAEAAFNKGETAM
TINGPWAWSNIDTSKVNYGVTVLPTFKGQPSKPFVGVLSAGINAA
SPNKELAKEFLENYLLTDEGLEAVNKDKPLGAVALKSYEEELVKD
PRIAATMENAQKGEIMPNIPQMSAFWYAVRTAVINAASGRQTVDE
ALKDAQTNSSSGSGGGGSGAGENLYFQSGSNGGAGQSGAGEGGGG
SGGGGSGGGGSQVQLAESGGGLVQAGGSLRLSCVVSGRTFSTSQM
GWFRQPPGKERELVARISWRGKQHYADSVKGRFTISRDYAKNTVY
LQMNGLKSEDTAVYYCAADRRRTYLGQQHDYWGQGTLVTVSSTSG
GGGSGGGGSGGGGSGGGGSGTSGSTGGGGSGGGGSGAGDIPATYE
FTDGKHYITNEPIPPKGAGENLYFQGHHHHHH

In some embodiments, the cargo comprises an anti-beta sheet single-chain variable fragment (scFv). The anti-beta sheet single-chain variable fragment (scFv) is an antibody fragment designed to specifically recognize and bind to amyloid beta-sheet structures of pathological oligomeric conformers, characteristic of many neurodegenerative diseases. Beta-sheets are common structural motifs in proteins, consisting of beta-strands connected laterally by at least two or three backbone hydrogen bonds, forming a generally twisted, pleated sheet. In certain disease states, dominant p-sheet secondary structures oligomerize into pathologic, fibrillogenic conformers, which lead to loss of function and toxicity. See e.g., Goni et al., “Production of Monoclonal Antibodies to Pathologic β-sheet Oligomeric Conformers in Neurodegenerative Diseases,” Scientific Reports 7:9881 (2017) and Goni et al., “Anti-β-sheet Conformation Monoclonal Antibody Reduces Tau and A3 Oligomer Pathology in an Alzheimer's Disease Model,” Alzheimer's Research and Therapy 10:10 (2018), each of which is hereby incorporated by reference in its entirety.

The scFv format includes the variable regions of the heavy (VH) and light (VL) chains of an antibody, connected by a short flexible linker, creating a single polypeptide chain. This configuration retains the antigen-binding specificity of the original antibody while being smaller and more stable.

In some embodiments the anti-beta sheet scFv aggregates binds to aggregates formed e.g., in Alzheimer's disease, Parkinson's disease, and amyloidosis..

The amino acid sequence of anti-beta sheet scFv is set forth as SEQ ID NO:96, as follows:

GEVQLQQSVAELVRPGASVKLSCTASGENIKNTYMHWVKQRPEQG
LEWIGRIDPANGNTKYAPKFQGKATITADTSSNTAYLQLSSLTSE
DTAIYYCARGSFYAMDYWGQGTSVTVSSGGGGSGGGGSGGGGSDV
QITQSPSYLAASPGETITINCRASKSINKYLAWYQEKPGKTNKLL
IYSGSTLQSGIPSRFSGSGSGTDFTLTISSLEPEDFAMYHCQQHN
EYPWTFGGGTKLEIK

In some embodiments, the anti-beta sheet scFv cargo is expressed as a propeptide fusion comprising one or more purification tags, one or more protease cleavage sites, and/or a peptide fusion tag. In some embodiments, the peptide fusion tag is DogTag. In some embodiments, the cargo comprises the sequence as set forth in SEQ ID NO:97, as follows:

MKIEEGKLVIWINGDKGYNGLAEVGKKFEKDTGIKVTVEHPDKLE
EKFPQVAATGDGPDIIFWAHDRFGGYAQSGLLAEITPDKAFQDKL
YPFTWDAVRYNGKLIAYPIAVEALSLIYNKDLLPNPPKTWEEIPA
LDKELKAKGKSALMFNLQEPYFTWPLIAADGGYAFKYENGKYDIK
DVGVDNAGAKAGLTFLVDLIKNKHMNADTDYSIAEAAFNKGETAM
TINGPWAWSNIDTSKVNYGVTVLPTFKGQPSKPFVGVLSAGINAA
SPNKELAKEFLENYLLTDEGLEAVNKDKPLGAVALKSYEEELVKD
PRIAATMENAQKGEIMPNIPQMSAFWYAVRTAVINAASGRQTVDE
ALKDAQTNSSSGSGGGGSGAGGENLYFQGEVQLQQSVAELVRPGA
SVKLSCTASGENIKNTYMHWVKQRPEQGLEWIGRIDPANGNTKYA
PKFQGKATITADTSSNTAYLQLSSLTSEDTAIYYCARGSFYAMDY
WGQGTSVTVSSGGGGSGGGGSGGGGSDVQITQSPSYLAASPGETI
TINCRASKSINKYLAWYQEKPGKTNKLLIYSGSTLQSGIPSRFSG
SGSGTDFTLTISSLEPEDFAMYHCQQHNEYPWTFGGGTKLEIKGA
GSGFANELGPRLMGKSGGGGSGSGDIPATYEFTDGKHYITNEPIP
PKGSGENLYFQGHHHHHHHH

In some embodiments, the cargo comprises an anti-pathological tau scFv antibody. For example, an anti-pathological tau can specifically target tau protein that has undergone phosphorylation, a post-translational modification where phosphate groups are added to the protein. Tau is a microtubule-associated protein primarily found in neurons, where it stabilizes microtubules and supports neuronal structure and function. However, in various neurodegenerative diseases, such as Alzheimer's disease, tau becomes abnormally hyperphosphorylated. Hyperphosphorylated tau tends to detach from microtubules and aggregate into insoluble fibrils, forming neurofibrillary tangles (NFTs), which are a hallmark of Alzheimer's disease and other tauopathies. These aggregates disrupt cellular function and contribute to neurodegeneration.

The amino acid sequence of anti-phosphorylated tau scFv antibody is set forth as SEQ ID NO:98, as follows:

EVQLVQSGAEVKKPGESLKISCKGSGYTFSNYWIEWVRQMPGKGLEWMGE
ILPGSDSIKYEKNEKGQVTISADKSISTAYLQWSSLKASDTAMYYCARRG
NYVDDWGQGTLVTVSSGGGGSGGGGSGGGGSEIVLTQSPGTLSLSPGERA
TLSCRSSQSLVHSNQNTYLHWYQQKPGQAPRLLIYKVDNRFSGIPDRESG
SGSGTDFTLTISRLEPEDFAVYYCSQSTLVPLTFGGGTKVEIK

In some embodiments, the anti-phosphorylated tau scFv cargo is expressed as a propeptide fusion comprising one or more purification tags, one or more protease cleavage sites, and/or a peptide fusion tag. In some embodiments, the peptide fusion tag is DogTag or SnoopTag Jr. In some embodiments, the cargo comprises the sequence as set forth in SEQ ID NO:99, as follows:

MKIEEGKLVIWINGDKGYNGLAEVGKKFEKDTGIKVTVEHPDKLEEKFPQ
VAATGDGPDIIFWAHDRFGGYAQSGLLAEITPDKAFQDKLYPFTWDAVRY
NGKLIAYPIAVEALSLIYNKDLLPNPPKTWEEIPALDKELKAKGKSALME
NLQEPYFTWPLIAADGGYAFKYENGKYDIKDVGVDNAGAKAGLTELVDLI
KNKHMNADTDYSIAEAAFNKGETAMTINGPWAWSNIDTSKVNYGVTVLPT
FKGQPSKPFVGVLSAGINAASPNKELAKEFLENYLLTDEGLEAVNKDKPL
GAVALKSYEEELVKDPRIAATMENAQKGEIMPNIPQMSAFWYAVRTAVIN
AASGRQTVDEALKDAQTNSSSGSGGGGSGAGGENLYFQEVQLVQSGAEVK
KPGESLKISCKGSGYTESNYWIEWVRQMPGKGLEWMGEILPGSDSIKYEK
NEKGQVTISADKSISTAYLQWSSLKASDTAMYYCARRGNYVDDWGQGTLV
TVSSGGGGSGGGGSGGGGSEIVLTQSPGTLSLSPGERATLSCRSSQSLVH
SNQNTYLHWYQQKPGQAPRLLIYKVDNRESGIPDRFSGSGSGTDFTLTIS
RLEPEDFAVYYCSQSTLVPLTFGGGTKVEIKGAGSGFANELGPRLMGKSG
GGGSGSGDIPATYEFTDGKHYITNEPIPPKGSGENLYFQGHHHHHHHH

### Nucleic Acid Molecules & Expression of Fusion Proteins

A further aspect of the present disclosure relates to an isolated nucleic acid molecule encoding the propeptide fusion described herein.

The wild type diphtheria toxin nucleic acid molecule has a nucleotide sequence as set forth in GenBank Accession No. MW833977.1, which is hereby incorporated by reference in its entirety is SEQ ID NO:100, as follows:

GTGAGCAGAAAACTGTTTGCGTCAATCTTAATAGGGGCGCTACTGGGGAT
AGGGGCCCCACCTTCAGCCCATGCAGGCGCTGATGATGTTGTTGATTCTT
CTAAATCTTTTGTGATGGAAAACTTTTCTTCGTACCACGGGACTAAACCT
GGTTATGTAGATTCCATTCAAAAAGGTATACAAAAGCCAAAATCTGGTAC
ACAAGGAAATTATGACGATGATTGGAAAGGGTTTTATAGTACCGACAATA
AATACGACGCTGCGGGATACTCTGTAGATAATGAAAACCCGCTCTCTGGA
AAAGCTGGAGGCGTGGTCAAAGTGACGTATCCAGGACTGACGAAGGTTCT
CGCACTAAAAGTGGATAATGCCGAAACTATTAAGAAAGAGTTAGGTTTAA
GTCTCACTGAACCGTTGATGGAGCAAGTCGGAACGGAAGAGTTTATCAAA
AGGTTCGGTGATGGTGCTTCGCGTGTAGTGCTCAGCCTTCCCTTCGCTGA
GGGGAGTTCTAGCGTTGAATATATTAATAACTGGGAACAGGCGAAAGCGT
TAAGCGTAGAACTTGAGATTAATTTTGAAACCCGTGGAAAACGTGGCCAA
GATGCGATGTATGAGTATATGGCTCAAGCCTGTGCAGGAAATCGTGTCAG
GCGATCAGTAGGTAGCTCATTGTCATGCATAAATCTTGATTGGGATGTCA
TAAGGGATAAAACTAAGACAAAGATAGAGTCTTTGAAAGAGCATGGCCCT
ATCAAAAATAAAATGAGCGAAAGTCCCAATAAAACAGTATCTGAGGAAAA
AGCTAAACAATACCTAGAAGAATTTCATCAAACGGCATTAGAGCATCCTG
AATTGTCAGAACTTAAAACCGTTACTGGGACCAATCCTGTATTCGCTGGG
GCTAACTATGCGGCGTGGGCAGTAAACGTTGCGCAAGTTATCGATAGCGA
AACAGCTGATAATTTGGAAAAGACAACTGCTGCTCTTTCGATACTTCCTG
GTATCGGTAGCGTAATGGGCATTGCAGACGGTGCCGTTCACCACAATACA
GAAGAGATAGTGGCACAATCAATAGCTTTATCGTCTTTAATGGTTGCTCA
AGCTATTCCATTGGTAGGAGAGCTAGTTGATATTGGTTTCGCTGCATATA
ATTTTGTAGAGAGTATTATCAATTTATTTCAAGTAGTTCATAATTCGTAT
AATCGTCCCGCGTATTCTCCGGGGCATAAAACGCAACCATTTCTTCATGA
CGGGTATGCTGTCAGTTGGAACACTGTTGAAGATTCGATAATCCGAACTG
GTTTTCAAGGGGAGAGTGGGCACGACATAAAAATTACTGCTGAAAATACC
CCGCTTCCAATCGCGGGTGTCCTACTACCGACTATTCCTGGAAAGCTGGA
CGTTAATAAGTCCAAGACTCATATTTCCGTAAATGGTCGGAAAATAAGGA
TGCGTTGCAGAGCTATAGACGGTGATGTAACTTTTTGTCGCCCTAAATCT
CCTGTTTATGTTGGTAATGGTGTGCATGCGAATCTTCACGTGGCATTTCA
CAGAAGCAGCTCGGAGAAAATTCATTCTAATGAAATTTCGTCGGATTCCA
TAGGCGTTCTTGGGTACCAGAAAACAGTAGATCACACCAAGGTTAATTCT
AAGCTATCGCTATTTTTTGAAATCAAAAGCTGA

The nucleotide sequence encoding the wild type DT-C domain encompasses nucleotides 76-642 of SEQ ID NO:100 and is shown below as SEQ ID NO:101.

GGCGCTGATGATGTTGTTGATTCTTCTAAATCTTTTGTGATGGAAAACTT
TTCTTCGTACCACGGGACTAAACCTGGTTATGTAGATTCCATTCAAAAAG
GTATACAAAAGCCAAAATCTGGTACACAAGGAAATTATGACGATGATTGG
AAAGGGTTTTATAGTACCGACAATAAATACGACGCTGCGGGATACTCTGT
AGATAATGAAAACCCGCTCTCTGGAAAAGCTGGAGGCGTGGTCAAAGTGA
CGTATCCAGGACTGACGAAGGTTCTCGCACTAAAAGTGGATAATGCCGAA
ACTATTAAGAAAGAGTTAGGTTTAAGTCTCACTGAACCGTTGATGGAGCA
AGTCGGAACGGAAGAGTTTATCAAAAGGTTCGGTGATGGTGCTTCGCGTG
TAGTGCTCAGCCTTCCCTTCGCTGAGGGGAGTTCTAGCGTTGAATATATT
AATAACTGGGAACAGGCGAAAGCGTTAAGCGTAGAACTTGAGATTAATTT
TGAAACCCGTGGAAAACGTGGCCAAGATGCGATGTATGAGTATATGGCTC
AAGCCTGTGCAGGAAAT

The nucleotide sequence encoding the wild type DT-T domain encompasses nucleotides 655-1206 of SEQ ID NO:100 and is shown below as SEQ ID NO:102.

TCAGTAGGTAGCTCATTGTCATGCATAAATCTTGATTGGGATGTCATAAG
GGATAAAACTAAGACAAAGATAGAGTCTTTGAAAGAGCATGGCCCTATCA
AAAATAAAATGAGCGAAAGTCCCAATAAAACAGTATCTGAGGAAAAAGCT
AAACAATACCTAGAAGAATTTCATCAAACGGCATTAGAGCATCCTGAATT
GTCAGAACTTAAAACCGTTACTGGGACCAATCCTGTATTCGCTGGGGCTA
ACTATGCGGCGTGGGCAGTAAACGTTGCGCAAGTTATCGATAGCGAAACA
GCTGATAATTTGGAAAAGACAACTGCTGCTCTTTCGATACTTCCTGGTAT
CGGTAGCGTAATGGGCATTGCAGACGGTGCCGTTCACCACAATACAGAAG
AGATAGTGGCACAATCAATAGCTTTATCGTCTTTAATGGTTGCTCAAGCT
ATTCCATTGGTAGGAGAGCTAGTTGATATTGGTTTCGCTGCATATAATTT
TGTAGAGAGTATTATCAATTTATTTCAAGTAGTTCATAATTCGTATAATC
GT

In some embodiments, the isolated nucleic acid molecule of the present disclosure comprises nucleotide sequences modified from the wild-type DT nucleic acid molecule, according to the genetic code, to encode propeptide fusions comprising the mutations described herein. Non-limiting examples of such modifications include optimization with respect to codon usage bias of the host used for production of polypeptides, exclusion of unwanted genetic features that affect transcription and translation, and introduction or exclusion of restriction sites. Thus, nucleic acid molecules of the present disclosure may have a nucleic acid sequence quite similar to the wild-type DT nucleic acid molecule, at least with respect to the DT-C and DT-T domains and comprising inactivating mutations and other mutations as described herein. The DT-C domain may be at least about 80%, 81%, 82%, 83%, 84%, 85%, 86%, 87%, 88%, 89%, 90%, 91%, 92%, 93%, 94%, 95%, 96%, 97%, 98%, 99% or more identical to the nucleic acid molecule of SEQ ID NO:101. The DT-T domain may be at least about 80%, 81%, 82%, 83%, 84%, 85%, 86%, 87%, 88%, 89%, 90%, 91%, 92%, 93%, 94%, 95%, 96%, 97%, 98%, 99% or more identical to the nucleic acid molecule of SEQ ID NO: 102.

The wild type Clostridium botulinum neurotoxin BoNT/A1 nucleic acid molecule has a nucleotide sequence as set forth in GenBank Accession No. EF506573.1, which is hereby incorporated by reference in its entirety (SEQ ID NO: 103), as shown below:

ATGCCATTTGTTAATAAACAATTTAATTATAAAGATCCTGTAAATGGTGT
TGATATTGCTTATATAAAAATTCCAAATGCAGGACAAATGCAACCAGTAA
AAGCTTTTAAAATTCATAATAAAATATGGGTTATTCCAGAAAGAGATACA
TTTACAAATCCTGAAGAAGGAGATTTAAATCCACCACCAGAAGCAAAACA
AGTTCCAGTTTCATATTATGATTCAACATATTTAAGTACAGATAATGAAA
AAGATAATTATTTAAAGGGAGTTACAAAATTATTTGAGAGAATTTATTCA
ACTGATCTTGGAAGAATGTTGTTAACATCAATAGTAAGGGGAATACCATT
TTGGGGTGGAAGTACAATAGATACAGAATTAAAAGTTATTGATACTAATT
GTATTAATGTGATACAACCAGATGGTAGTTATAGATCAGAAGAACTTAAT
CTAGTAATAATAGGACCCTCAGCTGATATTATACAGTTTGAATGTAAAAG
CTTTGGACATGAAGTTTTGAATCTTACGCGAAATGGTTATGGCTCTACTC
AATACATTAGATTTAGCCCAGATTTTACATTTGGTTTTGAGGAGTCACTT
GAAGTTGATACAAATCCTCTTTTAGGTGCAGGCAAATTTGCTACAGATCC
AGCAGTAACATTAGCACATGAACTTATACATGCTGGACATAGATTATATG
GAATAGCAATTAATCCAAATAGGGTTTTTAAAGTAAATACTAATGCCTAT
TATGAAATGAGTGGGTTAGAAGTAAGCTTTGAGGAACTTAGAACATTTGG
GGGACATGATGCAAAGTTTATAGATAGTTTACAGGAAAACGAATTTCGTC
TATATTATTATAATAAGTTTAAAGATATAGCAAGTACACTTAATAAAGCT
AAATCAATAGTAGGTACTACTGCTTCATTACAGTATATGAAAAATGTTTT
TAAAGAGAAATATCTCCTATCTGAAGATACATCTGGAAAATTTTCGGTAG
ATAAATTAAAATTTGATAAGTTATACAAAATGTTAACAGAGATTTACACA
GAGGATAATTTTGTTAAGTTTTTTAAAGTACTTAACAGAAAAACATATTT
GAATTTTGATAAAGCCGTATTTAAGATAAATATAGTACCTAAGGTAAATT
ACACAATATATGATGGATTTAATTTAAGAAATACAAATTTAGCAGCAAAC
TTTAATGGTCAAAATACAGAAATTAATAATATGAATTTTACTAAACTAAA
AAATTTTACTGGATTGTTTGAATTTTATAAGTTGCTATGTGTAAGAGGGA
TAATAACTTCTAAAACTAAATCATTAGATAAAGGATACAATAAGGCATTA
AATGATTTATGTATCAAAGTTAATAATTGGGACTTGTTTTTTAGTCCTTC
AGAAGATAATTTTACTAATGATCTAAATAAAGGAGAAGAAATTACATCTG
ATACTAATATAGAAGCAGCAGAAGAAAATATTAGTTTAGATTTAATACAA
CAATATTATTTAACCTTTAATTTTGATAATGAACCTGAAAATATTTCAAT
AGAAAATCTTTCAAGTGACATTATAGGCCAATTAGAACTTATGCCTAATA
TAGAAAGATTTCCTAATGGAAAAAAGTATGAGTTAGATAAATATACTATG
TTCCATTATCTTCGTGCTCAAGAATTTGAACATGGTAAATCTAGGATTGC
TTTAACAAATTCTGTTAACGAAGCATTATTAAATCCTAGTCGTGTTTATA
CATTTTTTTCTTCAGACTATGTAAAGAAAGTTAATAAAGCTACGGAGGCA
GCTATGTTTTTAGGCTGGGTAGAACAATTAGTATATGATTTTACCGATGA
AACTAGCGAAGTAAGTACTACGGATAAAATTGCGGATATAACTATAATTA
TTCCATATATAGGACCTGCTTTAAATATAGGTAATATGTTATATAAAGAT
GATTTTGTAGGTGCTTTAATATTTTCAGGAGCTGTTATTCTGTTAGAATT
TATACCAGAGATTGCAATACCTGTATTAGGTACTTTTGCACTTGTATCAT
ATATTGCGAATAAGGTTCTAACCGTTCAAACAATAGATAATGCTTTAAGT
AAAAGAAATGAAAAATGGGATGAGGTCTATAAATATATAGTAACAAATTG
GTTAGCAAAGGTTAATACACAGATTGATCTAATAAGAAAAAAAATGAAAG
AAGCTTTAGAAAATCAAGCAGAAGCAACAAAGGCTATAATAAACTATCAG
TATAATCAATATACTGAGGAAGAGAAAAATAATATTAATTTTAATATTGA
TGATTTAAGTTCGAAACTTAATGAGTCTATAAATAAAGCTATGATTAATA
TAAATAAATTTTTGAATCAATGCTCTGTTTCATATTTAATGAATTCTATG
ATCCCTTATGGTGTTAAACGGTTAGAAGATTTTGATGCTAGTCTTAAAGA
TGCATTATTAAAGTATATATATGATAATAGAGGAACTTTAATTGGTCAAG
TAGATAGATTAAAAGATAAAGTTAATAATACACTTAGTACAGATATACCT
TTTCAGCTTTCCAAATACGTAGATAATCAAAGATTATTATCTACATTTAC
TGAATATATTAAGAATATTATTAATACTTCTATATTGAATTTAAGATATG
AAAGTAATCATTTAATAGACTTATCTAGGTATGCATCAAAAATAAATATT
GGTAGTAAAGTAAATTTTGATCCAATAGATAAAAATCAAATTCAATTATT
TAATTTAGAAAGTAGTAAAATTGAGGTAATTTTAAAAAATGCTATTGTAT
ATAATAGTATGTATGAAAATTTTAGTACTAGCTTTTGGATAAGAATTCCT
AAGTATTTTAACAGTATAAGTCTAAATAATGAATATACAATAATAAATTG
TATGGAAAATAATTCAGGATGGAAAGTATCACTTAATTATGGTGAAATAA
TCTGGACTTTACAGGATACTCAGGAAATAAAACAAAGAGTAGTTTTTAAA
TACAGTCAAATGATTAATATATCAGATTATATAAACAGATGGATTTTTGT
AACTATCACTAATAATAGATTAAATAACTCTAAAATTTATATAAATGGAA
GATTAATAGATCAAAAACCAATTTCAAATTTAGGTAATATTCATGCTAGT
AATAATATAATGTTTAAATTAGATGGTTGTAGAGATACACATAGATATAT
TTGGATAAAATATTTTAATCTTTTTGATAAGGAATTAAATGAAAAAGAAA
TCAAAGATTTATATGATAATCAATCAAATTCAGGTATTTTAAAAGACTTT
TGGGGTGATTATTTACAATATGATAAACCATACTATATGTTAAATTTATA
TGATCCAAATAAATATGTCGATGTAAATAATGTAGGTATTAGAGGTTATA
TGTATCTTAAAGGGCCTAGAGGTAGCGTAATGACTACAAACATTTATTTA
AATTCAAGTTTGTATAGGGGGACAAAATTTATTATAAAAAAATATGCTTC
TGGAAATAAAGATAATATTGTTAGAAATAATGATCGTGTATATATTAATG
TAGTAGTTAAAAATAAAGAATATAGGTTAGCTACTAATGCATCACAGGCA
GGCGTAGAAAAAATACTAAGTGCATTAGAAATACCTGATGTAGGAAATCT
AAGTCAAGTAGTAGTAATGAAGTCAAAAAATGATCAAGGAATAACAAATA
AATGCAAAATGAATTTACAAGATAATAATGGGAATGATATAGGCTTTATA
GGATTTCATCAGTTTAATAATATAGCTAAACTAGTAGCAAGTAATTGGTA
TAATAGACAAATAGAAAGATCTAGTAGGACTTTGGGTTGCTCATGGGAAT
TTATTCCTGTAGATGATGGATGGGGAGAAAGGCCACTGTAA

The nucleotide sequence encoding the wild type receptor binding domain (RBD) of BoNT/A1 encompasses nucleotides 2675-3888 of SEQ ID NO:103 and is shown below as SEQ ID NO:104.

CTAGGTATGCATCAAAAATAAATATTGGTAGTAAAGTAAATTTTGATCCA
ATAGATAAAAATCAAATTCAATTATTTAATTTAGAAAGTAGTAAAATTGA
GGTAATTTTAAAAAATGCTATTGTATATAATAGTATGTATGAAAATTTTA
GTACTAGCTTTTGGATAAGAATTCCTAAGTATTTTAACAGTATAAGTCTA
AATAATGAATATACAATAATAAATTGTATGGAAAATAATTCAGGATGGAA
AGTATCACTTAATTATGGTGAAATAATCTGGACTTTACAGGATACTCAGG
AAATAAAACAAAGAGTAGTTTTTAAATACAGTCAAATGATTAATATATCA
GATTATATAAACAGATGGATTTTTGTAACTATCACTAATAATAGATTAAA
TAACTCTAAAATTTATATAAATGGAAGATTAATAGATCAAAAACCAATTT
CAAATTTAGGTAATATTCATGCTAGTAATAATATAATGTTTAAATTAGAT
GGTTGTAGAGATACACATAGATATATTTGGATAAAATATTTTAATCTTTT
TGATAAGGAATTAAATGAAAAAGAAATCAAAGATTTATATGATAATCAAT
CAAATTCAGGTATTTTAAAAGACTTTTGGGGTGATTATTTACAATATGAT
AAACCATACTATATGTTAAATTTATATGATCCAAATAAATATGTCGATGT
AAATAATGTAGGTATTAGAGGTTATATGTATCTTAAAGGGCCTAGAGGTA
GCGTAATGACTACAAACATTTATTTAAATTCAAGTTTGTATAGGGGGACA
AAATTTATTATAAAAAAATATGCTTCTGGAAATAAAGATAATATTGTTAG
AAATAATGATCGTGTATATATTAATGTAGTAGTTAAAAATAAAGAATATA
GGTTAGCTACTAATGCATCACAGGCAGGCGTAGAAAAAATACTAAGTGCA
TTAGAAATACCTGATGTAGGAAATCTAAGTCAAGTAGTAGTAATGAAGTC
AAAAAATGATCAAGGAATAACAAATAAATGCAAAATGAATTTACAAGATA
ATAATGGGAATGATATAGGCTTTATAGGATTTCATCAGTTTAATAATATA
GCTAAACTAGTAGCAAGTAATTGGTATAATAGACAAATAGAAAGATCTAG
TAGGACTTTGGGTTGCTCATGGGAATTTATTCCTGTAGATGATGGATGGG
GAGAAAGGCCACTG

In some embodiments, the isolated nucleic acid molecule of the present disclosure comprises nucleotide sequences modified from the wild-type BoNT nucleic acid molecule, according to the genetic code, to encode propeptide fusions comprising the mutations described herein. Non-limiting examples of such modifications include optimization with respect to codon usage bias of the host used for production of polypeptides, exclusion of unwanted genetic features that affect transcription and translation, and introduction or exclusion of restriction sites. Thus, nucleic acid molecules of the present disclosure may have a nucleic acid sequence quite similar to the wild-type BoNT nucleic acid molecule, at least with respect to the receptor binding domain (RBD). The RBD may be at least about 80%, 81%, 82%, 83%, 84%, 85%, 86%, 87%, 88%, 89%, 90%, 91%, 92%, 93%, 94%, 95%, 96%, 97%, 98%, 99% or more identical to the nucleic acid molecule of SEQ ID NO:104.

The nucleic acid molecules may have other modifications which take into account codon optimization in a host, facile placement of restriction sites, and absence of ambiguous sites elsewhere in the construct, and restricted specificity protease sites designed to ensure that they do not create any internal instability during expression and purification. Other modifications may include, without limitation, a mutation which renders the encoded propeptide resistant to low-specificity proteolysis, one or more silent mutations that inactivate putative internal DNA regulatory elements, and/or one or more unique restriction sites. Fusion protein stability and yield may be optimized by amino acid substitution of residues between the domains of the propeptide, thereby reducing susceptibility to non-specific proteolysis. Also, silent mutations may be introduced into DNA regulatory elements that can affect RNA transcription or expression of the propeptide fusions in the expression system of choice.

In some embodiments, the nucleic acid molecule encodes one or more of the following mutations in the DT-C domain: K₅₁>E, E₁₄₈>K, K₁₂₅>S, R₁₇₃>A and Q₁₈₄>S.

In some embodiments, the nucleic acid molecule encodes one or more of the following mutations in the DT-T domain: Q₂₄₅>S, E₂₉₂>S, and K₂₂₇>S. In some embodiments, the DT-T domain includes a linker comprising the K₃₈₅>G mutation.

Expression levels of DTnd delivery vehicles and cargo molecules may be influenced by the length and/or composition of a specific construct, including but not limited to the number, type, or spacing of detection tags, linkers, protease cleavage sites, or protein fusion tags. In some embodiments, the DTnd delivery vehicle and cargo molecule are expressed separately and attached via an isopeptide bond as discussed infra. In some embodiments, the cargo molecule is expressed as part of the DTnd propeptide fusion protein. In some embodiments, the cargo molecule is positioned upstream of the DT-C domain.

In some embodiments, the isolated nucleic acid molecule of the present disclosure comprises the DTnd propeptide fusion of SEQ ID NO:89 as set forth below (SEQ ID NO:105):

ATGGGTCACCATCATCACCATCATCACCACCACCACCACCACGACGTgTC
AGGTTTCGCTAACGAGCTGGGACCCAGGCTGATGGGCAAGGGAGCTGGTG
AGAACCTGTACTTCCAGGGTAAGCTGGGCTCAATTGAGTTCATCAAGGTT
AACAAAGGCGGTGGCGGTAGCGGCGGTGGCGGTAGCGGCGGTGGCGGATC
CGGTGCTGACGACGTCGTTGATAGCAGCAAGAGCTTCGTTATGGAAAACT
TCAGCAGCTACCACGGCACCAAACCGGGTTACGTGGACAGCATCCAGAAG
GGCATCCAGAAGCCGAAAAGCGGTACCCAGGGCAACTACGACGATGACTG
GGAAGGTTTCTACAGCACCGATAACAAGTACGACGCGGCTGGCTACAGCG
TTGATAACGAGAACCCGCTGAGCGGTAAAGCGGGTGGCGTGGTTAAGGTG
ACCTACCCAGGCCTGACCAAAGTGCTGGCTCTGAAGGTTGACAACGCGGA
AACCATCAAGAAAGAGCTGGGCCTGAGCCTGACCGAACCGCTGATGGAGC
AAGTGGGTACTGAGGAATTCATCAGCCGTTTCGGTGACGGCGCGAGCCGT
GTGGTTCTGAGCCTGCCGTTCGCGGAAGGTAGCAGCAGCGTTAAATACAT
CAACAACTGGGAGCAGGCGAAGGCCTTAAGCGTGGAGCTGGAAATCAACT
TCGAGACGCGTGGCAAGGCTGGCCAGGATGCTATGTACGAGTACATGGCG
AGCGCATGCGCTGGCAACCGTGTGCGTCGTAGCGTTGGTAGCAGCTTGAG
CTGCATCAACCTGGATTGGGACGTTATCCGTGACAAGACCAAAACCAAGA
TCGAAAGCTTGAAAGAGCACGGGCCCATCAGCAACAAGATGAGCGAGAGC
CCGAACAAGACCGTGAGCGAGGAAAAAGCTAAGAGCTACCTGGAGGAGTT
CCACCAGACCGCTCTGGAGCACCCGGAACTGAGCGAGCTGAAGACCGTGA
CCGGTACGAACCCGGTTTTCGCTGGTGCGAACTACGCGGCTTGGGCTGTG
AACGTTGCGCAGGTTATCGATAGCGGCACCGCTGACAACCTGGAGAAAAC
CACCGCGGCTCTGAGCATCCTGCCGGGTATCGGCAGCGTGATGGGCATCG
CTGATGGTGCGGTTCACCACAACACCGAGGAAATCGTGGCTCAGAGCATC
GCGCTGAGCAGCCTGATGGTTGCTCAGGCGATCCCGCTGGTTGGTGAACT
GGTTGACATCGGTTTCGCGGCTTACAACTTCGTGGAGAGCATCATCAACC
TGTTCCAGGTTGTACACAACAGCTACAACCGTCCGGCTTACAGCCCGGGT
CACGGCACCCAGCCGTTCCTGGAGGCGAGCGGTGGACCGGAGGCTAACAT
CATCAACACCTCCATCCTGAACCTCCGTTACGAGTCTAACCACCTCATCG
ACTTGAGCAGATACGCTAGCAAGATCAACATCGGTTCCAAGGTGAACTTC
GACCCAATCGATAAGAACCAGATCCAACTGTTCAACCTCGAATCCTCTAA
GATCGAAGTGATCCTGAAGAACGCTATCGTCTACAACTCCATGTACGAGA
ACTTCTCTACCAGCTTCTGGATCAGGATTCCGAAATACTTCAACTCAATC
TCGCTCAACAACGAGTACACTATCATCAACTGCATGGAAAACAACTCGGG
ATGGAAGGTGTCCCTCAACTACGGCGAGATCATCTGGACTTTGCAGGACA
CACAAGAAATCAAGCAGAGGGTCGTGTTCAAGTACAGCCAAATGATCAAC
ATCAGCGATTACATCAACCGTTGGATCTTCGTCACAATCACCAACAACCG
CCTGAACAACTCCAAGATTTACATCAACGGTAGACTGATCGACCAGAAGC
CAATCAGCAACCTCGGCAACATCCACGCCTCAAACAACATCATGTTCAAG
TTGGACGGCTGTAGGGATACACACAGATACATCTGGATCAAATACTTCAA
CCTGTTCGACAAGGAGCTCAACGAGAAGGAAATCAAGGACCTCTACGATA
ACCAGTCCAACTCTGGTATCTTGAAGGACTTCTGGGGCGATTACCTGCAA
TACGACAAGCCCTACTACATGTTGAACCTGTACGACCCTAACAAGTACGT
TGATGTGAACAACGTCGGTATCAGGGGCTACATGTACCTGAAGGGACCAC
GTGGTTCTGTTATGACCACTAACATCTACCTCAACAGCTCATTGTACCGT
GGCACAAAGTTCATCATCAAGAAGTACGCCTCCGGAAACAAGGACAACAT
CGTCCGTAACAACGATCGCGTTTACATCAACGTTGTGGTCAAGAACAAGG
AGTACAGACTGGCTACCAACGCTTCGCAGGCTGGAGTTGAGAAGATCCTG
TCTGCTCTGGAAATCCCTGACGTGGGCAACCTCTCACAGGTTGTGGTCAT
GAAGTCGAAGAACGATCAAGGCATCACTAACAAGTGCAAGATGAACTTGC
AGGACAACAACGGAAACGACATCGGCTTCATCGGATTCCACCAATTCAAC
AACATCGCCAAGTTGGTGGCCAGCAACTGGTACAACCGTCAGATCGAGCG
TTCGTCCCGCACCTTAGGATGCTCGTGGGAGTTCATTCCAGTCGATGACG
GATGGGGAGAGAGACCTTTGGGCGCAGGAGAGAACCTGTACTTCCAGGGT
GCAGGATGGTCCCACCCACAATTCGAGAAGGGTGCAGGATGGAGTCACCC
ACAGTTCGAGAAGTAA

In some embodiments, the isolated nucleic acid molecule of the present disclosure comprises the MBP-B8 propeptide fusion of SEQ ID NO:91 as set forth below (SEQ ID NO:106):

ATGAAAATCGAAGAAGGTAAACTGGTAATCTGGATTAACGGCGATAAAGG
CTATAACGGTCTCGCTGAAGTCGGTAAGAAATTCGAGAAAGATACCGGAA
TTAAAGTCACCGTTGAGCATCCGGATAAACTGGAAGAGAAATTCCCACAG
GTTGCGGCAACTGGCGATGGCCCTGACATTATCTTCTGGGCACACGACCG
CTTTGGTGGCTACGCTCAATCTGGCCTGTTGGCTGAAATCACCCCGGACA
AAGCGTTCCAGGACAAGCTGTATCCGTTTACCTGGGATGCCGTACGTTAC
AACGGCAAGCTGATTGCTTACCCGATCGCTGTTGAAGCGTTATCGCTGAT
TTATAACAAAGATCTGCTGCCGAACCCGCCAAAAACCTGGGAAGAGATCC
CGGCGCTGGATAAAGAACTGAAAGCGAAAGGTAAGAGCGCGCTGATGTTC
AACCTGCAAGAACCGTACTTCACCTGGCCGCTGATTGCTGCTGACGGGGG
TTATGCGTTCAAGTATGAAAACGGCAAGTACGACATTAAAGACGTGGGCG
TGGATAACGCTGGCGCGAAAGCGGGTCTGACCTTCCTGGTTGACCTGATT
AAAAACAAACACATGAATGCAGACACCGATTACTCCATCGCAGAAGCTGC
CTTTAATAAAGGCGAAACAGCGATGACCATCAACGGCCCGTGGGCATGGT
CCAACATCGACACCAGCAAAGTGAATTATGGTGTAACGGTACTGCCGACC
TTCAAGGGTCAACCATCCAAACCGTTCGTTGGCGTGCTGAGCGCAGGTAT
TAACGCCGCCAGTCCGAACAAAGAGCTGGCAAAAGAGTTCCTCGAAAACT
ATCTGCTGACTGATGAAGGTCTGGAAGCGGTTAATAAAGACAAACCGCTG
GGTGCCGTAGCGCTGAAGTCTTACGAGGAAGAGTTGGTGAAAGATCCGCG
TATTGCCGCCACTATGGAAAACGCCCAGAAAGGTGAAATCATGCCGAACA
TCCCGCAGATGTCCGCTTTCTGGTATGCCGTGCGTACTGCGGTGATCAAC
GCCGCCAGCGGTCGTCAGACTGTCGATGAAGCCCTGAAAGACGCGCAGAC
TAATTCGAGCTCGGGTAGCGGCGGTGGCGGTAGCGGTGCGGGCGAGAATC
TGTACTTCCAGTCCGGTTCGAACGGCGGGGCCGGCCAGTCGGGCGCCGGC
GAAGGCGGTGGCGGTAGCGGCGGTGGCGGTAGCGGCGGTGGCGGATCCCA
GGCGCACGTGCAACTGCAGCAGTCTGGAGGAGGCTTGGTGCAGCCTGGGG
GTTCCCTGCGCCTGTCATGTGCAGCCTCTGGAAGCATCTTCAGTATTTAC
GCTATGGGCTGGTACAGGCAGGCTCCTGGCAAGCAACGTGAACTGGTTGC
TGCCATCTCCAGCTACGGTAGTACCAACTACGCTGATTCGGTCAAGGGCA
GGTTCACCATCTCCCGCGACAATGCCAAGAATACCGTCTATTTGCAAATG
AACTCTCTGAAACCTGAGGATACGGCCGTCTACTACTGCAACGCTGACAT
TGCTACTATGACCGCGGTAGGCGGATTCGACTACTGGGGACAGGGAACTC
AGGTGACGGTCTCTTCCGCGCACCACTCCGAGGACCCTACTTCTCAAAGC
ACTAGTGGTGGCGGTGGCAGCGGTGGCGGTGGCAGCGGTGGCGGTGGCAG
CGGCGGAGGTGGCAGTGGGACGTCCGGGTCGACAGGGGGTGGCGGTAGCG
GTGGCGGTGGCAGCGGTGCAGGAGATATCCCGGCGACCTACGAGTTCACC
GACGGCAAGCACTACATCACCAACGAACCGATCCCGCCGAAAGGTGCAGG
AGAGAATCTGTACTTCCAGGGCCACCACCACCACCACCACTAA

In some embodiments, the isolated nucleic acid molecule of the present disclosure comprises the MBP-JSG-C1 propeptide fusion of SEQ ID NO:93 as set forth below (SEQ ID NO:107):

ATGAAAATCGAAGAAGGTAAACTGGTAATCTGGATTAACGGCGATAAAGG
CTATAACGGTCTCGCTGAAGTCGGTAAGAAATTCGAGAAAGATACCGGAA
TTAAAGTCACCGTTGAGCATCCGGATAAACTGGAAGAGAAATTCCCACAG
GTTGCGGCAACTGGCGATGGCCCTGACATTATCTTCTGGGCACACGACCG
CTTTGGTGGCTACGCTCAATCTGGCCTGTTGGCTGAAATCACCCCGGACA
AAGCGTTCCAGGACAAGCTGTATCCGTTTACCTGGGATGCCGTACGTTAC
AACGGCAAGCTGATTGCTTACCCGATCGCTGTTGAAGCGTTATCGCTGAT
TTATAACAAAGATCTGCTGCCGAACCCGCCAAAAACCTGGGAAGAGATCC
CGGCGCTGGATAAAGAACTGAAAGCGAAAGGTAAGAGCGCGCTGATGTTC
AACCTGCAAGAACCGTACTTCACCTGGCCGCTGATTGCTGCTGACGGGGG
TTATGCGTTCAAGTATGAAAACGGCAAGTACGACATTAAAGACGTGGGCG
TGGATAACGCTGGCGCGAAAGCGGGTCTGACCTTCCTGGTTGACCTGATT
AAAAACAAACACATGAATGCAGACACCGATTACTCCATCGCAGAAGCTGC
CTTTAATAAAGGCGAAACAGCGATGACCATCAACGGCCCGTGGGCATGGT
CCAACATCGACACCAGCAAAGTGAATTATGGTGTAACGGTACTGCCGACC
TTCAAGGGTCAACCATCCAAACCGTTCGTTGGCGTGCTGAGCGCAGGTAT
TAACGCCGCCAGTCCGAACAAAGAGCTGGCAAAAGAGTTCCTCGAAAACT
ATCTGCTGACTGATGAAGGTCTGGAAGCGGTTAATAAAGACAAACCGCTG
GGTGCCGTAGCGCTGAAGTCTTACGAGGAAGAGTTGGTGAAAGATCCGCG
TATTGCCGCCACTATGGAAAACGCCCAGAAAGGTGAAATCATGCCGAACA
TCCCGCAGATGTCCGCTTTCTGGTATGCCGTGCGTACTGCGGTGATCAAC
GCCGCCAGCGGTCGTCAGACTGTCGATGAAGCCCTGAAAGACGCGCAGAC
TAATTCGAGCTCGGGTAGCGGCGGTGGCGGTAGCGGTGCGGGCGAGAATC
TGTACTTCCAGTCCGGTTCGAACGGCGGGGCCGGCCAGTCGGGCGCCGGC
GAAGGCGGTGGCGGTAGCGGCGGTGGCGGTAGCGGCGGTGGCGGATCCGC
GGGGACGTCCCAGGTGCAGCTGGTTGAGAGCGGTGGCGGTCTGGTGCAGA
CCGGCGGTAGCCTGCGTCTGAGCTGCGCTGCTAGCGGTCGTACCTTCCGT
CGTAACACCATGGGTTGGTTCCGTCAGGCTCCGGGTAAAGTGCGTGAATT
CGTTGCGGCTATCAGCTGGAGCGGCGACCGTACCTACTGCGCTGATAGCG
TGAAGGGTCGTTTCACCATCAGCCGTGACAACGCTAAAAACACCGTTGAT
CTGCTGATGAACAGCCTGAAGCCGGAGGACACCGCGATCTACTACTGCGC
GGCTGATGGTACCGCTAGCGTTTTCAACAGCTACGCGAGCGCTGACCGTA
ACAAGTACAACTACTGGGGCCAGGGTACCCAGGTGACCGTTAGCAGCGGG
TCGACAGCCACTAGTGGTGGCGGTGGCAGCGGTGGCGGTGGCAGCGGTGG
CGGTGGCAGCGGCGGAGGTGGCAGTGGGACGTCCGGGTCGACAGGGGGTG
GCGGTAGCGGTGGCGGTGGCAGCGGTGCAGGAGATATCCCGGCGACCTAC
GAGTTCACCGACGGCAAGCACTACATCACCAACGAACCGATCCCGCCGAA
AGGTGCAGGAGAGAATCTGTACTTCCAGGGCCACCACCACCACCACCACT
AA

Further aspects of the present disclosure relate to an expression system comprising the nucleic acid molecule of the present disclosure in a heterologous vector and a host cell comprising the nucleic acid molecule of the present disclosure.

Yet another aspect of the present disclosure relates to a method of expressing a fusion protein. This method involves providing a nucleic acid construct comprising a nucleic acid molecule encoding the propeptide fusion of the present disclosure; a heterologous promoter operably linked to the nucleic acid molecule; and a 3′ regulatory region operably linked to the nucleic acid molecule. The nucleic acid construct is introduced into a host cell under conditions effective to express a propeptide of the fusion protein.

In some embodiments, the expression system comprises the nucleic acid molecule of the present disclosure in a heterologous vector.

Suitable expression systems and host cells for expressing the fusion protein are described in U.S. Pat. No. 7,785,606 to Ichtchenko and Band, which is hereby incorporated by reference in its entirety.

In some embodiments, the nucleic acid molecules of the present disclosure are capable of being expressed. Expression of a fusion protein described herein can be carried out by introducing a nucleic acid molecule described herein into an expression system of choice using conventional recombinant technology. Generally, this involves inserting the nucleic acid molecule into an expression system to which the molecule is heterologous (i.e., not normally present). The introduction of a particular foreign or native gene into a mammalian host is facilitated by first introducing the gene sequence into a suitable nucleic acid vector. “Vector” is used herein to mean any genetic element, such as a plasmid, phage, transposon, cosmid, chromosome, virus, virion, etc., which is capable of replication when associated with the proper control elements and which is capable of transferring gene sequences between cells. Thus, the term includes cloning and expression vectors, as well as viral vectors. The heterologous nucleic acid molecule is inserted into the expression system or vector in proper sense (5→3′) orientation and correct reading frame. The vector contains the necessary elements for the transcription and translation of the inserted propeptide fusion-coding sequences.

In general, expression vectors useful in recombinant DNA techniques are often in the form of plasmids. However, the present application is intended to include such other forms of expression vectors that are not technically plasmids, such as viral vectors, e.g., replication defective retroviruses, adenoviruses and adeno-associated viruses, which serve equivalent functions. Such viral vectors permit infection of a subject and expression in that subject of a compound. The expression control sequences are typically eukaryotic promoter systems in vectors capable of transforming or transfecting eukaryotic host cells. Once the vector has been incorporated into the appropriate host, the host is maintained under conditions suitable for high level expression of the nucleotide sequences encoding the target domain, and the collection and purification of the substrate binding agent, e.g., cross-reacting anti-substrate antibodies. See, generally, U.S. Patent Publication No. 2002/0199213, which is hereby incorporated by reference in its entirety. Vectors can also encode signal peptide, e.g., pectate lyase, useful to direct the secretion of extracellular antibody fragments. See U.S. Pat. No. 5,576,195, which is hereby incorporated by reference in its entirety.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

Recombinant genes may also be introduced into viruses, including vaccinia virus, adenovirus, and retroviruses, including lentivirus. Recombinant viruses can be generated by transfection of plasmids into cells infected with virus.

Suitable vectors include, but are not limited to, the following viral vectors such as lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK+/− or KS+/− (see “Stratagene Cloning Systems” Catalog (1993) from Stratagene, La Jolla, CA, which is hereby incorporated by reference in its entirety), pQE, pIH821, pGEX, pFastBac series (Invitrogen), pET series (Studier et. al., “Use of T7 RNA Polymerase to Direct Expression of Cloned Genes,” Gene Expression Technology Vol. 185 (1990), which is hereby incorporated by reference in its entirety), and any derivatives thereof. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Cold Springs Laboratory, Cold Springs Harbor, New York (1989), which is hereby incorporated by reference in its entirety.

In some embodiments, a host cell comprises the nucleic acid molecule of the present disclosure. In some embodiments, the nucleic acid molecule is inserted into a heterologous expression system. A variety of host-vector systems may be utilized to express the propeptide fusion-encoding sequence in a cell. Primarily, the vector system must be compatible with the host cell used. Host-vector systems include, but are not limited to, the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (“mRNA”) translation).

Transcription of DNA is dependent upon the presence of a promoter which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression see Roberts and Lauer, Methods in Enzymology 68:473 (1979), which is hereby incorporated by reference in its entirety.

Promoters vary in their “strength” (i.e., their ability to promote transcription). For the purposes of expressing a cloned gene, it is desirable to use strong promoters to obtain a high level of transcription and, hence, expression of the gene. Depending upon the host cell system utilized, any one of a number of suitable promoters may be used. For instance, when cloning in E. coli, its bacteriophages, or plasmids, promoters such as the PH promoter, T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires a Shine-Dalgarno (SD) sequence about 7-9 bases 5′ to the initiation codon (ATG) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B, or A genes. Additionally, any SD-ATG combination produced by recombinant DNA or other techniques involving incorporation of synthetic nucleotides may be used.

Depending on the vector system and host utilized, any number of suitable transcription and/or translation elements, including constitutive, inducible, and repressible promoters, as well as minimal 5′ promoter elements may be used.

The propeptide fusion-encoding nucleic acid, a promoter molecule of choice, a suitable 3′ regulatory region, and if desired, a reporter gene, are incorporated into a vector-expression system of choice to prepare a nucleic acid construct using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety.

The nucleic acid molecule encoding a propeptide fusion is inserted into a vector in the sense (i.e., 5→3′) direction, such that the open reading frame is properly oriented for the expression of the encoded propeptide fusion under the control of a promoter of choice. Single or multiple nucleic acids may be ligated into an appropriate vector in this way, under the control of a suitable promoter, to prepare a nucleic acid construct.

Once the isolated nucleic acid molecule encoding the propeptide fusion has been inserted into an expression vector, it is ready to be incorporated into a host cell. Recombinant molecules can be introduced into cells via transformation, particularly transduction, conjugation, lipofection, protoplast fusion, mobilization, particle bombardment, or electroporation. The nucleic acid sequences are incorporated into the host cell using standard cloning procedures known in the art, as described by Sambrook et al., Molecular Cloning: A Laboratory Manual, Second Edition, Cold Springs Laboratory, Cold Springs Harbor, New York (1989), which is hereby incorporated by reference in its entirety. Suitable hosts include, but are not limited to, bacteria, virus, yeast, fungi, mammalian cells, insect cells, plant cells, and the like. In some embodiments, the host cell is selected from the group consisting of a plant cell, mammalian cell, insect cell, yeast cell, and bacterial cell. In one embodiment, the host cells of the present disclosure include, but are not limited to, Escherichia coli, insect cells, and Pichia pastoris cells. In some embodiments, the first protease cleavage site is not cleavable by proteases endogenous to the host cell. In some embodiments, the first, second, or third protease site of the propeptide fusion is not cleavable by the proteases endogenous to the expression system of the host cell.

Typically, an antibiotic or other compound useful for selective growth of the transformed cells only is added as a supplement to the media. The compound to be used will be dictated by the selectable marker element present in the plasmid with which the host cell was transformed. Suitable genes are those which confer resistance to gentamycin, G418, hygromycin, puromycin, streptomycin, spectinomycin, tetracycline, chloramphenicol, and the like. Similarly, “reporter genes” which encode enzymes providing for production of an identifiable compound, or other markers which indicate relevant information regarding the outcome of gene delivery, are suitable. For example, various luminescent or phosphorescent reporter genes are also appropriate, such that the presence of the heterologous gene may be ascertained visually.

### Processing of Propeptide Fusion Proteins & Cargo Attachment

Another aspect of the present disclosure relates to a method of attaching a cargo polypeptide to a fusion protein. This method involves (i) a cargo protein comprising a first member of a peptide fusion tag binding pair and (ii) a DTnd fusion protein as described herein comprising a second member of a peptide fusion tag binding pair with (iii) a biotinylated SnoopLigase to form a complex; capturing the complex on a streptavidin matrix to immobilize the complex; and eluting the cargo protein attached to the fusion protein.

As disclosed herein, a cargo molecule may be attached to a DTnd delivery vehicle via an isopeptide bond. In some embodiments, the isopeptide bond is formed by protein fusion using members of a peptide fusion tag binding pair. In some embodiments, cargo molecule and the DTnd delivery vehicle each comprise a member of a peptide fusion tag binding pair.

A “peptide fusion tag binding pair” includes two members (e.g., a first peptide fusion tag) and a second cognate member (e.g., a second peptide fusion tag)) that interact to form a bond (e.g., a covalent bond between, e.g., proteins capable of forming isopeptide bonds). In some embodiments, the term “cognate” refers to components that function together. Thus, two proteins that react together efficiently to form an isopeptide bond under conditions that enable or facilitate isopeptide bond formation can also be referred to as being a “complementary” pair of proteins.

Specific peptide fusion tag binding pairs capable of interacting to form a covalent isopeptide bond are reviewed in Veggiani et al., Trends Biotechnol. 32:506 (2014), which is hereby incorporated by reference in its entirety. The first member and second cognate member of a peptide fusion tag binding pair can be a system such as SpyTag:SpyCatcher, SpyTag002:SpyCatcher002, SpyTag003:SpyCatcher003, SpyTag:KTag; Isopeptag:Pilin-C, Isopeptag:Pilin-N, SnoopTag:SnoopCatcher, SnoopTagJr:SnoopCatcher, SnoopTagJr:DogTag, DogTag:DogCatcher, SdyTag:SdyCatcher, Jo:In, 3kptTag:3kptCatcher, 4oq1Tag:4oq1Catcher, NGTag:NGCatcher, Rumtrunk:Mooncake, Snoop ligase, GalacTag, Cpe, Ececo, Corio, etc., and variants thereof. SpyTag002:SpyCatcher002 and SpyTag003:SpyCatcher003 are different iterations of SpyTag:SpyCatcher.

The term “isopeptide bond” refers to an amide bond between a carboxyl or carboxamide group and an amino group at least one of which is not derived from a protein main chain or alternatively viewed is not part of the protein backbone. An isopeptide bond may form within a single protein or may occur between two peptides or a peptide and a protein. Thus, an isopeptide bond may form intramolecularly within a single protein or intermolecularly i.e., between two peptide/protein molecules, e.g., between two peptide linkers. Typically, an isopeptide bond may occur between a lysine residue and an asparagine, aspartic acid, glutamine, or glutamic acid residue or the terminal carboxyl group of the protein or peptide chain or may occur between the alpha-amino terminus of the protein or peptide chain and an asparagine, aspartic acid, glutamine, or glutamic acid. Each residue of the pair involved in the isopeptide bond is referred to herein as a reactive residue. In preferred embodiments, an isopeptide bond may form between a lysine residue and an asparagine residue or between a lysine residue and an aspartic acid residue. Particularly, isopeptide bonds can occur between the side chain amine of lysine and carboxamide group of asparagine or carboxyl group of an aspartate.

The SnoopTag:SnoopCatcher system is described in Veggiani, PNAS 113:1202-07 (2016), which is hereby incorporated by reference in its entirety. The D4 Ig-like domain of RrgA, an adhesion from Streptococcus pneumoniae, was split to form SnoopTag (residues 734-745; KLGDIEFIKVNK (SEQ ID NO:108)) and SnoopCatcher (residues 749-860; MGSSHHHHHHSSGLVPRGSHMKPLRGAVFSLQKQHPDYPDIYGAIDQNGTYQNVRTGEDGKLTFK NLSDGKYRLFENSEPAGYKPVQNKPIVAFQIVNGEVRDVTSIVPQDIPATYEFTNGKHYITNEPI PPK (SEQ ID NO:109)). Incubation of SnoopTag and SnoopCatcher results in a spontaneous isopeptide bond that is specific between the complementary proteins.

In some embodiments, the specific peptide fusion tag binding pair is a SpyTag:SpyCatcher binding pair, wherein the first member is SpyTag, and wherein the second cognate member is SpyCatcher. In some embodiments, the specific peptide fusion tag binding pair is a SpyTag002:SpyCatcher002 binding pair, wherein the first member is SpyTag002, and wherein the second cognate member is SpyCatcher002. In some embodiments, the specific peptide fusion tag binding pair is a SpyTag003:SpyCatcher003 binding pair, wherein the first member is SpyTag003, and wherein the second cognate member is SpyCatcher003. In some embodiments, the specific peptide fusion tag binding pair is SpyTag:KTag, wherein the first member is SpyTag and wherein the second cognate member is KTag. In some embodiments, the specific peptide fusion tag binding pair is SpyTag:KTag, wherein the first member is KTag and wherein the second cognate member is SpyTag. In some embodiments, the specific peptide fusion tag binding pair is Isopeptag:Pilin-C, wherein the first member is Isopeptag, and wherein the second cognate member is Pilin-C, or a portion thereof. In some embodiments, the specific peptide fusion tag binding pair is Isopeptag:Pilin-N, wherein the first member is Isopeptag, and wherein the second cognate member is Pilin-N, or a portion thereof. In some embodiments, the specific peptide fusion tag binding pair is SnoopTag:SnoopCatcher, and the first member is SnoopTag, and the second cognate member is SnoopCatcher. In some embodiments, the specific peptide fusion tag binding pair is SnoopTagJr:SnoopCatcher, and the first member is SnoopTagJr, and the second cognate member is SnoopCatcher. In some embodiments, the specific peptide fusion tag binding pair is DogTag:DogCatcher, and the first member is DogTag, and the second cognate member is DogCatcher. In some embodiments, the specific peptide fusion tag binding pair is SnoopTagJr:DogTag, and the first member is SnoopTagJr, and the second cognate member is DogTag.

In some embodiments, the cargo molecule comprises the first member of the peptide fusion tag binding pair selected from SpyTag, Isopeptag, SnoopTag, SpyTag002, SpyTag003, DogTag, SnoopTagJr, or any biologically active portions or variants thereof. In some embodiments, the DTnd delivery vehicle comprises the second member of the peptide fusion tag binding pair selected from SpyCatcher, KTag, Pilin-C, Pilin-N, SnoopCatcher, SpyCatcher002, SpyCatcher003, DogCatcher, SnoopTagJr, DogTag, or any biologically active portions or variants thereof. In some embodiments, the DTnd delivery vehicle comprises the first member of the peptide fusion tag binding pair selected from SpyTag, Isopeptag, SnoopTag, SpyTag002, SpyTag003, DogTag, SnoopTagJr, or any biologically active portions or variants thereof. In some embodiments, the cargo molecule comprises the second member of the peptide fusion tag binding pair selected from SpyCatcher, KTag, Pilin-C, Pilin-N, SnoopCatcher, SpyCatcher002, SpyCatcher003, DogCatcher, SnoopTagJr, DogTag, or any biologically active portions or variants thereof.

In some embodiments, the peptide fusion tag binding pair is recognized by SnoopLigase. In some embodiments, the first member of the peptide fusion tag binding pair comprises DogTag (DIPATYEFTDGKHYITNEPIPPK; SEQ ID NO:88), and the second member of the peptide fusion tag binding pair comprises. SnoopTagJr (KLGSIEFIKVNK; SEQ ID NO:87). In some embodiments, the first member of the peptide fusion tag binding pair comprises SnoopTagJr (KLGSIEFIKVNK; SEQ ID NO:87), and the second member of the peptide fusion tag binding pair comprises DogTag (DIPATYEFTDGKHYITNEPIPPK; SEQ ID NO:88).

In some embodiments, protein fusion to produce an isopeptide bond comprises a SnoopLigase. SnoopLigase catalyzes the isopeptide bond between, e.g., SnoopTagJr and DogTag. SnoopLigase is described in Buldun et al., “SnoopLigase Catalyzes Peptide—Peptide Locking and Enables Solid-Phase Conjugate Isolation,” J. Am. Chem. Soc. 140:3008-3018 (2018) and Andersson et al., “SnoopLigase Peptide—Peptide Conjugation Enables Modular Vaccine Assembly,” Nature Scientific Reports 9:4625 (2019), which are hereby incorporated by reference in their entirety.

The amino acid sequence of SnoopLigase is set forth as SEQ ID NO:110, as follows:

VNKNDKKPLRGAVESLQKQHPDYPDIYGAIDQNGTYQNVRTGEDGKLTFK
NLSDGKYRLFENSEPPGYKPVQNKPIVAFQIVNGEVRDVTSIVPPGVPAT
YEFT

In some embodiments, the SnoopLigase is expressed as a propeptide fusion comprising one or more detection tags, one or more linker sequences, and/or one or more protease cleavage sites. In some embodiments, the SnoopLigase fusion protein comprises one or more biotinylation signals.

In some embodiments, the SnoopLigase fusion protein comprises an Avi-tag. The Avi tag is a 15 aa sequence (GLNDIFEAQKIEWHE; SEQ ID NO:79) comprising a lysine residue (indicated in bold text) that can be enzymatically biotinylated in celullo and/or in vitro. When biotinylated, the Avi tag allows the SnoopLigase fusion protein to be immobilized on a streptavidin matrix. In some embodiments, the Avi tag is an immobilization sequence. In some embodiments, the SnoopLigase fusion protein comprises a HaloTag7 sequence. The HaloTag7 sequence (SEQ ID NO:78) is a modified haloalkane dehalogenase that can be immobilized in a halogenated solid support such as Halolink® matrix (Promega). In some embodiments, the SnoopLigase fusion protein comprises a protease cleavage site positioned between the HaloTag7 and the Avi-tag.

In some embodiments, the SnoopLigase fusion protein is a HalobtnSnoopLigase that comprises the amino acid sequence as set forth below (SEQ ID NO:111):

MGSSHHHHHHSSGEIGTGFPFDPHYVEVLGERMHYVDVGPRDGTPVLFLH
GNPTSSYVWRNIIPHVAPTHRCIAPDLIGMGKSDKPDLGYFEDDHVREMD
AFIEALGLEEVVLVIHDWGSALGFHWAKRNPERVKGIAFMEFIRPIPTWD
EWPEFARETFQAFRTTDVGRKLIIDQNVFIEGTLPMGVVRPLTEVEMDHY
REPFLNPVDREPLWRFPNELPIAGEPANIVALVEEYMDWLHQSPVPKLLF
WGTPGVLIPPAEAARLAKSLPNCKAVDIGPGLNLLQEDNPDLIGSEIARW
LSTLEISGGGGGSGGGGASGENLYFQGGLNDIFEAQKIEWHEGGGGSGGG
GSGGGGSVNKNDKKPLRGAVESLQKQHPDYPDIYGAIDQNGTYQNVRTGE
DGKLTFKNLSDGKYRLFENSEPPGYKPVQNKPIVAFQIVNGEVRDVTSIV
PPGVPATYEFTGAGWSHPQFEKGAGWSHPQFEK

In some embodiments, the SnoopLigase fusion protein is a btnSnoopLigase that comprises the amino acid sequence as set forth below (SEQ ID NO:112):

GGLNDIFEAQKIEWHEGGGGSGGGGSGGGGSVNKNDKKPLRGAVESLQKQ
HPDYPDIYGAIDQNGTYQNVRTGEDGKLTFKNLSDGKYRLFENSEPPGYK
PVQNKPIVAFQIVNGEVRDVTSIVPPGVPATYEFTGAGWSHPQFEKGAGW
SHPQFEK

BtnSnoopLigase is produced by cleaving the TEV site on HalobtnSnoopLigase.

In some embodiments, the isolated nucleic acid molecule of the present disclosure comprises the Halo-btnSnoopLigase propeptide fusion of SEQ ID NO:111 as set forth below (SEQ ID NO:113):

ATGGGTAGCAGCCATCACCACCACCACCACAGCAGCGGTGAAATCGGTAC
CGGCTTCCCGTTTGACCCGCACTACGTTGAGGTGCTGGGTGAACGTATGC
ACTATGTGGACGTTGGTCCGCGTGATGGCACCCCGGTGCTGTTCCTGCAC
GGCAACCCGACCAGCAGCTACGTGTGGCGTAACATCATTCCGCATGTTGC
GCCGACCCACCGTTGCATTGCGCCGGATCTGATTGGTATGGGCAAGAGCG
ACAAACCGGATCTGGGTTATTTCTTTGACGATCACGTGCGTTTCATGGAC
GCGTTTATCGAGGCGCTGGGCCTGGAGGAAGTGGTTCTGGTTATTCACGA
TTGGGGTAGCGCGCTGGGCTTTCACTGGGCGAAGCGTAACCCGGAGCGTG
TTAAAGGTATCGCGTTCATGGAATTTATCCGTCCGATTCCGACCTGGGAC
GAGTGGCCGGAATTCGCGCGTGAAACCTTCCAGGCGTTTCGTACCACCGA
CGTGGGCCGTAAGCTGATCATCGATCAAAACGTTTTCATTGAGGGTACCC
TGCCGATGGGCGTGGTTCGTCCGCTGACCGAGGTGGAAATGGACCACTAC
CGTGAGCCGTTCCTGAACCCGGTTGATCGTGAACCGCTGTGGCGTTTTCC
GAACGAGCTGCCGATCGCGGGTGAACCGGCGAACATTGTGGCGCTGGTTG
AGGAATATATGGACTGGCTGCACCAGAGCCCGGTGCCGAAACTGCTGTTC
TGGGGTACCCCGGGCGTTCTGATCCCGCCGGCGGAAGCGGCGCGTCTGGC
GAAGAGCCTGCCGAACTGCAAAGCGGTGGATATTGGTCCGGGCCTGAACC
TGCTGCAAGAGGACAACCCGGATCTGATCGGTAGCGAAATTGCGCGTTGG
CTGAGCACCCTGGAGATCAGCGGTGGTGGAGGCGGAAGCGGCGGTGGCGG
AGCTAGCGGTGAGAACCTCTACTTCCAGGGTGGACTGAACGATATTTTCG
AAGCGCAAAAGATCGAATGGCACGAGGGTGGAGGCGGAAGCGGCGGTGGC
GGAAGCGGTGGTGGCGGATCCGTGAACAAGAACGACAAGAAACCGCTGCG
TGGTGCGGTTTTCAGCCTGCAGAAACAACACCCGGACTACCCGGATATCT
ATGGTGCGATTGATCAGAACGGCACCTACCAAAACGTGCGTACCGGCGAG
GACGGCAAGCTGACCTTCAAAAACCTGAGCGATGGCAAGTACCGTCTGTT
TGAGAACAGCGAACCGCCGGGCTATAAGCCGGTTCAGAACAAACCGATCG
TGGCGTTCCAAATTGTTAACGGTGAAGTGCGTGACGTTACCAGCATTGTG
CCGCCGGGCGTTCCGGCGACCTATGAATTTACCGGTGCGGGTTGGAGCCA
CCCGCAGTTTGAAAAGGGTGCGGGCTGGAGCCACCCGCAATTTGAGAAAT
AA

In some embodiments, the SnoopLigase fusion protein is biotinylated when expressed in a bacteria. In some embodiments, the SnoopLigase fusion protein is biotinylated in vitro. In some embodiments, the SnoopLigase fusion protein comprises a long flexible linker and a short immobilization sequence. In some embodiments, the long flexible linker is at least 8, 9, 10, 11, 12, 13, 14, or 15 amino acids. In some embodiments, the long flexible linker is at least 15 amino acids. In some embodiments the long flexible linker is any one of SEQ ID NOs:54-72. In some embodiments, the long flexible linker comprises one or more repeats of any one of SEQ ID NOs:54-72 and combinations thereof.

In some embodiments, the flexible linker and short immobilization sequence are at the N-terminus. In some embodiments, the flexible linker and short immobilization sequence are at the C-terminus. In some embodiments, the SnoopLigase fusion protein comprises a terminal cysteine for covalent immobilization on a resin via sulfhydryl coupling.

In some embodiments, the present disclosure relates to a method of attaching a therapeutic cargo and a DTnd fusion protein. This method involves forming an isopeptide bond between a therapeutic cargo protein comprising a first member of a peptide fusion tag binding pair and a DTnd fusion protein comprising a second member of a peptide fusion tag binding pair, wherein said attaching is carried out with SnoopLigase to form the attachment.

In some embodiments, the propeptide fusions or fusion proteins of the present disclosure are isolated and purified prior to formation of the isopeptide bond. In some embodiments, the SnoopLigase is isolated and purified. In some embodiments, the SnoopLigase is biotinylated.

Propeptide fusions and fusion proteins of the present disclosure may be isolated and purified by standard methods including, but not limited to, chromatography (e.g., ion exchange, affinity, size exclusion, and hydroxyapatite chromatography), gel filtration, centrifugation, or differential solubility, ethanol precipitation or by any other available technique for the purification of proteins. See, e.g., Scopes, “Protein Purification Principles and Practice” 2nd Edition, Springer-Verlag, New York (1987); Higgins, S. J. and Hames, B. D. (eds.), “Protein Expression: A Practical Approach”, Oxford Univ Press, (1999); and Deutscher, M. P et al., (eds.), “Guide to Protein Purification: Methods in Enzymology” Methods in Enzymology Series, Vol 182, Academic Press (1997), which are each hereby incorporated by reference in its entirety. For immunoaffinity chromatography in particular, the protein may be isolated by binding it to an affinity column comprising antibodies that were raised against that protein and were affixed to a stationary support. Alternatively, affinity purification tags such as an influenza coat sequence, poly-histidine, or glutathione-S-transferase can be attached to the protein by standard recombinant techniques to allow for easy purification by passage over the appropriate affinity column.

Protease inhibitors such as phenyl methyl sulfonyl fluoride (PMSF), leupeptin, pepstatin or aprotinin may be added at any or all stages in order to reduce or eliminate degradation of the polypeptide or protein during the purification process. Protease inhibitors are particularly desired when cells must be lysed in order to isolate and purify the expressed polypeptide or protein.

A peptide fusion or portion thereof can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, mixed mode chromatography (e.g., MEP Hypercel™), hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y. (1997-2003), which is hereby incorporated by reference in its entirety.

Exemplary technology platforms based on recombinant clostridial constructs that may also be employed with the DTnd delivery vehicle constructs of the present disclosure include a baculovirus expression system as described in U.S. Pat. No. 7,785,606 to Ichtchenko and Band, which is hereby incorporated by reference in its entirety. This platform allows the tools of modern molecular biology to be applied to bioengineering of recombinant botulinum neurotoxins that retain the structure and trafficking properties of the native toxin (Band et al., “Recombinant Derivatives of Botulinum Neurotoxin A Engineered for Trafficking Studies and Neuronal Delivery,” Protein Expr. Purif. 71:62-73 (2010) and Vazquez-Cintron et al., “Engineering Botulinum Neurotoxin C1 as a Molecular Vehicle for Intra-Neuronal Drug Delivery,” Scientific Reports 7:42923 (2017), each of which is hereby incorporated by reference in its entirety.

In some embodiments, the isolated cargo polypeptide (component A), the isolated DTnd propeptide fusion (component B), and the SnoopLigase fusion protein are mixed to form a mixture in solution. In some embodiments, the mixture comprises a molar ratio of A:B≥1.4 and SnoopLigase:B≥1.4. In some embodiments, the mixture in solution is contacted with a protease having a cleavage recognition site in the propeptide fusion protein, the cargo protein and the SnoopLigase under conditions effective to enable protease cleavage at the cleavage recognition site(s) to form a protease treated protein complex. In some embodiments, the protease treated protein complex is captured on a streptavidin maxtrix though the biotinylated SnoopLigase. In some embodiments, the fusion protein comprising the cargo attached to the DTnd delivery vehicle is eluted from the steptavidin matrix using a low pH buffer.

An exemplary fusion protein in which a cargo is attached to a DTnd fusion protein through an isopeptide bond is SEQ ID NO:114 as set forth below. The isopeptide bond is indicated by the N/K symbol. The cargo and DTnd delivery vehicle were cleaved at the TEV cleavage recognition sequences as described infra.

SGSNGGAGQSGAGEGGGGSGGGGSGGGGSQAHVQLQQSGGGLVQPGGSLR
LSCAASGSIFSIYAMGWYRQAPGKQRELVAAISSYGSTNYADSVKGRFTI
SRDNAKNTVYLQMNSLKPEDTAVYYCNADIATMTAVGGFDYWGQGTQVTV
SSAHHSEDPTSQSTSGGGGSGGGGSGGGGSGGGGSGTSGSTGGGGSGGGG
SGAGDIPATYEFTDGKHYITN/KVNKGGGGSGGGGSGGGGSGADDVVDSS
KSFVMENESSYHGTKPGYVDSIQKGIQKPKSGTQGNYDDDWEGFYSTDNK
YDAAGYSVDNENPLSGKAGGVVKVTYPGLTKVLALKVDNAETIKKELGLS
LTEPLMEQVGTEEFISREGDGASRVVLSLPFAEGSSSVKYINNWEQAKAL
SVELEINFETRGKAGQDAMYEYMASACAGNRVRRSVGSSLSCINLDWDVI
RDKTKTKIESLKEHGPISNKMSESPNKTVSEEKAKSYLEEFHQTALEHPE
LSELKTVTGTNPVFAGANYAAWAVNVAQVIDSGTADNLEKTTAALSILPG
IGSVMGIADGAVHHNTEEIVAQSIALSSLMVAQAIPLVGELVDIGFAAYN
FVESIINLFQVVHNSYNRPAYSPGHGTQPFLEASGGPEANIINTSILNLR
YESNHLIDLSRYASKINIGSKVNEDPIDKNQIQLENLESSKIEVILKNAI
VYNSMYENESTSFWIRIPKYENSISLNNEYTIINCMENNSGWKVSLNYGE
IIWTLQDTQEIKQRVVEKYSQMINISDYINRWIFVTITNNRLNNSKIYIN
GRLIDQKPISNLGNIHASNNIMFKLDGCRDTHRYIWIKYFNLEDKELNEK
EIKDLYDNQSNSGILKDFWGDYLQYDKPYYMLNLYDPNKYVDVNNVGIRG
YMYLKGPRGSVMTTNIYLNSSLYRGTKFIIKKYASGNKDNIVRNNDRVYI
NVVVKNKEYRLATNASQAGVEKILSALEIPDVGNLSQVVVMKSKNDQGIT
NKCKMNLQDNNGNDIGFIGFHQENNIAKLVASNWYNRQIERSSRTLGCSW
EFIPVDDGWGERPLGAGENLYFQ

### Therapeutic Agents

Another aspect of the present disclosure relates to a therapeutic agent comprising the fusion protein described herein. In some embodiments, the fusion protein is provided with a pharmaceutically acceptable carrier.

According to some embodiments, fusion proteins comprising a single chain antibody B8 or JSG-C1 are specific against a light chain of a wild-type Clostridium botulinum neurotoxin. According to this embodiment, the therapeutic agent is able to exert antidote activity after the light chain of a wild-type Clostridium botulinum neurotoxin has penetrated the cytoplasm of a neuron, thereby extending the time window post-exposure for exerting antidote activity. Developing these types of effective antidotes against Clostridial neurotoxins comprises targeting the neural cells using a fusion protein comprising a neuron specific receptor binding domain as disclosed herein.

In some embodiments, therapeutic cargos can comprise multiple therapeutic domains (e.g., 2 or more sdAb connected by linkers, a sdAb connected to PROTAC (small-molecule proteolysis-targeting chimeras), two sdAb and a PROTAC, as non-limiting examples). See e.g., Tsai et al., “The Degradation of Botulinum Neurotoxin Light Chains Using PROTACs,” Int. J. Mol. Sci. 25, 7472 (2024) and Kuo et al., “Accelerated Neuronal Cell Recovery from Botulinum Neurotoxin Intoxication by Targeted Ubiquitination, PLOS One 6(5) e20352 (2011), each of which is hereby incorporated by reference in its entirety.

Fusion proteins comprising the non-toxic derivatives of DT-C and DT-T described supra developed under the methods described herein. Parenteral routes of administration are tested first, followed by evaluation of oral and inhalational routes as applicable. Utility as an antidote can be evaluated in vitro by testing the ability of neurotoxin derivatives to prevent neuromuscular blockade in the mouse phrenic-nerve hemidiaphragm, or to inhibit cleavage in neuronal cultures of the respective serotypes' intracellular substrate.

Fusion proteins created using the non-toxic derivatives described supra may be superior to currently available antibody-based antidotes, because they effectively mimic native toxin absorption and trafficking pathways and can therefore be effective after the wild-type neurotoxin is sequestered inside intoxicated neurons, where traditional antibodies cannot effectively target the toxin. Antidote effectiveness in vivo can be evaluated using multiple dosing regimens. Additional dosage and timing parameters relevant to using antidotes under crisis situations is further evaluated for neurotoxin derivatives found to be effective when administered simultaneously with toxin. Dose-response analyses and challenge studies against active neurotoxin provide data that allows the best candidate antidotes to be selected for further development.

Efficacy of the DTnd fusion proteins targeting BoNT can be tested as described infra.

By “non-toxic” it is meant that the fusion proteins have a toxicity that is reduced from a wild-type diphtheria toxin by at least about 400,000-fold. In certain exemplary embodiments, the LD₅₀ of a fusion protein of the present disclosure is at least 1,000; 2,000; 5,000; 7,000; 9,000; 10,000; 20,000; 30,000; 40,000; 50,000; 60,000; 70,000; 80,000; 90,000; 100,000; 400,000, 500,000, or 750,000-fold or more higher than the LD₅₀ of wild-type diphteria toxin. The particular mode of administration (discussed infra) may also affect the LD₅₀ of the fusion protein

As used herein, maintaining structural conformation required for specific targeting of neurons by the fusion protein and maintaining structural conformation required for delivery of the fusion protein to the neuronal cytoplasm means one or more of the following: having an DT-T domain that is capable of forming a DT-C-transporting pore after endosome acidification and the DT-C and its associated cargo are able to pass through the DT-T pore where the VHH remains active for antigen binding. In some embodiments, the catalytic domain (DT-C), the translocation domain (DT-T), and receptor-binding domain (RBD) possess structural conformation required for (i) stability of the domain, (ii) specific targeting of neurons by the fusion protein, and (iii) delivery of the fusion protein to neuronal cytoplasm.

In some embodiments, the toxicity of the DTnd delivery vehicle has lower intrinsic toxicity than C1ad. In some embodiments, the DTnd delivery vehicle is capable of translocating larger or more rigid cargos than C1ad.

### Treating a Subject

A further aspect of the present disclosure relates to a method for treating a subject for toxic effects of a neurotoxin. This method involves administering the therapeutic agent described herein to the subject under conditions effective to treat the subject for toxic effects of the neurotoxin.

In carrying out this and other methods described herein, administering can be carried out orally, inhalationally, parenterally, for example, subcutaneously, intravenously, intramuscularly, intrarticularly, intraperitoneally, by intranasal instillation, or by application to mucous membranes, such as, that of the nose, throat, and bronchial tubes. The fusion protein (or therapeutic agent) may be administered alone or with suitable pharmaceutical carriers, and can be in solid or liquid form such as, tablets, capsules, powders, solutions, suspensions, or emulsions.

The fusion protein (or therapeutic agent) may be orally administered, for example, with an inert diluent, or with an assimilable edible carrier, or may be enclosed in hard or soft shell capsules, or may be compressed into tablets, or may be incorporated directly with the food of the diet. For oral therapeutic administration, the neurotoxin (along with any cargo) may be incorporated with excipients and used in the form of tablets, capsules, elixirs, suspensions, syrups, and the like. Such compositions and preparations should contain at least 0.001% of active compound. The percentage of the compound in these compositions may, of course, be varied and may conveniently be between about 0.01% to about 10% of the weight of the unit. The amount of active compound in such therapeutically useful compositions is such that a suitable dosage will be obtained. In one embodiment, compositions are prepared so that an oral dosage unit contains between about 1 μg and 1 g of active compound.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

The fusion protein (or therapeutic agent) may also be administered parenterally. Solutions or suspensions can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol, hyaluronan and its derivatives, carboxymethyl cellulose and other soluble polysaccharide derivatives, or polyethylene glycol, are preferred liquid carriers, particularly for injectable solutions. Under ordinary conditions of storage and use, these preparations contain a preservative to prevent the growth of microorganisms if they are not produced aseptically.

The pharmaceutical forms suitable for injectable use include sterile aqueous solutions or dispersions and sterile powders for the extemporaneous preparation of sterile injectable solutions or dispersions. The form must be sterile and must be fluid to the extent that easy syringability exists. It must be stable under the conditions of manufacture and storage and must be protected against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The fusion protein (or therapeutic agent) may also be administered directly to the airways in the form of an aerosol. For use as aerosols, the fusion protein (or therapeutic agent) in solution or suspension may be packaged in a pressurized aerosol container together with suitable propellants, for example, hydrocarbon propellants like propane, butane, or isobutane with conventional adjuvants. The fusion protein (or therapeutic agent) also may be administered in a non-pressurized form such as in a nebulizer or atomizer.

Targeting the central nervous system (“CNS”) may require intra-thecal or intra-ventricular administration. Administration may occur directly to the CNS. Alternatively, administration to the CNS may involve retrograde transport from peripheral neurons (motor neurons, nociceptors) to spinal ganglia (see Caleo et al., “A Reappraisal of the Central Effects of Botulinum Neurotoxin Type A: By What Mechanism?” Journal of Neurochemistry 109:15-24 (2009), which is hereby incorporated by reference in its entirety).

In some embodiments, the fusion protein can be administered in a larger therapeutic dose than a delivery vehicle comprising a BoNT light chain.

Fusion proteins (or therapeutic agents) can be administered as a conjugate with a pharmaceutically acceptable water-soluble polymer moiety. By way of example, a polyethylene glycol conjugate is useful to increase the circulating half-life of the treatment compound, and to reduce the immunogenicity of the molecule. Specific PEG conjugates are described in U.S. Patent Application Publication No. 2006/0074200 to Daugs et al., which is hereby incorporated by reference in its entirety. Other materials that effect the functionality include hyaluronic acid (“HA”), as described in, e.g., U.S. Pat. No. 7,879,341 to Taylor and U.S. Patent Application Publication No. 2012/0141532 to Blanda et al., each of which is hereby incorporated by reference in its entirety. Liquid forms, including liposome-encapsulated formulations, are illustrated by injectable solutions and suspensions. Exemplary solid forms include capsules, tablets, and controlled-release forms, such as a mini-osmotic pump or an implant. Other dosage forms can be devised by those skilled in the art, as shown, for example, by Ansel & Popovich, Pharmaceutical Dosage Forms and Drug Delivery Systems, 5^{th} Edition (Lea & Febiger 1990), Gennaro (ed.); Remington 's Pharmaceutical Sciences, 19^{th} Edition (Mack Publishing Company 1995); and Ranade & Hollinger, Drug Delivery Systems (CRC Press 1996), which are hereby incorporated by reference in their entirety.

In some embodiments, treating a subject further involves selecting a subject in need of treatment prior to administering.

Subjects to be treated pursuant to the methods described herein include, without limitation, human and non-human primates, or other animals such as dog, cat, horse, cow, goat, sheep, rabbit, or rodent (e.g., mouse or rat).

Single chain antibodies developed to target treatment of specific conditions are known and include, for example, those that target Huntington's Protein for treatment of Huntington's disease, synuclein for treatment of Parkinson disease, upregulated cell-division genes in malignant neurons, upregulated genes in non-malignant neuronal pathologies, genes responsible for excess accumulation of amyloid fibrils in Alzheimer's disease, dormant neurotrophic virus species, herpes virus activated during pathogenesis of shingles, prion diseases, neuropathic pain (to down-regulate pain pathways), and inducers of chronic pain. The therapeutic targets of these single chain antibodies are inside the neuron and, there has been limited success in non-viral delivery of single chain antibodies to the inside of cells in a therapeutic context. The treatment methods described herein overcome these deficiencies and provide for delivery of functional antibodies to targets exposed to the cytoplasm of neurons by fusing an antibody to a DTnd delivery vehicle that directs single chain antibodies to neurons and translocates the antibodies from an internalized endosome into the cytoplasm.

Another aspect of the present disclosure relates to a method of treating a neurological condition. This method involves administering a fusion protein of the present disclosure to a subject under conditions effective to provide treatment to the subject.

The following examples are provided to illustrate embodiments of the present disclosure but they are by no means intended to limit its scope.

### EXAMPLES

### Example 1—Development of a DTnd Delivery Vehicle

A DTnd delivery vehicle was bioengineered with the sequence elements as shown in FIG. 4 (SEQ ID NO:89). Inactivating mutations K₅₁>E and E₁₄₈>K were introduced into the Diphteria toxin DT-C domain to create a non-toxic derivative (nd) of the Diphtheria toxin. (see Kimura et al., “Transgenic Mice Expressing a Fully Nontoxic Diphtheria Toxin Mutant, not CRM197 Mutant, Acquire Immune Tolerance against Diphtheria Toxin,” Journal of Biochemistry 142(1):105-112 (2007), which is hereby incorporated by reference in its entirety). The mutated amino acid corresponding to K₅₁>E of DT-Cnd is position 51 of SEQ ID NO:2, and the mutated amino acid corresponding to E₁₄₈>K of DT-Cnd is position 148 of SEQ ID NO:2.

Additionally, mutations K₁₂₅>S, R₁₇₃>A, and Q₁₈₄>S were introduced in the DT-C domain and K₂₂₇>S, Q₂₄₅>S, E₂₉₂>S, and K₃₈₅>G were included in the Diphtheria toxin translocation DT-T domain in the delivery vehicle. These additional mutations were designed to suppress the immune response of the human/animal treatment subject caused by repeated use of the native DT sequence (see Schmohl et al., “Mutagenic Deimmunization of Diphtheria Toxin for Use in Biologic Drug Development,” Toxins (Basel) 7(10):4067-4082 (2015), which is hereby incorporated by reference in its entirety).

The native Diphtheria toxin Receptor-Binding Domain (DT-R domain) was replaced with the Receptor-Binding Domain (RBD) of the Botulinum Neurotoxin A1 (BoNT/A1; SEQ ID NO:10). The BoNT/A1 RBD specifically binds two receptors on the neuronal surface—a high affinity protein receptor—Synaptic Vesicle glycoprotein 2 (SV2), and low affinity lipid receptor, ganglioside (sialic acid containing glycosphingolipid). These receptors are the main entity responsible to conferring BoNT/A1 its superior cell target specificity. Altogether, the designed fusion protein represents a diphtheria-based intraneural delivery vehicle named DTnd as shown in FIG. 2D.

A protein cargo was attached to the DTnd vehicle and was able to be delivered to the neural cytoplasm by attaching the C-terminally fused sequence of the cargo protein of interest with the N-terminus of the DT inactivated catalytic (DT-C) domain. (FIGS. 2D and 3). The protein cargo can be either genetically fused to the sequence of DTnd such as by providing a nucleotide sequence encoding the cargo protein sequence and the DTnd delivery vehicle sequence in the order described in FIG. 2D and FIG. 3 in an expression vector, followed by the expression of the entire protein in the system of choice. Alternatively, the cargo can be enzymatically fused to the delivery vehicle after production and purification of the delivery vehicle and cargo when expressed separately, optionally using separate expression platforms as described in Example 2.

For example, the therapeutic cargo can be fused to the delivery vehicle via a linker 1 to the delivery vehicle comprising the inactivated DT-C domain fused via linker 2 to the DT-T domain, and the DT-T domain fused via linker 3 to a neuron-specific receptor-binding domain such as BoNT/A1 RBD (FIG. 3).

The first example of a therapeutic cargo attached to the DTnd delivery vehicle is B8DTnd (FIG. 2D). B8 is a single chain VHH camelid anti-BoNT/A antibody.

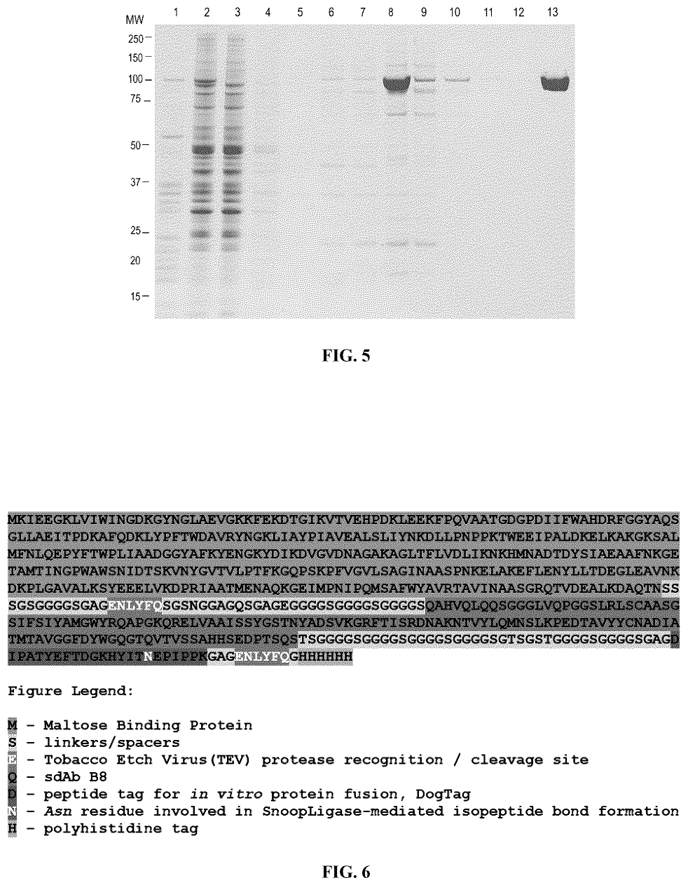

To create the B8DTnd fusion protein, the propeptide fusion protein encoding the delivery vehicle DTnd (FIG. 4; SEQ ID NO:89) was expressed in baculovirus-driven insect Sf9 cells and purified using Ni²⁺/StrepTactin tandem affinity chromatography as shown in FIG. 5.

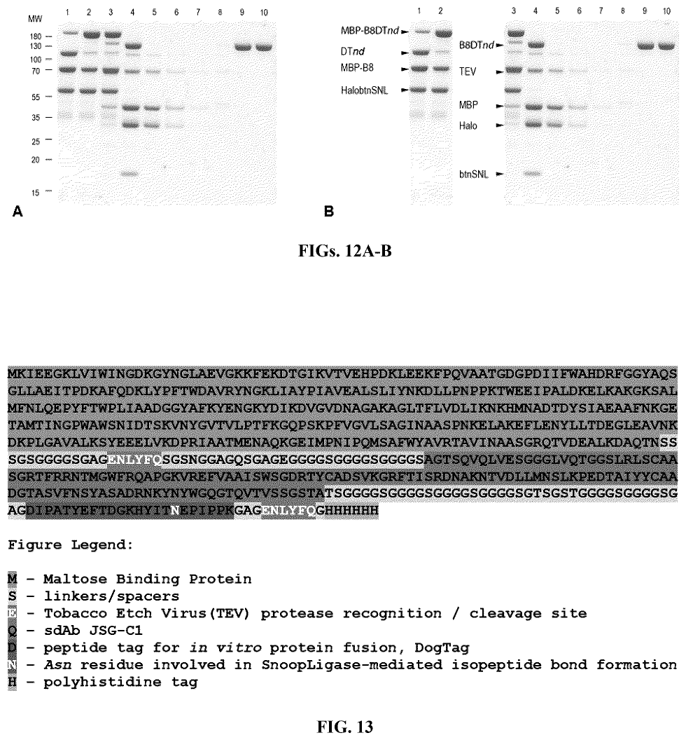

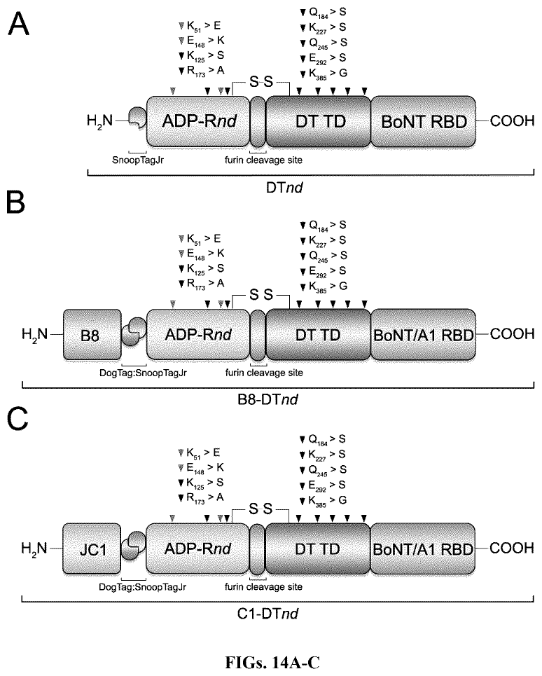

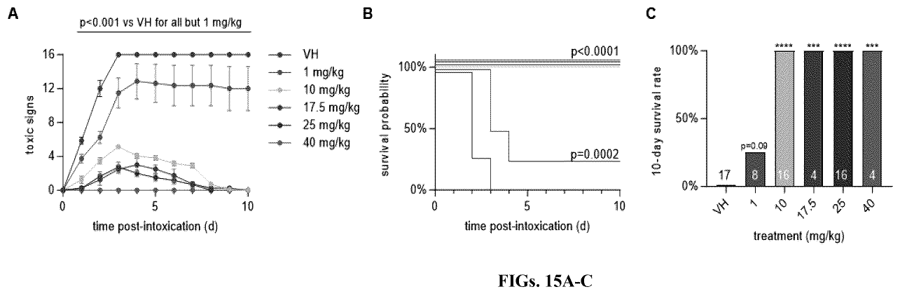

Following the same principle, the therapeutic cargo sdAb JSG-C1 against BoNT/B1 was expressed, purified and covalently conjugated with DTnd as previously described, thereby creating C1DTnd (FIGS. 13 and FIGS. 14A-C). Animal studies of C1DTnd have shown safety and efficacy profiles similar to B8DTnd.

### Example 2—SnoopLigase Attachment of Cargo to the DTnd Delivery Vehicle

The SnoopLigase-mediated isopeptide fusion reaction allows expression of individual components of the reaction, such as: 1) therapeutic cargo, “component A”; and 2) delivery vehicle DTnd, “component B” in different expression systems. The ability to provide individual components A and B separately can be important when the cargo size affects the production yield of protein in an expression system, in situations when the cargo or the combination of cargo and delivery vehicle is toxic, unstable, or denatured when expressed in an expression host, or when the cargo is not able to be produced in living cells.

The SnoopLigase-mediated isopeptide fusion reaction/technology is described in Buldin et al., “SnoopLigase Catalyzes Peptide-Peptide Locking and Enables Solid-Phase Conjugate Isolation,” Journal of the American Chemical Society 140(8):3008-3018 (2018) and U.S. Pat. No. 10,889,622, each of which is hereby incorporated by reference in its entirety.

First, the enzyme binding to the substrates (components “A” and “B”) mediate formation of the covalent isopeptide bond between them. Through optimization, a yield of the fusion product ˜80-95% was achieved. However, it was also found that non-covalent complex formed between the fusion reaction product and SnoopLigase was highly stable and particularly hard to disrupt.

Due to the size of the Dtnd delivery vehicle, the eluant had accessibility issues to the protein-protein interface of the SnoopLigase complex that was to be disrupted. Initially, the fusion protein Halotag-SnoopLigase was made to immobilize SnoopLigase via Halolink™ Resin. The ligation reaction was fully reproduced but repeated attempts failed to disrupt the complex and recover a SnoopLigase-free product.

As described above, a particular combination of circumstances in the SnoopLigase design allowed a successful ligation and elution step for the toxin-based drug delivery system.

It was determined that the methods of enzyme/product separation cited in the original paper (Buldin et al., “SnoopLigase Catalyzes Peptide—Peptide Locking and Enables Solid-Phase Conjugate Isolation,” Journal of the American Chemical Society 140(8):3008-3018 (2018), which is hereby incorporated by reference in its entirety) were not working, i.e., the fusion product remained contaminated/in a complex with the enzyme that catalyzed fusion reaction (SnoopLigase). After more than two years of research, a particular set of conditions was found that allowed the effective and successful separation of the SnoopLigase enzyme from the fusion reaction product.

Initial tests of a first version of SnoopLigase included a recombinant protein with Halotag7, fused to the N-terminus of the enzyme (Halotag7-SnoopLigase), a modified haloalkane dehalogenase that can be immobilized in a halogenated solid support such as Halolink® matrix (Promega). Halotag-SnoopLigase (49 kDa) was used to fuse delivery vehicle DTnd (100 kDa) to B8 sdAb cargo (66 kDa). After completion of the fusion reaction it was found that the SnoopLigase enzyme could not be separated from the formed fusion protein by the methods described by Buldin et al., “SnoopLigase Catalyzes Peptide-Peptide Locking and Enables Solid-Phase Conjugate Isolation,” Journal of the American Chemical Society 140(8):3008-3018 (2018) and U.S. patent Ser. No. 10/889,622, each of which is hereby incorporated by reference in its entirety). That is, the application of the buffers with high imidazole concentration, small peptide conjugate mimicking properties of the fusion protein, buffers with high salts concentration, as well as buffers with acidic pH (4-5) used for elution). Separation of the enzyme from the fusion product was not possible even by the extreme methods that should result in the denaturation of the fusion protein (i.e. chaotropic salts, denaturing agents, high temperature, and combinations thereof). By testing the above-mentioned conditions for separation of the enzyme from the fusion protein, it was noticed that fusion protein tend to degrade after application of the buffers containing high (3-4 M) imidazole concentrations.

In another attempt, six different variants of SnoopLigase with incorporation of TEV recognition/cleavage sites in different positions were created to ultimately cleave SnoopLigase into relatively small pieces after completion of the fusion reaction. This process assumed that these fragments could be separated from the formed fusion product relatively easily (because TEV cleavage was being used for removal of purification tags from the created fusion protein). It was hypothesized that it could also be used at the same time for the cleavage of the SnoopLigase having incorporated internal TEV recognition/cleavage sites). Although incorporation of the TEV recognition/cleavage sites reduced the catalytic activity of the enzyme, it was still functional for creation of the fusion product. However, it was found that TEV recognition/cleavage sites introduced into SnoopLigase were inaccessible to TEV in the complex formed between fusion protein and the SnoopLigase after completion of the conjugation reaction.

In another attempt, a version of SnoopLigase carrying the S-tag (15 aa tag originated from RNAse A) fused to the N-terminus of the enzyme (S-tag-SnoopLigase) was created. The molecular weight of the S-tag-SnoopLigase (17 kDa) was lower than Halotag7-SnoopLigase (49 kDa). This version of the enzyme was able to bind the S-protein affinity matrix, an alternative commercially available method of immobilization. Despite ability of S-tag-SnoopLigase to bind S-protein matrix along with the fusion protein formed, all tested elution conditions resulted in simultaneous elution of the enzyme in complex with fusion protein after binding of the complex to the affinity matrix. Multiple immobilization/elution conditions tested in this strategy were unsuccessful.

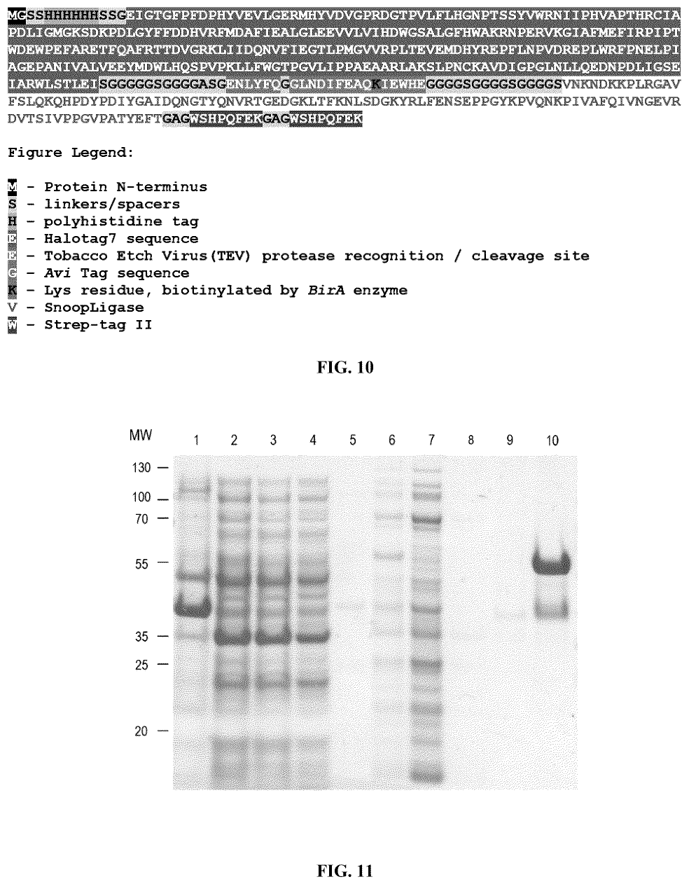

In the next attempt, a significantly longer flexible peptide linker between Halotag7 and SnoopLigase was incorporated in comparison to the construct created and tested earlier. In the central part of the linker, a 15 aa Avi tag was introduced containing lysine residue that could be enzymatically biotinylated in vitro and in vivo, if the expression host was supplemented with BirA enzyme and biotin. (HalobtnSNL, FIG. 10; SEQ ID NO:111). Although the HalobtnSNL (53 kDa) enzyme was active and able to catalyze the creation of the fusion protein from the components “A” (cargo) and “B” (delivery vehicle), and also able to bind with high affinity to streptavidin matrix, the enzyme (HalobtnSNL) was unable to be separated from the fusion product under numerous elution conditions tested.

In another attempt, the SnoopLigase fused at the N-terminus of the enzyme with 15 aa Avi tag peptide, followed by the short flexible amino acid linker (btnSnoopLigase, 17 kDa; SEQ ID NO:112) was expressed and purified. BtnSnoopLigase was produced by cleaving the TEV site on HalobtnSnoopLigase. Attempts to biotinylate the enzyme in the expression host and separate a 100% biotinylated fraction were successful. The btnSnoopLigase was enzymatically active, able to catalyze the isopeptide fusion reaction and able to bind immobilized streptavidin matrix in complex with the fusion protein created. In contrast with HalobtnSNL, the complex of streptavidin-immobilized SnoopLigase from the fusion protein was able to be disrupted by washes of the streptavidin matrix with the glycine-HCl buffer pH 3, followed by elution of un-contaminated fusion protein with the glycine-HCl buffer pH 2, that was quickly neutralized with the excess of the basic biological buffer to the final pH 8. The ability to disrupt complex of btnSnoopLigase with the fusion protein by removal of the Halotag7 domain from the prior version of the SnoopLigase (HalobtnSNL) under acidic conditions indicated the potential role of the bulky Halotag7 domain was a steric barrier restricting the access of the acidified washes/eluents to the interface between the SnoopLigase and the fusion protein product. The long flexible linker allowed accessibility of the elution agent to the SnoopLigase:product complex. The interface accessibility issue may be particularly important when the isopeptide fusion reaction substrates have a relatively high molecular weight (100 kDa—DTnd; SEQ ID NO:89 and 66 kDa—MBP-B8; SEQ ID NO:91 in this case) in comparison with relatively small proteins (<28 kDa) described in the publication by Buldin et al., “SnoopLigase Catalyzes Peptide-Peptide Locking and Enables Solid-Phase Conjugate Isolation,” Journal of the American Chemical Society 140(8):3008-3018 (2018), which is hereby incorporated by reference in its entirety. This insight leading to successful preparation and isolation of un-contaminated homogeneous isopeptide-bonded fusion protein, along with the results included in this disclosure is absent from the currently available knowledge related to mechanism of SnoopLigase-mediated isopeptide fusion reaction and could not be deduced without significant research efforts.

The advantages developed with the SnoopLigase-mediated technology applied to the DTnd delivery vehicle described in this disclosure include the following. SnoopLigase-mediated isopeptide fusion conjugation allowed simple and simultaneous change of therapeutic cargo protein fused to DTnd neuronal delivery vehicle, and simplified and expedited the process of multiple novel therapeutic entities generation. SnoopLigase-mediated isopeptide fusion conjugation allowed generation of therapeutic recombinant proteins with high yield and MW that usually resulted in low yield if the entire recombinant protein was expressed as a whole in any given expression system. For example, incorporation of a second LC/A-neutralizing nanobody (JPU-A5) into the B8C1ad construct reduced expression from 35 mg/L culture to 12.5 mg/L in Sf9 cells, while addition of a third nanobody reduced expression below 5 mg/L. SnoopLigase-mediated isopeptide fusion conjugation allowed generation of therapeutic recombinant proteins in two different expression systems, when either component “A” or component “B” can be toxic, unstable (expedited degradation) or insoluble (inclusion bodies) in a single given expression system.

In order to achieve this, specific modification of the SnoopLigase enzyme incorporating biotinylation and flexible linkers, followed by the solid-phase immobilization of the enzyme/fusion product complex on streptavidin affinity matrix allow removal of the un-incorporated reaction components and other byproducts. This process allowed elution of a highly pure fusion product without the SnoopLigase enzyme.

An example of the process of producing a SnoopLigase-mediated fusion product follow the principles of a one-pot synthesis and comprise the following steps 1-12.
1) Component A. Expression of the therapeutic cargo recombinant protein in E. coli was followed by tandem affinity purification of the product. Tandem affinity purification included affinity chromatography on amylose matrix, which binds maltose binding protein (MBP) positioned N-terminally as part of the entire recombinant protein, and Ni²⁺-affinity chromatography matrix, which binds His₆ tag incorporated into the C-terminus of the recombinant protein. As mentioned, these two tags were positioned on the N- and C-termini of the recombinant protein, with two added Tobacco etch virus protease sequences ENLYFQ↑G positioned downstream of the N-terminal purification tag and upstream of the C-terminal purification tag. The ENLYFQ↑G sequence represents the recognition/cleavage site of TEV protease used for subsequent removal of affinity purification tags in the process described below. This recombinant protein also incorporates sequence of therapeutic entity, such as single domain Antibody (sdAb, a.k.a. nanobody) that binds and inactivates target protein in the neuronal cytoplasm and DogTag (a 23 aa peptide fusion tag with asparagine residue involved in formation of isopeptide bond (FIG. 8)) on the C-terminus of recombinant protein, upstream the His₆ affinity tag used for purification and TEV recognition/cleavage sequence (component “A”). FIG. 6 illustrates the positions of the various sequence components (SEQ ID NO:91). Another example is shown in FIG. 13 (cargo sdAb JSG-C1; SEQ ID NO:93).
2) Component B. Generation of the delivery vehicle (DTnd) through baculovirus-mediated expression in insect (Sf9) cells followed by tandem affinity purification of the recombinant protein product. Tandem affinity purification included affinity chromatography on Ni²⁺ matrix (which binds His₁₂ tag incorporated into N-terminus of the recombinant protein) and StrepTactin affinity chromatography matrix (which binds two copies of 8 aa Strep Tag II incorporated into C-terminus of the recombinant protein). These two tags were positioned on N- and C-termini of the recombinant protein with two added copies of TEV protease sequence ENLYFQ↑G downstream of the N-terminal purification tag and upstream of the C-terminal purification tag. This sequence represents the recognition/cleavage sequence of TEV protease used for subsequent removal of affinity purification tags in the process described below. This recombinant protein incorporates features and sequences derived from Diphtheria toxin and Botulinum neurotoxin A1 and SnoopTagJr. (a 12 aa peptide fusion tag with lysine residue involved in formation of isopeptide bond) at the N terminus of recombinant protein, downstream the affinity tag used for protein purification and TEV recognition/cleavage sequence (component “B”). FIG. 4 illustrates the positions of the various sequence components (SEQ ID NO:89).
3) Biotinylated SnoopLigase. Expression of recombinant SnoopLigase in E. coli with incorporated sequence of Avi tag, a 15 aa peptide tag that undergoes intracellular biotinylation by birA ligase, co-expressed in the same E. coli strain through incorporation of pBirAcm plasmid, followed by Ni²⁺/StrepTactin tandem affinity chromatography purification and separation of biotinylated species from non-biotinylated (SnoopLigase, enzyme). FIG. 10 illustrates the positions of the various sequence components (SEQ ID NO:111). HaloTag7 is a modified haloalkane dehalogenase that can be immobilized in a halogenated solid support such as Halolink® matrix (Promega).
4) Mix component “A”, component “B” and enzyme (SnoopLigase) at a molar ratio of A:B≥1.4, Enzyme:B≥1.4 in the buffer that maintains a pH range of 7-8 through addition of 25 mM Tris-HCl, EPPS, HEPPSO, or other compatible biological buffer without any salts.
5) Incubate reaction mixture at 4° C. for 24 h. The step that allows the ligation is the incubation of DogTag, SnoopTagJr and SnoopLigase in no-salt buffer and cold temperature (4-15° C.) for at least 2 hours and for 24 hours to obtain higher yields.
6) Perform TEV cleavage of the fusion protein by adding TEV to the reaction mixture at molar ratio fusion product/enzyme complex:TEV of 3:1, and keeping redox potential required by TEV (Cys protease) by supplementing the reaction mixture with reduced/oxidized glutathione (GSH/GSSG redox combination) in the buffer at a final concentration 3 mM GSH and 0.3 mM GSSG.
7) Incubate the reaction at 28° C. for 3 h.
8) Capture the fusion protein/enzyme complex on streptavidin matrix at a ratio of 1 mL streptavidin matrix bed volume per 230 nmol of SnoopLigase in the reaction.
9) Wash the streptavidin matrix with 20 bed volumes using high salt buffer (1M NaCl, 0.1% Tween, pH 8.0).
10) Wash the streptavidin matrix with 10 bed volumes of 50 mM glycine-HCl buffer, pH 3.
11) Elute fusion protein with 10 bed volumes of 50 mM glycine-HCl buffer, pH 2.
12) Adjust the pH of the solution to pH 8.0 with a compatible high capacity biological buffer.

Multiple methods of SnoopLigase capture/immobilization from the reaction mixture to separate the enzyme from the fusion protein product were tested. These methods proved to be ineffective. In the elution step, the SnoopLigase system needed to be designed in a way that, when in a complex with the toxin-based delivery vehicle and its therapeutic cargo, the enzyme/product interface was accessible to the elution agent. In the studies described herein, a viable iteration to accomplish this included a SnoopLigase comprising an N-terminal flexible sequence connected to a biotinylated Avitag for immobilization on Streptavidin and subsequent elution with acidic pH.

The addition of 200 mM TriMethylAmine N-Oxide (TMAO, osmolyte, chemical chaperone) to the reaction mixture increased the kinetics of the reaction and the yield of the fusion protein, particularly in situations when concentration of substrates and enzyme in the mixture was low (≤1 mg/mL).

The components “A” and “B” can be designed to be purified with different methods or tags, which may eliminate the need of a step to proteolytically cleavage the tags. It is also possible to pre-process all the components (Components “A”, “B” and SnoopLigase) before the conjugation reaction, in which case the reaction yield is higher, and the molar ratios necessary to achieve such high yields is decreased. This is explained due to the increased exposure of DogTag and SnoopTagJr, as well as the increased accessibility of SnoopLigase.

SnoopLigase should be accessible to carry out the conjugation reaction, and most importantly, to be able to release the conjugation product after its immobilization. To guarantee the accessibility of SnoopLigase and use it in solid-phase applications, an N-terminus flexible sequence (G4S)³ with a short immobilization sequence (Avi tag, 15 amino acids) was used. Alternative designs varying in linker length and immobilization method may render similar results. For example, with this principle it is possible to envision a SnoopLigase design with a flexible or rigid linker sequence at the N or C terminus, followed by a terminal cysteine for covalent immobilization on a resin via sulfhydryl coupling. A design like this would avoid steric effects or other unexpected interactions of the immobilization substrate that could affect the accessibility of the SnoopLigase:Product complex. An alternative method comprises increasing the length or rigidity of the linker that connects SnoopTagJr to the toxin-based delivery vehicle in an effort to spatially separate the bulky part of the molecule from the tripartite complex, although this may result in a less desirable solution due to the inclusion in the the final therapeutic product of an this excessively long or rigid linker, with potential repercussions in the intracellular delivery efficiency. This new knowledge and specific examples greatly facilitate and guide the successful implementation of the SnoopLigase technology into production processes.

Furthermore, SnoopLigase-mediated isopeptide fusion conjugation allows generation of therapeutic cargos that are not produced in living cells, such as small molecules and DNA, and its incorporation into the DTnd delivery vehicle. See e.g., Kakimoto et al., “The Conjugation of Diphtheria Toxin T domain to Poly(Ethylenimine) Based Vectors for Enhanced Endosomal Escape During Gene Transfection,” Biomaterials, 30(3):402-408 (2009), which is hereby incorporated by reference in its entirety. For example, the SNAPtag fused with a protein conjugation tag (e.g., Dogtag) can also be used to couple dyes and other compatible small molecules into the delivery vehicle (see e.g., Kolberg et al., “SNAP-tag Technology: A General Introduction,” Curr. Pharm. Des. 19(30)5406-13 (2013), which is hereby incorporated by reference in its entirety. Additional non-limiting exemplary cargos include fluorescent proteins (GFP, Wasabi, mCherry, etc.), SNAPtag conjugated with fluorescent dyes, Halotag conjugated with fluorescent Dyes or PROTACs, molecular glues attached to cleavable linkers, single domain antibodies (sdAb) against BoNT serotypes, single-chain variable fragment (scFv) antibodies, chaperones, enzymes (e.g. SOD, catalases), RNA molecules encoding sdAb, scFv, chaperones or enzymes, DNA molecules encoding sdAb or scFv, chaperones or enzymes.

### Example 3—SnoopLigase Attachment of B8 Cargo to the DTnd Delivery Vehicle

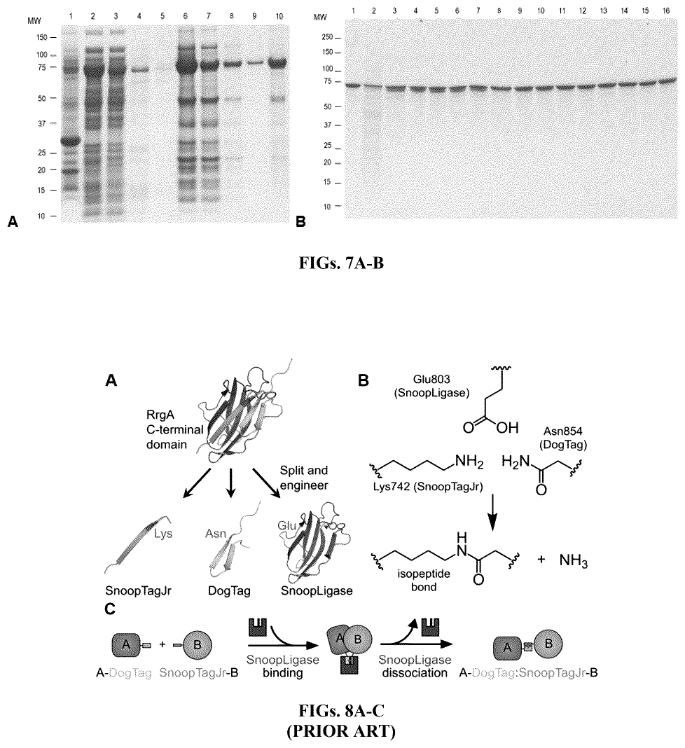

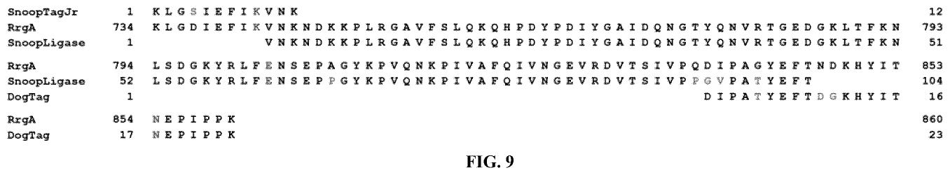

In order to add therapeutic cargo B8 (SEQ ID NO:90) to the DTnd Delivery Vehicle, a Maltose-Binding Protein B8 fusion protein (SEQ ID NO:91) was expressed in E. coli and purified using Maltose-Binding Protein (MBP)/Ni²⁺ tandem affinity chromatography as shown in FIGS. 7A-B. The DTnd delivery vehicle and cargo were covalently fused via isopeptide bond through a SnoopLigase-mediated conjugation system. The SnoopLigase-mediated conjugation system is illustrated in FIG. 8A and FIG. 9.

A biotinylated Halotag7 SnoopLigase as shown in FIG. 10 (SEQ ID NO:111) was developed for these experiments and was expressed and purified by tandem affinity chromatography as shown in FIG. 11.

The SnoopLigase-mediated isopeptide fusion of the B8 cargo to the DTnd Delivery Vehicle and subsequent Tobacco Etch Virus (TEV) protease cleavage for removal of affinity purification tags was performed in liquid phase as shown in FIGS. 12A-B. SEQ ID NO:114 shows the TEV processed B8DTnd fusion protein sequence. Alternatively, the ligase reaction can be done by immobilizing the ligase on a solid support and performing the reaction/immobilization simultaneously.

FIGS. 12A-B shows the process of biotinylated HaloTag-SnoopLigase-mediated isopeptide conjugation of the components “A” (e.g., cargo sdAb B8) and “B” (e.g., DTnd Delivery Vehicle), followed by removal of the HaloTag-SnoopLigase enzyme from the reaction mixture and purification of the fusion protein (B8 DTnd Delivery Vehicle; SEQ ID NO:114). HalobtnSNL becomes btnSNL after TEV cleavage. For example, in FIG. 12A, lane 4, at the end of TEV reaction almost all HalobtnSNL has been cleaved, producing btnSNL.

The biotinylated SnoopLigase conjugation enzyme, still non-covalently attached to the fusion product B8DTnd, was then captured on a streptavidin immobilized solid support matrix. The solid support was then subjected to stringent washes and as final step B8DTnd eluted as a pure product (lanes 9-10 of FIG. 12B).

### Example 3—Comparison and Discussion of BoNT C1ad with DTnd

(In Background) (BoNT LC accumulated in the neuronal cytoplasm proteolytically cleaves Soluble N-ethylmaleimide-sensitive factor Attachment protein REceptor (SNARE) proteins, preventing functional assembly of the tripartite complex of SNAP25/VAMP/Syntaxin required for synaptic transmission, and caused the flaccid paralysis characteristic of clinical botulism (McNutt et al., “Neuronal Delivery of Antibodies has Therapeutic Effects in Animal Models of Botulism,” Science Translational Medicine 13(575) (2021), which is hereby incorporated by reference in its entirety).

Applicant's research group successfully developed B8C1ad, a biotherapeutic consisting on a single domain antibody (sdAb; B8) cargo (Tremblay et al., “Camelid Single Domain Antibodies (VHHs) as Neuronal Cell Intrabody Binding Agents and Inhibitors of Clostridium botulinum Neurotoxin (BoNT) Proteases,” Toxicon 56(6):990-998 (2010), which is hereby incorporated by reference in its entirety) genetically fused to C1ad—a botulinum neurotoxin-based delivery vehicle (Vazquez-Cintron et al., “Engineering Botulinum Neurotoxin C1 as a Molecular Vehicle for Intra-Neuronal Drug Delivery,” Sci Rep 7:42923 (2017), which is hereby incorporated by reference in its entirety) that can enter neurons and protect SNARE proteins by inhibiting LC/A1 catalytic activity in situ. Post-symptomatic administration of B8C1ad produced antidotal rescue in mice, guinea pigs, and nonhuman primates after a lethal BoNT/A1 botulism challenge (McNutt et al., “Neuronal Delivery of Antibodies has Therapeutic Effects in Animal Models of Botulism,” Science Translational Medicine 13(575) (2021), which is hereby incorporated by reference in its entirety).

A critical limitation of B8C1ad has been the intrinsic latent toxicity of the delivery vehicle C1ad, which decreases the therapeutic window of B8C1ad (NO Adverse Events Level (NOAEL): 0.4 mg/kg, EC₅₀: 0.025 mg/kg, LD₅₀: 5 mg/kg). Although the available dose ranges have proven effective, the C1ad toxicity has limited the administration of larger therapeutic doses. Notably, the maximum therapeutic dose that has been administered corresponds to the NOAEL value. This dose also corresponds to the maximum observed therapeutic effect. Below the NOAEL dose, the therapeutic effect behaves in a dose-dependent manner. Although the therapeutic effects at greater doses than the NOAEL are expected to be higher, the intrinsic toxicity of the treatment prevents the use of higher doses. These facts led to the hypothesis that delivery vehicles with improved safety profiles could be more effective.

Another important limitation of C1ad, a botulinum neurotoxin-based delivery vehicle, is its inability to translocate a large variety of protein cargos that do not share the same properties as the native botulinum toxin light chain metalloprotease, which is able to undergo globular melting during translocation through the endosomal pore followed by refolding/restoration of enzymatic activity after LC entry into neuronal cytosol. Multiple experiments have shown that the efficiency of the cargo delivery fused to N-terminus of metalloprotease-inactivated LC substantially decreases as the cargo increases in size and rigidity. Interestingly, single domain antibodies such as B8 is able to share, at least in part, the above-mentioned properties of BoNT light chain and have been shown to be active after translocation to the cytoplasm. However, protein cargos such as eGFP (27 kDa) and Halotag7 (33 kDa) seem to have a negative effect on translocation efficiency of C1ad (data not shown). See also, Bade et al., “Botulinum Neurotoxin Type D Enables Cytosolic Delivery of Enzymatically Active Cargo Proteins to Neurones Via Unfolded Translocation Intermediates,” J. Neurochemistry 91(6):1461-1472 (2004), which is hereby incorporated by reference in its entirety.

The neuronal delivery vehicle DTnd and fully processed ligated B8DTnd did not show any signs of toxicity in vivo (mice) up to 40 mg/kg (NOAEL >40 mg/kg), a safety profile superior to B8C1ad (NOAEL=0.4 mg/kg; LD₅₀=1.45 mg/kg). Higher doses of B8DTnd were not tested due to potential glycerol toxicity (all recombinant proteins tested represent a solution with 40% glycerol, 100 mM NaCl, 25 mM EPPS, pH 8.0; glycerol used as a cryo-preservative for the recombinant proteins stored at −80° C.).

To illustrate the superior properties of DTnd the efficacy of B8DTnd and B8C1ad produced with the SnoopLigase-mediated isopeptide bond formation method were compared. As a more stringent test, both molecules were produced without removal of the enzyme (SnoopLigase) from the fusion complex.

Animal studies indicated that B8DTnd had the same efficacy as B8DTnd/SnoopLigase complex, and 100% animals survived after 2 LD₅₀ BoNT/A1 intoxication with a single 10 mg/kg dose. In contrast the B8C1a/SnoopLigase complex was unable to elicit a therapeutic effect within the previously established (expressed as a single pro-protein from genetically fused construct) B8C1ad therapeutic window (FIGS. 15A-C).

It is important to stress that delivery a SnoopLigase-mediated fusion cargo to the cytoplasm should be viewed as a more complex task in comparison with the delivery of the protein translated as a whole, from genetically fused cargo, due to the additional extra N- and C-terminal sequences formed as a result of the isopeptide conjugation (see examples of extra N- and C-terminal sequences in SnoopLigase-mediated fusion reaction in FIG. 4 and FIG. 6). The extra N-terminal sequence upstream of isopeptide-bonded lysine residue in the SnoopTagJr ( . . . KLGSIEFIK-Component “B”), and the C-terminal sequence downstream of isopeptide-bonded asparagine residue in the DogTag (Component “A”-NEPIPPK . . . ) create extra branches that increase the size and geometrical complexity of the fusion protein. The unconventional conformation of the fusion protein, as a consequence of the isopeptide bond formation, presence of non-covalently bound SnoopLigase (extra ˜17 kDa) in the complex with the fusion protein and additional N- and C-terminal peptide sequences branches mentioned above should seemingly complicate translocation of the fusion protein through endosomal pore.

The in vivo tests of B8C1ad/SnoopLigase complex after mice was challenged with BoNT/A1 show absence of efficacy in wide range of concentrations tested. We assumed that SnoopLigase tightly bound to B8C1ad conjugate prevents passage of therapeutic entity from the endosome to the neuronal cytoplasm through the pore formed by the C1ad translocation domain, thus cargo entrapped in the endosome cannot prevent BoNT/A1-induced cleavage of SNAP25 in neuronal cytoplasm and therefore lacks properties of previously described version of B8C1ad expressed as a single chain pro-protein (McNutt et al., “Neuronal Delivery of Antibodies has Therapeutic Effects in Animal Models of Botulism,” Science Translational Medicine 13(575) (2021), which is hereby incorporated by reference in its entirety).

No difference in efficacy of B8DTnd alone or in complex with SnoopLigase in mice was detected when both administered recombinant protein entities contain the same dose of B8DTnd. These results indicate a superior ability of DT translocation domain for the passage of large/rigid/multimeric proteins from the endosome to neuronal cytoplasm.

The efficacy of the SnoopLigase-mediated fusion protein, B8DTnd is surprising and unexpected. In terms of production, the BoNT/A1 RBD replacing the native DT RBD did not generate any observable incompatibility in terms of protein stability and solubility. In terms of its biotherapeutic properties, at the quarter of the NOAEL dose 100% of animals survived after 2 LD₅₀ BoNT/A1 intoxication with 2 hours post-challenge treatment with B8DTnd, compared to 93.3% at the NOAEL dose with genetically fused B8C1ad expressed as a single pro-protein (McNutt et al., “Neuronal Delivery of Antibodies has Therapeutic Effects in Animal Models of Botulism,” Science Translational Medicine 13(575) (2021), which is hereby incorporated by reference in its entirety). In contrast to B8C1ad, the therapeutic dose of B8DTnd can be significantly increased due to its superior safety profile. Post-exposure administration of B8DTnd not only achieves 100% survival after BoNT/A1 challenge, but as dose increases, the toxic signs of intoxication in tested animals progressively diminish. At NOAEL dose (40 mg/kg) no BoNT-intoxication symptoms in tested animal group were detected for the duration of the study (FIGS. 15A-C).

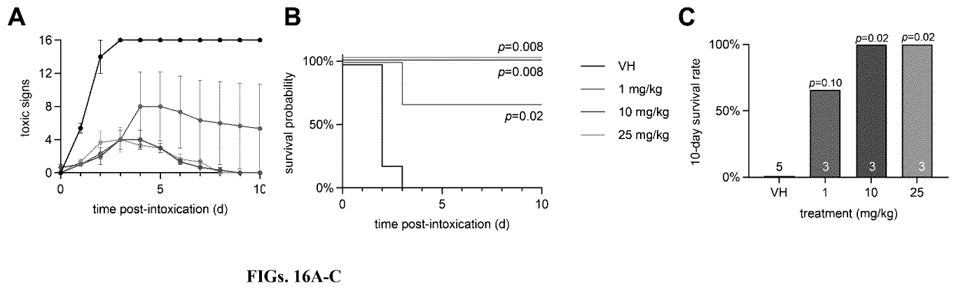

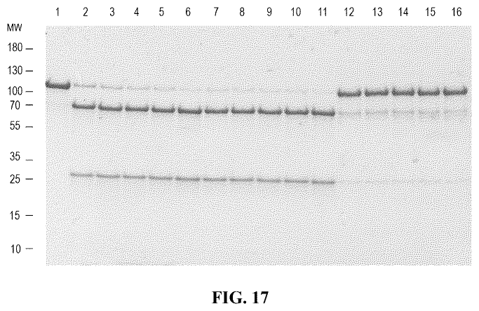

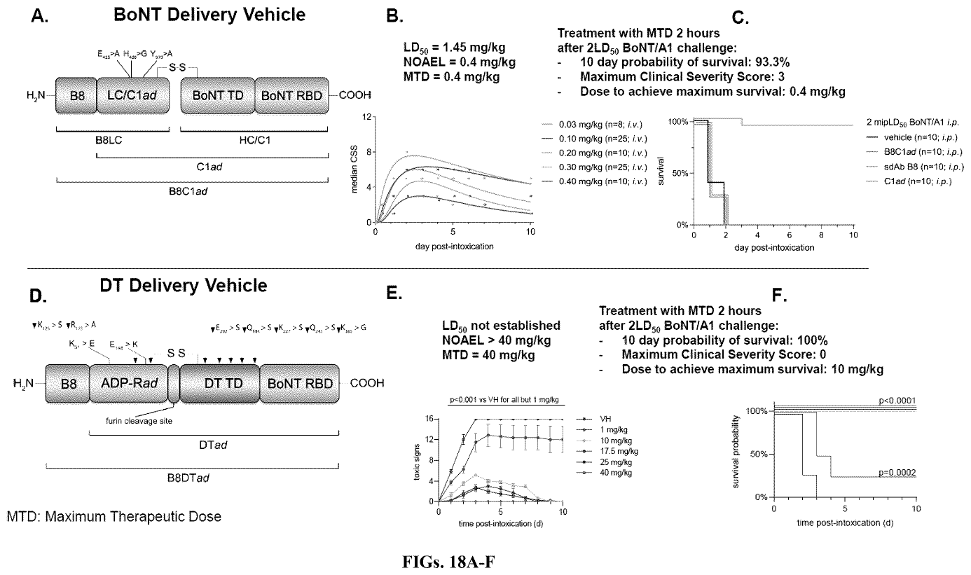

The C1DTnd conjugate was also created and tested for safety and efficacy in a post-symptomatic mice model challenged with 2 LD₅₀ of wt BoNT/B1 and treated 2 h post-intoxication. Toxic signs and mice survival increased in a dose dependent manner, achieving maximal post-exposure survival at a dose of 10 mg/kg (FIGS. 16A-C). Although the 10-day survival of mice treated with 1 mg/kg of C1DTnd was higher in comparison with B8DTnd, the 25 mg/kg dose was not high enough to eliminate toxic symptoms of BoNT/B1 intoxication.

Collectively, these findings highlight the therapeutic potential of DTnd as a delivery platform for diverse therapeutic cargos, achieving successful safety and efficacy outcomes for BoNT/A1 and BoNT/B1 intoxication in animal models.

Safety studies in mice of DTnd have indicated the delivery vehicle is safe in doses up to 40 mg/kg.

Safety studies in mice of ligated B8DTnd/SnoopLigase complex have indicated the biotherapeutic is safe in doses up to 40 mg/kg.

Effectiveness studies in mice have indicated B8DTnd is 100% at preventing animal death 2 hours post-treatment after challenge with 2 LD₅₀ BoNT/A1 with a single 10 mg/kg dose (FIGS. 15A-C).

Effectiveness studies in mice have indicated B8DTnd neutralizes clinical signs of toxicity 2 hours post-treatment after challenge with 2 LD₅₀ BoNT/A1 with a single 40 mg/kg dose (FIGS. 15A-C).

Effectiveness studies in mice have indicated C1DTnd is 100% at preventing animal death 2 hours post-treatment after challenge with 2 LD₅₀ BoNT/B1 with a single 10 mg/kg dose (FIGS. 16A-C).

There is a way, which potentially could lead to improved efficacy of DTnd to deliver cargos into neuronal targets. As mentioned above, after DT internalization into target cells, before DT-T domain insertion into endosomal membrane and translocation of the DT-C to the cytoplasm, to reach its intracellular target, the peptide linker located between DT-C and DT-T is proteolytically cleaved. Furin, abundantly present in many types of mammalian cells, including neurons is a protease responsible for this cleavage. (FIG. 2C-D). The functional role of DT and BoNT domains is very much alike, as many other AB-type bacterial toxins (FIGS. 2A-D), with the exception that the linker between the catalytic and the translocation domains in BoNT/A1 is endogenously proteolytically processed in BoNT bacterial host. If BoNT/A1 pro-protein is not cleaved by the endogenous protease, its potency can be at least 10 folds lower in comparison with the processed, disulfide-bonded BoNT heterodimer. To test in vivo comparative toxicity and protective efficacy of B8DTnd vs B8DTnd in furin-processed form, as a disulfide-bonded heterodimer we pre-cleaved B8DTnd with recombinant soluble version of furin (FIGS. 16A-C).

### Prophetic Example 4—Additional Studies

Additional optimization of conditions is being carried out. New designs of component A (cargo) may increase product yield and avoid the need to use a redox environment to cleave the purification tags by using a protease different from TEV. New designs of Component A may increase product yield and avoid the need to use a redox environment to cleave the purification tags by using a protease different from TEV. The safety and effectiveness profile of furin-cleaved B8DTnd and ligated B8DTnd will be compared. The safety and effectiveness profile of genetically fused B8DTnd and ligated B8DTnd will be compared. Preclinical data for B8DTnd and C1DTnd clinical studies will be performed. The complexity and functionality of the therapeutic cargo through the inclusion of a protein degradation domain for accelerated degradation of the intraneuronal target will be studied. Examples of these domains are full-length (parkin) or truncated E3 ubiquitin ligases. Additional different intracellular targets to treat neurological diseases not related to botulism, such as tauopathies, Parkinson's ALS, and prion diseases are contemplated. Additional studies include treatment of different forms of botulism (serotype A, B and E, and combinations thereof), delivering non-protein cargo such as small molecules, DNA, and RNA, and evaluating the efficacy of the non-protein cargo delivery.

### Example 5—Additional Sequences

Sequences of additional BoNT serotypes and TeNT toxin are provided in Table 1 below. The GenBank Accession No. of each sequence is provided, each of which is hereby incorporated by reference in its entirety. The receptor binding domains (RBD) are highlighted in bold text.

TABLE 1
Sequences of Clostridial Neurotoxins

| Identity | Amino Acid Sequence (RBD in bold text) |
|---|---|
| BoNT/A1 | >CAL82360.1 botulinum neurotoxin type A precursor |
| (SEQ ID | [Clostridium botulinum A str. ATCC 3502] |
| NO: 9) | MPFVNKQFNYKDPVNGVDIAYIKIPNAGQMQPVKAFKIHNKIWVIPERDTFTNPEE |
| | GDLNPPPEAKQVPVSYYDSTYLSTDNEKDNYLKGVTKLFERIYSTDLGRMLLTSIV |
| | RGIPFWGGSTIDTELKVIDTNCINVIQPDGSYRSEELNLVIIGPSADIIQFECKSF |
| | GHEVLNLTRNGYGSTQYIRFSPDFTFGFEESLEVDTNPLLGAGKFATDPAVTLAHE |
| | LIHAGHRLYGIAINPNRVFKVNTNAYYEMSGLEVSFEELRTFGGHDAKFIDSLQEN |
| | EFRLYYYNKFKDIASTLNKAKSIVGTTASLQYMKNVFKEKYLLSEDTSGKFSVDKL |
| | KFDKLYKMLTEIYTEDNFVKFFKVLNRKTYLNFDKAVFKINIVPKVNYTIYDGENL |
| | RNTNLAANFNGQNTEINNMNFTKLKNFTGLFEFYKLLCVRGIITSKTKSLDKGYNK |
| | ALNDLCIKVNNWDLFFSPSEDNFTNDLNKGEEITSDTNIEAAEENISLDLIQQYYL |
| | TFNFDNEPENISIENLSSDIIGQLELMPNIERFPNGKKYELDKYTMFHYLRAQEFE |
| | HGKSRIALTNSVNEALLNPSRVYTFFSSDYVKKVNKATEAAMFLGWVEQLVYDFTD |
| | ETSEVSTTDKIADITIIIPYIGPALNIGNMLYKDDFVGALIFSGAVILLEFIPEIA |
| | IPVLGTFALVSYIANKVLTVQTIDNALSKRNEKWDEVYKYIVTNWLAKVNTQIDLI |
| | RKKMKEALENQAEATKAIINYQYNQYTEEEKNNINFNIDDLSSKLNESINKAMINI |
| | NKFLNQCSVSYLMNSMIPYGVKRLEDFDASLKDALLKYIYDNRGTLIGQVDRLKDK |
| | VNNTLSTDIPFQLSKYVDNQRLLSTFTEYIKNIINTSILNLRYESNHLIDLSRYAS |
| | KINIGSKVNFDPIDKNQIQLFNLESSKIEVILKNAIVYNSMYENFSTSFWIRIPKY |
| | FNSISLNNEYTIINCMENNSGWKVSLNYGEIIWTLQDTQEIKQRVVFKYSQMINIS |
| | DYINRWIFVTITNNRLNNSKIYINGRLIDQKPISNLGNIHASNNIMFKLDGCRDTH |
| | RYIWIKYFNLFDKELNEKEIKDLYDNQSNSGILKDFWGDYLQYDKPYYMLNLYDPN |
| | KYVDVNNVGIRGYMYLKGPRGSVMTTNIYLNSSLYRGTKFIIKKYASGNKDNIVRN |
| | NDRVYINVVVKNKEYRLATNASQAGVEKILSALEIPDVGNLSQVVVMKSKNDQGIT |
| | NKCKMNLQDNNGNDIGFIGFHQFNNIAKLVASNWYNRQIERSSRTLGCSWEFIPVD |
| | DGWGERPL |
| BoNT/A2 | >CAA51824.1 botulinum neurotoxin type A [Clostridium |
| SEQ ID | botulinum A2 str. Kyoto] |
| NO: 11 | MPFVNKQFNYKDPVNGVDIAYIKIPNAGQMQPVKAFKIHNKIWVIPERDTFTNPEE |
| | GDLNPPPEAKQVPVSYYDSTYLSTDNEKDNYLKGVTKLFERIYSTDLGRMLLTSIV |
| | RGIPFWGGSTIDTELKVIDTNCINVIQPDGSYRSEELNLVIIGPSADIIQFECKSF |
| | GHDVLNLTRNGYGSTQYIRFSPDFTFGFEESLEVDTNPLLGAGKFATDPAVTLAHE |
| | LIHAEHRLYGIAINPNRVFKVNTNAYYEMSGLEVSFEELRTFGGHDAKFIDSLQEN |
| | EFRLYYYNKFKDVASTLNKAKSIIGTTASLQYMKNVFKEKYLLSEDTSGKFSVDKL |
| | KFDKLYKMLTEIYTEDNFVNFFKVINRKTYLNFDKAVFRINIVPDENYTIKDGENL |
| | KGANLSTNFNGQNTEINSRNFTRLKNFTGLFEFYKLLCVRGIIPFKTKSLDEGYNK |
| | ALNDLCIKVNNWDLFFSPSEDNFTNDLDKVEEITADTNIEAAEENISLDLIQQYYL |
| | TFDEDNEPENISIENLSSDIIGQLEPMPNIERFPNGKKYELDKYTMFHYLRAQEFE |
| | HGDSRIILTNSAEEALLKPNVAYTFFSSKYVKKINKAVEAFMFLNWAEELVYDFTD |
| | ETNEVTTMDKIADITIIVPYIGPALNIGNMLSKGEFVEAIIFTGVVAMLEFIPEYA |
| | LPVFGTFAIVSYIANKVLTVQTINNALSKRNEKWDEVYKYTVTNWLAKVNTQIDLI |
| | REKMKKALENQAEATKAIINYQYNQYTEEEKNNINFNIDDLSSKLNESINSAMINI |
| | NKFLDQCSVSYLMNSMIPYAVKRLKDFDASVRDVLLKYIYDNRGTLVLQVDRLKDE |
| | VNNTLSADIPFQLSKYVDNKKLLSTFTEYIKNIVNTSILSIVYKKDDLIDLSRYGA |
| | KINIGDRVYYDSIDKNQIKLINLESSTIEVILKNAIVYNSMYENFSTSFWIKIPKY |
| | FSKINLNNEYTIINCIENNSGWKVSLNYGEIIWTLQDNKQNIQRVVFKYSQMVNIS |
| | DYINRWIFVTITNNRLTKSKIYINGRLIDQKPISNLGNIHASNKIMFKLDGCRDPR |
| | RYIMIKYFNLEDKELNEKEIKDLYDSQSNSGILKDFWGNYLQYDKPYYMLNLEDPN |
| | KYVDVNNIGIRGYMYLKGPRGSVVTTNIYLNSTLYEGTKFIIKKYASGNEDNIVRN |
| | NDRVYINVVVKNKEYRLATNASQAGVEKILSALEIPDVGNLSQVVVMKSKDDQGIR |
| | NKCKMNLQDNNGNDIGFIGFHLYDNIAKLVASNWYNRQVGKASRTFGCSWEFIPVD |
| | DGWGESSL |
| BoNT/A3 | >ACA57525.1 bontoxilysin A (plasmid) [Clostridium |
| SEQ ID | botulinum A3 str. Loch Maree] |
| NO: 12 | MPFVNKQFNYRDPVNGVDIAYIKI PNAGQMQPVKAFKIHEGVWVIPERDTFTNPEE |
| | GDLNPPPEAKQVPVSYYDSTYLSTDNEKDNYLKGVIKLFDRIYSTGLGRMLLSFIV |
| | KGIPFWGGSTIDTELKVIDTNCINVIEPGGSYRSEELNLVITGPSADIIQFECKSF |
| | GHDVFNLTRNGYGSTQYIRFSPDFTFGFEESLEVDTNPLLGAGTFATDPAVTLAHE |
| | LIHAAHRLYGIAINPNRVLKVKTNAYYEMSGLEVSFEELRTFGGNDTNFIDSLWQK |
| | KFSRDAYDNLQNIARILNEAKTIVGTTTPLQYMKNIFIRKYFLSEDASGKISVNKA |
| | AFKEFYRVLTRGFTELEFVNPFKVINRKTYLNFDKAVFRINIVPDENYTINEGENL |
| | EGANSNGQNTEINSRNFTRLKNFTGLFEFYKLLCVRGIIPFKTKSLDEGYNKALND |
| | LCIKVNNWDLFFSPSEDNFTNDLDKVEEITADTNIEAAEENISSDLIQQYYLTEDE |
| | DNEPENISIENLSSDIIGQLEPMPNIERFPNGKKYELDKYTMFHYLRAQEFEHGDS |
| | RIILTNSAEEALLKPNVAYTFFSSKYVKKINKAVEAVIFLSWAEELVYDFTDETNE |
| | VTTMDKIADITIIVPYIGPALNIGNMVSKGEFVEAILFTGVVALLEFIPEYSLPVF |
| | GTFAIVSYIANKVLTVQTINNALSKRNEKWDEVYKYTVTNWLAKVNTQIDLIREKM |
| | KKALENQAEATRAIINYQYNQYTEEEKNNINFNIDDLSSKLNRSINRAMININKEL |
| | DQCSVSYLMNSMIPYAVKRLKDFDASVRDVLLKYIYDNRGTLILQVDRLKDEVNNT |
| | LSADIPFQLSKYVNDKKLLSTFTEYIKNIVNTSILSIVYKKDDLIDLSRYGAKINI |
| | GDRVYYDSIDKNQIKLINLESSTIEVILKNAIVYNSMYENFSTSFWIKIPKYFSKI |
| | NLNNEYTIINCIENNSGWKVSLNYGEIIWTLQDNKQNIQRVVFKYSQMVNISDYIN |
| | RWMFVTITNNRLTKSKIYINGRLIDQKPISNLGNIHASNKIMFKLDGCRDPRRYIM |
| | IKYFNLFDKELNEKEIKDLYDSQSNPGILKDFWGNYLQYDKPYYMLNLFDPNKYVD |
| | VNNIGIRGYMYLKGPRGSVMTTNIYLNSTLYMGTKFIIKKYASGNEDNIVRNNDRV |
| | YINVVVKNKEYRLATNASQAGVEKILSALEIPDVGNLSQVVVMKSKDDQGIRNKCK |
| | MNLQDNNGNDIGFVGFHLYDNIAKLVASNWYNRQVGKASRTFGCSWEFIPVDDGWG |
| | ESSL |
| BoNT/A4 | >ACQ51417.1 botulinum neurotoxin type BvA4, BoNT/BvA4 |
| SEQ ID | (plasmid) [Clostridium botulinum Ba4 str. 657] |
| NO: 13 | MPFVNKQFNYNDPENGVDIAYIKIPNAGKMQPVKAFKIHNKVWVIPERDIFTNPEE |
| | VDLNPPPEAKQVPISYYDSAYLSTDNEKDNYLKGVIKLFERIYSTDLGRMLLISIV |
| | RGIPFWGGGKIDTELKVIDTNCINIIQLDDSYRSEELNLAIIGPSANIIESQCSSF |
| | RDDVLNLTRNGYGSTQYIRFSPDFTVGFEESLEVDTNPLLGAGKFAQDPAVALAHE |
| | LIHAEHRLYGIAINTNRVFKVNTNAYYEMAGLEVSLEELITEGGNDAKFIDSLQKK |
| | EFSLYYYNKEKDIASTINKAKSIVGTTASLQYMKNVFKEKYLLSEDATGKFLVDRL |
| | KFDELYKLLTEIYTEDNFVKFFKVINRKTYLNEDKAVFKINIVPDVNYTIHDGENL |
| | RNTNLAANENGQNIEINNKNEDKLKNFTGLFEFYKLLCVRGIITSKTKSLDEGYNK |
| | ALNELCIKVNNWDLFFSPSEDNFTNDLDKVEEITSDTNIEAAEENISLDLIQQYYL |
| | NFNFDNEPENTSIENLSSDIIGQLEPMPNIERFPNGKKYELNKYTMFHYLRAQEFK |
| | HSNSRIILINSAKEALLKPNIVYTFFSSKYIKAINKAVEAVTFVNWIENLVYDETD |
| | ETNEVSTMDKIADITIVIPYIGPALNIGNMIYKGEFVEAIIFSGAVILLEIVPEIA |
| | LPVLGTFALVSYVSNKVLTVQTIDNALSKRNEKWDEVYKYIVTNWLAIVNTQINLI |
| | REKMKKALENQAEATKAIINYQYNQYTEEEKNNINFNIDDLSSKINESINSAMINI |
| | NKFLDQCSVSYLMNSMIPYAVKRLKDFDASVRDVLLKYIYDNRGTLIGQVNRLKDK |
| | VNNTLSADIPFQLSKYVDNKKLLSTFTEYIKNITNASILSIVYKDDDLIDLSRYGA |
| | EIYNGDKVYYNSIDKNQIRLINLESSTIEVILKKAIVYNSMYENFSTSFWIRIPKY |
| | FNSISINNEYTIINCMENNSGWKVSLNYGEIIWTLQDTQEIKQRVVFKYSQMINIS |
| | DYINRWIFVTITNNRITKSKIYINGRLIDQKPISNLGNIHASNKIMFKLDGCRDPH |
| | RYIVIKYFNLEDKELSEKEIKDLYDNQSNSGILKDEWGDYLQYDKSYYMLNLYDPN |
| | KYVDVNNVGIRGYMYLKGPRDNVMTTNIYLNSSLYMGTKFIIKKYASGNKDNIVRN |
| | NDRVYINVVVKNKEYRLATNASQAGVEKILSALEIPDVGNLSQVVVMKSKNDQGIT |
| | NKCKMNLQDNNGNDIGFIGFHQFNNIAKLVASNWYNRQIERSSRTLGCSWEFIPVD |
| | DGWRERPL |
| BoNT/A5 | >ACG50065.1 botulinum neurotoxin sub-type A5 [Clostridium |
| SEQ ID | botulinum H04402 065] |
| NO: 14 | MLFVNKQFNYKDPVNGVDIAYIKIPNAGQMQPVKAFKIHNKIWVIPERDTFTNPEE |
| | GDLNPPPEAKQVPVSYYDSTYLSTDNEKDNYLKGVTKLFERIYSTELGRMLLTSIV |
| | RGIPFWGGSTIDTELKVIDTNCINVIQPDGSYRSEELNLVIIGPSADIIQFECKSF |
| | GHDVLNLTRNGYGSTQYIRFSPDFTFGFEESLEVDTNPLLGAGKFATDPAVTLAHE |
| | LIHAGHRLYGIAINPNRVFKVNTNAYYEMSGLEVSFEELRTFGEHDAKFIDSLQEN |
| | EFRLYYYNKFKDIASTLNKAKSIVGTTASLQYMKNVFKEKYLLSEDTSGKFSVDKL |
| | KFDKLYKMLTEIYTEDNFVKFFKVLNRKTYLNFDKAVFKINIVPEVNYTIYDGENL |
| | RNTNLAANFNGQNTEINNMNFTKLKNFTGLFEFYKLLCVRGIITSKTKSLDEGYNK |
| | ALNDLCIKVNNWDLFFSPSEDNFTNDLNKGEEITSDTNIEAAEENISLDLIQQYYL |
| | TFNEDNEPENISIENLSSDIIGQLELMPNIERFPNGKKYELDKYTMFHYLRAQEFE |
| | HGKSRIVLTNSVNEALLNPSSVYTFFSSDYVRKVNKATEAAMFLGWVEQLVYDFTD |
| | ETSEVSTTDKIADITIIIPYIGPALNIGNMLYKDDFVGALIFSGAVILLEFIPEIA |
| | IPVLGTFALVSYIANKVLTVQTIDNALSKRNEKWGEVYKYIVTNWLAKVNTQIDLI |
| | RKKMKEALENQAEATKAIINYQYNQYTEEEKNNINFNIGDLSSKLNDSINKAMINI |
| | NKFLNQCSVSYLMNSMIPYGVKRLEDFDASLKDALLKYIYDNRGTLIGQVDRLKDK |
| | VNNTLSTDIPFQLSKYVDNQRLLSTFTEYIKNIINTSILNLRYESNHLIDLSRYAS |
| | EINIGSKVNFDPIDKNQIQLENLESSKIEIILKNAIVYNSMYENFSTSFWIKIPKY |
| | FSKINLNNEYTIINCIENNSGWKVSLNYGEIIWTLQDNKQNIQRVVFKYSQMVAIS |
| | DYINRWIFITITNNRLNNSKIYINGRLIDQKPISNLGNIHASNNIMFKLDGCRDPH |
| | RYIWIKYFNLEDKELNEKEIKDLYDNQSNSGILKDFWGNYLQYDKPYYMLNLYDPN |
| | KYVDVNNVGIRGYMYLKGPRGSIVTTNIYLNSSLYMGTKFIIKKYASGNKDNIVRN |
| | NDRVYINVVVKNKEYRLATNASQAGVEKILSVLEIPDVGNLSQVVVMKSKNDQGIR |
| | NKCKMNLQDNNGNDIGFIGFHQFNNIDKLVASNWYNRQIERSSRTFGCSWEFIPVD |
| | DGWGESPL |
| BoNT/A6 | >ACW83608.1 botulinum neurotoxin type A [Clostridium |
| SEQ ID | botulinum] |
| NO: 15 | MPFVNKQFNYKDPVNGVDIAYIKIPNAGQMQPVKAFKIHNKIWVIPERDTFTNPEE |
| | GDLNPPPEAKQVPVSYYDSTYLSTDNEKDNYLKGVTKLFERIYSTDLGRMLLTSIV |
| | RGIPFWGGSTIDTELKVIDTNCINVIQPDGSYRSEELNLVIIGPSADIIQFECKSF |
| | GHEVLNLTRNGYGSTQYIRFSPDFTFGFEESLEVDTNPLLGAGKFATDPAVTLAHE |
| | LIHAGHRLYGIAINPNRVFKVNTNAYYEMSGLEVSFEELRTFGGHDAKFIDSLQEN |
| | EFRLYYYNKFKDIASTLNKAKSIVGTTASLQYMKNVFKEKYLLSEDTSGKFSVDKL |
| | KFDKLYKMLTEIYTEDNFVKFFKVLNRKTYLNFDKAVFKINIVPKVNYTIYDGENL |
| | RNTNLAANFNGQNTEINNMNFAKLKNFTGLFEFYKLLCVRGIITSKTKSLDKGYNK |
| | ALNDLCIKVNNWDLFFSPSEDNFTNDLNKGEEITSDTNIEAAEENISLDLIQQYYL |
| | TFNFDNEPENISIENLSSDIIGQLELMPNIERFPNGKKYELDKYTMFHYLSAQEFE |
| | HGKSRIDLTNSVNEALLNPSHVYTFFSSDYVKKVNKATEAAMFLGWVEQLVYDFTD |
| | ETSEVSTTDKIADITIIIPYIGPALNIGNMLYKDDFVGALIFSGAVILLEFIPEIA |
| | IPVLGTFAIVSYIANKVLTVQTINNALSKRNEKWDEVYKYTVTNWLAKVNTQIDLI |
| | REKMKKALENQAEATKAIINYQYNQYTEEEKNNINFNIDDLSSKLNESINSAMINI |
| | NKFLDQCSVSYLMNSMIPYAVKRLKDFDASVRDVLLKYIYDNRGTLIGQVDRLKDK |
| | VNNTLSTDIPFQLSKYVDNQRLLSTFTEYIKNIINTSILSLRYENNHLIDLSRYAS |
| | KINIGSRVNFDPIDKNQIQLENLESSKIEVILKNAIVYNSMYENFSTSFWIKIPKY |
| | FSEISLNNEYTIINCIENNSGWKVSLNYGEIIWTLQDNKQNIQRVVFKYSQMVAIS |
| | DYINRWIFITITNNRLTKSKIYINGRLIDQKPISNLGNIHASNKIMFKLDGCRDPR |
| | RYIMIKYFNLFDKELNEKEIKDLYDSQSNSGILKDFWGNYLQYDKPYYMLNLEDPN |
| | KYVDVNNVGIRGYMYLKGSRSTLLTTNIYLNSGLYMGTKFIIKKYASGNKDNIVRN |
| | NDRVYINVVVNNKEYRLATNASQAGVEKILSALEIPDIGNLSQVVVMKSKNDQGIR |
| | NKCKMNLQDNNGNDIGFIGFHKENDIYKLVASNWYNRQIEISSRTFGCSWEFIPVD |
| | DGWGEKPL |
| BoNT/A7 | >AFV13854.1 botulinum neurotoxin [Clostridium botulinum] |
| SEQ ID | MPFVNKQFNYKDPVNGVDIAYIKIPNAGQMQPVKAFKIHNKIWVIPERDIFTNPEE |
| NO: 16 | GDLNPPPEAKQVPVSYYDSTYLSTDNEKDNYLKGVTKLFERIYSTDLGRMLLTSIV |
| | RGIPFWGGSTIDTELKVIDTNCINVIQPDGSYRSEELNLVIIGPSADIINFECKSF |
| | GHDVLNLTRNGYGSTQYIRFSPDFTFGFEESLEVDTNPLLGAGKFAIDPAVTLAHE |
| | LIHAGHRLYGIAINPNRVFKVNTNAYYEMSGLEVSFEELRTFGGHDAKFIDSLQEN |
| | EFRLYYYNKFKEVASILNKAKSIIGTTASLQYMKNVFKEKYLLSEDTSGKFSVDKL |
| | RFDKLYKMLTEIYTEDNFVKFFKVLNRKTYLNFDKAVFKMNIVPEVNYTIYDGENL |
| | RNTNLAANFNGQNTEINNMNFTKLKNFTGLFEFYKLLCVRGIITSKTKSLDEGYNK |
| | ALNDLCIKVNNWDLFFSPSEDNFTNDLNKGEEITSDTNIEAAEENISSDLIQQYYL |
| | TFNEDNEPENISIENLSSDIIGQLELMPNIERFPNGKKYELDKYTMFHYLRAQEFE |
| | YGNSRIVLINSVNEALLNPSSVYTFFSSDYVKKANEATEAAMFLGWVEQLVYDFTD |
| | ETSEVSTMDKIADITIIVPYIGPALNIGNMVYKKKFEEALIFSGAVILLEFVPEIV |
| | LPILGTFALVSYTSNKVLTVRTIDNALSKRNEKWEEVYKYIVTNWLAKVNTQINLI |
| | RKKMKEALENQAEATKAIINYQYNQYTEEEKNNINFNIGDLSSKLNDSINKAMINI |
| | NKFLDQCSVSYLMNSMIPQGVKQLKDFDTSLRDSLLKYIYDNRGTLIGQVDRLKDK |
| | VNNTLSTDIPFQLSKYADNQRLLSTFTEYIKNIINTSILNLRYESNHLIDLSRYAS |
| | KINIGSRVNFDPIDKNQIQLENLESSKIEVILKNAIVYNSMYENFSTSFWIKIPKY |
| | FSKINLNNEYTIINCIENNSGWKVSLNYGEIIWTLQDNEQNIQRVVFKYSQMVNIS |
| | DYINRWIFVTITNNRLTKSKIYINGRLIDQKPISNLGNIHASNKIMFKLDGCRDPH |
| | RYILIKYFNLEDKELNEKEIKDLYDNQSNSGILKDFWGDYLQYDKPYYMLNLYDPN |
| | KYIDVNNIGIRGYMYLKGPRGSVTTTNIYLNSMLYMGTKFIIKKHASGNKDNIVRN |
| | NDRVYINVLVKNKEYRLATNASQAGGEKILSAVEIPDVGNLSQVVVMKSKNDQGIR |
| | NKCKMNLQDNNGNDIGFIGFHQFNNIAKLVASNWYNRQIGKTSVTLGCSWELIPVD |
| | YGWGESSL |
| BoNT/A8 | >AJA05787.1 BoNT/A8 [Clostridium botulinum A] |
| SEQ ID | MPFVNKQFNYKDTVNGIDIAYIKIPNAGQMQPVKAFKIHNKIWVIPERDTFTNPKE |
| NO: 17 | GDLNPPPEAKQVPVSYYDSTYLSTDNEKDNYLKGVTKLFERIYSTDLGRMLLTSIV |
| | RGIPFWGGSTIDTELKVIDTNCINVIQPDGSYRSEELNLVIIGPSADIIQFECKSF |
| | GHDVLNLTRNGYGSTQYIRFSPDFTFGFEESLEVDTNPLLGAGKFATDPAVTLAHE |
| | LIHAEHRLYGIAINPNRVFKVNTNAYYEMSGLEVSFEELRTFGGHNAKFIDSLQEN |
| | EFRLYYYNKFKDIASTLNKAKSIVGTTASLQYMKNVFKEKYLLSEDTSGKESVDKL |
| | KFDKLYKMLTEIYTEDNFVKFFKVLNRKTYLNFDKAVFKINIVPDENYTIKDGENL |
| | KNTNLAANFNGQNTEINSRNFTKLKNFTGLFEFYKLLCVRGIIPFKTKSLDEGYNK |
| | ALNDLCIKVNNWDLFFSPSEDNFTNDLDKVEEITSDTNIEAAEENISLDLIQQYYL |
| | TFDEDNEPENISIENLSSDIIGQLEPMPNIERFPNGKKYELDKYTMFHYLRAQEFE |
| | HSKSRIALTNSVNEALLNPSRVYTFFSSDYVKKVNKATEAAMFLGWVEQLVYDFTD |
| | ETSEVSTTDKIADITIIIPYIGPALNIGNMLYKDDFVGALIFSGAVILLEFIPEIA |
| | IPVLGTFALVSYIANKVLTVQTIDNALSKRNEKWDEVYKYIVTNWLAKVNTQIDLV |
| | RKKMKEALENQAEATKAIINYQYNQYTEEEKNNINFNIDDLSSKLNESINSAMTNI |
| | NKFLDQCSVSYLMNSMIPYAVKRLKDFDASVREVLLKYIYDNRGTLILQVDRLKDK |
| | VNNTLSADIPFQLSKYVDNKKLLSTFTEYIKNITNTSILSIVVDKDGRLIDLSRYG |
| | AEIYNGDKVSYNSIDKNQIKLINLESSAIEVILKNAIVYNSMYENFSTSFWIKIPK |
| | YFSKINLNNEYTIINCIENNSGWKVSLNYGEIIWTLQDNQQNIQRVVFKYSQMVNI |
| | SDYINRWIFVTITNNRLDKSKIYINGRLIDQKPISNLGNIHASNNIMFKLDGCRDP |
| | RRYIVIKYFNLFDKELNEKEIKDLYDNQSNSGILKDFWGDYLQYDKPYYMLNLYDP |
| | NKYVDVNNIGIRGYMYLKGPRGSVVTTNIYLNSTLYMGTKFIIKKYASGNKDNIVR |
| | NNDRVYINVVVKNKEYRLATNALQAGVEKILSALEIPDVGNLSQVVVMKSKNDQGI |
| | RNKCKMNLQDNNGNDIGLIGFHQFNNIAKLVASNWYNRQVGKASRTFGCSWEFIPV |
| | DDGWGESSQ |
| BoNT/B1 | >ACA46990.1 bontoxilysin A (plasmid) [Clostridium |
| SEQ ID | botulinum B1 str. Okra] |
| NO: 18 | MPVTINNENYNDPIDNNNIIMMEPPFARGTGRYYKAFKITDRIWIIPERYTFGYKP |
| | EDFNKSSGIFNRDVCEYYDPDYLNTNDKKNIFLQTMIKLFNRIKSKPLGEKLLEMI |
| | INGIPYLGDRRVPLEEFNTNIASVTVNKLISNPGEVERKKGIFANLIIFGPGPVLN |
| | ENETIDIGIQNHFASREGFGGIMQMKFCPEYVSVENNVQENKGASIFNRRGYFSDP |
| | ALILMHELIHVLHGLYGIKVDDLPIVPNEKKFFMQSTDAIQAEELYTFGGQDPSII |
| | TPSTDKSIYDKVLQNFRGIVDRLNKVLVCISDPNININIYKNKFKDKYKFVEDSEG |
| | KYSIDVESFDKLYKSLMFGFTETNIAENYKIKTRASYFSDSLPPVKIKNLLDNEIY |
| | TIEEGFNISDKDMEKEYRGQNKAINKQAYEEISKEHLAVYKIQMCKSVKAPGICID |
| | VDNEDLFFIADKNSESDDLSKNERIEYNTQSNYIENDFPINELILDTDLISKIELP |
| | SENTESLTDFNVDVPVYEKQPAIKKIFTDENTIFQYLYSQTFPLDIRDISLTSSED |
| | DALLFSNKVYSFFSMDYIKTANKVVEAGLFAGWVKQIVNDFVIEANKSNTMDKIAD |
| | ISLIVPYIGLALNVGNETAKGNFENAFEIAGASILLEFIPELLIPVVGAFLLESYI |
| | DNKNKIIKTIDNALTKRNEKWSDMYGLIVAQWLSTVNTQFYTIKEGMYKALNYQAQ |
| | ALEEIIKYRYNIYSEKEKSNINIDENDINSKLNEGINQAIDNINNFINGCSVSYLM |
| | KKMIPLAVEKLLDFDNTLKKNLLNYIDENKLYLIGSAEYEKSKVNKYLKTIMPFDL |
| | SIYTNDTILIEMFNKYNSEILNNIILNLRYKDNNLIDLSGYGAKVEVYDGVELNDK |
| | NQFKLTSSANSKIRVTQNQNIIFNSVFLDFSVSFWIRIPKYKNDGIQNYIHNEYTI |
| | INCMKNNSGWKISIRGNRIIWTLIDINGKTKSVFFEYNIREDISEYINRWFFVTIT |
| | NNLNNAKIYINGKLESNTDIKDIREVIANGEIIFKLDGDIDRTQFIWMKYFSIENT |
| | ELSQSNIEERYKIQSYSEYLKDFWGNPLMYNKEYYMFNAGNKNSYIKLKKDSPVGE |
| | ILTRSKYNQNSKYINYRDLYIGEKFIIRRKSNSQSINDDIVRKEDYIYLDFFNLNQ |
| | EWRVYTYKYFKKEEEKLFLAPISDSDEFYNTIQIKEYDEQPTYSCQLLEKKDEEST |
| | DEIGLIGIHRFYESGIVFEEYKDYFCISKWYLKEVKRKPYNLKLGCNWQFIPKDEG |
| | WTE |
| BoNT/B2 | >BAC22064.1 neurotoxin (plasmid) [Clostridium botulinum] |
| SEQ ID | MPVTINNFNYNDPIDNNNIIMMEPPFARGTGRYYKAFKITDRIWIIPERYTFGYKP |
| NO: 19 | EDFNKSSGIFNRDVCEYYDPDYLNTNDKKNIFLQTMIKLENRIKSKPLGEKLLEMI |
| | INGIPYLGDRRVPLEEFNTNIASVTVNKLISNPGEVERKKGIFANLIIFGPGPVLN |
| | ENETIDIGIQNHFASREGFGGIMQMKFCPEYVSVENNVQENKGASIFNRRGYFSDP |
| | ALILMHELIHVLHGLYGIKVDDLPIVPNEKKFFMQSTDAIQAEELYTFGGQDPSII |
| | TPSTDKSIYDKVLQNFRGIVDRLNKVLVCISDPNININIYKNKFKDKYKFVEDSEG |
| | KYSIDVESFDKLYKSLMFGFTETNIAENYKIKTRASYFSDSLPPVKIKNLLDNEIY |
| | TIEEGFNISDKNMEKEYRGQNKAINKQAYEEISKEHLAVYKIQMCKSVRAPGICID |
| | VDNEDLFFIADKNSFSDDLSKNERIEYDTQSNYIENRSSIDELILDTNLISKIELP |
| | SENTESLTDFNVDVPVYEKQPAIKKIFTDENTIFQYLYSQTFPLDIRDISLTSSED |
| | DALLFSNKVYSFFSMDYIKTANKVVEAGLFAGWVKQIVDDFVIEANKSSTMDKIAD |
| | ISLIVPYIGLALNVGNETAKGNFENAFEIAGASILLEFIPELLIPVVGAFLLESYI |
| | DNKNKIIKTIDNALTKRDEKWIDMYGLIVAQWLSTVNTQFYTIKEGMYKALNYQAQ |
| | ALEEIIKYKYNIYSEKEKSNINIDENDINSKLNEGINQAVDNINNFINECSVSYLM |
| | KKMIPLAVEKLLDFDNTLKKNLLNYIDENKLYLIGSAEYEKSKVDKHLKTIIPFDL |
| | SMYTNNTILIEIFNKYNSEILNNIILNLRYRDNNLIDLSGYGANVEVYDGVELNDK |
| | NQFKLTSSTNSEIRVTQNQNIIFNSMFLDFSVSFWIRIPKYKNDGIQNYIHNEYTI |
| | INCIKNNSGWKISIRGNRIIWTLTDINGKTKSVFFEYSIREDISDYINRWFFVTIT |
| | NNSDNAKIYINGKLESNIDIKDIGEVIANGEIIFKLDGDIDRTQFIWMKYFSIENT |
| | ELSQSNIKEIYKIQSYSEYLKDFWGNPLMYNKEYYMFNAGNKNSYIKLKKDSSVGE |
| | ILTRSKYNQNSNYINYRNLYIGEKFIIRRKSNSQSINDDIVRKEDYIYLDFFNSNR |
| | EWRVYAYKDFKEEEKKLFLANIYDSNEFYKTIQIKEYDEQPTYSCQLLFKKDEEST |
| | DEIGLIGIHRFYESGIVLKDYKNYFCISKWYLKEVKRKPYNPNLGCNWQFIPKDEG |
| | WIE |
| BoNT/B3 | >ABM73977.1 neurotoxin B [Clostridium botulinum] |
| SEQ ID | MPVTINNFNYNDPIDNDNIIMMEPPFARGTGRYYKAFKITDRIWIIPERYTFGYKP |
| NO: 20 | EDFNKSSGIFNRDVCEYYDPDYLNTNDKKNIFLQTMIKLENRIKSKPLGEKLLEMI |
| | INGIPYLGDRRVPLEEFNTNIASVTVNKLISNPGEVERKKGIFANLIIFGPGPVLN |
| | ENETIDIGIQNHFASREGFGGIMQMKFCPEYVSVENNVQENKGASIFNRRGYFSDP |
| | ALILMHELIHVLHGLYGIKVDDLPIVPNEKKFFMQSTDAIQAEELYTFGGQDPRII |
| | TPSTDKSIYDKVLQNFRGIVDRLNKVLVCISDPNININIYKNKFKDKYKFVEDSEG |
| | KYSIDVESFDKLYKSLMFGFTETNIAENYKIKTRASYFSDSLPPVKIKNLLDNEIY |
| | TIEEGFNISDKNMEKEYRGQNKAINKQAYEEISKEHLAVYKIQMCKSVRAPGICID |
| | VDNEDLFFIADKNSESDDLSKNERIEYDTQSNYIENRSSIDELILDTNLISKIELP |
| | SENTESLTDFNVDVPVYEKQPAIKKIFTDENTIFQYLYSQTFPLDIRDISLTSSFD |
| | DALLFSNKVYSFFSMDYIKTANKVVEAGLFAGWVKQIVDDFVIEANKSSTMDKIAD |
| | ISLIVPYIGLALNVGNETAKGNFENAFEIAGASILLEFIPELLIPVVGAFLLESYI |
| | DNKNKIIKTIDNALTKRDEKWIDMYGLIVAQWLSTVNTQFYTIKEGMYKALNYQAQ |
| | ALEEIIKYKYNIYSEKEKSNINIDENDINSKLNEGINQAIDNINNFINECSVSYLM |
| | KKMIPLAVEKLLDFDNTLKKNLLNYIDENKLYLIGSAEYEKSKVDKHLKTIIPFDL |
| | SMYTNNTILIEIFNKYNSEILNNIILNLRYRDNNLIDLSGYGAKVEVYNGVELNDK |
| | NQFKLTSSANSKIRVTQNQDIIFNSMFLDFSVSFWIRIPKYKNDGIQNYIHNEYTI |
| | INCIKNNSGWKISIRGNKIIWTLTDINGKTKSVFFEYSIRKDVSEYINRWFFVTIT |
| | NNSDNAKIYINGKLESNIDIKDIGEVIANGEIIFKLDGDIDRTQFIWMKYFSIENT |
| | ELSQSNIKETYKIQSYSEYLKDFWGNPLMYNKEYYMFNAGNKNSYIKLKKDSSVGE |
| | ILTRSKYNQNSNYINYRNLYIGEKFIIRRKSNSQSINDDIVRKEDYIYLDFFNLNQ |
| | EWRVYAYKDFKKKEEKLFLANIYDSNEFYNTIQIKEYDEQPTYSCQLLFKKDEEST |
| | DEIGLIGIHRFYESGIVEKDYKDYFCISKWYLKEVKRKPYNPNLGCNWQFIPKDEG |
| | WIE |
| BoNT/B4 | >ABM73987.1 neurotoxin B [Clostridium botulinum B str. |
| SEQ ID | Eklund 17B (NRP)] |
| NO: 21 | MPVTINNENYNDPIDNDNIIMMEPPFARGTGRYYKAFKITDRIWIIPERYTFGYKP |
| | EDFNKSSGIFNRDVCEYYDPDYLNTNDKKNIFLQTMIKLENRIKSKPLGEKLLEMI |
| | INGIPYLGDRRVPLEEFNTNIASVTVNKLISNPGEVEQKKGIFANLIIFGPGPVLN |
| | ENETIDIGIQNHFASREGFGGIMQMKFCPEYVSVENNVQENKGASIFNRRGYFSDP |
| | ALILMHELIHVLHGLYGIKVDDLPIVPNEKKFFMQSTDTIQAEELYTFGGQDPSII |
| | SPSTDKSIYDKVLQNFRGIVDRLNKVLVCISDPNININIYKNKFKDKYKFVEDSEG |
| | KYSIDVESFNKLYKSLMFGFTEINIAENYKIKTRASYFSDSLPPVKIKNLLDNEIY |
| | TIEEGENISDKNMGKEYRGQNKAINKQAYEEISKEHLAVYKIQMCKSVKVPGICID |
| | VDNENLFFIADKNSFSDDLSKNERVEYNTQNNYIGNDFPINELILDTDLISKIELP |
| | SENTESLTDFNVDVPVYEKQPAIKKVFTDENTIFQYLYSQTFPLNIRDISLTSSED |
| | DALLVSSKVYSFFSMDYIKTANKVVEAGLFAGWVKQIVDDFVIEANKSSTMDKIAD |
| | ISLIVPYIGLALNVGDETAKGNFESAFEIAGSSILLEFIPELLIPVVGVFLLESYI |
| | DNKNKIIKTIDNALTKRVEKWIDMYGLIVAQWLSTVNTQFYTIKEGMYKALNYQAQ |
| | ALEEIIKYKYNIYSEEEKSNININFNDINSKLNDGINQAMDNINDFINECSVSYLM |
| | KKMIPLAVKKLLDFDNTLKKNLLNYIDENKLYLIGSVEDEKSKVDKYLKTIIPFDL |
| | STYTNNEILIKIFNKYNSEILNNIILNLRYRDNNLIDLSGYGAKVEVYDGVKLNDK |
| | NQFKLTSSADSKIRVTQNQNIIFNSMFLDFSVSFWIRIPKYRNDDIQNYIHNEYTI |
| | INCMKNNSGWKISIRGNRIIWTLIDINGKTKSVFFEYNIREDISEYINRWFFVTIT |
| | NNLDNAKIYINGTLESNMDIKDIGEVIVNGEITFKLDGDVDRTQFIWMKYFSIFNT |
| | QLNQSNIKETYKIQSYSEYLKDFWGNPLMYNKEYYMFNAGNKNSYIKLVKDSSVGE |
| | ILIRSKYNQNSNYINYRNLYIGEKFIIRRKSNSQSINDDIVRKEDYIHLDFVNSNE |
| | EWRVYAYKNFKEQEQKLFLSIIYDSNEFYKTIQIKEYDEQPTYSCQLLFKKDEEST |
| | DDIGLIGIHRFYESGVLRKKYKDYFCISKWYLKEVKRKPYKSNLGCNWQFIPKDEG |
| | WTE |
| BoNT/B5 | >ACQ51206.1 botulinum neurotoxin type BvB, BoNT/BvB |
| SEQ ID | (plasmid) [Clostridium botulinum Ba4 str. 657] |
| NO: 22 | MPVTINNENYNDPIDNNNIIMMEPPFARGMGRYYKAFKITDRIWIIPERYTFGYKP |
| | EDENKSSGIFNRDVCEYYDPDYLNTNDKKNIFLQTMIKLENRIKSKPLGEKLLEMI |
| | INGIPYLGDRRVPLEEFNTNIASVTVNKLISNPGEVERKKGIFANLIIFGPGPVLN |
| | ENETIDIGIQNHFASREGFGGIMQMKFCPEYVSVENNVQENKGASIFNRRGYFSDP |
| | ALILMHELIHVLHGLYGIKVNDLPIVPNEKKFFMQSTDAIQAEELYTFGGQDPSII |
| | SPSTDKSIYDKVLQNFRGIVDRINKVLVCISDPNININIYKNKFKDKYKFVEDSEG |
| | KYSIDVESFDKLYKSLMFGFTETNIAENYKIKTRASYFSDSLPPVKIKNLLDNEIY |
| | TIEEGENISDKNMEKEYRGQNKAINKQAYEEISKEHLAVYKIQMCKSVKAPGICID |
| | VDNEDLFFIADKNSESDDLSKNERIAYNTQNNYIENDFSINELILDTDLISKIELP |
| | SENTESLTDENVYVPVYKKQPAIKKIFTDENTIFQYLYSQTEPLDIRDISLTSSFD |
| | DALLFSNKVYSFFSMDYIKTANKVVEAGLFAGWVKQIVDDFVIEANKSSTMDKIAD |
| | ISLIVPYIGLALNVGNETAKGNFENAFEIAGASILLEFIPELLIPVVGAFLLESYI |
| | DNKNKIIETINSALTKRDEKWIDMYGLIVAQWISTVNTQFYTIKEGMYKALNYQAQ |
| | ALEEIIKYKYNIYSEKERSNINIDENDVNSKLNEGINQAIDNINNFINECSVSYLM |
| | KKMIPLAVEKLLDEDNTLRKNLLNYIDENKLYLIGSAEYEKSKVDKYLKTSIPFDL |
| | STYTNNTILIEIENKYNSDILNNIILNLRYRDNKLIDLSGYGAKVEVYDGVKLNDK |
| | NQFKLTSSANSKIRVIQNQNIIFNSMELDFSVSFWIRIPKYKNDGIQNYIHNEYTI |
| | INCMKNNSGWKISIRGNMIIWTLIDINGKIKSVFFEYSIKEDISEYINRWFFVTIT |
| | NNSDNAKIYINGKLESHIDIRDIREVIANDEIIFKLDGNIDRTQFIWMKYFSIFNT |
| | ELSQSNIEETYKIQSYSEYLKDEWGNPLMYNKEYYMFNAGNKNSYIKLKKDSSVGE |
| | ILTRSKYNQNSKYINYRDLYIGEKFIIRRKSNSQSINDDIVRKEDYIYLDFFNLNQ |
| | EWRVYMYKYFKKEEEKLFLAPISDSDEFYNTIQIKEYDEQPTYSCQLLFKKDEEST |
| | DEIGLIGIHRFYESGIVEKEYKDYFCISKWYLKEVKRKPYNSKLGCNWQFIPKDEG |
| | WTE |
| BoNT/B6 | >BAF91946.1 neurotoxin type B [Clostridium botulinum] |
| SEQ ID | MPVTINNENYNDPIDNNNIIMMEPPFARGTGRYYKAFKITDRIWIIPERYTFGYKP |
| NO: 23 | EDENKSSGIFNRDVCEYYDPDYLNTNDKKNIFLQTMIKLENRIKSKPLGEKLLEMI |
| | INGIPYLGDRRVPLEEFNTNIASVTVNKLISNPGEVERKKGIFANLIIFGPGPVLN |
| | ENETIDIGIQNHFASREGFGGIMQMKFCPEYVSVENNVQENKGASIFNRRGYFSDP |
| | ALILMHELIHVLHGLYGIKVDDLPIVPNEKKFFMQSTDAIQAEELYTFGGQDPSII |
| | TPSTDKSIYDKVLQNFRGIVDRLNKVLVCISDPNININIYKNKFKDKYKFVEDSEG |
| | KYSIDVESFDKLYKSLMFGFTETNIAENYKIKTRASYFSDSLPPVKIKNLLDNEIY |
| | TIEEGFNISDKNMEKEYRGQNKAINKQAYEEISKEHLAVYKIQMCKSVRAPGICID |
| | VDNEDLFFIADKNSESDDLSKNERIEYDTQSNYIENRSSIDELILDTNLISKIELP |
| | SENTESLTDFNVDVPVYEKQPAIKKFFTDENTIFQYLYSQTFPLDIRDISLTSSFD |
| | DALLFSNKVYSFFSMDYIKTANKVVEAGLFAGWVKQIVDDFVIEANKSNTMDKLAD |
| | ISLIVPYIGLALNVGNETAKGNFENAFEIAGASILLEFIPELLIPVVGAFLLESYI |
| | DNKNKIIKTIDNALTKRDEKWRDMYGLIVAQWLSTVNTQFYTIKEGMYKALNYQAQ |
| | ALEEIIKYKYNIYSEKEKSNINIDENDINSKLNEGINQAIDNINNFINECSVSYLM |
| | KKMIPLAVEKLLDFDNTLKKNLLNYIDENKLYLIGSAEYEKSKVDKHLKTIIPFDL |
| | SMYTNNTILIEIFKKYNSEILNNIILNLRYRDNNLIDLSGYGANVEVYDGVELNDK |
| | NQFKLTSSTNSEIRVTQNQNIIFNSMFLDFSVSFWIRIPKYKNDGIQNYIHNEYTI |
| | INCIKNNSGWKISIRGNRIIWTLTDINGKTKSVFFEYSIREDISDYINRWFFVTIT |
| | NNSDNAKIYINGKLESNIDIKDIGEVIANGEIIFKLDGDIDRTQFIWMKYFSIENT |
| | ELSQSNIKEIYKIQSYSEYLKDFWGNPLMYNKEYYMFNAGNKNSYIKLKKDSPVGE |
| | ILTRSKYNQNSNYINYRNLYIGEKFIIRRKSNSQSINDDIVRKEDYIYLDFFNLNQ |
| | EWRVYALKNFKKKEEKLFLAPISDSDEFYNTIQIKEYDEQPTYSCQLLFKKDEEST |
| | DEIGLIGIHRFYESGIVFKDYKYYFCISKWYLKEVKRKPYNPNLGCNWQFIPKDEG |
| | WIE |
| BoNT/B7 | >AFD33678.1 botulinum neurotoxin B subtype/variant B7 |
| SEQ ID | [Clostridium botulinum] |
| NO: 24 | MPVTINNENYNDPIDNNNIIMMEPPFARGTGRYYKAFKITDRIWIIPERYTFGYKP |
| | EDFNKSSGIFNRDVCEYYDPDYLNTNDKKNIFLQTMIKLFNRIKSKPLGEKLLEMI |
| | INGIPYLGDRRVPLEEFNTNIASVTVNKLISNPGEVERKKGIFANLIIFGPGPVLN |
| | ENETIDIGIQNHFASREGFGGIMQMKFCPEYVSVENNVQENKGASIFNRRGYFSDP |
| | ALILMHELIHVLHGLYGIKVDDLPIVPNEKKFFMQSTDAIQAEELYTFGGQDPSII |
| | TPSTDKSIYDKVLQNFRGIVDRLNKVLVCISDPNININIYKNKFKDKYKFVEDSEG |
| | KYSIDVESFDKLYKSLMFGFTETNIAENYKIKTRASYFSDSLPPVKIKNLLDNEIY |
| | TIEEGENISDKDMEKEYRGQNKAINKQAYEEISKEHLAVYKIQMCKSVKAPGICID |
| | VDNEDLFFIADKNSESDDLSKNERIEYNTKNIYIENYESINELILDTDLISGIELP |
| | SENTESLTDFNVDVPVYEKQPAIKKIFTDENTIFQYLYSQTFPLDIRDISLTSSFD |
| | DALLFSNKVYSFFSMDYIKTANKVVEAGLFAGWVKQIIDDFVIEANKSSTMDKIAD |
| | ISLIVPYIGLALNVGNETAKGNFENAFEIAGASILLEFIPELLIPVVGAFLLESYI |
| | DNKNKIIKTIDNALTKRVEKWIDMYGLIVAQWLSTVNTQFYTIKEGMYKALNYQAQ |
| | ALEEIIKYKYNIYSEKEKLNINIDENDINSKLNEGINQAIDNINNFINECSVSYLM |
| | KKMIPLAIEKLLDFDNALKKNLLNYIDENKLYLIGSVEEEKSKVDKFFKTIIPFDL |
| | SMYTNNTILIEMVNKYNSEILNNIILNLRYRDNNLIDSSGYGAKVEVYNGVELNDK |
| | NQFKLTSSANSKIKVTQNQNITFNSMFLDFSVSFWIRIPKYKNDGIQNYIHNEYTI |
| | INCMKNNSGWKISIRGNRIIWTLTDINGKTKSVFFEYSIREDISDYINRWFFVTIT |
| | NNLDNAKIYINGKLESNIDIRDIREVIVNGEIIFKLDGEIDRTQFIWMKYFSIENT |
| | ELSQSNVKEIYKIQSYSKYLKDFWGNPLMYNKEYYMFNAGNKNSYIKLVKDSSVGE |
| | ILTRSKYNQNSNYINYRNLYIGEKFIIRRKSSSQSISDDIVRKEDYIYLDFENSNR |
| | EWRVYAYKNFKGQEEKLFLANIYDSNEFYKTIQIKEYDEQPTYSCQLLEKKDEEST |
| | DEIGLIGIHNFYESGILFKDYKDYFCISKWYLKEVKKKPYSSNLGCNWQFIPKDEG |
| | WTE |
| BoNT/B8 | >AFN61309.1 neurotoxin B8 [Clostridium botulinum] |
| SEQ ID | MPVTINNENYNDPIDNNNIIMMEPPFARGTGRYYKAFKITDRIWIIPERYTFGYKP |
| NO: 25 | EDENKSSGIFNRDVCEYYDPDYLNTNDKKNIFLQTMIKLENRIKSKPLGEKLLEMI |
| | INGIPYLGDRRVPLEEFNTNIASVTVNKLISNPGGEERKEGIFANLIIFGPGPVLN |
| | ENETIDIGIQNHFASREGFGGIMQMKFCPEYVSVENNVQENKGASIFNRRGYFSDP |
| | ALILMHELIHVLHGLYGIKVDDLPIVPNGKKFFMQSTDAIQAEELYTFGGQDPSII |
| | TPSTDKSIYDKVLQNFRGIVDRLNKVLVCISDPNININIYKNKFKDKYKFVEDSEG |
| | KYSIDVESFDKLYKSLMFGFTETNIAENYKIKTRASYFSDSLPPVKIKNLLDDEIY |
| | TIEEGFNISDKNMGKEYRGQNKAINKQAYEEISKEHLAVYKIQMCKSVRAPGICID |
| | VDNEDLFFIADKNSESDDLSKNERIEYNTQSNYIENDESINELILDTDLISKIELP |
| | SENTESLTDFNVDVPVYEKQPAIKKIFTDENTIFQYLYSQTFPLDIRDISLTSSFD |
| | DALLFSNKVYSFFSMDYIKTANKVVEAGLFAGWVKQIVDDFVIEANKSNTMDKIAD |
| | ISLIVPYIGLALNVGNETAKGNFENAFEIAGSSILLEFIPELLIPVVGAFLLESYI |
| | DNKNKIIKTIDNALTKRDEKWIDMYGLIVAQWLSTVNTQFYTIKEGMYKALNYQAQ |
| | ALEEIIKYKYNIYSEKEKSNISIDENDINSKLNEGINQAIDNINDFINECSVSYLM |
| | KKMIPLAVEKLLDFDNTLKKNLLNYIDENKLYLIGSAEYEKSKVDKHLKTIMTEDL |
| | SMYTNNTILIKMVNKYNSEILNNIILNLRYRDNNLIDLSGYGANVEVYDGVELNDK |
| | NQFKLTSSTNSEIRVTQNQNIIVNSMFLDFSVSFWIRIPKYKNDGIQNYIHNEYTI |
| | INCMKNNSGWKISIRGNRIIWTLIDINGKIKSVFFEYSIRKDVSEYINRWFFVTIT |
| | NNLDNAKIYINGKLESNMDIRDIREVIANGEIIFKLDGDIDRTQFIWMKYFSIENT |
| | ELSQSNIEETYKIQSYSEYLKDFWGNPLMYNKEYYMFNAGSKNSYIKLKKDSSVGE |
| | ILTRSKYNQNSQYINYRDLYIGEKFIIKRKSNSQSINDDIVRKEDYIYLDFFNLNQ |
| | EWRVYAYKDFKGQKEQKLFLANIHDSNEFYKTIQIKEYDEQPTYSCQLLFKKDEES |
| | TDEIGLIGIHRFYESGFVFQEYKYYFCISKWYLKEVKKKPYNPDLGCNWQFIPKDE |
| | GWTE |
| BoNT/C1 | >BAA14235.1 botulinum C1 neurotoxin [Clostridium phage |
| SEQ ID | c-st] |
| NO: 26 | MPITINNFNYSDPVDNKNILYLDTHLNTLANEPEKAFRITGNIWVIPDRFSRNSNP |
| | NLNKPPRVTSPKSGYYDPNYLSTDSDKDTFLKEIIKLFKRINSREIGEELIYRLST |
| | DIPFPGNNNTPINTFDFDVDFNSVDVKTRQGNNWVKTGSINPSVIITGPRENIIDP |
| | ETSTFKLTNNTFAAQEGFGALSIISISPRFMLTYSNATNDVGEGRFSKSEFCMDPI |
| | LILMHELNHAMHNLYGIAIPNDQTISSVTSNIFYSQYNVKLEYAEIYAFGGPTIDL |
| | IPKSARKYFEEKALDYYRSIAKRLNSITTANPSSENKYIGEYKQKLIRKYRFVVES |
| | SGEVTVNRNKEVELYNELTQIFTEFNYAKIYNVQNRKIYLSNVYTPVTANILDDNV |
| | YDIQNGFNIPKSNLNVLFMGQNLSRNPALRKVNPENMLYLFTKFCHKAIDGRSLYN |
| | KTLDCRELLVKNTDLPFIGDISDVKTDIFLRKDINEETEVIYYPDNVSVDQVILSK |
| | NTSEHGQLDLLYPSIDSESEILPGENQVFYDNRTQNVDYLNSYYYLESQKLSDNVE |
| | DFTFTRSIEEALDNSAKVYTYFPTLANKVNAGVQGGLFLMWANDVVEDFTTNILRK |
| | DTLDKISDVSAIIPYIGPALNISNSVRRGNFTEAFAVTGVTILLEAFPEFTIPALG |
| | AFVIYSKVQERNEIIKTIDNCLEQRIKRWKDSYEWMMGTWLSRIITQFNNISYQMY |
| | DSLNYQAGAIKAKIDLEYKKYSGSDKENIKSQVENLKNSLDVKISEAMNNINKFIR |
| | ECSVTYLFKNMLPKVIDELNEFDRNTKAKLINLIDSHNIILVGEVDKLKAKVNNSF |
| | QNTIPFNIFSYTNNSLLKDIINEYFNNINDSKILSLQNRKNTLVDTSGYNAEVSEE |
| | GDVQLNPIFPFDFKLGSSGEDRGKVIVTQNENIVYNSMYESFSISFWIRINKWVSN |
| | LPGYTIIDSVKNNSGWSIGIISNFLVFTLKQNEDSEQSINFSYDISNNAPGYNKWF |
| | FVTVTNNMMGNMKIYINGKLIDTIKVKELTGINFSKTITFEINKIPDTGLITSDSD |
| | NINMWIRDFYIFAKELDGKDINILENSLQYTNVVKDYWGNDLRYNKEYYMVNIDYL |
| | NRYMYANSRQIVFNTRRNNNDFNEGYKIIIKRIRGNTNDTRVRGGDILYFDMTINN |
| | KAYNLEMKNETMYADNHSTEDIYAIGLREQTKDINDNIIFQIQPMNNTYYYASQIF |
| | KSNFNGENISGICSIGTYRFRLGGDWYRHNYLVPTVKQGNYASLLESTSTHWGFVP |
| | VSE |
| BoNT/CD | >BAA08418.1 neurotoxin [Clostridium botulinum C] |
| SEQ ID | MPITINNFNYSDPVDNKNILYLDTHLNTLANEPEKAFRIIGNIWVIPDRESRDSNP |
| NO: 27 | NLNKPPRVTSPKSGYYDPNYLSTDSEKDTFLKEIIKLFKRINSREIGEELIYRLAT |
| | DIPFPGNNNTPINTFDFDVDFNSVDVKTRQGNNWVKTGSINPSVIITGPRENIIDP |
| | ETSTFKLTNNTFAAQEGFGALSIISISPREMLTYSNATNNVGEGRFSKSEFCMDPI |
| | LILMHELNHTMHNLYGIAIPNDQRISSVTSNIFYSQYKVKLEYAEIYAFGGPTIDL |
| | IPKSGRKYFEEKALDYYRSIAKRLNSITTANPSSENKYIGEYKQKLIRKYRFVVES |
| | SGEVAVDRNKFAELYKELTQIFTEFNYAKIYNVQNRKIYLSNVYTPVTANILDDNV |
| | YDIQNGFNIPKSNLNVLFMGQNLSRNPALRKVNPENMLYLFTKFCHKAIDGRSLYN |
| | KTLDCRELLVKNTDLPFIGDISDIKTDIFLSKDINVETEVIDYPDNVSVDQVILSK |
| | NTSEHGQLDLLYPIIEGESQVLPGENQVFYDNRTQNVDYLNSYYYLESQKLSDNVE |
| | DFTFTTSIEEALDNSGKVYTYFPKLADKVNTGVQGGLFLMWANDVVEDFTTNILRK |
| | DTLDKISDVSAIIPYIGPALNISNSVRRENFTEAFAVTGVTILLEAFQEFTIPALG |
| | AFVIYSKVQERNEIIKTIDNCLEQRIKRWKDSYEWMIGTWLSRITTQFNNISYQMY |
| | DSLNYQADAIKDKIDLEYKKYSGSDKENIKSQVENLKNSLDIKISEAMNNINKFIR |
| | ECSVTYLFKNMLPKVIDELNKFDLKTKTELINLIDSHNIILVGEVDRLKAKVNESF |
| | ENTIPFNIFSYTNNSLLKDIINEYENSINDSKILSLQNKKNALVDTSGYNAEVRLE |
| | GDVQVNTIYTNDFKLSSSGDKIIVNLNNNILYSAIYENSSVSFWIKISKDLTNSHN |
| | EYTIINSIKQNSGWKLCIRNGNIEWILQDINRKYKSLIFDYSESLSHTGYTNKWFF |
| | VTITNNIMGYMKLYINGELKQSERIEDLNEVKLDKTIVEGIDENIDENQMLWIRDF |
| | NIFSKELSNEDINIVYEGQILRNVIKDYWGNPLKFDTEYYIINDNYIDRYIAPKSN |
| | ILVLVQYPDRSKLYTGNPITIKSVSDKNPYSRILNGDNIMFHMLYNSGKYMIIRDT |
| | DTIYAIEGRECSKNCVYALKLQSNLGNYGIGIFSIKNIVSQNKYCSQIFSSEMKNT |
| | MLLADIYKPWRFSFENAYTPVAVTNYETKLLSTSSFWKFISRDPGWVE |
| BoNT/D | >EES90380.1 botulinum neurotoxin type D, BoNT/D, partial |
| SEQ ID | [Clostridium phage D-1873] |
| NO: 28 | MTWPVKDFNYSDPVNDNDILYLRIPQNKLITTPVKAFMITQNIWVIPERFSSDTNP |
| | SLSKPPRPTSKYQSYYDPSYLSTDEQKDTFLKGIIKLFKRINERDIGKKLINYLVV |
| | GSPFMGDSSTPEDTFDFTRHTTNIAVEKFENGSWKVTNIITPSVLIFGPLPNILDY |
| | TASLTLQGQQSNPSFEGFGTLSILKVAPEFLLTFSDVTSNQSSAVLGKSIFCMDPV |
| | IALMHELTHSLHQLYGINIPSDKRIRPQVSEGFFSQDGPNVQFEELYTFGGLDVEI |
| | IPQIERSQLREKALGHYKDIAKRLNNINKTIPSSWISNIDKYKKIFSEKYNFDKDN |
| | TGNFVVNIDKFNSLYSDLTNVMSEVVYSSQYNVKNRTHYFSRHYLPVFANILDDNI |
| | YTIRDGENLINKGENIENSGQNIERNPALQKLSSESVVDLFTKVCLRLTKNSRDDS |
| | TCIKVKNNRLPYVADKDSISQEIFENKIITDETNVQNYSDKFSLDESILDGQVPIN |
| | PEIVDPLLPNVNMEPLNLPGEEIVFYDDITKYVDYLNSYYYLESQKLSNNVENITL |
| | TTSVEEALGYSNKIYTFLPSLAEKVNKGVQAGLFLNWANEVVEDFTTNIMKKDTLD |
| | KISDVSVIIPYIGPALNIGNSALRGNENQAFATAGVAFLLEGFPEFTIPALGVFTF |
| | YSSIQEREKIIKTIENCLEQRVKRWKDSYQWMVSNWLSRITTQFNHINYQMYDSLS |
| | YQADAIKAKIDLEYKKYSGSDKENIKSQVENLKNSLDVKISEAMNNINKFIRECSV |
| | TYLFKNMLPKVIDELNKFDLRTKTELINLIDSHNIILVGEVDRLKAKVNESFENTM |
| | PFNIFSYTNNSLLKDIINEYFNSINDSKILSLQNKKNALVDTSGYNAEVRVGDNVQ |
| | LNTIYTNDFKLSSSGDKIIVNLNNNILYSAIYENSSVSFWIKISKDLTNSHNEYTI |
| | INSIEQNSGWKLCIRNGNIEWILQDVNRKYKSLIFDYSESLSHTGYTNKWFFVTIT |
| | NNIMGYMKLYINGELKQSQKIEDLDEVKLDKTIVFGIDENIDENQMLWIRDENIFS |
| | KELSNEDINIVYEGQILRNVIKDYWGNPLKFDTEYYIINDNYIDRYIAPESNVLVL |
| | VQYPDRSKLYTGNPITIKSVSDKNPYSRILNGDNIILHMLYNSRKYMIIRDTDTIY |
| | ATQGGECSQNCVYALKLQSNLGNYGIGIFSIKNIVSKNKYCSQIFSSFRENTMLLA |
| | DIYKPWRFSFKNAYTPVAVTNYET |
| BoNT/DC | >ABP48747.1 neurotoxin [Clostridium botulinum] |
| SEQ ID | MTWPVKDFNYSDPVNDNDILYLRIPQNKLITTPVKAFMITQNIWVIPERFSSDTNP |
| NO: 29 | SLSKPPRPTSKYQSYYDPSYLSTDEQKDTFLKGIIKLFKRINERDIGKKLINYLVV |
| | GSPFMGDSSTPEDTFDFTRHTTNIAVEKFENGSWKVTNIITPSVLIFGPLPNILDY |
| | TASLTLQGQQSNPSFEGFGTLSILKVAPEFLLTFSDVTSNQSSAVLGKSIFCMDPV |
| | IALMHELTHSLHQLYGINIPSDKRIRPQVSEGFFSQDGPNVQFEELYTFGGSDVEI |
| | IPQIERLQLREKALGHYKDIAKRLNNINKTIPSSWSSNIDKYKKIFSEKYNEDKDN |
| | TGNFVVNIDKFNSLYSDLTNVMSEVVYSSQYNVKNRTHYFSKHYLPVFANILDDNI |
| | YTIINGENLTTKGFNIENSGQNIERNPALQKLSSESVVDLFTKVCLRLTRNSRDDS |
| | TCIQVKNNTLPYVADKDSISQEIFESQIITDETNVENYSDNFSLDESILDAKVPTN |
| | PEAVDPLLPNVNMEPLNVPGEEEVFYDDITKDVDYLNSYYYLEAQKLSNNVENITL |
| | TTSVEEALGYSNKIYTFLPSLAEKVNKGVQAGLFLNWANEVVEDFTTNIMKKDTLD |
| | KISDVSAIIPYIGPALNIGNSALRGNFKQAFATAGVAFLLEGFPEFTIPALGVFTF |
| | YSSIQEREKIIKTIENCLEQRVKRWKDSYQWMVSNWLSRITTQFNHISYQMYDSLS |
| | YQADAIKAKIDLEYKKYSGSDKENIKSQVENLKNSLDVKISEAMNNINKFIRECSV |
| | TYLFKNMLPKVIDELNKFDLKTKTELINLIDSHNIILVGEVDRLKAKVNESFENTI |
| | PFNIFSYTNNSLLKDMINEYFNSINDSKILSLQNKKNTLMDTSGYNAEVRVEGNVQ |
| | LNPIFPFDFKLGSSGDDRGKVIVTQNENIVYNAMYESFSISFWIRINKWVSNLPGY |
| | TIIDSVKNNSGWSIGIISNFLVFTLKQNENSEQDINFSYDISKNAAGYNKWFFVTI |
| | TTNMMGNMMIYINGKLIDTIKVKELTGINFSKTITFQMNKIPNTGLITSDSDNINM |
| | WIRDFYIFAKELDDKDINILENSLQYTNVVKDYWGNDLRYDKEYYMINVNYMNRYM |
| | SKKGNGIVENTRKNNNDFNEGYKIIIKRIRGNTNDTRVRGENVLYENTTIDNKQYS |
| | LGMYKPSRNLGTDLVPLGALDQPMDEIRKYGSFIIQPCNTFDYYASQLFLSSNATT |
| | NRLGILSIGSYSFKLGDDYWFNHEYLIPVIKIEHYASLLESTSTHWVFVPASE |
| BoNT/E1 | >CAA43999.1 botulinum neurotoxin type E [Clostridium |
| SEQ ID | botulinum] |
| NO: 30 | MPKINSFNYNDPVNDRTILYIKPGGCQEFYKSFNIMKNIWIIPERNVIGTTPQDFH |
| | PPTSLKNGDSSYYDPNYLQSDEEKDRFLKIVTKIFNRINNNLSGGILLEELSKANP |
| | YLGNDNTPDNQFHIGDASAVEIKFSNGSQDILLPNVIIMGAEPDLFETNSSNISLR |
| | NNYMPSNHRFGSIAIVTFSPEYSFRENDNCMNEFIQDPALTLMHELIHSLHGLYGA |
| | KGITTKYTITQKQNPLITNIRGTNIEEFLTFGGTDLNIITSAQSNDIYTNLLADYK |
| | KIASKLSKVQVSNPLLNPYKDVFEAKYGLDKDASGIYSVNINKENDIFKKLYSFTE |
| | FDLRTKFQVKCRQTYIGQYKYFKLSNLLNDSIYNISEGYNINNLKVNFRGQNANLN |
| | PRIITPITGRGLVKKIIRFCKNIVSVKGIRKSICIEINNGELFFVASENSYNDDNI |
| | NTPKEIDDTVTSNNNYENDLDQVILNENSESAPGLSDEKLNLTIQNDAYIPKYDSN |
| | GTSDIEQHDVNELNVFFYLDAQKVPEGENNVNLTSSIDTALLEQPKIYTFFSSEFI |
| | NNVNKPVQAALFVSWIQQVLVDFTTEANQKSTVDKIADISIVVPYIGLALNIGNEA |
| | QKGNFKDALELLGAGILLEFEPELLIPTILVFTIKSFLGSSDNKNKVIKAINNALK |
| | ERDEKWKEVYSFIVSNWMTKINTQFNKRKEQMYQALQNQVNAIKTIIESKYNSYTL |
| | EEKNELTNKYDIKQIENELNQKVSIAMNNIDRELTESSISYLMKIINEVKINKLRE |
| | YDENVKTYLLNYIIQHGSILGESQQELNSMVTDTLNNSIPFKLSSYTDDKILISYF |
| | NKFFKRIKSSSVLNMRYKNDKYVDTSGYDSNININGDVYKYPTNKNQFGIYNDKLS |
| | EVNISQNDYIIYDNKYKNFSISFWVRIPNYDNKIVNVNNEYTIINCMRDNNSGWKV |
| | SLNHNEIIWTFEDNRGINQKLAFNYGNANGISDYINKWIFVTITNDRLGDSKLYIN |
| | GNLIDQKSILNLGNIHVSDNILFKIVNCSYTRYIGIRYFNIFDKELDETEIQTLYS |
| | NEPNTNILKDFWGNYLLYDKEYYLLNVLKPNNFIDRRKDSTLSINNIRSTILLANR |
| | LYSGIKVKIQRVNNSSTNDNLVRKNDQVYINFVASKTHLFPLYADTATTNKEKTIK |
| | ISSSGNRFNQVVVMNSVGNCTMNFKNNNGNNIGLLGFKADTVVASTWYYTHMRDHT |
| | NSNGCFWNFISEEHGWQEK |
| BoNT/E2 | >EF028404.1 |
| SEQ ID | MPKINSFNYNDPVNDRTILYIKPGGCQEFYKSFNIMKNIWIIPERNVIGTTPQDFH |
| NO: 31 | PPTSLKNGDSSYYDPNYLQSDEEKDRFLKIVTKIFNRINNNLSGGILLEELSKANP |
| | YLGNDNTPDNQFHIGDASAVEIKFSNGIQDILLPNVIIMGAEPDLFETNSSNISLR |
| | NNYMPSNHGFGSIAIVTFSPEYSFRENDNSMNEFIQDPALTLMHELIHSLHGLYGA |
| | KGITTKYTITQKQNPLITNIRGTNIEEFLTFGGTDLNIITSAQSNDIYTNLLADYK |
| | KIASKLSKVQVSNPLLNPYKDVFEAKYGLDKDASGIYSVNINKENDIFKKLYSFTE |
| | FDLATKFQVKCRQTYIGQYKYFKLSNLLNDSIYNISEGYNINNLKVNFRGQNANLN |
| | PRIITPITGRGLVKKIIRFCKNIVSVKGIRKSICIEINNGELFFVASENSYNDDNI |
| | NTPKEIDDTVTSNNNYENDLDQVILNENSESAPGLSDEKLNLTIQNDAYIPKYDSN |
| | GTSDIEQHDVNELNVFFYLDAQKVPEGENNVNLTSSIDTALLEQPKIYTFFSSEFI |
| | NNVNKPVQAALFVSWIQQVLVDETTEANQKSTVDKIADISIVVPYIGLALNIGNEA |
| | QKGNFKDALELLGAGILLEFEPELLIPTILVFTIKSFLGSSDNKNKVIKAINNALK |
| | ERDEKWKEVYSFIVSNWMTKINTQFNKRKEQMYQALQNQVNAIKTIIESKYNSYTL |
| | EEKNELTNKYDIKQIENELNQKVSIAMNNIDRELTESSISYLMKLINEVKINKLRE |
| | YDENVKTYLLNYIIQHGSILGESQQELNSMVTDTLNNSIPFKLSSYTDDKILISYF |
| | NKFFKRIKSSSVLNMRYKNDKYVDTSGYDSNININGDVYKYPTNKNQFGIYNDKLS |
| | EVNISQNDYIIYDNKYKNFSISFWVRIPNYDNKIVNVNNEYTIINCMRDNNSGWKV |
| | SLNHNEIIWTLQDNAGINQKLAFNYGNANGISDYINKWIFVTITNDRLGDSKLYIN |
| | GNLIDQKSILNLGNIHVSDNILFKIVNCSYTRYIGIRYFNIFDKELDETEIQTLYN |
| | NEPNANILKDFWGNYLLYDKEYYLLNVLKPNNFIDRRTDSTLSINNIRSTILLANR |
| | LYSGIKVKIQRVNNSSTNDNLVRKNDQVYINFVASKTHLFPLYADTNTTNKEKTIK |
| | SSSSGNRFNQVVVMNSVGNNCTMNFKNNNGNNIGMLGFKDNTLVASTWYYTHMRDN |
| | TNSNGCFWNFISEEHGWQEK |
| BoNT/E3 | >ABM73980.1 neurotoxin E [Clostridium botulinum] |
| SEQ ID | MPKINSFNYNDPVNDRTILYIKPGGCQEFYKSFNIMKNIWIIPERNVIGTTPQDFH |
| NO: 32 | PPTSLKNGDSSYYDPNYLQSDEEKDRFLKIVTKIFNRINNNLSGGILLEELSKANP |
| | YLGNDNTPDNQFHIGDASAVEIKFSNGSQHILLPNVIIMGAEPDLFETNSSNISLR |
| | NNYMPSNHGFGSIAIVTFSPEYSFRENDNSINEFIQDPALTLMHELIHSLHGLYGA |
| | KGITTTCIITQQQNPLITNRKGINIEEFLTFGGNDLNIITVAQYNDIYTNLLNDYR |
| | KIASKLSKVQVSNPQLNPYKDIFQEKYGLDKDASGIYSVNINKEDDILKKLYSFTE |
| | FDLATKFQVKCRETYIGQYKYFKLSNLLNDSIYNISEGYNINNLKVNERGQNANLN |
| | PRIIKPITGRGLVKKIIRFCKNIVSVKGIRKSICIEINNGELFFVASENSYNDDNI |
| | NTPKEIDDTVTSNNNYENDLDQVILNENSESAPGLSDEKLNLTIQNDAYIPKYDSN |
| | GTSDIEQHDVNELNVFFYLDAQKVPEGENNVNLTSSIDTALLEQPKIYTFFSSEFI |
| | NNVNKPVQAALFVSWIQQVLVDETTEANQKSTVDKIADISIVVPYIGLALNIGNEA |
| | QKGNFKDALELLGAGILLEFEPELLIPTILVFTIKSFLGSSDNKNKVIKAINNALK |
| | ERDEKWKEVYSFIVSNWMTKINTQFNKRKEQMYQALQNQVNAIKTIIESKYNSYTL |
| | EEKNELTNKYDIKQIENELNQKVSIAMNNIDRFLTESSISYLMKLINEVKINKLRE |
| | YDENVKTYLLNYIIQHGSILGESQQELNSMVTDTLNNSIPFKLSSYTDDKILISYF |
| | NKFFKRIKSSSVLNMRYKNDKYVDTSGYDSNININGDVYKYPTNKNQFGIYNDKLS |
| | EVNISQNDYIIYDNKYKNFSISFWVRIPNYDNKIVNVNNEYTIINCMRDNNSGWKV |
| | SLNHNEIIWTLQDNAGINQKLAFNYGNANGISDYINKWIFVTITNDRLGDSKLYIN |
| | GNLIDQKSILNLGNIHVSDNILFKIVNCSYTRYIGIRYFNIFDKELDETEIQTLYS |
| | NEPNTNILKDFWGNYLLYDKEYYLLNVLKPNNFIDRRKDSTLSINNIRSTILLANR |
| | LYSGIKVKIQRVNNSSTNDNLVRKNDQVYINFVASKTHLFPLYADTATTNKEKTIK |
| | ISSSGNRFNQVVVMNSVGNNCTMNFKNNNGNNIGLLGFKADTVVASTWYYTHMRDH |
| | TNSNGCFWNFISEEHGWQEK |
| BoNT/E4 | >BAC05434.1 type E botulinum toxin [Clostridium |
| SEQ ID | butyricum] |
| NO: 33 | MPTINSFNYNDPVNNRTILYIKPGGCQQFYKSFNIMKNIWIIPERNVIGTIPQDEL |
| | PPTSLKNGDSSYYDPNYLQSDQEKDKFLKIVTKIFNRINDNLSGRILLEELSKANP |
| | YLGNDNTPDGDFIINDASAVPIQFSNGSQSILLPNVIIMGAEPDLFETNSSNISLR |
| | NNYMPSNHGFGSIAIVTFSPEYSFRFKDNSMNEFIQDPALTLMHELIHSLHGLYGA |
| | KGITTKYTITQKQNPLITNIRGTNIEEFLTFGGTDLNIITSAQSNDIYTNLLADYK |
| | KIASKLSKVQVSNPLLNPYKDVFEAKYGLDKDASGIYSVNINKENDIFKKLYSFTE |
| | FDLATKFQVKCRQTYIGQYKYFKLSNLLNDSIYNISEGYNINNLKVNFRGQNANLN |
| | PRIITPITGRGLVKKIIRFCKNIVSVKGIRKSICIEINNGELFFVASENSYNDDNI |
| | NTPKEIDDTVTSNNNYENDLDQVILNENSESAPGLSDEKLNLTIQNDAYIPKYDSN |
| | GTSDIEQHDVNELNVFFYLDAQKVPEGENNVNLTSSIDTALLEQPKIYTFFSSEFI |
| | NNVNKPVQAALFVGWIQQVLVDFTTEANQKSTVDKIADISIVVPYIGLALNIGNEA |
| | QKGNFKDALELLGAGILLEFEPELLIPTILVFTIKSFLGSSDNKNKVIKAINNALK |
| | ERDEKWKEVYSFIVSNWMTKINTQFNKRKEQMYQALQNQVNALKAIIESKYNSYTL |
| | EEKNELTNKYDIEQIENELNQKVSIAMNNIDRFLTESSISYLMKLINEVKINKLRE |
| | YDENVKTYLLDYIIKHGSILGESQQELNSMVIDTLNNSIPFKLSSYTDDKILISYF |
| | NKFFKRIKSSSVLNMRYKNDKYVDTSGYDSNININGDVYKYPTNKNQFGIYNDKLS |
| | EVNISQNDYIIYDNKYKNFSISFWVRIPNYDNKIVNVNNEYTIINCMRDNNSGWKV |
| | SLNHNEIIWTLQDNSGINQKLAFNYGNANGISDYINKWIFVTITNDRLGDSKLYIN |
| | GNLIDKKSILNLGNIHVSDNILFKIVNCSYTRYIGIRYFNIFDKELDETEIQTLYN |
| | NEPNANILKDFWGNYLLYDKEYYLLNVLKPNNFINRRTDSTLSINNIRSTILLANR |
| | LYSGIKVKIQRVNNSSTNDNLVRKNDQVYINFVASKTHLLPLYADTATTNKEKTIK |
| | ISSSGNRFNQVVVMNSVGNNCTMNFKNNNGNNIGLLGFKADTVVASTWYYTHMRDN |
| | TNSNGFFWNFISEEHGWQEK |
| BoNT/E5 | >AB037704.1 Clostridium butyricum |
| SEQ ID | MPKINSFNYNDPVNDRTILYIKPGGCQEFYKSFNIMKNIWIIPERNVIGTTPQDFH |
| NO: 34 | PPTSLKNGDSSYYDPNYLQSDEEKDRFLKIVTKIFNRINNNLSGGILLEELSKANP |
| | YLGNDNTPDNQFHIGDASAVEIKFSNGSQDILLPNVIIMGAEPDLFETNSSNISLR |
| | NNYMPSNHGFGSIAIVTFSPEYSFRFNDNSMNEFIQDPALTLMHELIHSLHGLYGA |
| | KGITTKYTITQKQNPLITNIRGTNIEEFLTFGGTDLNIITSAQSNDIYTNLLADYK |
| | KIASKLSKVQVSNPLLNPYKDVFEAKYGLDKDASGIYSVNINKENDIFKKLYSFTE |
| | FDLATKFQVKCRQTYIGQYKYFKLSNLLNDSIYNISEGYNINNLKVNFRGQNANLN |
| | PRIITPITGRGLVKKIIRFCKNIVSVKGIRKSICIEINNGELFFVASENSYNDDNI |
| | NTPKEIDDTVTSNNNYENDLDQVILNENSESAPGLSDEKLNLTIQNDAYIPKYDSN |
| | GTSDIEQHDVNELNVFFYLDAQKVPEGENNVNLTSSIDTALLEQPKIYTFFSSEFI |
| | NNVNKPVQAALFVSWIQQVLVDFTTEANQKSTVDKIADISIVVPYIGLALNIGNEA |
| | QKGNFKDALELLGAGILLEFVPELLIPTILVFTIKSFLGSSDNKNKVIKAINNALK |
| | ERDEKWKEVYSFIVSNWMTKINTQFNKRKEQMYQALQNQVNALKTIIEFKYNSYTL |
| | EEKKELKNNYDIEQIENELNQKVSIAMNNIDRFLTESSISYLMKLINEVKINKLRE |
| | YDENVKTYLLDYIIQHGSILGESQQELNSMVIDTLNNSIPFKLSSYTDDKILISYF |
| | NKFFKRIKSSSVLNMRYKNDKYVDTSGYDSNININGEIFIYPTNKNQFTIENSKPS |
| | EVNISQNDYIIYDNKYKNFSISFWVRIPNYDNKIVNINNEYTIINCMRDNNSGWKV |
| | SLNHNEIIWTLQDNARINQKLVFKYGNANGISDYINKWIFVTITNDRLGDSKLYIN |
| | GHLIDQKSILNLGNIHVSDNILFKIVNCSYTRYIGIRYENIFDKELDETEIQTLYS |
| | NEPNTNILKDFWGNYLLYDKGYYLLNVLKPNNFIDRRKDSTLSINNIRSTILLANR |
| | LYSGIKVKIQRVNDSSTNDRFVRKNDQVYINYISNSSSYSLYADTNTTDKEKTIKS |
| | SSSGNRFNQVVVMNSVGNNCTMNFKNNNGNNIGLLGFKADTVVASTWYYTHMRDHT |
| | NSNGCFWNFISEEHGWQEK |
| BoNT/E6 | >CAM91125.1 botulinum neurotoxin [Clostridium |
| SEQ ID | botulinum E] |
| NO: 35 | MPTINSFNYNDPVNDRTILYIKPGGCQEFYKSFNIMKNIWIIPERNVIGTTPQDFH |
| | PPTSLKNGDSSYYDPNYLQSYEEKDRFLKIVTKIFNRINNNLSGGILLEELSKANP |
| | YLGNDNTPDNQFHIGDASAVEIKFSNGSQDILLPNVIIMGAEPDLFETNSSNISLR |
| | NNYMPSNHGFGSIAIVTFSPEYSFRFKDNSMNEFIQDPALTLMHELIHSLHGLYGA |
| | KGITTQYTITQQQNPLITNIKGTNIEEFLTFGGTDLNIITSAQYNDIYTNLLADYK |
| | KIASKLSKVQVSNPLLNPYKDVFEKKYGLDKDASGIYSVNINKENDIFKKLYSFTE |
| | FDLATKFQVKCRQTYIGQYKYFKLSNLLNNSIYNISEGYNINTLKVNFRGQNTNLN |
| | PRIITPLTGRGLVKKIIRFCKNIVFSKGIRKSICIEINNGELFFVASDNSYNDDNI |
| | NTPKEIDDTVTSNNNYENDLDQVILNENSESAPGLSDEKLNLTIQNDAYIPKYDSN |
| | GTSDIEQHDVNELNVFFYLDAQKVPEGENNIDFTSSIDTALLEQPKIYTFFSSEFI |
| | NNVNKPVQAALFVSWIQQVLVDFTTEANQKSTVDKIADISIVVPYIGLALNIGNEA |
| | QKGNFKDALELLGAGILLEFEPELLIPTILVFTIKSFLGSSDNKNKVIKAINNALK |
| | ERDEKWKEVYSFIVSNWMTKINTQFNKRKEQMYQALQNQVNALKTIIESKYNSYTL |
| | EEKNELTNKYNIEQIENELNQKVSIAMNNIEIFLTESSISYLMKLINEVKINKLRE |
| | YDENVKTYLLDYIIKHGSILGESQQELNSMVIDTLNNSIPFKLSSYTDDKILISYF |
| | NKFFKRIKSSSVLNMRYKNDKYVDTSGYDSNININGDVYKYPTNKNQFGIYNNKLS |
| | EVNISQNDYIIYDNKYKNFSISFWVRIPNYDNKIVNVNNEYTIINCMRDNNSGWKV |
| | SLNHNEIIWTLQDNSGINQKLAFNYGNANGISDYINKWIFVTITNDRLGDSKLYIN |
| | GNLIDKKSILNLGNIHVSDNILFKIVNCSYTRYIGIRYFNIFDKELDETEIQTLYN |
| | NEPNANILNDFWGNYLLYDKEYYLLNVLKPNNFINRRTDSTLSINNIRSTILLANR |
| | LYSGIKVKIQRVNNSSTNDNLVRKNDQVYINFVDSKTHLLPLYADTATTNKEKTIK |
| | ISSSGNRFNQVVVMNSVGNNCTMNFKNNNGNNIGLLGFKADTVVASTWYYTHMRDN |
| | TNSNGFFWNFISEEHGWQEK |
| BoNT/E7 | >AER11391.1 botulinum neurotoxin E [Clostridium |
| SEQ ID | botulinum] |
| NO: 36 | MPKINSFNYNDPVNDKTILYIKPGGCQQFYKSENIMKNIWIIPERNVIGTIPQDEL |
| | PPTSLKNGDSSYYDPNYLQSNEEKDRFLKIVTKIFNRINDNLSGRILLEELSKANP |
| | YLGNDNTPDNQFHIGDASAVEIKFSNGNQSILLPNVIIMGAEPDLFETNSSNISLR |
| | NNYMPSNHGFGSIAIVTFSPEYSFRENDNSMNEFIQDPALTLMHELIHSLHGLYGA |
| | KRITTKYTITQQQNPLITNIRGTNIEEFLTFGGTDLNIITSAQYNDIYTNLLADYK |
| | KIASKLSKVQVSNPQLNPYKDIFQEKYGLDKNASGIYSVNINKEDDIFKKLYSFTE |
| | FDLATKFQVKCRQTYIGQYKYFKLSNLLNNSIYNISEGYNINTLKVNFRGQNTNLN |
| | PRIITQLTGRGLVKKIIRFCKNIVESKGITKSICIEINNGELFFVASENSYNDDNI |
| | NTPKEIDDTVTSNNNYENDLDQVILNENSESAPGLSDEKLNLTIQNDAYIPKYDSN |
| | GTSDIEQHDVNELNVFFYLDAQKVPEGENNVNLTSSIDTALLEQPKIYTFFSSEFI |
| | NNVNKPVQAALFVSWIQQVLVDFTTEANQKSTVDKIADISIVVPYIGLALNIGNEA |
| | QKGNFKDALELLGAGILLEFEPELLIPTILVFTIKSFLGSSDNKNKVIKAINNALK |
| | ERDEKWKEVYSFIVSNWMTKINTQFNKRKEQMYQALQNQVNAIKTIIESKYNSYTL |
| | EEKNELTNKYDIKQIENELNQKVSIAMNNIDRELTESSISYLMKLINEVKINKLRE |
| | YDENVKTYLLNYIIQHGSILGESQQELNSMVTDTLNNSIPFKLSSYTDDKILISYF |
| | NKFFKRIKSSSVLNMRYKNDKYVDTSGYDSNININGDVYKYPTNKNQFGIYNDKLS |
| | EVNISQNDYIIYDNKYKNFSISFWVRIPNYDNKIVNVNNEYTIINCMRDNNSGWKV |
| | SLNHNEIIWTLQDNAGINQKLAFNYGNANGISDYINKWIFVTITNDRLGDSKLYIN |
| | GNLIDQKSILNLGNIHVSDNILFKIVNCSYTRYIGIRYENIFDKELDETEIQTLYS |
| | NEPNTNILKDFWGNYLLYDKEYYLLNVLKPNNFIDRRKDSTLSINNIRSTILLANR |
| | LYSGIKVKIQRVNNSSTNDNLVRKNDQVYINFVASKTHLFPLYADTATTNKEKTIK |
| | ISSSGNRFNQVVVMNSVGNNCTMNFKNNNGNNIGLLGFKADTVVASTWYYTHMRDH |
| | TNSNGCFWNFISEEHGWQEK |
| BoNT/E8 | >AER11392.1 botulinum neurotoxin E [Clostridium |
| SEQ ID | botulinum] |
| NO: 37 | MPKINSFNYNDPVNDKTILYIKPGGCQQFYKSFNIMKNIWIIPERNVIGTIPQDEL |
| | PPTSLKNGDSSYYDPNYLQSNEEKDRFLKIVTKIFNRINDNLSGRILLEELSKANP |
| | YLGNDNTPDNQFHIGDASAVEIKFSNGNQSILLPNVIIMGAEPDLFETNSSNISLR |
| | NNYMPSNHGFGSIAIVTFSPEYSFRENDNSMNEFIQDPALTLMHELIHSLHGLYGA |
| | KRITTKYTITQQQNPLITNIRGTNIEEFLTFGGTDLNIITSAQYNDIYTNLLADYK |
| | KIASKLSKVQVSNPQLNPYKDIFQEKYGLDKNASGIYSVNINKEDDIFKKLYSFTE |
| | FDLATKFQVKCRQTYIGQYKYFKLSNLLNNSIYNISEGYNINTLKVNFRGQNTNLN |
| | PRIITQLTGRGLVKKIIRFCKNIVFSKGITKSICIEINNGELFFVASENSYNDDNI |
| | NTPKEIDDTVTSNNNYENDLDQVILNENSESAPGLSDEKLNLTIQNDAYIPKYDSN |
| | GTSYIEQHDVNELNVFFYLDAQKVPEGENNVNLTSSIDTALLEQPKIYTFFSSEFI |
| | NNVNKTVQAALFVSWIQQVLVDFTTEANQKSTVDKIADISIVVPYIGLALNIGNEA |
| | QKGNFKDALELLGAGILLEFEPELLIPTILVFTIKSFLGSSDNKNKVIKAINNALK |
| | ERDEKWKEVYSFIVSNWMTKINTQFNKRKEQMYQALQNQVNALKTIIESKYNSYTL |
| | EEKNELTNKYNIEQIENELNQKVSIAMNNIEIFLTESSISYLMKLINEVKINKLRE |
| | YDENVKTYLLDYIIKHGSILGESQQELNSMVIDTLNNSIPFKLSSYTDDKILISYF |
| | NKFFKRIKSSSVLNMRYKNDKYVDTSGYDSNININGDVYKYPTNKNQFGIYNDKLS |
| | EVNISQNDYIIYDNKYKNFSISFWVRIPNYDNKIVNVNNEYTIINCMRDNNSGWKV |
| | SLNHNEIIWTLQDNAGINQKLAFNYGNANGISDYINKWIFVTITNDRLGDSKLYIN |
| | GNLIDKKSILNLGNIHVSDNILFKIVNCSYTRYIGIRYFNIFDKELDETEIQTLYN |
| | NEPNANILKDFWGNYLLYDKEYYLLNVLKPNNFIDRRTDSTLSINNIRSTILLANR |
| | LYSGIKVKIQRVNNSSTNDNLVRKNDQVYINFVASKTHLFPLYADTNTTNKEKTIK |
| | SSSSGNRFNQVVVMNSVGNNCTMNFKNNNGNNIGMLGFKDNTLVASTWYYTHMRDN |
| | TNSNGCFWNFISEEHGWQEK |
| BoNT/E9 | >AFV91339.1 botulinum neurotoxin type E [Clostridium |
| SEQ ID | botulinum] |
| NO: 38 | MPKINSFNYNDPVNDNTILYIKPGGCQQFYKSENIMKNIWIIPERNVIGTIPQNFL |
| | PPTSLKNGDSSYYDPNYLQNDQEKDRFLKIVTKVENRINDNLSGRILLEELSKANP |
| | YLGNDNTRDDDFIINDGSAVPIQFSNGSQSILLPTVIIMGAEPDLFETNSSNVSLI |
| | NNYSPSNHGFGSIAIVTFSPEYSFRENDNSMNEFIQDPALTLIHELIHSLHGLYGA |
| | KGITTKYTITQQQNPLITNIRGINIEEFLTFGGNNLNIITSSQLNDIYTNLLDDYK |
| | KIASKLSKVQVSNPQLNPYKDVFQEKYGLDKNASGIYSVNINKENDIFKKLYSFTE |
| | FDLATKFQVKCRETYIGQYKYFKLSNLLNDSIYNISEGYNINTLNVNERGQNPNLN |
| | PRIITPITDRGLVKKIIRFCKNIVSVKGIRKSICIEVNNGDLFFVASEKSYNNDSI |
| | NIPKEIDDTVTLNNNYENDLDQVILNENSESAPGLSDKKLNISIQDDVYIPKYDSN |
| | GTSDIEQYDVSELNVFFYLDAQKVPEGENNVNLTSSIDTALLEQSKIYTFFSSEFI |
| | NNVNKPVQAALFVGWIQQVLVDFTTEATQKSTVDKIADISIVVPYIGLALNIGNES |
| | QKGNFKDALELLGAGILLEFVPELLIPTILVFTIKSFLGSSDNKNKVIKAINNALK |
| | ERDEKWKEVYSFIVSNWITKINTQFNKRKEQMYQALQNQVNALKTIIESKYNSYTL |
| | EEKNELTNKYDIEQIENELNQKVSIAMNNIDRFLTESSISYLMKLINEVKINKLRE |
| | YDENVKTYLLDYITKHGSILGESQQELNSMIIDTLNNSIPFKLSSYTDDKILISYF |
| | NKFFKTIKSSSVLSMRYKNDKYIDTSGYDSNININGDVFIYPTNKNQFGIYNSKLS |
| | EVNISQNDYIIYDNKYKNFSISFWVRIPNYNNKIVNVNNEYTIINCMRDNNSGWKI |
| | SLNHNEIIWTLQDNAGINQKLVFKYGNANGISDYINKWIFVTITNDRLGYSKLYIN |
| | GHLIDQKSILNLGNIHVSDNILFKIVNCSYTRYIGMRYFNIFDKELDETEIQTLYN |
| | NEPNANVLKDFWGNYLLYNKEYYLLNMLKPSKTISHNRDLTFSIYNNRNIVNGLYR |
| | LYSGIKVKIQKINDSDTRDNIVRDNDQVYVNYINGNVYYSLYADTNATNKEKTIKS |
| | STSGNRFNQVVVMNSVRNNCTMNFKNNNGHDIGLLGFKSNALVASTWYYTNMRDHT |
| | NSNGCFWSFIPEENGWQEH |
| BoNT/E10 | >KF861920.1 Clostridium botulinum |
| SEQ ID | MPKINSFNYNDPVNDKTILYIKPGGCQQFYKSFNIMKNIWIIPERNVIGTIPQDEL |
| NO: 39 | PPTSLKNGDSSYYDPNYLQSNEEKDRFLKIVTKIFNRINDNLSGGILLEELSKANP |
| | YLGNDNTPNNQFHIGDASAVEIKFSNGSQSILLPTVIIMGAEPDLFETNSSNISLK |
| | NNYMPSNHGFGSIAIVTFSPEYSFRENDNSMNEFIQDPALTLMHELIHSLHGLYGA |
| | KGITTKYTITQQQNPLITNIRGTNIEEFLTFGGTDLNIITNAQSNDIYTNLLADYK |
| | KIASKLSQVQVSNPQLNPYKDIFQEKYGLDKNASGIYSVNINKEDDIFKKLYSFTE |
| | FDLATKFQVKCRQTYIGQYKYFKLSNLLNNSIYNISEGYNINTLKVNFRGQNTNLN |
| | PRIITQLTGRGLVKKIIRFCKNIVESKGITKSICIEINNGELFFVASENSYNDDNI |
| | NTPKEIDDTVTSNNNYENDLDQVILNENSESAPGLSDEKLNLTIQNDAYIPKYDSN |
| | GTSDIEQHDVNELNVFFYLDAQKVPEGENNVNLTSSIDTALLEQPKIYTFFSSEFI |
| | NNVNKPVQAALFVSWIQQVLVDETTEANQKSTVDKIADISIVVPYIGLALNIGNEA |
| | QKGNFKDALELLGAGILLEFEPELLIPTILVFTIKSFLGSSDNKNKVIKAINNALK |
| | ERDEKWKEVYSFIVSNWMTKINTQFNKRKEQMYQALQNQVNALKTIIESKYNSYTL |
| | EEKNELTNKYNIEQIENELNQKVSIAMNNIEIFLTESSISYLMKLINEVKINKLRE |
| | YDENVKTYLLDYIIKHGSILGESQQELNSMVIDTLNNSIPFKLSSYTDDKILISYF |
| | NKFFKRIKSSSVLNMRYKNDKYVDTSGYDSNININGDVYKYPTNKNQFGIYNDKLS |
| | EVNISQNDYIIYDNKYKNFSISFWVRIPNYDNKIVNVNNEYTIINCMRDNNSGWKV |
| | SLNHNEIIWTLQDNAGINQKLAFNYGNANGISDYINKWIFVTITNDRLGDSKLYIN |
| | GNLIDKKSILNLGNIHVSDNILFKIVNCSYTRYIGMRYFNIFDKELDKTEIETLYN |
| | NEPNTNILKDFWGNYLLYDKEYYLLNVLKPNNVIDSNRDSTFSIHNIRSTIVLANK |
| | LYLGIKVKIQRVNNSSTNDNLVRKNDQVYINFVPIKTHLFPLYADTNTTNKEKTIK |
| | SSSSGNRFNQVVVMNSVGNNCTMNFKNNNGNNIGMLGFKDNTLVASTWYYTHMRDN |
| | TNSNGCFWNFISEEHGWQEK |
| BoNT/E11 | >KF861879.1 Clostridium botulinum |
| SEQ ID | MPKINSFNYNDPVNDKTILYIKPGGCQQFYKSENIMKNIWIIPERNVIGTIPQDEL |
| NO: 40 | PPTSLKNGDSSYYDPNYLQSNEEKDRFLKIVTKIFNRINDNLSGGILLEELSKANP |
| | YLGNDNTPNNQFHIGDASAVEIKFSNGSQSILLPTVIIMGAEPDLFETNSSNISLK |
| | NNYMPSNHGFGSIAIVTFSPEYSFRENDNSMNEFIQDPALTLMHELIHSLHGLYGA |
| | KGITTKYTITQQQNPLITNIRGTNIEEFLTFGGTDLNIITNAQSNDIYTNLLDDYK |
| | KIASKLSQVQVSNPQLNPYKDVFQEKYGLDKDANGIYSVNINKENDIFKKLYSFTE |
| | FDLATKFQVKCRKTYIGHHKYFRLSDLLNDSIYNISDGYNINTLKVNFRGQNTNLN |
| | TRIITPITGRGVVRKIIRFCTNIFSPKGIRKSICIEVNNGELFFVASENSYNDDNI |
| | NTSKEIDDTVTSNNNYENDLDQVILNFNSESAPGLSDEKLNLTIQDDAYIPKYDSN |
| | GTSDIEQYDVSELNVFFYLDAQKVPEGENNVDFTSSIDTALLEQPKIYTFFSSKFI |
| | SNLNKTMQAALFVSWIQQVLVDFTTEATQKSTVDKIADISIVVPYIGLALNIGNEA |
| | QKGNFKDALELLGAGILLEFEPELLIPIILVFTIKSFLGSSDNKNKVIKAINNALK |
| | ERDENWKEVYSFIVSNWMTKINTQFNKRKEQMYQALQNQVNAIKTIIESKYNSYTL |
| | EEKNELKNKYDIEQIENELNQTVSIAMNNIEIFLTESSISYLMKLINEVKINKLKE |
| | YDENVKTYLLDYIIKHGSILGESQQELNSMVIDTLNNSIPFKLSSYTDDKILISYF |
| | NKFFKTIKSSSVLNMRYKNDKYIDTSGYDSNINIKGDVFIYPTNKNQFGIYNNKLS |
| | EVNISQNDYIIYDNKYKNFSISFWVRIPNYDNKIVNVNNEYTIINCMRDNNSGWKV |
| | SLNHNEIIWTLQDNAGINQKLVFKYGNANGISDYINKWIFVTITNDRLGDSKLYIN |
| | GNLIDKKSILNLGNIHVSDNILFKIVNCSYTRYIGMRYENIFDKELDKTEIETLYN |
| | NEPNTNILKDFWGNYLLYDKEYYLLNVLKPNNVIDSNRDSTFSIHNIRSTIVLANR |
| | LYSGIKVKIQRVNNSSTNDNLVRKNDQVYINFVASKTHLFPLYADTNTTNKEKTIK |
| | SSSSGNRFNQVVVMNSVGNNCTMNFKNNNGNNIGMLGFKDNTLVASTWYYTHMRDN |
| | TNSNGCFWNFISEEHGWQEK |
| BoNT/E12 | >KF929215.1 Clostridium botulinum |
| SEQ ID | MPKINSFNYNDPVNDRTILYIKPGGCQQFYKSFNIMKNIWIIPERNVIGTIPQDFQ |
| NO: 41 | PPTSLKNGDSSYYDPNYLQSNEEKDRFLKIVTKIFNRINDNLSGGILLEELSKANP |
| | YLGNDNTPDGDFIINDASAVPIQFSNGSQSILLPNVIIMGAEPDLFETNSSNISLI |
| | NNYRPSNHGFGSIAIVTFSPEYSFRENDNSMNEFIQDPALTLMHELIHSLHGLYGA |
| | KGITTKYTITQQQNSLITNIRGINIEEFLTFGGNDLNIITSSQFNDIYTNLLDDYK |
| | KIASKLSQVRVSNPQLNPYKDVFQEKYGLDKDASGIYSVNINKENDIFKKLYSFTE |
| | FDLATKFQVKCRETYIGQYKYFQLSNLLNDSIYNISEGYNINNLKVNFRGQNANLN |
| | PRIITPITGRGLVKKIIRFCKNIVSVKGIRKSICIEVNNGELFFVASENSYNDDNI |
| | NTPKEIDDTVTSNNNYENDLDQVILNENSESAPGLSDEKLNLTIQDDAYIPKYDSN |
| | GTSDIEQHDVNELNVFFYLDAQKVPEGENNVNLTSSIDTALLEQPKIYTFFSSEFI |
| | NNVNKPVQAVLFVSWIQQVLVDFTTEATQKSTVDKIADISIVVPYIGLALNIGNEA |
| | QKGNFKDALELLGAGILLEFVPELLIPTILVFTIKSFLGSSDNKNKIIKAINNALK |
| | ERDEKWKEVYSFIVSNWITKINTQFNKRKEQMYQALQNQVNAIKTIIESKYNSYTL |
| | EEKNELTNKYDIKQIENELNQKVSIAMNNIDRELTESSISYLMKLINEVKINKLRE |
| | YDENVKTYLLNYIIQHGSTLGESQQELNSMVINTLNNSIPFKLSSYTDDKILISYF |
| | NKFFKRIKSSSVLNMRYKNDKYVDTSGYDSNININGEIFIYPTNKNQFSIFNSKPS |
| | EVNISQNDYIIYDNKYKNFSISFWVRIPNYDNKIVNVNNEYTIINCMRDNNSGWKV |
| | SLNHNEIIWTLQDNAGINQKLAFNYGNSNGISDYINKWIFVTITNDRLGDSKLYIN |
| | GNLIDQKSILNLGNIHVSDNILFKIVNCSYTRYIGIRYENIFDKELDETEIQTLYS |
| | NEPNTNILKDFWGNYLLYDKEYYLLNVLKPNSIISHRRDLTFSFYNHRYIVNGLYR |
| | LYSGIKVKIQRVNDSSTNDQFVRKNDQVYINYIYNNLSYSLYADTNIKDKEKTIKS |
| | SLSGNIFNQVVVMNSVGNNCTMNFKNNNGNNIGLLGFKDNTLVASTWYYTHMRDNT |
| | NSNGCFWNFISEEHGWQEK |
| BoNT/F1 | >ABS41202.1 botulinum neurotoxin type F, BoNT/F |
| SEQ ID | [Clostridium botulinum F str. Langeland] |
| NO: 42 | MPVVINSFNYNDPVNDDTILYMQIPYEEKSKKYYKAFEIMRNVWIIPERNTIGTDP |
| | SDFDPPASLENGSSAYYDPNYLTTDAEKDRYLKTTIKLFKRINSNPAGEVLLQEIS |
| | YAKPYLGNEHTPINEFHPVTRTTSVNIKSSTNVKSSIILNLLVLGAGPDIFENSSY |
| | PVRKLMDSGGVYDPSNDGFGSINIVTFSPEYEYTENDISGGYNSSTESFIADPAIS |
| | LAHELIHALHGLYGARGVTYKETIKVKQAPLMIAEKPIRLEEFLTFGGQDLNIITS |
| | AMKEKIYNNLLANYEKIATRLSRVNSAPPEYDINEYKDYFQWKYGLDKNADGSYTV |
| | NENKFNEIYKKLYSFTEIDLANKFKVKCRNTYFIKYGFLKVPNLLDDDIYTVSEGF |
| | NIGNLAVNNRGQNIKLNPKIIDSIPDKGLVEKIVKFCKSVIPRKGTKAPPRLCIRV |
| | NNRELFFVASESSYNENDINTPKEIDDTTNLNNNYRNNLDEVILDYNSETIPQISN |
| | QTLNTLVQDDSYVPRYDSNGTSEIEEHNVVDLNVFFYLHAQKVPEGETNISLTSSI |
| | DTALSEESQVYTFFSSEFINTINKPVHAALFISWINQVIRDFTTEATQKSTEDKIA |
| | DISLVVPYVGLALNIGNEVQKENFKEAFELLGAGILLEFVPELLIPTILVFTIKSF |
| | IGSSENKNKIIKAINNSLMERETKWKEIYSWIVSNWLTRINTQFNKRKEQMYQALQ |
| | NQVDAIKTVIEYKYNNYTSDERNRLESEYNINNIREELNKKVSLAMENIERFITES |
| | SIFYLMKLINEAKVSKLREYDEGVKEYLLDYISEHRSILGNSVQELNDLVTSTLNN |
| | SIPFELSSYTNDKILILYENKLYKKIKDNSILDMRYENNKFIDISGYGSNISINGD |
| | VYIYSTNRNQFGIYSSKPSEVNIAQNNDIIYNGRYQNFSISFWVRIPKYFNKVNLN |
| | NEYTIIDCIRNNNSGWKISLNYNKIIWTLQDTAGNNQKLVENYTQMISISDYINKW |
| | IFVTITNNRLGNSRIYINGNLIDEKSISNLGDIHVSDNILFKIVGCNDTRYVGIRY |
| | FKVFDTELGKTEIETLYSDEPDPSILKDFWGNYLLYNKRYYLLNLLRTDKSITQNS |
| | NFLNINQQRGVYQKPNIFSNTRLYTGVEVIIRKNGSTDISNTDNFVRKNDLAYINV |
| | VDRDVEYRLYADISIAKPEKIIKLIRTSNSNNSLGQIIVMDSIGNNCTMNFQNNNG |
| | GNIGLLGFHSNNLVASSWYYNNIRKNTSSNGCFWSFISKEHGWQEN |
| BoNT/F2 | >CAA73972.1 bonT [Clostridium botulinum] |
| SEQ ID | MPVVINSENYNDPVNDETILYMQKPYEERSRKYYKAFEIMPNVWIMPERDTIGTKP |
| NO: 43 | DEFQVPDSLKNGSSAYYDPNYLTTDAEKDRYLKIMIKLENRINSNPTGKVLLEEVS |
| | NARPYLGDDDTLINEFLPVNVTTSVNIKESTDVESSIISNLLVLGAGPDIFKAYCT |
| | PLVRENKSDKLIEPSNHGEGSINILTESPEYEHIENDISGGNHNSTESFIADPAIS |
| | LAHELIHALHGLYGAKAVTHKESLVAERGPLMIAEKPIRLEEFLTFGGEDLNIIPS |
| | AMKEKIYNDLLANYEKIATRLREVNTAPPGYDINEYKDYFQWKYGLDRNADGSYTV |
| | NRNKENEIYKKLYSFTEIDLANKFKVKCRNTYFIKYGFVKVPNLLDDDIYTVSEGE |
| | NIGNLAVNNRGQNINLNPKIIDSIPDKGLVEKIIKFCKSIIPRKGTKQSPSLCIRV |
| | NNREIFFVASESSYNESDINTPKEIDDTTNLNNNYRNNLDEVILDYNSETIPQISN |
| | RTLNTLVQDNSYVPRYDSNGTSEIEEYDVVDENVFFYLHAQKVPEGETNISLTSSI |
| | DTALLEESKVYTFFSSEFIDTINKPVNAALFIDWISKVIRDETTEATQKSTVDKIA |
| | DISLIVPYVGLALNIVIEAEKGNFEEAFELLGAGILLEFVPELTIPVILVFTIKSY |
| | IDSYENKNKAIKAINNSLIEREAKWKEIYSWIVSNWLTRINTQFNKRKEQMYQALQ |
| | NQVDAIKTAIEYKYNNYTSDEKNRLESKYNINNIEEELNKKVSLAMKNIERFMTES |
| | SISYLMKLINEAEVGKLKEYDKHVKSDLLDYILYHKLILGEQTKELIDLVTSTINS |
| | SIPFELSSYINDKILIIYENRLYKKIKDSSILDMRYENNKFIDISGYGSNISINGN |
| | VYIYSTNRNQFGIYSGRLSEVNIAQNNDIIYNSRYQNFSISFWVTIPKHYRPMNRN |
| | REYTIINCMGNNNSGWKISLRTIRDCEIIWTLQDTSGNKEKLIFRYEELASISDYI |
| | NKWIFVTITNNRLGNSRIYINGNLIVEKSISNLGDIHVSDNILFKIVGCDDETYVG |
| | IRYFKVENTELDKTEIETLYSNEPDPSILKDYWGNYLLYNKKYYLFNLLRKDKYIT |
| | RNSGILNINQQRGVTGGISVFLNYKLYEGVEVIIRKNAPIDISNIDNEVRKNDLAY |
| | INVVDHGVEYRLYADISITKSEKIIKLIRTSNPNDSLGQIIVMDSIGNNCTMNFQN |
| | NDGSNIGLLGFHSDDLVASSWYYNHIRRNTSSNGCFWSFISKEHGWKE |
| BoNT/F3 | >ADA79575.1 botulinum neurotoxin type F [Clostridium |
| SEQ ID | botulinum] |
| NO: 44 | MPVVINSFNYNDPVNDETILYMQKPYEERSRKYYKAFEIMPNVWIMPERDTIGTKP |
| | DDFQVPDSLKNGSSAYYDPNYLTTDAEKDRYLKTMIKLFNRINSNPTGKVLLEEVS |
| | NARPYLGDDDTLINEFFPVNVTTSVNIKFSTDVESSIISNLLVLGAGPDIFKAYCT |
| | PLVRENKSDKLIEPSNHGFGSINILTFSPEYEHIFNDISGGDHNSTESFIADPAIS |
| | LAHELIHALHGLYGAKAVTHKETIEVKRGPLMIAEKPIRLEEFLTFGGEDLNIIPS |
| | AMKEKIYNDLLANYEKIATRLREVNTAPPEYDINEYKDYFQWKYGLDRNADGSYTV |
| | NRNKFNGIYKKLYSFTEIDLANKFKVKCRNTYFIKYGFVKVPDLLDDDIYTVSEGF |
| | NIGNLAVNNRGQNINLNPKIIDSIPDKGLVEKIIKFCKSIIPRKGTKQSPSLCIRV |
| | NNRELFFVASESSYNESDINTPKEIDDTTNLNNNYRNNLDEVILDYNSETIPQISN |
| | RTLNTLVQDNSYVPRYDSNGTSEIEEYDVVDENVFFYLHAQKVPEGETNISLTSSI |
| | DTALLEKSKVYTFFSSEFIDTINESVNAALFIDWINKVIRDFTTEATQKSTVDKIA |
| | DISLIVPYVGLALNIVIDAEKGNFQEAFELLGAGILLEFVPELTIPVILVFTIKSY |
| | IDSYENKNKAIKAINNALIEREAKWKEIYSWIVSNWLTKINTQFNKRKEQMYQALQ |
| | NQVDAIKTAIEYKYNNYTSDEKNRLESEYNINNIEEELNKKVSLAMKNIERFMTES |
| | SISYLMKLINEAEVGKLKKYDRHVKSDLLDYILYHKLILGDQTKELIDLVTSTINS |
| | SIPFELSSYTNDKILIIYFNRLYKKIKDSSILDMRYENNKFIDISGYGSNISINGN |
| | VYIYSTNRNQFGIYSDRLSEVNIAQNNDIIYNSRYQNFSISFWVRIPKHYGPMNRN |
| | REYTIINCMGNNNSGWKISLRNIRDCEIIWTLQDTSGNKEKLIFRYEELANISDYI |
| | NKWIFVTITNNRLGNSRIYINGNLIVEKSISNLGDIHVSDNILFKIVGCDDKTYVG |
| | IRYFKVENTELDKTEIETLYSNEPDPSILKDYWGNYLLYNKKYYLFNLLRKDKYIT |
| | RNSGILNINQQRGVTEGSVFLNYKLYEGVEVIIRKNGPIDISNTDNFVRKNDLAYI |
| | NVVYHDVEYRLYADISITKPEKIIKLIRTSNPNDSLGQIIVMDSIGNNCTMNFQNN |
| | NGGNIGLLGFHSDNLVASSWYYNNIRRNTSSNGCFWSFISKEHGWQE |
| BoNT/F4 | >GU213221.1 Clostridium botulinum |
| SEQ ID | MPVVINSFNYDDPVNDDTILYMQIPYEEKSKKYYKAFEIMRNVWIMPERNTIGTNP |
| NO: 45 | SDFDPPASLKNGSSAYYDPNYLTTDAEKDRYLKTTIKLFKRINSNPAGEVLLQEIS |
| | YAKPYLGNDHTPINEFHPVTRTTSVNIKSSTNVESSIILNLLVLGAGPNIFENSSY |
| | PVRKLMNSGEVYDPSNDGFGSINIVTFSPEYEYTENDISGGHNSSTESFIADPAIS |
| | LAHELIHALHGLYGARGVTYKETIKVKQAPLMIAEKPIRLEEFLTFGGQDLNIITS |
| | AMKEKIYNDLLANYEKIATRLSEVNSAPPEYDINEYKNYFQWKYGLDKNADGSYTV |
| | NENKFNEIYKKLYSFTEIDLANKFKVKCRNTYFIKYGFLKVPNLLDDDIYTVSEGF |
| | NIGNLAVNNRGQNINLNPKIIDSIPDKGLVEKIVKLCKSIIPRKGTKAPPRLCIRV |
| | NNRELFFVASESSYNENDINTPKEIDDTTNLNNNYRNNLDEVILDYNSETIPQISS |
| | QTLNTLVQDDSYVPRYDSNGTSEIEEHNVVDLNAFFYLHAQKVPEGETNISLTSSI |
| | DTALSEESKVYTFFSSEFINNINKPVHAALFIGWISQVIRDFTTESTQKSTVDKIA |
| | DISLIVPYVGLALNIGNDARKGNFKEAFELLGAAILLEVVPELLIPVILVFTIKSF |
| | IDSSKNEDKIIKAINNSLIEREAKWKEVYSWIVSNWLTRINTQFNKRKEQMYQALQ |
| | NQVDAIKTVIEYKYNSYTSDEKNRLESEYNINNIEEELNKKVSLAMKNIERFIAES |
| | SISYLMKLINEAKVSELREYDEGVKEYLLDYILKNGSILGDHVQELNDLVTSTINS |
| | SIPFELSSYTNDKILIIYFNKLYKKIKDNCILDMRYENNKFIDISGYGSNISINGE |
| | LYIYTTNRNQFTIYSGKLSEVNIAQNNDIIYNSRYQNFSISFWVRIPRYSNIVNLN |
| | NEYTIINCMGNNNSGWKISLNYNKIIWTLQDTAGNNEKLVFNYTQMISISDYINKW |
| | IFVTITNNRLGNSRIYINGNLIDQKSISNLGDIHVSDNILFKIVGCNDTRYVGIRY |
| | FKVEDTELDKTEIETLYSDEPDPSILKDFWGNYLLYNKRYYLLNLLRKDNAITQSS |
| | TFLSISRARGVDRKANIFSNKRLYKGVEVIIRKNEPIDISNTDNFVRKGDLAYINV |
| | VDRDVEYRLYANTSNAQPEKTIKLIRTSNSNDSLDQIIVMDSIGNNCTMNFQNNNG |
| | GNIGLLGFHSNTLVASSWYYNNIRRNTSSNGCFWSFISKEHGWQE |
| BoNT/F5 | >GU213212.1 Clostridium botulinum |
| SEQ ID | MPVEINSFNYDDLVNDNTILYIRPPYYERSNTYFKAFNIMENVWIIPERYRLGIEA |
| NO: 46 | SKFDPPDSLKAGSDGYFDPNYLSTNTEKNRYLQIMIKLFKRINSNEAGKILLNQIK |
| | DAIPYLGNSYTAEDQFTTNNRTISFNVRLANGTIEQEMANLIIWGPGPDLTTNRTG |
| | GTTYTPAQSLEAIPYKEGFGSIMTIEFSPEYATAFNDISLTSHAPSLFIKDPALIL |
| | MHELIHVLHGLYGTYTTGFKIKPNITEPYMEVTKPITSGEFLTFGGNDVNKIPQLI |
| | QSQLRSKVLDDYEKIASRLNKVNRATAEINIDKFKYSYQLKYQFVKDSNGVYSVDL |
| | DKFNKLYDKIYSFTEFNLAHEFKIKTRNSYLAKNFGPFYLPNLLDNSIYNEADGEN |
| | IGDLSVNYKGQVIGSDIDSIKKLEGQGVVSRVVRLCLNSSFKKNTKKPLCITVNNG |
| | DLFFIASEDSYGEDTINTPKEIDDTTTLVPSFKNILDKVILDENKQVTPQIPNRRI |
| | RTDIQEDNYIPEYDSNGTSEIEEYNVVDLNAFFYLHAQKVPEGETNISLTSSIDTA |
| | LSEESKVYTFFSSEFIDTINEPVNAALFIDWISKVIRDFTTEATQKSTVDKIADIS |
| | LIVPYVGLALNIVNETEKGNFKEAFELLGAGILLEFVPELAIPVILVFTIKSYIDS |
| | YENKNKIIKAINNSLIEREAKWKEIYSWIVSNWLTRINTQFNKRKEQMYQALQNQV |
| | DAIKTAIEYKYNNYTSDEKNRLESEYNINNIEEELNKKVSLAMKNIERFITESSIS |
| | YLMKLINEAEVGKLKEYDKRVKRHLLEYIFDYRLILGEQGGELIDLVTSTLNTSIP |
| | FELSSYTNDKILIIYFNRLYKKIKDSSILDMRYENNKFIDISGYGSNISINGNVYI |
| | YSTNRNQFGIYDDRLSEVNIAQNNDIIYNSRYQNFSISFWVRIPKHYRPMNHNREY |
| | TIINCMGNNNSGWKISLRTTGDCEIIWTLQDTSGNKKKLIFRYSQLGGISDYINKW |
| | IFVTITNNRLGNSRIYINGNLIVEKSISNLGDIHVSDNILFKIVGCDDKMYVGIRY |
| | FKVENTELDKTEIEILYSNEPDPSILKDYWGNYLLYNKKYYLLNLLRNDKYITRNS |
| | DILNISHQRGVTKDLFIFSNYKLYEGVEVIIRKNGPIDISNTDNFVRKNDLAYINV |
| | VDHGVEYRLYADISITKPEKIIKLIRRSNPDDSLGQIIVMDSIGNNCTMNFQNNNG |
| | GNIGLLGFHSDNLVASSWYYNNIRRNTSSNGCFWSFISKEHGWQE |
| BoNT/F6 | >AAA23263.1 neurotoxin type F [Clostridium botulinum] |
| SEQ ID | MPVAINSFNYNDPVNDDTILYMQIPYEEKSKKYYKAFEIMRNVWIIPERNTIGTNP |
| NO: 47 | SDFDPPASLKNGSSAYYDPNYLTTDAEKDRYLKTTIKLFKRINSNPAGKVLLQEIS |
| | YAKPYLGNDHTPIDEFSPVTRTTSVNIKLSTNVESSMLLNLLVLGAGPDIFESCCY |
| | PVRKLIDPDVVYDPSNYGFGSINIVTFSPEYEYTENDISGGHNSSTESFIADPAIS |
| | LAHELIHALHGLYGARGVTYEETIEVKQAPLMIAEKPIRLEEFLTFGGQDLNIITS |
| | AMKEKIYNNLLANYEKIATRLSEVNSAPPEYDINEYKDYFQWKYGLDKNADGSYTV |
| | NENKFNEIYKKLYSFTESDLANKFKVKCRNTYFIKYEFLKVPNLLDDDIYTVSEGF |
| | NIGNLAVNNRGQSIKLNPKIIDSIPDKGLVEKIVKFCKSVIPRKGTKAPPRLCIRV |
| | NNSELFFVASESSYNENDINTPKEIDDTTNLNNNYRNNLDEVILDYNSQTIPQISN |
| | RTLNTLVQDNSYVPRYDSNGTSEIEEYDVVDENVFFYLHAQKVPEGETNISLTSSI |
| | DTALLEESKDIFFSSEFIDTINKPVNAALFIDWISKVIRDFTTEATQKSTVDKIAD |
| | ISLIVPYVGLALNIIIEAEKGNFEEAFELLGVGILLEFVPELTIPVILVFTIKSYI |
| | DSYENKNKAIKAINNSLIEREAKWKEIYSWIVSNWLTRINTQFNKRKEQMYQALQN |
| | QVDAIKTAIEYKYNNYTSDEKNRLESEYNINNIEEELNKKVSLAMKNIERFMTESS |
| | ISYLMKLINEAKVGKLKKYDNHVKSDLLNYILDHRSILGEQTNELSDLVTSTLNSS |
| | IPFELSSYTNDKILIIYFNRLYKKIKDSSILDMRYENNKFIDISGYGSNISINGNV |
| | YIYSTNRNQFGIYNSRLSEVNIAQNNDIIYNSRYQNFSISFWVRIPKHYKPMNHNR |
| | EYTIINCMGNNNSGWKISLRTVRDCEIIWTLQDTSGNKENLIFRYEELNRISNYIN |
| | KWIFVTITNNRLGNSRIYINGNLIVEKSISNLGDIHVSDNILFKIVGCDDETYVGI |
| | RYFKVENTELDKTEIETLYSNEPDPSILKNYWGNYLLYNKKYYLENLLRKDKYITL |
| | NSGILNINQQRGVTEGSVFLNYKLYEGVEVIIRKNGPIDISNTDNFVRKNDLAYIN |
| | VVDRGVEYRLYADTKSEKEKIIRTSNLNDSLGQIIVMDSIGNNCTMNFQNNNGSNI |
| | GLLGFHSNNLVASSWYYNNIRRNTSSNGCFWSSISKENGWKE |
| BoNT/F7 | >ADK48765.1 botulinum neurotoxin type F [Clostridium |
| SEQ ID | baratii] |
| NO: 48 | MPVNINNFNYNDPINNTTILYMKMPYYEDSNKYYKAFEIMDNVWIIPERNIIGKKP |
| | SDFYPPISLDSGSSAYYDPNYLTTDAEKDRFLKTVIKLENRINSNPAGQVLLEEIK |
| | NGKPYLGNDHTAVNEFCANNRSTSVEIKESKGTTDSMLLNLVILGPGPNILECSTF |
| | PVRIFPNNIAYDPSEKGFGSIQLMSFSTEYEYAFNDNTDLFIADPAISLAHELIHV |
| | LHGLYGAKGVTNKKVIEVDQGALMAAEKDIKIEEFITFGGQDLNIITNSTNQKIYD |
| | NLLSNYTAIASRLSQVNINNSALNTTYYKNFFQWKYGLDQDSNGNYTVNISKENAI |
| | YKKLFSFTECDLAQKFQVKNRSNYLFHFKPFRLLDLLDDNIYSISEGENIGSLRVN |
| | NNGQNINLNSRIVGPIPDNGLVERFVGLCKSIVSKKGTKNSLCIKVNNRDLFFVAS |
| | ESSYNENGINSPKEIDDTTITNNNYKKNLDEVILDYNSDAIPNLSSRLLNTTAQND |
| | SYVPKYDSNGTSEIKEYTVDKLNVFFYLYAQKAPEGESAISLTSSVNTALLDASKV |
| | YTFFSSDFINTVNKPVQAALFISWIQQVINDFTTEATQKSTIDKIADISLVVPYVG |
| | LALNIGNEVQKGNFKEAIELLGAGILLEFVPELLIPTILVFTIKSFINSDDSKNKI |
| | IKAINNALRERELKWKEVYSWIVSNWLTRINTQFNKRKEQMYQALQNQVDGIKKII |
| | EYKYNNYTLDEKNRLKAEYNIYSIKEELNKKVSLAMQNIDRFLTESSISYLMKLIN |
| | EAKINKLSEYDKRVNQYLLNYILENSSTLGTSSVQELNNLVSNTLNNSIPFELSEY |
| | TNDKILISYFNRFYKRIIDSSILNMKYENNRFIDSSGYGSNISINGDIYIYSTNRN |
| | QFGIYSSRLSEVNITQNNTIIYNSRYQNFSVSFWVRIPKYNNLKNLNNEYTIINCM |
| | RNNNSGWKISLNYNNIIWTLQDTTGNNQKLVFNYTQMIDISDYINKWTFVTITNNR |
| | LGHSKLYINGNLTDQKSILNLGNIHVDDNILFKIVGCNDTRYVGIRYFKIFNMELD |
| | KTEIETLYHSEPDSTILKDFWGNYLLYNKKYYLLNLLKPNMSVTKNSDILNINRQR |
| | GIYSKTNIFSNARLYTGVEVIIRKVGSTDTSNTDNFVRKNDTVYINVVDGNSEYQL |
| | YADVSTSAVEKTIKLRRISNSNYNSNQMIIMDSIGDNCTMNFKTNNGNDIGLLGFH |
| | LNNLVASSWYYKNIRNNTRNNGCFWSFISKEHGWQE |
| BoNT/F8 | >WP 076177537.1 botulinum neurotoxin subtype F8 |
| SEQ ID | [Clostridium botulinum] |
| NO: 49 | MPVVINSFNYNDPVNDDTILYMQIPYEEKSKKYYKAFEIMRNVWIIPERNTIGTDP |
| | SDFDPPASLKNGSSAYYDPNYLTTDAEKDKYLKTTIKLFKRINSNPAGEVLLQEIS |
| | YAKPYLGNEHTPINEFHPVTRTTSVNIKSSTNVKSSIILNLLVLGAGPNIFENSCY |
| | PVRKLMDSGEVYDPSNDGFGSINIVTFSPEYEYTENDISGGHNSSTESFIADPAIS |
| | LAHELIHALHGLYGARGVTYKETIKVKQAPLMIAEKPIRLEEFLTFGGQDLNIITS |
| | AMKEKIYNNLLANYEKIATRLSEVTSAPPEYDINEYKDYFQWKYGLDKNADGSYTV |
| | NENKFNEIYKKLYSFTENDLANKFKVKCRNTYFIKYGFLKVPNLLDDDIYTVSEGF |
| | NIGNLAINNRGQNIKLNPKIIDSIPDKGLVEKIVKFCKSVIPRKGTKAPPRLCIRV |
| | NNRELFFVASESSYNENDINTPKEIDDTTNLNNNYRNNLDEVILDYNSETIPQISN |
| | QTLNTLVQDDSYVPRYDSNGTSEIEEHNVVDLNVFFYLHAQKVPEGETNISLTSSI |
| | DTALSEESQVYTFFSSEFINTINKPVHAALFISWINQVIRDFTTEATQKSTEDKIA |
| | DISLIVPYVGLALNIGNDVSKGDEKKAFELFGAAILLEVAPELLIPVILVFTIKSF |
| | IDSSENEDKIIKAIIKAINNSLMEREAKWQEIYGWIVSNWLTRINTQFNKRKEQMY |
| | QALQNQVDAIKTVIEYKYNNYTSDEKNRLESEYNINNIEEELNKKVSLAMKNIERF |
| | IAESSISYLMKLINEAKVSKLREYDEGVKEYLLDYILKHGSILGDRVQELNDLVTS |
| | TLNSSIPFELSSYTNDKILIIYFNKLYEKIKDNSILDMRYKNNKFIDISGYGSNIS |
| | INGDVYIYSTNRNQFGIYSNKPSEVNIAQNNDIIYNSRYQNFSISFWVRIPKYENK |
| | VNLNNEYTIIDCIRNNNSGWKISLNYNKIIWTLQDTAGNNQKLVENYTQMISISDY |
| | INKWIFVTITNNRLGNSRIYINGNLIDEKSISNLGDIHVSDNILFKIVGCNDTRYV |
| | GIRYFKVEDTELDKTEIETLYSDEPDPSILKDFWGNYLLYNKRYYLLNLLRTDKSI |
| | TQNSNFLNINQQRGVYQKPNIFSNTRLYTGVEVIIRKNGSTDISNTDDFVRKNDLA |
| | YINVVDHGVEYRLYADISIAKSEKIIKLIRTSNSNNSLGQIIVMDSIGNNCTMNFQ |
| | NNNGGNIGLLGFHSNNLVASSWYYNNIRKNTSSNGCFWSFISKEHGWQE |
| BoNT/G | >KIE44899.1 botulinum neurotoxin type G [Clostridium |
| SEQ ID | argentinense CDC 2741] |
| NO: 50 | MPVNIKNFNYNDPINNDDIIMMEPENDPGPGTYYKAFRIIDRIWIVPERFTYGFQP |
| | DQFNASTGVFSKDVYEYYDPTYLKTDAEKDKFLKTMIKLFNRINSKPSGQRLLDMI |
| | VDAIPYLGNASTPPDKFAANVANVSINKKIIQPGAEDQIKGLMTNLIIFGPGPVLS |
| | DNFTDSMIMNGHSPISEGFGARMMIRFCPSCLNVENNVQENKDTSIFSRRAYFADP |
| | ALTLMHELIHVLHGLYGIKISNLPITPNTKEFFMQHSDPVQAEELYTFGGHDPSVI |
| | SPSTDMNIYNKALQNFQDIANRLNIVSSAQGSGIDISLYKQIYKNKYDFVEDPNGK |
| | YSVDKDKFDKLYKALMFGFTETNLAGEYGIKTRYSYFSEYLPPIKTEKLLDNTIYT |
| | QNEGENIASKNLKTEFNGQNKAVNKEAYEEISLEHLVIYRIAMCKPVMYKNTGKSE |
| | QCIIVNNEDLFFIANKDSFSKDLAKAETIAYNTQNNTIENNFSIDQLILDNDLSSG |
| | IDLPNENTEPFTNEDDIDIPVYIKQSALKKIFVDGDSLFEYLHAQTFPSNIENLQL |
| | TNSLNDALRNNNKVYTFFSTNLVEKANTVVGASLFVNWVKGVIDDFTSESTQKSTI |
| | DKVSDVSIIIPYIGPALNVGNETAKENFKNAFEIGGAAILMEFIPELIVPIVGFFT |
| | LESYVGNKGHIIMTISNALKKRDQKWTDMYGLIVSQWLSTVNTQFYTIKERMYNAL |
| | NNQSQAIEKIIEDQYNRYSEEDKMNINIDENDIDFKLNQSINLAINNIDDFINQCS |
| | ISYLMNRMIPLAVKKLKDFDDNLKRDLLEYIDTNELYLLDEVNILKSKVNRHLKDS |
| | IPFDLSLYTKDTILIQVENNYISNISSNAILSLSYRGGRLIDSSGYGATMNVGSDV |
| | IFNDIGNGQFKLNNSENSNITAHQSKFVVYDSMFDNESINFWVRTPKYNNNDIQTY |
| | LQNEYTIISCIKNDSGWKVSIKGNRIIWTLIDVNAKSKSIFFEYSIKDNISDYINK |
| | WFSITITNDRLGNANIYINGSLKKSEKILNLDRINSSNDIDFKLINCTDTTKFVWI |
| | KDFNIFGRELNATEVSSLYWIQSSTNTLKDFWGNPLRYDTQYYLFNQGMQNIYIKY |
| | FSKASMGETAPRTNFNNAAINYQNLYLGLRFIIKKASNSRNINNDNIVREGDYIYL |
| | NIDNISDESYRVYVLVNSKEIQTQLFLAPINDDPTFYDVLQIKKYYEKTTYNCQIL |
| | CEKDTKTFGLFGIGKFVKDYGYVWDTYDNYFCISQWYLRRISENINKLRLGCNWQF |
| | IPVDEGWTE |
| BoNT/FA (H) | >KG015617.1 peptidase M27 [Clostridium botulinum] |
| SEQ ID | MPVVINSFNYDDPVNDNTIIYIRPPYYETSNTYFKAFQIMDNVWIIPERYRLGIDP |
| NO: 51 | SLFNPPVSLKAGSDGYFDPNYLSTNTEKNKYLQIMIKLFKRINSKPAGQILLEEIK |
| | NAIPYLGNSYTQEEQFTTNNRTVSFNVKLANGNIVQQMANLIIWGPGPDLTINKTG |
| | GIIYSPYQSMEATPYKDGFGSIMTVEFSPEYATAFNDISIASHSPSLFIKDPALIL |
| | MHELIHVLHGLYGTYITEYKITPNVVQSYMKVTKPITSAEFLTFGGRDRNIVPQSI |
| | QSQLYNKVLSDYKRIASRLNKVNTATALINIDEFKNLYEWKYQFAKDSNGVYSVDL |
| | NKFEQLYKKIYSFTEFNLAYEFKIKTRLGYLAENFGPFYLPNLLDDSIYTEVDGEN |
| | IGALSINYQGQNIGSDINSIKKLQGQGVVSRVVRLCSNSNTKNSLCITVNNRDLFF |
| | IASQESYGENTINTYKEIDDTTTLDPSFEDILDKVILNFNEQVIPQMPNRNVSTDI |
| | QKDNYIPKYDYNRTDIIDSYEVGRNYNTFFYLNAQKFSPNESNITLTSSFDTGLLE |
| | GSKVYTFFSSDFINNINKPVQALLFIEWVKQVIRDETTEATKTSTVDKLKDISLVV |
| | PYIGLALNIGDEIYKQHFAEAVELVGAGLLLEFSPEFLIPTLLIFTIKGYLTGSIR |
| | DKDKIIKTLDNALNVRDQKWKELYRWVVSKWLTTINTQFNKRKEQMYKALKNQATA |
| | IKKIIENKYNNYTTDEKSKIDSSYNINEIERTLNEKINLAMKNIEQFITESSIAYL |
| | INIINNETIQKLKSYDDLVRRYLLGYIRNHSSILGNSVEELNSKVNNHLDNGIPFE |
| | LSSYTNDSLLIRYFNKNYGELKYNCILNIKYEMDRDKLVDSSGYRSRINIGTGVKF |
| | SEIDKNQVQLSNLESSKIEVILNNGVIYNSMYENFSTSFWIRIPKYFRNINNEYKI |
| | ISCMQNNSGWEVSLNFSNMNSKIIWTLQDTEGIKKTVVFQYTQNINISDYINRWIF |
| | VTITNNRLSNSKIYINGRLINEESISDLGNIHASNNIMFKLDGCRDPHRYIWIKYF |
| | NLFDKELNKKEIKDLYDNQSNSGILKDFWGDYLQYDKPYYMLNLYDPNKYLDVNNV |
| | GIRGYMYLKGPRGRIVTTNIYLNSTLYMGTKFIIKKYASGNKDNIVRNNDRVYINV |
| | VVKNKEYRLATNASQAGVEKILSAVEIPDVGNLSQVVVMKSENDQGIRNKCKMNLQ |
| | DNNGNDIGFIGFHQFNNIAKLVASNWYNRQIGKASRTFGCSWEFIPVDDGWGESSL |
| BoNT/X | UniProtKB/Swiss-Prot: P0DPK1.1 >sp\|P0DPK1.1\| BXX_CLOBO |
| SEQ ID | RecName: Full = Botulinum neurotoxin type X |
| NO: 52 | MKLEINKFNYNDPIDGINVITMRPPRHSDKINKGKGPFKAFQVIKNIWIVPERYNF |
| | TNNTNDLNIPSEPIMEADAIYNPNYLNTPSEKDEFLQGVIKVLERIKSKPEGEKLL |
| | ELISSSIPLPLVSNGALTLSDNETIAYQENNNIVSNLQANLVIYGPGPDIANNATY |
| | GLYSTPISNGEGTLSEVSFSPFYLKPFDESYGNYRSLVNIVNKFVKREFAPDPAST |
| | LMHELVHVTHNLYGISNRNFYYNFDTGKIETSRQQNSLIFEELLTFGGIDSKAISS |
| | LIIKKIIETAKNNYTTLISERLNTVTVENDLLKYIKNKIPVQGRLGNFKLDTAEFE |
| | KKLNTILFVLNESNLAQRFSILVRKHYLKERPIDPIYVNILDDNSYSTLEGFNISS |
| | QGSNDFQGQLLESSYFEKIESNALRAFIKICPRNGLLYNAIYRNSKNYLNNIDLED |
| | KKTTSKTNVSYPCSLLNGCIEVENKDLFLISNKDSLNDINLSEEKIKPETTVFFKD |
| | KLPPQDITLSNYDFTEANSIPSISQQNILERNEELYEPIRNSLFEIKTIYVDKLTT |
| | FHFLEAQNIDESIDSSKIRVELTDSVDEALSNPNKVYSPFKNMSNTINSIETGITS |
| | TYIFYQWLRSIVKDESDETGKIDVIDKSSDTLAIVPYIGPLLNIGNDIRHGDFVGA |
| | IELAGITALLEYVPEFTIPILVGLEVIGGELAREQVEAIVNNALDKRDQKWAEVYN |
| | ITKAQWWGTIHLQINTRLAHTYKALSRQANAIKMNMEFQLANYKGNIDDKAKIKNA |
| | ISETEILLNKSVEQAMKNTEKFMIKLSNSYLTKEMIPKVQDNLKNFDLETKKTLDK |
| | FIKEKEDILGTNLSSSLRRKVSIRLNKNIAFDINDIPFSEFDDLINQYKNEIEDYE |
| | VLNLGAEDGKIKDLSGTTSDINIGSDIELADGRENKAIKIKGSENSTIKIAMNKYL |
| | RFSATDNFSISFWIKHPKPTNLLNNGIEYTLVENFNQRGWKISIQDSKLIWYLRDH |
| | NNSIKIVTPDYIAFNGWNLITITNNRSKGSIVYVNGSKIEEKDISSIWNTEVDDPI |
| | IFRLKNNRDTQAFTLLDQFSTYRKELNQNEVVKLYNYYFNSNYIRDIWGNPLQYNK |
| | KYYLQTQDKPGKGLIREYWSSFGYDYVILSDSKTITFPNNIRYGALYNGSKVLIKN |
| | SKKLDGLVRNKDFIQLEIDGYNMGISADRFNEDTNYIGTTYGTTHDLTTDFEIIQR |
| | QEKYRNYCQLKTPYNIFHKSGLMSTETSKPTFHDYRDWVYSSAWYFQNYENLNLRK |
| | HTKTNWYFIPKDEGWDED |
| Clostridium | >WP_011100836.1 tetanus neurotoxin TetX [Clostridium |
| tetani | tetani] |
| TeNT | MPITINNFRYSDPVNNDTIIMMEPPYCKGLDIYYKAFKITDRIWIVPERYEFGTKP |
| neurotoxin | EDFNPPSSLIEGASEYYDPNYLRTDSDKDRFLQTMVKLENRIKNNVAGEALLDKII |
| SEQ ID | NAIPYLGNSYSLLDKFDTNSNSVSFNLLEQDPSGATTKSAMLINLIIFGPGPVLNK |
| NO: 53 | NEVRGIVLRVDNKNYFPCRDGFGSIMQMAFCPEYVPTFDNVIENITSLTIGKSKYF |
| | QDPALLLMHELIHVLHGLYGMQVSSHEIIPSKQEIYMQHTYPISAEELFTFGGQDA |
| | NLISIDIKNDLYEKTLNDYKAIANKLSQVTSCNDPNIDIDSYKQIYQQKYQFDKDS |
| | NGQYIVNEDKFQILYNSIMYGFTEIELGKKENIKTRLSYFSMNHDPVKIPNLLDDT |
| | IYNDTEGFNIESKDLKSEYKGQNMRVNTNAFRNVDGSGLVSKLIGLCKKIIPPTNI |
| | RENLYNRTASLTDLGGELCIKIKNEDLTFIAEKNSFSEEPFQDEIVSYNTKNKPLN |
| | FNYSLDKIIVDYNLQSKITLPNDRTTPVTKGIPYAPEYKSNAASTIEIHNIDDNTI |
| | YQYLYAQKSPTTLQRITMTNSVDDALINSTKIYSYFPSVISKVNQGAQGILFLQWV |
| | RDIIDDFTNESSQKTTIDKISDVSTIVPYIGPALNIVKQGYEGNFIGALETTGVVL |
| | LLEYIPEITLPVIAALSIAESSTQKEKIIKTIDNFLEKRYEKWIEVYKLVKAKWLG |
| | TVNTQFQKRSYQMYRSLEYQVDAIKKIIDYEYKIYSGPDKEQIADEINNLKNKLEE |
| | KANKAMININIFMRESSRSFLVNQMINEAKKQLLEFDTQSKNILMQYIKANSKFIG |
| | ITELKKLESKINKVESTPIPESYSKNLDCWVDNEEDIDVILKKSTILNLDINNDII |
| | SDISGENSSVITYPDAQLVPGINGKAIHLVNNESSEVIVHKAMDIEYNDMENNFTV |
| | SFWLRVPKVSASHLEQYGTNEYSIISSMKKHSLSIGSGWSVSLKGNNLIWTLKDSA |
| | GEVRQITFRDLPDKFNAYLANKWVFITITNDRLSSANLYINGVLMGSAEITGLGAI |
| | REDNNITLKLDRCNNNNQYVSIDKFRIFCKALNPKEIEKLYTSYLSITFLRDFWGN |
| | PLRYDTEYYLIPVASSSKDVQLKNITDYMYLTNAPSYTNGKLNIYYRRLYNGLKFI |
| | IKRYTPNNEIDSFVKSGDFIKLYVSYNNNEHIVGYPKDGNAFNNLDRILRVGYNAP |
| | GIPLYKKMEAVKLRDLKTYSVQLKLYDDKNASLGLVGTHNGQIGNDPNRDILIASN |
| | WYFNHLKDKILGCDWYFVPTDEGWTND |

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A fusion protein comprising:
a catalytic domain of a diphtheria toxin (DT-C), wherein the catalytic domain (DT-C) comprises one or more mutations that inactivate the catalytic domain (DT-C);
a translocation domain of a diphtheria toxin (DT-T), wherein the catalytic domain (DT-C) and the translocation domain (DT-T) are linked by a disulfide bond; and
a receptor-binding domain (RBD) of a Clostridium neurotoxin protein positioned downstream of the translocation domain (DT-T), wherein the receptor-binding domain (RBD) possesses neuron-specific binding activity, and wherein the fusion protein is capable of delivering a cargo to neural cytoplasm of a cell.

2. The fusion protein of claim 1, wherein the one or more mutations of the catalytic domain (DT-C) comprise K₅₁>E and E₁₄₈>K mutations of SEQ ID NO:4.

3. The fusion protein of claim 1, wherein the catalytic domain (DT-C) and/or the translocation domain (DT-T) further comprise one or more mutations that suppress an immune response of a human subject administered the fusion protein.

4. The fusion protein of claim 3, wherein the one or more mutations that suppress an immune response of a human subject administered the fusion protein comprise one or more of K₁₂₅>S, R₁₇₃>A, and Q₁₈₄>S mutations of SEQ ID NO:5 and/or one or more of Q₂₄₅>S, K₃₈₅>G, E₂₉₂>S, and K₂₂₇>S of SEQ ID NO:7 or SEQ ID NO:8.

5. (canceled)

6. The fusion protein of claim 1, wherein the catalytic domain (DT-C) is devoid of ADP ribosylation activity.

7. The fusion protein of claim 1, wherein the catalytic domain (DT-C) comprises SEQ ID NO:4, a sequence having at least 95% sequence identity to SEQ ID NO:4, SEQ ID NO:5, or a sequence having at least 95% sequence identity to SEQ ID NO:5.

8. (canceled)

9. The fusion protein of claim 1, wherein the translocation domain (DT-T) domain comprises SEQ ID NO:7 or SEQ ID NO:8, or a sequence having at least 95% sequence identity to SEQ ID NO:7 or SEQ ID NO:8.

10. The fusion protein of claim 1, wherein the receptor-binding domain (RBD) of a Clostridium neurotoxin protein comprises an RBD of any one of Clostridium neurotoxin (BoNT) serotypes A1-A8, B1-B8, C1, CD, D, DC, E1-E12, F1-F8, G, FA, or X, or a Clostridium tetani neurotoxin.

11. The fusion protein of claim 1, wherein the receptor-binding domain (RBD) domain comprises SEQ ID NO:10, or a sequence having at least 95% sequence identity to SEQ ID NO:10.

12. (canceled)

13. The fusion protein of claim 1 further comprising:
a cargo positioned upstream of the catalytic domain (DT-C).

14. The fusion protein of claim 13, wherein the cargo comprises a polypeptide, an RNA molecule, a DNA molecule, or a small molecule.

15. The fusion protein of claim 14, wherein the cargo is a polypeptide, wherein the polypeptide comprises a B8 single domain antibody, a JSG-C1 single domain antibody (JC1), or an anti-tau single domain antibody 2B8.

16.-19. (canceled)

20. A propeptide fusion comprising:
a catalytic domain of a diphtheria toxin (DT-C), wherein the catalytic domain (DT-C) comprises one or more mutations that inactivate the catalytic domain (DT-C);
a translocation domain of a diphtheria toxin (DT-T), wherein the catalytic domain (DT-C) and the translocation domain (DT-T) are linked by a disulfide bond;
a first protease cleavage site between the catalytic domain (DT-C) and the translocation domain (DT-T); and
a receptor-binding domain (RBD) of a Clostridium botulinum neurotoxin protein positioned downstream of the translocation domain (DT-T), wherein the receptor-binding domain (RBD) possesses neuron-specific binding activity, and wherein the fusion protein is capable of delivering a cargo to neural cytoplasm of a cell.

21.-30. (canceled)

31. An isolated nucleic acid molecule encoding the propeptide fusion of claim 20.

32.-37. (canceled)

38. A method of expressing a fusion protein, said method comprising:
providing a nucleic acid construct comprising:
the nucleic acid molecule of claim 31;
a heterologous promoter operably linked to the nucleic acid molecule;
a 3′ regulatory region operably linked to the nucleic acid molecule; and
introducing the nucleic acid construct into a host cell under conditions effective to express a propeptide of the fusion protein.

39.-42. (canceled)

43. A method of attaching a cargo polypeptide to a fusion protein, said method comprising:
contacting (i) a cargo protein comprising a first member of a peptide fusion tag binding pair and (ii) the fusion protein of claim 1 comprising a second member of a peptide fusion tag binding pair with (iii) a biotinylated SnoopLigase to form a complex;
capturing the complex on a streptavidin matrix to immobilize the complex; and
eluting the cargo protein attached to the fusion protein.

44. The method of claim 43, wherein the SnoopLigase comprises an N-terminal flexible sequence and a short immobilization sequence.

45. A therapeutic agent comprising the fusion protein of claim 1 and a pharmaceutically acceptable carrier.

46. A method for treating a subject for toxic effects of a neurotoxin, said method comprising:
administering the therapeutic agent of claim 45 to the subject under conditions effective to treat the subject for toxic effects of the neurotoxin.

47. A treatment method of a neurological condition comprising:
administering the fusion protein of claim 1 to a subject under conditions effective to provide treatment to the subject.

48.-50. (canceled)


SEQUENCE LISTING (114 sequences)

SEQ ID NO: 1 - protein, 567 aa, Corynebacterium diphtheriae
  REGION 1..32: note = signal peptide
  REGION 1..567: note = Diphtheria toxin
MLVRGYVVSRKLFASILIGALLGIGAPPSAHAGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 2 - protein, 535 aa, Corynebacterium diphtheriae
  REGION 1..535: note = Diphtheria toxin
  SITE 186: note = Cys 186 forms a disulfide bond with Cys 201
  SITE 201: note = Cys 186 forms a disulfide bond with Cys 201
  REGION 190..193: note = furin protease cleavage site
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 3 - protein, 189 aa, synthetic construct
  REGION 1..189: note = Diphtheria toxin catalytic domain (DT-C)
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGN

SEQ ID NO: 4 - protein, 189 aa, synthetic construct
  REGION 1..189: note = Diphtheria toxin catalytic domain (DT-C)
  SITE 51: note = K51>E
  SITE 148: note = E148>K
GADDVVDSSKSFVMENFSSYHGTKPGYVDSIQKGIQKPKSGTQGNYDDDWEGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVKYINNWEQAKALSVELEINFETRGKRGQDAMYEYMAQACAGN

SEQ ID NO: 5 - protein, 189 aa, synthetic construct
  REGION 1..189: note = Diphtheria toxin catalytic domain (DT-C)
  SITE 51: note = K51>E
  SITE 125: note = K125>S
  SITE 148: note = E148>K
  SITE 173: note = R173>A
  SITE 184: note = Q184>S
GADDVVDSSKSFVMENFSSYHGTKPGYVDSIQKGIQKPKSGTQGNYDDDWEGFYSTDNKYDAAGYSVDNENPLSGKAGGVVKVTYPGLTKVLALKVDNAETIKKELGLSLTEPLMEQVGTEEFISRFGDGASRVVLSLPFAEGSSSVKYINNWEQAKALSVELEINFETRGKAGQDAMYEYMASACAGN

SEQ ID NO: 6 - protein, 184 aa, synthetic construct
  REGION 1..184: note = Diphtheria toxin translocation domain (DT-T)
SVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNR

SEQ ID NO: 7 - protein, 184 aa, synthetic construct
  REGION 1..184: note = Diphtheria toxin translocation domain (DT-T)
  SITE 34: note = K227>S
  SITE 52: note = Q245>S
  SITE 99: note = E292>S
SVGSSLSCINLDWDVIRDKTKTKIESLKEHGPISNKMSESPNKTVSEEKAKSYLEEFHQTALEHPELSELKTVTGTNPVFAGANYAAWAVNVAQVIDSSTADNLEKTTAALSILPGIGSVMGIADGAVHHNTEEIVAQSIALSSLMVAQAIPLVGELVDIGFAAYNFVESIINLFQVVHNSYNR

SEQ ID NO: 8 - protein, 196 aa, synthetic construct
  REGION 1..196: note = Diphtheria toxin translocation domain (DT-T)
  SITE 34: note = K227>S
  SITE 52: note = Q245>S
  SITE 99: note = E292>S
  SITE 192: note = K385>G
SVGSSLSCINLDWDVIRDKTKTKIESLKEHGPISNKMSESPNKTVSEEKAKSYLEEFHQTALEHPELSELKTVTGTNPVFAGANYAAWAVNVAQVIDSSTADNLEKTTAALSILPGIGSVMGIADGAVHHNTEEIVAQSIALSSLMVAQAIPLVGELVDIGFAAYNFVESIINLFQVVHNSYNRPAYSPGHGTQPF

SEQ ID NO: 9 - protein, 1296 aa, Clostridium botulinum
  REGION 1..1296: note = BoNT/A1
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 10 - protein, 425 aa, synthetic construct
  REGION 1..425: note = BoNT/A1 RBD
NIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 11 - protein, 1296 aa, Clostridium botulinum
  REGION 1..1296: note = BoNT/A2
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 12 - protein, 1292 aa, Clostridium botulinum
  REGION 1..1292: note = BoNT/A3
MPFVNKQFNYRDPVNGVDIAYIKIPNAGQMQPVKAFKIHEGVWVIPERDTFTNPEEGDLNPPPEAKQVPVSYYDSTYLSTDNEKDNYLKGVIKLFDRIYSTGLGRMLLSFIVKGIPFWGGSTIDTELKVIDTNCINVIEPGGSYRSEELNLVITGPSADIIQFECKSFGHDVFNLTRNGYGSTQYIRFSPDFTFGFEESLEVDTNPLLGAGTFATDPAVTLAHELIHAAHRLYGIAINPNRVLKVKTNAYYEMSGLEVSFEELRTFGGNDTNFIDSLWQKKFSRDAYDNLQNIARILNEAKTIVGTTTPLQYMKNIFIRKYFLSEDASGKISVNKAAFKEFYRVLTRGFTELEFVNPFKVINRKTYLNFDKAVFRINIVPDENYTINEGFNLEGANSNGQNTEINSRNFTRLKNFTGLFEFYKLLCVRGIIPFKTKSLDEGYNKALNDLCIKVNNWDLFFSPSEDNFTNDLDKVEEITADTNIEAAEENISSDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAVIFLSWAEELVYDFTDETNEVTTMDKIADITIIVPYIGPALNIGNMVSKGEFVEAILFTGVVALLEFIPEYSLPVFGTFAIVSYIANKVLTVQTINNALSKRNEKWDEVYKYTVTNWLAKVNTQIDLIREKMKKALENQAEATRAIINYQYNQYTEEEKNNINFNIDDLSSKLNRSINRAMININKFLDQCSVSYLMNSMIPYAVKRLKDFDASVRDVLLKYIYDNRGTLILQVDRLKDEVNNTLSADIPFQLSKYVNDKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWMFVTITNNRLTKSKIYINGRLIDQKPISNLGNIHASNKIMFKLDGCRDPRRYIMIKYFNLFDKELNEKEIKDLYDSQSNPGILKDFWGNYLQYDKPYYMLNLFDPNKYVDVNNIGIRGYMYLKGPRGSVMTTNIYLNSTLYMGTKFIIKKYASGNEDNIVRNNDRVYINVVVKNKEYRLATNASQAGVEKILSALEIPDVGNLSQVVVMKSKDDQGIRNKCKMNLQDNNGNDIGFVGFHLYDNIAKLVASNWYNRQVGKASRTFGCSWEFIPVDDGWGESSL

SEQ ID NO: 13 - protein, 1296 aa, Clostridium botulinum
  REGION 1..1296: note = BoNT/A4
MPFVNKQFNYNDPENGVDIAYIKIPNAGKMQPVKAFKIHNKVWVIPERDIFTNPEEVDLNPPPEAKQVPISYYDSAYLSTDNEKDNYLKGVIKLFERIYSTDLGRMLLISIVRGIPFWGGGKIDTELKVIDTNCINIIQLDDSYRSEELNLAIIGPSANIIESQCSSFRDDVLNLTRNGYGSTQYIRFSPDFTVGFEESLEVDTNPLLGAGKFAQDPAVALAHELIHAEHRLYGIAINTNRVFKVNTNAYYEMAGLEVSLEELITFGGNDAKFIDSLQKKEFSLYYYNKFKDIASTLNKAKSIVGTTASLQYMKNVFKEKYLLSEDATGKFLVDRLKFDELYKLLTEIYTEDNFVKFFKVLNRKTYLNFDKAVFKINIVPDVNYTIHDGFNLRNTNLAANFNGQNIEINNKNFDKLKNFTGLFEFYKLLCVRGIITSKTKSLDEGYNKALNELCIKVNNWDLFFSPSEDNFTNDLDKVEEITSDTNIEAAEENISLDLIQQYYLNFNFDNEPENTSIENLSSDIIGQLEPMPNIERFPNGKKYELNKYTMFHYLRAQEFKHSNSRIILTNSAKEALLKPNIVYTFFSSKYIKAINKAVEAVTFVNWIENLVYDFTDETNEVSTMDKIADITIVIPYIGPALNIGNMIYKGEFVEAIIFSGAVILLEIVPEIALPVLGTFALVSYVSNKVLTVQTIDNALSKRNEKWDEVYKYIVTNWLAIVNTQINLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLIGQVNRLKDKVNNTLSADIPFQLSKYVDNKKLLSTFTEYIKNITNASILSIVYKDDDLIDLSRYGAEIYNGDKVYYNSIDKNQIRLINLESSTIEVILKKAIVYNSMYENFSTSFWIRIPKYFNSISLNNEYTIINCMENNSGWKVSLNYGEIIWTLQDTQEIKQRVVFKYSQMINISDYINRWIFVTITNNRITKSKIYINGRLIDQKPISNLGNIHASNKIMFKLDGCRDPHRYIVIKYFNLFDKELSEKEIKDLYDNQSNSGILKDFWGDYLQYDKSYYMLNLYDPNKYVDVNNVGIRGYMYLKGPRDNVMTTNIYLNSSLYMGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWRERPL

SEQ ID NO: 14 - protein, 1296 aa, Clostridium botulinum
  REGION 1..1296: note = BoNT/A5
MLFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTELGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGEHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPEVNYTIYDGFNLRNTNLAANFNGQNTEINNMNFTKLKNFTGLFEFYKLLCVRGIITSKTKSLDEGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIVLTNSVNEALLNPSSVYTFFSSDYVRKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWGEVYKYIVTNWLAKVNTQIDLIRKKMKEALENQAEATKAIINYQYNQYTEEEKNNINFNIGDLSSKLNDSINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASEINIGSKVNFDPIDKNQIQLFNLESSKIEIILKNAIVYNSMYENFSTSFWIKIPKYFSKINLNNEYTIINCIENNSGWKVSLNYGEIIWTLQDNKQNIQRVVFKYSQMVAISDYINRWIFITITNNRLNNSKIYINGRLIDQKPISNLGNIHASNNIMFKLDGCRDPHRYIWIKYFNLFDKELNEKEIKDLYDNQSNSGILKDFWGNYLQYDKPYYMLNLYDPNKYVDVNNVGIRGYMYLKGPRGSIVTTNIYLNSSLYMGTKFIIKKYASGNKDNIVRNNDRVYINVVVKNKEYRLATNASQAGVEKILSVLEIPDVGNLSQVVVMKSKNDQGIRNKCKMNLQDNNGNDIGFIGFHQFNNIDKLVASNWYNRQIERSSRTFGCSWEFIPVDDGWGESPL

SEQ ID NO: 15 - protein, 1296 aa, Clostridium botulinum
  REGION 1..1296: note = BoNT/A6
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFAKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLSAQEFEHGKSRIDLTNSVNEALLNPSHVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLIGQVDRLKDKVNNTLSTDIPFQLSKYVDNQRLLSTFTEYIKNIINTSILSLRYENNHLIDLSRYASKINIGSRVNFDPIDKNQIQLFNLESSKIEVILKNAIVYNSMYENFSTSFWIKIPKYFSEISLNNEYTIINCIENNSGWKVSLNYGEIIWTLQDNKQNIQRVVFKYSQMVAISDYINRWIFITITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNVGIRGYMYLKGSRSTLLTTNIYLNSGLYMGTKFIIKKYASGNKDNIVRNNDRVYINVVVNNKEYRLATNASQAGVEKILSALEIPDIGNLSQVVVMKSKNDQGIRNKCKMNLQDNNGNDIGFIGFHKFNDIYKLVASNWYNRQIEISSRTFGCSWEFIPVDDGWGEKPL

SEQ ID NO: 16 - protein, 1296 aa, Clostridium botulinum
  REGION 1..1296: note = BoNT/A7
MPFVNKQFNYKDPVNGVDIAYIKIPNAGQMQPVKAFKIHNKIWVIPERDIFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIINFECKSFGHDVLNLTRNGYGSTQYIRFSPDFTFGFEESLEVDTNPLLGAGKFAIDPAVTLAHELIHAGHRLYGIAINPNRVFKVNTNAYYEMSGLEVSFEELRTFGGHDAKFIDSLQENEFRLYYYNKFKEVASILNKAKSIIGTTASLQYMKNVFKEKYLLSEDTSGKFSVDKLRFDKLYKMLTEIYTEDNFVKFFKVLNRKTYLNFDKAVFKMNIVPEVNYTIYDGFNLRNTNLAANFNGQNTEINNMNFTKLKNFTGLFEFYKLLCVRGIITSKTKSLDEGYNKALNDLCIKVNNWDLFFSPSEDNFTNDLNKGEEITSDTNIEAAEENISSDLIQQYYLTFNFDNEPENISIENLSSDIIGQLELMPNIERFPNGKKYELDKYTMFHYLRAQEFEYGNSRIVLINSVNEALLNPSSVYTFFSSDYVKKANEATEAAMFLGWVEQLVYDFTDETSEVSTMDKIADITIIVPYIGPALNIGNMVYKKKFEEALIFSGAVILLEFVPEIVLPILGTFALVSYTSNKVLTVRTIDNALSKRNEKWEEVYKYIVTNWLAKVNTQINLIRKKMKEALENQAEATKAIINYQYNQYTEEEKNNINFNIGDLSSKLNDSINKAMININKFLDQCSVSYLMNSMIPQGVKQLKDFDTSLRDSLLKYIYDNRGTLIGQVDRLKDKVNNTLSTDIPFQLSKYADNQRLLSTFTEYIKNIINTSILNLRYESNHLIDLSRYASKINIGSRVNFDPIDKNQIQLFNLESSKIEVILKNAIVYNSMYENFSTSFWIKIPKYFSKINLNNEYTIINCIENNSGWKVSLNYGEIIWTLQDNEQNIQRVVFKYSQMVNISDYINRWIFVTITNNRLTKSKIYINGRLIDQKPISNLGNIHASNKIMFKLDGCRDPHRYILIKYFNLFDKELNEKEIKDLYDNQSNSGILKDFWGDYLQYDKPYYMLNLYDPNKYIDVNNIGIRGYMYLKGPRGSVTTTNIYLNSMLYMGTKFIIKKHASGNKDNIVRNNDRVYINVLVKNKEYRLATNASQAGGEKILSAVEIPDVGNLSQVVVMKSKNDQGIRNKCKMNLQDNNGNDIGFIGFHQFNNIAKLVASNWYNRQIGKTSVTLGCSWELIPVDYGWGESSL

SEQ ID NO: 17 - protein, 1297 aa, Clostridium botulinum
  REGION 1..1297: note = BoNT/A8
MPFVNKQFNYKDTVNGIDIAYIKIPNAGQMQPVKAFKIHNKIWVIPERDTFTNPKEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHNAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPDENYTIKDGFNLKNTNLAANFNGQNTEINSRNFTKLKNFTGLFEFYKLLCVRGIIPFKTKSLDEGYNKALNDLCIKVNNWDLFFSPSEDNFTNDLDKVEEITSDTNIEAAEENISLDLIQQYYLTFDFDNEPENISIENLSSDIIGQLEPMPNIERFPNGKKYELDKYTMFHYLRAQEFEHSKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLVRKKMKEALENQAEATKAIINYQYNQYTEEEKNNINFNIDDLSSKLNESINSAMTNINKFLDQCSVSYLMNSMIPYAVKRLKDFDASVREVLLKYIYDNRGTLILQVDRLKDKVNNTLSADIPFQLSKYVDNKKLLSTFTEYIKNITNTSILSIVVDKDGRLIDLSRYGAEIYNGDKVSYNSIDKNQIKLINLESSAIEVILKNAIVYNSMYENFSTSFWIKIPKYFSKINLNNEYTIINCIENNSGWKVSLNYGEIIWTLQDNQQNIQRVVFKYSQMVNISDYINRWIFVTITNNRLDKSKIYINGRLIDQKPISNLGNIHASNNIMFKLDGCRDPRRYIVIKYFNLFDKELNEKEIKDLYDNQSNSGILKDFWGDYLQYDKPYYMLNLYDPNKYVDVNNIGIRGYMYLKGPRGSVVTTNIYLNSTLYMGTKFIIKKYASGNKDNIVRNNDRVYINVVVKNKEYRLATNALQAGVEKILSALEIPDVGNLSQVVVMKSKNDQGIRNKCKMNLQDNNGNDIGLIGFHQFNNIAKLVASNWYNRQVGKASRTFGCSWEFIPVDDGWGESSQ

SEQ ID NO: 18 - protein, 1291 aa, Clostridium botulinum
  REGION 1..1291: note = BoNT/B1
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 19 - protein, 1291 aa, Clostridium botulinum
  REGION 1..1291: note = BoNT/B2
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVRAPGICIDVDNEDLFFIADKNSFSDDLSKNERIEYDTQSNYIENRSSIDELILDTNLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVDDFVIEANKSSTMDKIADISLIVPYIGLALNVGNETAKGNFENAFEIAGASILLEFIPELLIPVVGAFLLESYIDNKNKIIKTIDNALTKRDEKWIDMYGLIVAQWLSTVNTQFYTIKEGMYKALNYQAQALEEIIKYKYNIYSEKEKSNINIDFNDINSKLNEGINQAVDNINNFINECSVSYLMKKMIPLAVEKLLDFDNTLKKNLLNYIDENKLYLIGSAEYEKSKVDKHLKTIIPFDLSMYTNNTILIEIFNKYNSEILNNIILNLRYRDNNLIDLSGYGANVEVYDGVELNDKNQFKLTSSTNSEIRVTQNQNIIFNSMFLDFSVSFWIRIPKYKNDGIQNYIHNEYTIINCIKNNSGWKISIRGNRIIWTLTDINGKTKSVFFEYSIREDISDYINRWFFVTITNNSDNAKIYINGKLESNIDIKDIGEVIANGEIIFKLDGDIDRTQFIWMKYFSIFNTELSQSNIKEIYKIQSYSEYLKDFWGNPLMYNKEYYMFNAGNKNSYIKLKKDSSVGEILTRSKYNQNSNYINYRNLYIGEKFIIRRKSNSQSINDDIVRKEDYIYLDFFNSNREWRVYAYKDFKEEEKKLFLANIYDSNEFYKTIQIKEYDEQPTYSCQLLFKKDEESTDEIGLIGIHRFYESGIVLKDYKNYFCISKWYLKEVKRKPYNPNLGCNWQFIPKDEGWIE

SEQ ID NO: 20 - protein, 1291 aa, Clostridium botulinum
  REGION 1..1291: note = BoNT/B3
MPVTINNFNYNDPIDNDNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPRIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVRAPGICIDVDNEDLFFIADKNSFSDDLSKNERIEYDTQSNYIENRSSIDELILDTNLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVDDFVIEANKSSTMDKIADISLIVPYIGLALNVGNETAKGNFENAFEIAGASILLEFIPELLIPVVGAFLLESYIDNKNKIIKTIDNALTKRDEKWIDMYGLIVAQWLSTVNTQFYTIKEGMYKALNYQAQALEEIIKYKYNIYSEKEKSNINIDFNDINSKLNEGINQAIDNINNFINECSVSYLMKKMIPLAVEKLLDFDNTLKKNLLNYIDENKLYLIGSAEYEKSKVDKHLKTIIPFDLSMYTNNTILIEIFNKYNSEILNNIILNLRYRDNNLIDLSGYGAKVEVYNGVELNDKNQFKLTSSANSKIRVTQNQDIIFNSMFLDFSVSFWIRIPKYKNDGIQNYIHNEYTIINCIKNNSGWKISIRGNKIIWTLTDINGKTKSVFFEYSIRKDVSEYINRWFFVTITNNSDNAKIYINGKLESNIDIKDIGEVIANGEIIFKLDGDIDRTQFIWMKYFSIFNTELSQSNIKEIYKIQSYSEYLKDFWGNPLMYNKEYYMFNAGNKNSYIKLKKDSSVGEILTRSKYNQNSNYINYRNLYIGEKFIIRRKSNSQSINDDIVRKEDYIYLDFFNLNQEWRVYAYKDFKKKEEKLFLANIYDSNEFYNTIQIKEYDEQPTYSCQLLFKKDEESTDEIGLIGIHRFYESGIVFKDYKDYFCISKWYLKEVKRKPYNPNLGCNWQFIPKDEGWIE

SEQ ID NO: 21 - protein, 1291 aa, Clostridium botulinum
  REGION 1..1291: note = BoNT/B4
MPVTINNFNYNDPIDNDNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVEQKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDTIQAEELYTFGGQDPSIISPSTDKSIYDKVLQNFRGIVDRLNKVLVCISDPNININIYKNKFKDKYKFVEDSEGKYSIDVESFNKLYKSLMFGFTEINIAENYKIKTRASYFSDSLPPVKIKNLLDNEIYTIEEGFNISDKNMGKEYRGQNKAINKQAYEEISKEHLAVYKIQMCKSVKVPGICIDVDNENLFFIADKNSFSDDLSKNERVEYNTQNNYIGNDFPINELILDTDLISKIELPSENTESLTDFNVDVPVYEKQPAIKKVFTDENTIFQYLYSQTFPLNIRDISLTSSFDDALLVSSKVYSFFSMDYIKTANKVVEAGLFAGWVKQIVDDFVIEANKSSTMDKIADISLIVPYIGLALNVGDETAKGNFESAFEIAGSSILLEFIPELLIPVVGVFLLESYIDNKNKIIKTIDNALTKRVEKWIDMYGLIVAQWLSTVNTQFYTIKEGMYKALNYQAQALEEIIKYKYNIYSEEEKSNININFNDINSKLNDGINQAMDNINDFINECSVSYLMKKMIPLAVKKLLDFDNTLKKNLLNYIDENKLYLIGSVEDEKSKVDKYLKTIIPFDLSTYTNNEILIKIFNKYNSEILNNIILNLRYRDNNLIDLSGYGAKVEVYDGVKLNDKNQFKLTSSADSKIRVTQNQNIIFNSMFLDFSVSFWIRIPKYRNDDIQNYIHNEYTIINCMKNNSGWKISIRGNRIIWTLIDINGKTKSVFFEYNIREDISEYINRWFFVTITNNLDNAKIYINGTLESNMDIKDIGEVIVNGEITFKLDGDVDRTQFIWMKYFSIFNTQLNQSNIKEIYKIQSYSEYLKDFWGNPLMYNKEYYMFNAGNKNSYIKLVKDSSVGEILIRSKYNQNSNYINYRNLYIGEKFIIRRKSNSQSINDDIVRKEDYIHLDFVNSNEEWRVYAYKNFKEQEQKLFLSIIYDSNEFYKTIQIKEYDEQPTYSCQLLFKKDEESTDDIGLIGIHRFYESGVLRKKYKDYFCISKWYLKEVKRKPYKSNLGCNWQFIPKDEGWTE

SEQ ID NO: 22 - protein, 1291 aa, Clostridium botulinum
  REGION 1..1291: note = BoNT/B5
MPVTINNFNYNDPIDNNNIIMMEPPFARGMGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVNDLPIVPNEKKFFMQSTDAIQAEELYTFGGQDPSIISPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVKAPGICIDVDNEDLFFIADKNSFSDDLSKNERIAYNTQNNYIENDFSINELILDTDLISKIELPSENTESLTDFNVYVPVYKKQPAIKKIFTDENTIFQYLYSQTFPLDIRDISLTSSFDDALLFSNKVYSFFSMDYIKTANKVVEAGLFAGWVKQIVDDFVIEANKSSTMDKIADISLIVPYIGLALNVGNETAKGNFENAFEIAGASILLEFIPELLIPVVGAFLLESYIDNKNKIIETINSALTKRDEKWIDMYGLIVAQWLSTVNTQFYTIKEGMYKALNYQAQALEEIIKYKYNIYSEKERSNINIDFNDVNSKLNEGINQAIDNINNFINECSVSYLMKKMIPLAVEKLLDFDNTLRKNLLNYIDENKLYLIGSAEYEKSKVDKYLKTSIPFDLSTYTNNTILIEIFNKYNSDILNNIILNLRYRDNKLIDLSGYGAKVEVYDGVKLNDKNQFKLTSSANSKIRVIQNQNIIFNSMFLDFSVSFWIRIPKYKNDGIQNYIHNEYTIINCMKNNSGWKISIRGNMIIWTLIDINGKIKSVFFEYSIKEDISEYINRWFFVTITNNSDNAKIYINGKLESHIDIRDIREVIANDEIIFKLDGNIDRTQFIWMKYFSIFNTELSQSNIEEIYKIQSYSEYLKDFWGNPLMYNKEYYMFNAGNKNSYIKLKKDSSVGEILTRSKYNQNSKYINYRDLYIGEKFIIRRKSNSQSINDDIVRKEDYIYLDFFNLNQEWRVYMYKYFKKEEEKLFLAPISDSDEFYNTIQIKEYDEQPTYSCQLLFKKDEESTDEIGLIGIHRFYESGIVFKEYKDYFCISKWYLKEVKRKPYNSKLGCNWQFIPKDEGWTE

SEQ ID NO: 23 - protein, 1291 aa, Clostridium botulinum
  REGION 1..1291: note = BoNT/B6
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVRAPGICIDVDNEDLFFIADKNSFSDDLSKNERIEYDTQSNYIENRSSIDELILDTNLISKIELPSENTESLTDFNVDVPVYEKQPAIKKFFTDENTIFQYLYSQTFPLDIRDISLTSSFDDALLFSNKVYSFFSMDYIKTANKVVEAGLFAGWVKQIVDDFVIEANKSNTMDKLADISLIVPYIGLALNVGNETAKGNFENAFEIAGASILLEFIPELLIPVVGAFLLESYIDNKNKIIKTIDNALTKRDEKWRDMYGLIVAQWLSTVNTQFYTIKEGMYKALNYQAQALEEIIKYKYNIYSEKEKSNINIDFNDINSKLNEGINQAIDNINNFINECSVSYLMKKMIPLAVEKLLDFDNTLKKNLLNYIDENKLYLIGSAEYEKSKVDKHLKTIIPFDLSMYTNNTILIEIFKKYNSEILNNIILNLRYRDNNLIDLSGYGANVEVYDGVELNDKNQFKLTSSTNSEIRVTQNQNIIFNSMFLDFSVSFWIRIPKYKNDGIQNYIHNEYTIINCIKNNSGWKISIRGNRIIWTLTDINGKTKSVFFEYSIREDISDYINRWFFVTITNNSDNAKIYINGKLESNIDIKDIGEVIANGEIIFKLDGDIDRTQFIWMKYFSIFNTELSQSNIKEIYKIQSYSEYLKDFWGNPLMYNKEYYMFNAGNKNSYIKLKKDSPVGEILTRSKYNQNSNYINYRNLYIGEKFIIRRKSNSQSINDDIVRKEDYIYLDFFNLNQEWRVYALKNFKKKEEKLFLAPISDSDEFYNTIQIKEYDEQPTYSCQLLFKKDEESTDEIGLIGIHRFYESGIVFKDYKYYFCISKWYLKEVKRKPYNPNLGCNWQFIPKDEGWIE

SEQ ID NO: 24 - protein, 1291 aa, Clostridium botulinum
  REGION 1..1291: note = BoNT/B7
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTKNIYIENYFSINELILDTDLISGIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIIDDFVIEANKSSTMDKIADISLIVPYIGLALNVGNETAKGNFENAFEIAGASILLEFIPELLIPVVGAFLLESYIDNKNKIIKTIDNALTKRVEKWIDMYGLIVAQWLSTVNTQFYTIKEGMYKALNYQAQALEEIIKYKYNIYSEKEKLNINIDFNDINSKLNEGINQAIDNINNFINECSVSYLMKKMIPLAIEKLLDFDNALKKNLLNYIDENKLYLIGSVEEEKSKVDKFFKTIIPFDLSMYTNNTILIEMVNKYNSEILNNIILNLRYRDNNLIDSSGYGAKVEVYNGVELNDKNQFKLTSSANSKIKVTQNQNITFNSMFLDFSVSFWIRIPKYKNDGIQNYIHNEYTIINCMKNNSGWKISIRGNRIIWTLTDINGKTKSVFFEYSIREDISDYINRWFFVTITNNLDNAKIYINGKLESNIDIRDIREVIVNGEIIFKLDGEIDRTQFIWMKYFSIFNTELSQSNVKEIYKIQSYSKYLKDFWGNPLMYNKEYYMFNAGNKNSYIKLVKDSSVGEILTRSKYNQNSNYINYRNLYIGEKFIIRRKSSSQSISDDIVRKEDYIYLDFFNSNREWRVYAYKNFKGQEEKLFLANIYDSNEFYKTIQIKEYDEQPTYSCQLLFKKDEESTDEIGLIGIHNFYESGILFKDYKDYFCISKWYLKEVKKKPYSSNLGCNWQFIPKDEGWTE

SEQ ID NO: 25 - protein, 1292 aa, Clostridium botulinum
  REGION 1..1292: note = BoNT/B8
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGGEERKEGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNGKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDDEIYTIEEGFNISDKNMGKEYRGQNKAINKQAYEEISKEHLAVYKIQMCKSVRAPGICIDVDNEDLFFIADKNSFSDDLSKNERIEYNTQSNYIENDFSINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVDDFVIEANKSNTMDKIADISLIVPYIGLALNVGNETAKGNFENAFEIAGSSILLEFIPELLIPVVGAFLLESYIDNKNKIIKTIDNALTKRDEKWIDMYGLIVAQWLSTVNTQFYTIKEGMYKALNYQAQALEEIIKYKYNIYSEKEKSNISIDFNDINSKLNEGINQAIDNINDFINECSVSYLMKKMIPLAVEKLLDFDNTLKKNLLNYIDENKLYLIGSAEYEKSKVDKHLKTIMTFDLSMYTNNTILIKMVNKYNSEILNNIILNLRYRDNNLIDLSGYGANVEVYDGVELNDKNQFKLTSSTNSEIRVTQNQNIIVNSMFLDFSVSFWIRIPKYKNDGIQNYIHNEYTIINCMKNNSGWKISIRGNRIIWTLIDINGKIKSVFFEYSIRKDVSEYINRWFFVTITNNLDNAKIYINGKLESNMDIRDIREVIANGEIIFKLDGDIDRTQFIWMKYFSIFNTELSQSNIEEIYKIQSYSEYLKDFWGNPLMYNKEYYMFNAGSKNSYIKLKKDSSVGEILTRSKYNQNSQYINYRDLYIGEKFIIKRKSNSQSINDDIVRKEDYIYLDFFNLNQEWRVYAYKDFKGQKEQKLFLANIHDSNEFYKTIQIKEYDEQPTYSCQLLFKKDEESTDEIGLIGIHRFYESGFVFQEYKYYFCISKWYLKEVKKKPYNPDLGCNWQFIPKDEGWTE

SEQ ID NO: 26 - protein, 1291 aa, Clostridium botulinum
  REGION 1..1291: note = BoNT/C1
MPITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 27 - protein, 1280 aa, Clostridium botulinum
  REGION 1..1280: note = BoNT/CD
MPITINNFNYSDPVDNKNILYLDTHLNTLANEPEKAFRIIGNIWVIPDRFSRDSNPNLNKPPRVTSPKSGYYDPNYLSTDSEKDTFLKEIIKLFKRINSREIGEELIYRLATDIPFPGNNNTPINTFDFDVDFNSVDVKTRQGNNWVKTGSINPSVIITGPRENIIDPETSTFKLTNNTFAAQEGFGALSIISISPRFMLTYSNATNNVGEGRFSKSEFCMDPILILMHELNHTMHNLYGIAIPNDQRISSVTSNIFYSQYKVKLEYAEIYAFGGPTIDLIPKSGRKYFEEKALDYYRSIAKRLNSITTANPSSFNKYIGEYKQKLIRKYRFVVESSGEVAVDRNKFAELYKELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDIKTDIFLSKDINVETEVIDYPDNVSVDQVILSKNTSEHGQLDLLYPIIEGESQVLPGENQVFYDNRTQNVDYLNSYYYLESQKLSDNVEDFTFTTSIEEALDNSGKVYTYFPKLADKVNTGVQGGLFLMWANDVVEDFTTNILRKDTLDKISDVSAIIPYIGPALNISNSVRRENFTEAFAVTGVTILLEAFQEFTIPALGAFVIYSKVQERNEIIKTIDNCLEQRIKRWKDSYEWMIGTWLSRITTQFNNISYQMYDSLNYQADAIKDKIDLEYKKYSGSDKENIKSQVENLKNSLDIKISEAMNNINKFIRECSVTYLFKNMLPKVIDELNKFDLKTKTELINLIDSHNIILVGEVDRLKAKVNESFENTIPFNIFSYTNNSLLKDIINEYFNSINDSKILSLQNKKNALVDTSGYNAEVRLEGDVQVNTIYTNDFKLSSSGDKIIVNLNNNILYSAIYENSSVSFWIKISKDLTNSHNEYTIINSIKQNSGWKLCIRNGNIEWILQDINRKYKSLIFDYSESLSHTGYTNKWFFVTITNNIMGYMKLYINGELKQSERIEDLNEVKLDKTIVFGIDENIDENQMLWIRDFNIFSKELSNEDINIVYEGQILRNVIKDYWGNPLKFDTEYYIINDNYIDRYIAPKSNILVLVQYPDRSKLYTGNPITIKSVSDKNPYSRILNGDNIMFHMLYNSGKYMIIRDTDTIYAIEGRECSKNCVYALKLQSNLGNYGIGIFSIKNIVSQNKYCSQIFSSFMKNTMLLADIYKPWRFSFENAYTPVAVTNYETKLLSTSSFWKFISRDPGWVE

SEQ ID NO: 28 - protein, 1256 aa, Clostridium botulinum
  REGION 1..1256: note = BoNT/D
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYET

SEQ ID NO: 29 - protein, 1285 aa, Clostridium botulinum
  REGION 1..1285: note = BoNT/DC
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGSDVEIIPQIERLQLREKALGHYKDIAKRLNNINKTIPSSWSSNIDKYKKIFSEKYNFDKDNTGNFVVNIDKFNSLYSDLTNVMSEVVYSSQYNVKNRTHYFSKHYLPVFANILDDNIYTIINGFNLTTKGFNIENSGQNIERNPALQKLSSESVVDLFTKVCLRLTRNSRDDSTCIQVKNNTLPYVADKDSISQEIFESQIITDETNVENYSDNFSLDESILDAKVPTNPEAVDPLLPNVNMEPLNVPGEEEVFYDDITKDVDYLNSYYYLEAQKLSNNVENITLTTSVEEALGYSNKIYTFLPSLAEKVNKGVQAGLFLNWANEVVEDFTTNIMKKDTLDKISDVSAIIPYIGPALNIGNSALRGNFKQAFATAGVAFLLEGFPEFTIPALGVFTFYSSIQEREKIIKTIENCLEQRVKRWKDSYQWMVSNWLSRITTQFNHISYQMYDSLSYQADAIKAKIDLEYKKYSGSDKENIKSQVENLKNSLDVKISEAMNNINKFIRECSVTYLFKNMLPKVIDELNKFDLKTKTELINLIDSHNIILVGEVDRLKAKVNESFENTIPFNIFSYTNNSLLKDMINEYFNSINDSKILSLQNKKNTLMDTSGYNAEVRVEGNVQLNPIFPFDFKLGSSGDDRGKVIVTQNENIVYNAMYESFSISFWIRINKWVSNLPGYTIIDSVKNNSGWSIGIISNFLVFTLKQNENSEQDINFSYDISKNAAGYNKWFFVTITTNMMGNMMIYINGKLIDTIKVKELTGINFSKTITFQMNKIPNTGLITSDSDNINMWIRDFYIFAKELDDKDINILFNSLQYTNVVKDYWGNDLRYDKEYYMINVNYMNRYMSKKGNGIVFNTRKNNNDFNEGYKIIIKRIRGNTNDTRVRGENVLYFNTTIDNKQYSLGMYKPSRNLGTDLVPLGALDQPMDEIRKYGSFIIQPCNTFDYYASQLFLSSNATTNRLGILSIGSYSFKLGDDYWFNHEYLIPVIKIEHYASLLESTSTHWVFVPASE

SEQ ID NO: 30 - protein, 1251 aa, Clostridium botulinum
  REGION 1..1251: note = BoNT/E1
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 31 - protein, 1252 aa, Clostridium botulinum
  REGION 1..1252: note = BoNT/E2
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGIQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYNNEPNANILKDFWGNYLLYDKEYYLLNVLKPNNFIDRRTDSTLSINNIRSTILLANRLYSGIKVKIQRVNNSSTNDNLVRKNDQVYINFVASKTHLFPLYADTNTTNKEKTIKSSSSGNRFNQVVVMNSVGNNCTMNFKNNNGNNIGMLGFKDNTLVASTWYYTHMRDNTNSNGCFWNFISEEHGWQEK

SEQ ID NO: 32 - protein, 1252 aa, Clostridium botulinum
  REGION 1..1252: note = BoNT/E3
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQHILLPNVIIMGAEPDLFETNSSNISLRNNYMPSNHGFGSIAIVTFSPEYSFRFNDNSINEFIQDPALTLMHELIHSLHGLYGAKGITTTCIITQQQNPLITNRKGINIEEFLTFGGNDLNIITVAQYNDIYTNLLNDYRKIASKLSKVQVSNPQLNPYKDIFQEKYGLDKDASGIYSVNINKFDDILKKLYSFTEFDLATKFQVKCRETYIGQYKYFKLSNLLNDSIYNISEGYNINNLKVNFRGQNANLNPRIIKPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 33 - protein, 1252 aa, Clostridium butyricum
  REGION 1..1252: note = BoNT/E4
MPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVGWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKAIIESKYNSYTLEEKNELTNKYDIEQIENELNQKVSIAMNNIDRFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNSGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILKDFWGNYLLYDKEYYLLNVLKPNNFINRRTDSTLSINNIRSTILLANRLYSGIKVKIQRVNNSSTNDNLVRKNDQVYINFVASKTHLLPLYADTATTNKEKTIKISSSGNRFNQVVVMNSVGNNCTMNFKNNNGNNIGLLGFKADTVVASTWYYTHMRDNTNSNGFFWNFISEEHGWQEK

SEQ ID NO: 34 - protein, 1251 aa, Clostridium butyricum
  REGION 1..1251: note = BoNT/E5
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFVPELLIPTILVFTIKSFLGSSDNKNKVIKAINNALKERDEKWKEVYSFIVSNWMTKINTQFNKRKEQMYQALQNQVNALKTIIEFKYNSYTLEEKKELKNNYDIEQIENELNQKVSIAMNNIDRFLTESSISYLMKLINEVKINKLREYDENVKTYLLDYIIQHGSILGESQQELNSMVIDTLNNSIPFKLSSYTDDKILISYFNKFFKRIKSSSVLNMRYKNDKYVDTSGYDSNININGEIFIYPTNKNQFTIFNSKPSEVNISQNDYIIYDNKYKNFSISFWVRIPNYDNKIVNINNEYTIINCMRDNNSGWKVSLNHNEIIWTLQDNARINQKLVFKYGNANGISDYINKWIFVTITNDRLGDSKLYINGHLIDQKSILNLGNIHVSDNILFKIVNCSYTRYIGIRYFNIFDKELDETEIQTLYSNEPNTNILKDFWGNYLLYDKGYYLLNVLKPNNFIDRRKDSTLSINNIRSTILLANRLYSGIKVKIQRVNDSSTNDRFVRKNDQVYINYISNSSSYSLYADTNTTDKEKTIKSSSSGNRFNQVVVMNSVGNNCTMNFKNNNGNNIGLLGFKADTVVASTWYYTHMRDHTNSNGCFWNFISEEHGWQEK

SEQ ID NO: 35 - protein, 1252 aa, Clostridium botulinum
  REGION 1..1252: note = BoNT/E6
MPTINSFNYNDPVNDRTILYIKPGGCQEFYKSFNIMKNIWIIPERNVIGTTPQDFHPPTSLKNGDSSYYDPNYLQSYEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFKDNSMNEFIQDPALTLMHELIHSLHGLYGAKGITTQYTITQQQNPLITNIKGTNIEEFLTFGGTDLNIITSAQYNDIYTNLLADYKKIASKLSKVQVSNPLLNPYKDVFEKKYGLDKDASGIYSVNINKFNDIFKKLYSFTEFDLATKFQVKCRQTYIGQYKYFKLSNLLNNSIYNISEGYNINTLKVNFRGQNTNLNPRIITPLTGRGLVKKIIRFCKNIVFSKGIRKSICIEINNGELFFVASDNSYNDDNINTPKEIDDTVTSNNNYENDLDQVILNFNSESAPGLSDEKLNLTIQNDAYIPKYDSNGTSDIEQHDVNELNVFFYLDAQKVPEGENNIDFTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKTIIESKYNSYTLEEKNELTNKYNIEQIENELNQKVSIAMNNIEIFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNNKLSEVNISQNDYIIYDNKYKNFSISFWVRIPNYDNKIVNVNNEYTIINCMRDNNSGWKVSLNHNEIIWTLQDNSGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILNDFWGNYLLYDKEYYLLNVLKPNNFINRRTDSTLSINNIRSTILLANRLYSGIKVKIQRVNNSSTNDNLVRKNDQVYINFVDSKTHLLPLYADTATTNKEKTIKISSSGNRFNQVVVMNSVGNNCTMNFKNNNGNNIGLLGFKADTVVASTWYYTHMRDNTNSNGFFWNFISEEHGWQEK

SEQ ID NO: 36 - protein, 1252 aa, Clostridium botulinum
  REGION 1..1252: note = BoNT/E7
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGRILLEELSKANPYLGNDNTPDNQFHIGDASAVEIKFSNGNQSILLPNVIIMGAEPDLFETNSSNISLRNNYMPSNHGFGSIAIVTFSPEYSFRFNDNSMNEFIQDPALTLMHELIHSLHGLYGAKRITTKYTITQQQNPLITNIRGTNIEEFLTFGGTDLNIITSAQYNDIYTNLLADYKKIASKLSKVQVSNPQLNPYKDIFQEKYGLDKNASGIYSVNINKFDDIFKKLYSFTEFDLATKFQVKCRQTYIGQYKYFKLSNLLNNSIYNISEGYNINTLKVNFRGQNTNLNPRIITQLTGRGLVKKIIRFCKNIVFSKGITKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 37 - protein, 1252 aa, Clostridium botulinum
  REGION 1..1252: note = BoNT/E8
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGRILLEELSKANPYLGNDNTPDNQFHIGDASAVEIKFSNGNQSILLPNVIIMGAEPDLFETNSSNISLRNNYMPSNHGFGSIAIVTFSPEYSFRFNDNSMNEFIQDPALTLMHELIHSLHGLYGAKRITTKYTITQQQNPLITNIRGTNIEEFLTFGGTDLNIITSAQYNDIYTNLLADYKKIASKLSKVQVSNPQLNPYKDIFQEKYGLDKNASGIYSVNINKFDDIFKKLYSFTEFDLATKFQVKCRQTYIGQYKYFKLSNLLNNSIYNISEGYNINTLKVNFRGQNTNLNPRIITQLTGRGLVKKIIRFCKNIVFSKGITKSICIEINNGELFFVASENSYNDDNINTPKEIDDTVTSNNNYENDLDQVILNFNSESAPGLSDEKLNLTIQNDAYIPKYDSNGTSYIEQHDVNELNVFFYLDAQKVPEGENNVNLTSSIDTALLEQPKIYTFFSSEFINNVNKTVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKTIIESKYNSYTLEEKNELTNKYNIEQIENELNQKVSIAMNNIEIFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILKDFWGNYLLYDKEYYLLNVLKPNNFIDRRTDSTLSINNIRSTILLANRLYSGIKVKIQRVNNSSTNDNLVRKNDQVYINFVASKTHLFPLYADTNTTNKEKTIKSSSSGNRFNQVVVMNSVGNNCTMNFKNNNGNNIGMLGFKDNTLVASTWYYTHMRDNTNSNGCFWNFISEEHGWQEK

SEQ ID NO: 38 - protein, 1251 aa, Clostridium botulinum
  REGION 1..1251: note = BoNT/E9
MPKINSFNYNDPVNDNTILYIKPGGCQQFYKSFNIMKNIWIIPERNVIGTIPQNFLPPTSLKNGDSSYYDPNYLQNDQEKDRFLKIVTKVFNRINDNLSGRILLEELSKANPYLGNDNTRDDDFIINDGSAVPIQFSNGSQSILLPTVIIMGAEPDLFETNSSNVSLINNYSPSNHGFGSIAIVTFSPEYSFRFNDNSMNEFIQDPALTLIHELIHSLHGLYGAKGITTKYTITQQQNPLITNIRGINIEEFLTFGGNNLNIITSSQLNDIYTNLLDDYKKIASKLSKVQVSNPQLNPYKDVFQEKYGLDKNASGIYSVNINKFNDIFKKLYSFTEFDLATKFQVKCRETYIGQYKYFKLSNLLNDSIYNISEGYNINTLNVNFRGQNPNLNPRIITPITDRGLVKKIIRFCKNIVSVKGIRKSICIEVNNGDLFFVASEKSYNNDSINIPKEIDDTVTLNNNYENDLDQVILNFNSESAPGLSDKKLNISIQDDVYIPKYDSNGTSDIEQYDVSELNVFFYLDAQKVPEGENNVNLTSSIDTALLEQSKIYTFFSSEFINNVNKPVQAALFVGWIQQVLVDFTTEATQKSTVDKIADISIVVPYIGLALNIGNESQKGNFKDALELLGAGILLEFVPELLIPTILVFTIKSFLGSSDNKNKVIKAINNALKERDEKWKEVYSFIVSNWITKINTQFNKRKEQMYQALQNQVNALKTIIESKYNSYTLEEKNELTNKYDIEQIENELNQKVSIAMNNIDRFLTESSISYLMKLINEVKINKLREYDENVKTYLLDYITKHGSILGESQQELNSMIIDTLNNSIPFKLSSYTDDKILISYFNKFFKTIKSSSVLSMRYKNDKYIDTSGYDSNININGDVFIYPTNKNQFGIYNSKLSEVNISQNDYIIYDNKYKNFSISFWVRIPNYNNKIVNVNNEYTIINCMRDNNSGWKISLNHNEIIWTLQDNAGINQKLVFKYGNANGISDYINKWIFVTITNDRLGYSKLYINGHLIDQKSILNLGNIHVSDNILFKIVNCSYTRYIGMRYFNIFDKELDETEIQTLYNNEPNANVLKDFWGNYLLYNKEYYLLNMLKPSKTISHNRDLTFSIYNNRNIVNGLYRLYSGIKVKIQKINDSDTRDNIVRDNDQVYVNYINGNVYYSLYADTNATNKEKTIKSSTSGNRFNQVVVMNSVRNNCTMNFKNNNGHDIGLLGFKSNALVASTWYYTNMRDHTNSNGCFWSFIPEENGWQEH

SEQ ID NO: 39 - protein, 1252 aa, Clostridium botulinum
  REGION 1..1252: note = BoNT/E10
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGGILLEELSKANPYLGNDNTPNNQFHIGDASAVEIKFSNGSQSILLPTVIIMGAEPDLFETNSSNISLKNNYMPSNHGFGSIAIVTFSPEYSFRFNDNSMNEFIQDPALTLMHELIHSLHGLYGAKGITTKYTITQQQNPLITNIRGTNIEEFLTFGGTDLNIITNAQSNDIYTNLLADYKKIASKLSQVQVSNPQLNPYKDIFQEKYGLDKNASGIYSVNINKFDDIFKKLYSFTEFDLATKFQVKCRQTYIGQYKYFKLSNLLNNSIYNISEGYNINTLKVNFRGQNTNLNPRIITQLTGRGLVKKIIRFCKNIVFSKGITKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKTIIESKYNSYTLEEKNELTNKYNIEQIENELNQKVSIAMNNIEIFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDKKSILNLGNIHVSDNILFKIVNCSYTRYIGMRYFNIFDKELDKTEIETLYNNEPNTNILKDFWGNYLLYDKEYYLLNVLKPNNVIDSNRDSTFSIHNIRSTIVLANKLYLGIKVKIQRVNNSSTNDNLVRKNDQVYINFVPIKTHLFPLYADTNTTNKEKTIKSSSSGNRFNQVVVMNSVGNNCTMNFKNNNGNNIGMLGFKDNTLVASTWYYTHMRDNTNSNGCFWNFISEEHGWQEK

SEQ ID NO: 40 - protein, 1252 aa, Clostridium botulinum
  REGION 1..1252: note = BoNT/E11
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGGILLEELSKANPYLGNDNTPNNQFHIGDASAVEIKFSNGSQSILLPTVIIMGAEPDLFETNSSNISLKNNYMPSNHGFGSIAIVTFSPEYSFRFNDNSMNEFIQDPALTLMHELIHSLHGLYGAKGITTKYTITQQQNPLITNIRGTNIEEFLTFGGTDLNIITNAQSNDIYTNLLDDYKKIASKLSQVQVSNPQLNPYKDVFQEKYGLDKDANGIYSVNINKFNDIFKKLYSFTEFDLATKFQVKCRKTYIGHHKYFRLSDLLNDSIYNISDGYNINTLKVNFRGQNTNLNTRIITPITGRGVVRKIIRFCTNIFSPKGIRKSICIEVNNGELFFVASENSYNDDNINTSKEIDDTVTSNNNYENDLDQVILNFNSESAPGLSDEKLNLTIQDDAYIPKYDSNGTSDIEQYDVSELNVFFYLDAQKVPEGENNVDFTSSIDTALLEQPKIYTFFSSKFISNLNKTMQAALFVSWIQQVLVDFTTEATQKSTVDKIADISIVVPYIGLALNIGNEAQKGNFKDALELLGAGILLEFEPELLIPIILVFTIKSFLGSSDNKNKVIKAINNALKERDENWKEVYSFIVSNWMTKINTQFNKRKEQMYQALQNQVNAIKTIIESKYNSYTLEEKNELKNKYDIEQIENELNQTVSIAMNNIEIFLTESSISYLMKLINEVKINKLKEYDENVKTYLLDYIIKHGSILGESQQELNSMVIDTLNNSIPFKLSSYTDDKILISYFNKFFKTIKSSSVLNMRYKNDKYIDTSGYDSNINIKGDVFIYPTNKNQFGIYNNKLSEVNISQNDYIIYDNKYKNFSISFWVRIPNYDNKIVNVNNEYTIINCMRDNNSGWKVSLNHNEIIWTLQDNAGINQKLVFKYGNANGISDYINKWIFVTITNDRLGDSKLYINGNLIDKKSILNLGNIHVSDNILFKIVNCSYTRYIGMRYFNIFDKELDKTEIETLYNNEPNTNILKDFWGNYLLYDKEYYLLNVLKPNNVIDSNRDSTFSIHNIRSTIVLANRLYSGIKVKIQRVNNSSTNDNLVRKNDQVYINFVASKTHLFPLYADTNTTNKEKTIKSSSSGNRFNQVVVMNSVGNNCTMNFKNNNGNNIGMLGFKDNTLVASTWYYTHMRDNTNSNGCFWNFISEEHGWQEK

SEQ ID NO: 41 - protein, 1251 aa, Clostridium botulinum
  REGION 1..1251: note = BoNT/E12
MPKINSFNYNDPVNDRTILYIKPGGCQQFYKSFNIMKNIWIIPERNVIGTIPQDFQPPTSLKNGDSSYYDPNYLQSNEEKDRFLKIVTKIFNRINDNLSGGILLEELSKANPYLGNDNTPDGDFIINDASAVPIQFSNGSQSILLPNVIIMGAEPDLFETNSSNISLINNYRPSNHGFGSIAIVTFSPEYSFRFNDNSMNEFIQDPALTLMHELIHSLHGLYGAKGITTKYTITQQQNSLITNIRGINIEEFLTFGGNDLNIITSSQFNDIYTNLLDDYKKIASKLSQVRVSNPQLNPYKDVFQEKYGLDKDASGIYSVNINKFNDIFKKLYSFTEFDLATKFQVKCRETYIGQYKYFQLSNLLNDSIYNISEGYNINNLKVNFRGQNANLNPRIITPITGRGLVKKIIRFCKNIVSVKGIRKSICIEVNNGELFFVASENSYNDDNINTPKEIDDTVTSNNNYENDLDQVILNFNSESAPGLSDEKLNLTIQDDAYIPKYDSNGTSDIEQHDVNELNVFFYLDAQKVPEGENNVNLTSSIDTALLEQPKIYTFFSSEFINNVNKPVQAVLFVSWIQQVLVDFTTEATQKSTVDKIADISIVVPYIGLALNIGNEAQKGNFKDALELLGAGILLEFVPELLIPTILVFTIKSFLGSSDNKNKIIKAINNALKERDEKWKEVYSFIVSNWITKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSTLGESQQELNSMVINTLNNSIPFKLSSYTDDKILISYFNKFFKRIKSSSVLNMRYKNDKYVDTSGYDSNININGEIFIYPTNKNQFSIFNSKPSEVNISQNDYIIYDNKYKNFSISFWVRIPNYDNKIVNVNNEYTIINCMRDNNSGWKVSLNHNEIIWTLQDNAGINQKLAFNYGNSNGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNSIISHRRDLTFSFYNHRYIVNGLYRLYSGIKVKIQRVNDSSTNDQFVRKNDQVYINYIYNNLSYSLYADTNIKDKEKTIKSSLSGNIFNQVVVMNSVGNNCTMNFKNNNGNNIGLLGFKDNTLVASTWYYTHMRDNTNSNGCFWNFISEEHGWQEK

SEQ ID NO: 42 - protein, 1278 aa, Clostridium botulinum
  REGION 1..1278: note = BoNT/F1
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 43 - protein, 1280 aa, Clostridium botulinum
  REGION 1..1280: note = BoNT/F2
MPVVINSFNYNDPVNDETILYMQKPYEERSRKYYKAFEIMPNVWIMPERDTIGTKPDEFQVPDSLKNGSSAYYDPNYLTTDAEKDRYLKTMIKLFNRINSNPTGKVLLEEVSNARPYLGDDDTLINEFLPVNVTTSVNIKFSTDVESSIISNLLVLGAGPDIFKAYCTPLVRFNKSDKLIEPSNHGFGSINILTFSPEYEHIFNDISGGNHNSTESFIADPAISLAHELIHALHGLYGAKAVTHKESLVAERGPLMIAEKPIRLEEFLTFGGEDLNIIPSAMKEKIYNDLLANYEKIATRLREVNTAPPGYDINEYKDYFQWKYGLDRNADGSYTVNRNKFNEIYKKLYSFTEIDLANKFKVKCRNTYFIKYGFVKVPNLLDDDIYTVSEGFNIGNLAVNNRGQNINLNPKIIDSIPDKGLVEKIIKFCKSIIPRKGTKQSPSLCIRVNNRELFFVASESSYNESDINTPKEIDDTTNLNNNYRNNLDEVILDYNSETIPQISNRTLNTLVQDNSYVPRYDSNGTSEIEEYDVVDFNVFFYLHAQKVPEGETNISLTSSIDTALLEESKVYTFFSSEFIDTINKPVNAALFIDWISKVIRDFTTEATQKSTVDKIADISLIVPYVGLALNIVIEAEKGNFEEAFELLGAGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESKYNINNIEEELNKKVSLAMKNIERFMTESSISYLMKLINEAEVGKLKEYDKHVKSDLLDYILYHKLILGEQTKELIDLVTSTLNSSIPFELSSYTNDKILIIYFNRLYKKIKDSSILDMRYENNKFIDISGYGSNISINGNVYIYSTNRNQFGIYSGRLSEVNIAQNNDIIYNSRYQNFSISFWVTIPKHYRPMNRNREYTIINCMGNNNSGWKISLRTIRDCEIIWTLQDTSGNKEKLIFRYEELASISDYINKWIFVTITNNRLGNSRIYINGNLIVEKSISNLGDIHVSDNILFKIVGCDDETYVGIRYFKVFNTELDKTEIETLYSNEPDPSILKDYWGNYLLYNKKYYLFNLLRKDKYITRNSGILNINQQRGVTGGISVFLNYKLYEGVEVIIRKNAPIDISNTDNFVRKNDLAYINVVDHGVEYRLYADISITKSEKIIKLIRTSNPNDSLGQIIVMDSIGNNCTMNFQNNDGSNIGLLGFHSDDLVASSWYYNHIRRNTSSNGCFWSFISKEHGWKE

SEQ ID NO: 44 - protein, 1279 aa, Clostridium botulinum
  REGION 1..1279: note = BoNT/F3
MPVVINSFNYNDPVNDETILYMQKPYEERSRKYYKAFEIMPNVWIMPERDTIGTKPDDFQVPDSLKNGSSAYYDPNYLTTDAEKDRYLKTMIKLFNRINSNPTGKVLLEEVSNARPYLGDDDTLINEFFPVNVTTSVNIKFSTDVESSIISNLLVLGAGPDIFKAYCTPLVRFNKSDKLIEPSNHGFGSINILTFSPEYEHIFNDISGGDHNSTESFIADPAISLAHELIHALHGLYGAKAVTHKETIEVKRGPLMIAEKPIRLEEFLTFGGEDLNIIPSAMKEKIYNDLLANYEKIATRLREVNTAPPEYDINEYKDYFQWKYGLDRNADGSYTVNRNKFNGIYKKLYSFTEIDLANKFKVKCRNTYFIKYGFVKVPDLLDDDIYTVSEGFNIGNLAVNNRGQNINLNPKIIDSIPDKGLVEKIIKFCKSIIPRKGTKQSPSLCIRVNNRELFFVASESSYNESDINTPKEIDDTTNLNNNYRNNLDEVILDYNSETIPQISNRTLNTLVQDNSYVPRYDSNGTSEIEEYDVVDFNVFFYLHAQKVPEGETNISLTSSIDTALLEKSKVYTFFSSEFIDTINESVNAALFIDWINKVIRDFTTEATQKSTVDKIADISLIVPYVGLALNIVIDAEKGNFQEAFELLGAGILLEFVPELTIPVILVFTIKSYIDSYENKNKAIKAINNALIEREAKWKEIYSWIVSNWLTKINTQFNKRKEQMYQALQNQVDAIKTAIEYKYNNYTSDEKNRLESEYNINNIEEELNKKVSLAMKNIERFMTESSISYLMKLINEAEVGKLKKYDRHVKSDLLDYILYHKLILGDQTKELIDLVTSTLNSSIPFELSSYTNDKILIIYFNRLYKKIKDSSILDMRYENNKFIDISGYGSNISINGNVYIYSTNRNQFGIYSDRLSEVNIAQNNDIIYNSRYQNFSISFWVRIPKHYGPMNRNREYTIINCMGNNNSGWKISLRNIRDCEIIWTLQDTSGNKEKLIFRYEELANISDYINKWIFVTITNNRLGNSRIYINGNLIVEKSISNLGDIHVSDNILFKIVGCDDKTYVGIRYFKVFNTELDKTEIETLYSNEPDPSILKDYWGNYLLYNKKYYLFNLLRKDKYITRNSGILNINQQRGVTEGSVFLNYKLYEGVEVIIRKNGPIDISNTDNFVRKNDLAYINVVYHDVEYRLYADISITKPEKIIKLIRTSNPNDSLGQIIVMDSIGNNCTMNFQNNNGGNIGLLGFHSDNLVASSWYYNNIRRNTSSNGCFWSFISKEHGWQE

SEQ ID NO: 45 - protein, 1277 aa, Clostridium botulinum
  REGION 1..1277: note = BoNT/F4
MPVVINSFNYDDPVNDDTILYMQIPYEEKSKKYYKAFEIMRNVWIMPERNTIGTNPSDFDPPASLKNGSSAYYDPNYLTTDAEKDRYLKTTIKLFKRINSNPAGEVLLQEISYAKPYLGNDHTPINEFHPVTRTTSVNIKSSTNVESSIILNLLVLGAGPNIFENSSYPVRKLMNSGEVYDPSNDGFGSINIVTFSPEYEYTFNDISGGHNSSTESFIADPAISLAHELIHALHGLYGARGVTYKETIKVKQAPLMIAEKPIRLEEFLTFGGQDLNIITSAMKEKIYNDLLANYEKIATRLSEVNSAPPEYDINEYKNYFQWKYGLDKNADGSYTVNENKFNEIYKKLYSFTEIDLANKFKVKCRNTYFIKYGFLKVPNLLDDDIYTVSEGFNIGNLAVNNRGQNINLNPKIIDSIPDKGLVEKIVKLCKSIIPRKGTKAPPRLCIRVNNRELFFVASESSYNENDINTPKEIDDTTNLNNNYRNNLDEVILDYNSETIPQISSQTLNTLVQDDSYVPRYDSNGTSEIEEHNVVDLNAFFYLHAQKVPEGETNISLTSSIDTALSEESKVYTFFSSEFINNINKPVHAALFIGWISQVIRDFTTESTQKSTVDKIADISLIVPYVGLALNIGNDARKGNFKEAFELLGAAILLEVVPELLIPVILVFTIKSFIDSSKNEDKIIKAINNSLIEREAKWKEVYSWIVSNWLTRINTQFNKRKEQMYQALQNQVDAIKTVIEYKYNSYTSDEKNRLESEYNINNIEEELNKKVSLAMKNIERFIAESSISYLMKLINEAKVSELREYDEGVKEYLLDYILKNGSILGDHVQELNDLVTSTLNSSIPFELSSYTNDKILIIYFNKLYKKIKDNCILDMRYENNKFIDISGYGSNISINGELYIYTTNRNQFTIYSGKLSEVNIAQNNDIIYNSRYQNFSISFWVRIPRYSNIVNLNNEYTIINCMGNNNSGWKISLNYNKIIWTLQDTAGNNEKLVFNYTQMISISDYINKWIFVTITNNRLGNSRIYINGNLIDQKSISNLGDIHVSDNILFKIVGCNDTRYVGIRYFKVFDTELDKTEIETLYSDEPDPSILKDFWGNYLLYNKRYYLLNLLRKDNAITQSSTFLSISRARGVDRKANIFSNKRLYKGVEVIIRKNEPIDISNTDNFVRKGDLAYINVVDRDVEYRLYANTSNAQPEKTIKLIRTSNSNDSLDQIIVMDSIGNNCTMNFQNNNGGNIGLLGFHSNTLVASSWYYNNIRRNTSSNGCFWSFISKEHGWQE

SEQ ID NO: 46 - protein, 1277 aa, Clostridium botulinum
  REGION 1..1277: note = BoNT/F5
MPVEINSFNYDDLVNDNTILYIRPPYYERSNTYFKAFNIMENVWIIPERYRLGIEASKFDPPDSLKAGSDGYFDPNYLSTNTEKNRYLQIMIKLFKRINSNEAGKILLNQIKDAIPYLGNSYTAEDQFTTNNRTISFNVRLANGTIEQEMANLIIWGPGPDLTTNRTGGTTYTPAQSLEAIPYKEGFGSIMTIEFSPEYATAFNDISLTSHAPSLFIKDPALILMHELIHVLHGLYGTYTTGFKIKPNITEPYMEVTKPITSGEFLTFGGNDVNKIPQLIQSQLRSKVLDDYEKIASRLNKVNRATAEINIDKFKYSYQLKYQFVKDSNGVYSVDLDKFNKLYDKIYSFTEFNLAHEFKIKTRNSYLAKNFGPFYLPNLLDNSIYNEADGFNIGDLSVNYKGQVIGSDIDSIKKLEGQGVVSRVVRLCLNSSFKKNTKKPLCITVNNGDLFFIASEDSYGEDTINTPKEIDDTTTLVPSFKNILDKVILDFNKQVTPQIPNRRIRTDIQEDNYIPEYDSNGTSEIEEYNVVDLNAFFYLHAQKVPEGETNISLTSSIDTALSEESKVYTFFSSEFIDTINEPVNAALFIDWISKVIRDFTTEATQKSTVDKIADISLIVPYVGLALNIVNETEKGNFKEAFELLGAGILLEFVPELAIPVILVFTIKSYIDSYENKNKIIKAINNSLIEREAKWKEIYSWIVSNWLTRINTQFNKRKEQMYQALQNQVDAIKTAIEYKYNNYTSDEKNRLESEYNINNIEEELNKKVSLAMKNIERFITESSISYLMKLINEAEVGKLKEYDKRVKRHLLEYIFDYRLILGEQGGELIDLVTSTLNTSIPFELSSYTNDKILIIYFNRLYKKIKDSSILDMRYENNKFIDISGYGSNISINGNVYIYSTNRNQFGIYDDRLSEVNIAQNNDIIYNSRYQNFSISFWVRIPKHYRPMNHNREYTIINCMGNNNSGWKISLRTTGDCEIIWTLQDTSGNKKKLIFRYSQLGGISDYINKWIFVTITNNRLGNSRIYINGNLIVEKSISNLGDIHVSDNILFKIVGCDDKMYVGIRYFKVFNTELDKTEIEILYSNEPDPSILKDYWGNYLLYNKKYYLLNLLRNDKYITRNSDILNISHQRGVTKDLFIFSNYKLYEGVEVIIRKNGPIDISNTDNFVRKNDLAYINVVDHGVEYRLYADISITKPEKIIKLIRRSNPDDSLGQIIVMDSIGNNCTMNFQNNNGGNIGLLGFHSDNLVASSWYYNNIRRNTSSNGCFWSFISKEHGWQE

SEQ ID NO: 47 - protein, 1274 aa, Clostridium botulinum
  REGION 1..1274: note = BoNT/F6
MPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRKGTKAPPRLCIRVNNSELFFVASESSYNENDINTPKEIDDTTNLNNNYRNNLDEVILDYNSQTIPQISNRTLNTLVQDNSYVPRYDSNGTSEIEEYDVVDFNVFFYLHAQKVPEGETNISLTSSIDTALLEESKDIFFSSEFIDTINKPVNAALFIDWISKVIRDFTTEATQKSTVDKIADISLIVPYVGLALNIIIEAEKGNFEEAFELLGVGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESEYNINNIEEELNKKVSLAMKNIERFMTESSISYLMKLINEAKVGKLKKYDNHVKSDLLNYILDHRSILGEQTNELSDLVTSTLNSSIPFELSSYTNDKILIIYFNRLYKKIKDSSILDMRYENNKFIDISGYGSNISINGNVYIYSTNRNQFGIYNSRLSEVNIAQNNDIIYNSRYQNFSISFWVRIPKHYKPMNHNREYTIINCMGNNNSGWKISLRTVRDCEIIWTLQDTSGNKENLIFRYEELNRISNYINKWIFVTITNNRLGNSRIYINGNLIVEKSISNLGDIHVSDNILFKIVGCDDETYVGIRYFKVFNTELDKTEIETLYSNEPDPSILKNYWGNYLLYNKKYYLFNLLRKDKYITLNSGILNINQQRGVTEGSVFLNYKLYEGVEVIIRKNGPIDISNTDNFVRKNDLAYINVVDRGVEYRLYADTKSEKEKIIRTSNLNDSLGQIIVMDSIGNNCTMNFQNNNGSNIGLLGFHSNNLVASSWYYNNIRRNTSSNGCFWSSISKENGWKE

SEQ ID NO: 48 - protein, 1268 aa, Clostridium baratii
  REGION 1..1268: note = BoNT/F7
MPVNINNFNYNDPINNTTILYMKMPYYEDSNKYYKAFEIMDNVWIIPERNIIGKKPSDFYPPISLDSGSSAYYDPNYLTTDAEKDRFLKTVIKLFNRINSNPAGQVLLEEIKNGKPYLGNDHTAVNEFCANNRSTSVEIKESKGTTDSMLLNLVILGPGPNILECSTFPVRIFPNNIAYDPSEKGFGSIQLMSFSTEYEYAFNDNTDLFIADPAISLAHELIHVLHGLYGAKGVTNKKVIEVDQGALMAAEKDIKIEEFITFGGQDLNIITNSTNQKIYDNLLSNYTAIASRLSQVNINNSALNTTYYKNFFQWKYGLDQDSNGNYTVNISKFNAIYKKLFSFTECDLAQKFQVKNRSNYLFHFKPFRLLDLLDDNIYSISEGFNIGSLRVNNNGQNINLNSRIVGPIPDNGLVERFVGLCKSIVSKKGTKNSLCIKVNNRDLFFVASESSYNENGINSPKEIDDTTITNNNYKKNLDEVILDYNSDAIPNLSSRLLNTTAQNDSYVPKYDSNGTSEIKEYTVDKLNVFFYLYAQKAPEGESAISLTSSVNTALLDASKVYTFFSSDFINTVNKPVQAALFISWIQQVINDFTTEATQKSTIDKIADISLVVPYVGLALNIGNEVQKGNFKEAIELLGAGILLEFVPELLIPTILVFTIKSFINSDDSKNKIIKAINNALRERELKWKEVYSWIVSNWLTRINTQFNKRKEQMYQALQNQVDGIKKIIEYKYNNYTLDEKNRLKAEYNIYSIKEELNKKVSLAMQNIDRFLTESSISYLMKLINEAKINKLSEYDKRVNQYLLNYILENSSTLGTSSVQELNNLVSNTLNNSIPFELSEYTNDKILISYFNRFYKRIIDSSILNMKYENNRFIDSSGYGSNISINGDIYIYSTNRNQFGIYSSRLSEVNITQNNTIIYNSRYQNFSVSFWVRIPKYNNLKNLNNEYTIINCMRNNNSGWKISLNYNNIIWTLQDTTGNNQKLVFNYTQMIDISDYINKWTFVTITNNRLGHSKLYINGNLTDQKSILNLGNIHVDDNILFKIVGCNDTRYVGIRYFKIFNMELDKTEIETLYHSEPDSTILKDFWGNYLLYNKKYYLLNLLKPNMSVTKNSDILNINRQRGIYSKTNIFSNARLYTGVEVIIRKVGSTDTSNTDNFVRKNDTVYINVVDGNSEYQLYADVSTSAVEKTIKLRRISNSNYNSNQMIIMDSIGDNCTMNFKTNNGNDIGLLGFHLNNLVASSWYYKNIRNNTRNNGCFWSFISKEHGWQE

SEQ ID NO: 49 - protein, 1281 aa, Clostridium botulinum
  REGION 1..1281: note = BoNT/F8
MPVVINSFNYNDPVNDDTILYMQIPYEEKSKKYYKAFEIMRNVWIIPERNTIGTDPSDFDPPASLKNGSSAYYDPNYLTTDAEKDKYLKTTIKLFKRINSNPAGEVLLQEISYAKPYLGNEHTPINEFHPVTRTTSVNIKSSTNVKSSIILNLLVLGAGPNIFENSCYPVRKLMDSGEVYDPSNDGFGSINIVTFSPEYEYTFNDISGGHNSSTESFIADPAISLAHELIHALHGLYGARGVTYKETIKVKQAPLMIAEKPIRLEEFLTFGGQDLNIITSAMKEKIYNNLLANYEKIATRLSEVTSAPPEYDINEYKDYFQWKYGLDKNADGSYTVNENKFNEIYKKLYSFTENDLANKFKVKCRNTYFIKYGFLKVPNLLDDDIYTVSEGFNIGNLAINNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLIVPYVGLALNIGNDVSKGDFKKAFELFGAAILLEVAPELLIPVILVFTIKSFIDSSENEDKIIKAIIKAINNSLMEREAKWQEIYGWIVSNWLTRINTQFNKRKEQMYQALQNQVDAIKTVIEYKYNNYTSDEKNRLESEYNINNIEEELNKKVSLAMKNIERFIAESSISYLMKLINEAKVSKLREYDEGVKEYLLDYILKHGSILGDRVQELNDLVTSTLNSSIPFELSSYTNDKILIIYFNKLYEKIKDNSILDMRYKNNKFIDISGYGSNISINGDVYIYSTNRNQFGIYSNKPSEVNIAQNNDIIYNSRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELDKTEIETLYSDEPDPSILKDFWGNYLLYNKRYYLLNLLRTDKSITQNSNFLNINQQRGVYQKPNIFSNTRLYTGVEVIIRKNGSTDISNTDDFVRKNDLAYINVVDHGVEYRLYADISIAKSEKIIKLIRTSNSNNSLGQIIVMDSIGNNCTMNFQNNNGGNIGLLGFHSNNLVASSWYYNNIRKNTSSNGCFWSFISKEHGWQE

SEQ ID NO: 50 - protein, 1297 aa, Clostridium argentinense
  REGION 1..1297: note = BoNT/G
MPVNIKNFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 51 - protein, 1288 aa, Clostridium botulinum
  REGION 1..1288: note = BoNT/FA(H)
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHELIHVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSNTKNSLCITVNNRDLFFIASQESYGENTINTYKEIDDTTTLDPSFEDILDKVILNFNEQVIPQMPNRNVSTDIQKDNYIPKYDYNRTDIIDSYEVGRNYNTFFYLNAQKFSPNESNITLTSSFDTGLLEGSKVYTFFSSDFINNINKPVQALLFIEWVKQVIRDFTTEATKTSTVDKLKDISLVVPYIGLALNIGDEIYKQHFAEAVELVGAGLLLEFSPEFLIPTLLIFTIKGYLTGSIRDKDKIIKTLDNALNVRDQKWKELYRWVVSKWLTTINTQFNKRKEQMYKALKNQATAIKKIIENKYNNYTTDEKSKIDSSYNINEIERTLNEKINLAMKNIEQFITESSIAYLINIINNETIQKLKSYDDLVRRYLLGYIRNHSSILGNSVEELNSKVNNHLDNGIPFELSSYTNDSLLIRYFNKNYGELKYNCILNIKYEMDRDKLVDSSGYRSRINIGTGVKFSEIDKNQVQLSNLESSKIEVILNNGVIYNSMYENFSTSFWIRIPKYFRNINNEYKIISCMQNNSGWEVSLNFSNMNSKIIWTLQDTEGIKKTVVFQYTQNINISDYINRWIFVTITNNRLSNSKIYINGRLINEESISDLGNIHASNNIMFKLDGCRDPHRYIWIKYFNLFDKELNKKEIKDLYDNQSNSGILKDFWGDYLQYDKPYYMLNLYDPNKYLDVNNVGIRGYMYLKGPRGRIVTTNIYLNSTLYMGTKFIIKKYASGNKDNIVRNNDRVYINVVVKNKEYRLATNASQAGVEKILSAVEIPDVGNLSQVVVMKSENDQGIRNKCKMNLQDNNGNDIGFIGFHQFNNIAKLVASNWYNRQIGKASRTFGCSWEFIPVDDGWGESSL

SEQ ID NO: 52 - protein, 1306 aa, Clostridium botulinum
  REGION 1..1306: note = BoNT/X
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 53 - protein, 1315 aa, Clostridium tetani
  REGION 1..1315: note = Tetanus neurotoxin
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 54 - protein, 6 aa, synthetic construct
  source 1..6: note = Linker Sequence
GSGGSG

SEQ ID NO: 55 - protein, 19 aa, synthetic construct
  source 1..19: note = Linker Sequence
AAASGGSGGGGSGGGGSGP

SEQ ID NO: 56 - protein, 17 aa, synthetic construct
  source 1..17: note = Linker Sequence
AAASGGSGGGGSGGGGS

SEQ ID NO: 57 - protein, 20 aa, synthetic construct
  source 1..20: note = Linker Sequence
GGGGSGGGGSGGGGSGGGGS

SEQ ID NO: 58 - protein, 21 aa, synthetic construct
  source 1..21: note = Linker Sequence
GGGGSGGGGSGGGGSGGGGSG

SEQ ID NO: 59 - protein, 11 aa, synthetic construct
  source 1..11: note = Linker Sequence
SGSGGGGSGAG

SEQ ID NO: 60 - protein, 29 aa, synthetic construct
  source 1..29: note = Linker Sequence
SGSNGGAGQSGAGEGGGGSGGGGSGGGGS

SEQ ID NO: 61 - protein, 41 aa, synthetic construct
  source 1..41: note = Linker Sequence
TSGGGGSGGGGSGGGGSGGGGSGTSGSTGGGGSGGGGSGAG

SEQ ID NO: 62 - protein, 13 aa, synthetic construct
  source 1..13: note = Linker Sequence
SSSGSGGGGSGAG

SEQ ID NO: 63 - protein, 15 aa, synthetic construct
  source 1..15: note = Linker Sequence
GGGGSGGGGSGGGGS

SEQ ID NO: 64 - protein, 10 aa, synthetic construct
  source 1..10: note = Linker Sequence
GGGGSGGGGS

SEQ ID NO: 65 - protein, 5 aa, synthetic construct
  source 1..5: note = Linker Sequence
GGGGS

SEQ ID NO: 66 - protein, 8 aa, synthetic construct
  source 1..8: note = Linker Sequence
GGGGGGGG

SEQ ID NO: 67 - protein, 6 aa, synthetic construct
  source 1..6: note = Linker Sequence
GGGGGG

SEQ ID NO: 68 - protein, 12 aa, synthetic construct
  source 1..12: note = Linker Sequence
GSAGSAAGSGEF

SEQ ID NO: 69 - protein, 10 aa, synthetic construct
  source 1..10: note = Linker Sequence
VPGVGVPGVG

SEQ ID NO: 70 - protein, 21 aa, synthetic construct
  source 1..21: note = Linker Sequence
PAYSPGHGTQPFLEASGGPEA

SEQ ID NO: 71 - protein, 14 aa, synthetic construct
  source 1..14: note = Linker Sequence
SGGGGGSGGGGASG

SEQ ID NO: 72 - protein, 7 aa, synthetic construct
  source 1..7: note = Linker Sequence
ARGGASG

SEQ ID NO: 73 - protein, 6 aa, synthetic construct
  source 1..6: note = Histidine Tag
HHHHHH

SEQ ID NO: 74 - protein, 12 aa, synthetic construct
  source 1..12: note = Histidine Tag
HHHHHHHHHHHH

SEQ ID NO: 75 - protein, 14 aa, synthetic construct
  source 1..14: note = OLLAS tag
SGFANELGPRLMGK

SEQ ID NO: 76 - protein, 8 aa, synthetic construct
  source 1..8: note = STREP-tag II tag
WSHPQFEK

SEQ ID NO: 77 - protein, 368 aa, synthetic construct
  source 1..368: note = Maltose Binding Protein (MBP) tag
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTN

SEQ ID NO: 78 - protein, 293 aa, synthetic construct
  source 1..293: note = Halotag7
EIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEI

SEQ ID NO: 79 - protein, 15 aa, synthetic construct
  source 1..15: note = Avi Tag
GLNDIFEAQKIEWHE

SEQ ID NO: 80 - protein, 6 aa, synthetic construct
  source 1..6: note = thrombin cleavage site
LVPRGS

SEQ ID NO: 81 - protein, 4 aa, synthetic construct
  source 1..4: note = Factor Xa cleavage site
IEGR

SEQ ID NO: 82 - protein, 4 aa, synthetic construct
  source 1..4: note = Factor Xa cleavage site
IDGR

SEQ ID NO: 83 - protein, 5 aa, synthetic construct
  source 1..5: note = enterokinase cleavage site
DDDDK

SEQ ID NO: 84 - protein, 7 aa, synthetic construct
  source 1..7: note = Tobacco Etch Virus (TEV) protease site
  SITE 7: note = X at position 7 is G or S
ENLYFQX

SEQ ID NO: 85 - protein, 8 aa, synthetic construct
  source 1..8: note = PreScissionTM protease site
LEVLPQGP

SEQ ID NO: 86 - protein, 4 aa, synthetic construct
  source 1..4: note = furin protease cleavage site
RVRR

SEQ ID NO: 87 - protein, 12 aa, synthetic construct
  source 1..12: note = SnoopTagJr
KLGSIEFIKVNK

SEQ ID NO: 88 - protein, 23 aa, synthetic construct
  source 1..23: note = DogTag
DIPATYEFTDGKHYITNEPIPPK

SEQ ID NO: 89 - protein, 923 aa, synthetic construct
  source 1..923: note = DTnd delivery vehicle
MGHHHHHHHHHHHHDVSGFANELGPRLMGKGAGENLYFQGGGKLGSIEFIKVNKGGGGSGGGGSGGGGSGADDVVDSSKSFVMENFSSYHGTKPGYVDSIQKGIQKPKSGTQGNYDDDWEGFYSTDNKYDAAGYSVDNENPLSGKAGGVVKVTYPGLTKVLALKVDNAETIKKELGLSLTEPLMEQVGTEEFISRFGDGASRVVLSLPFAEGSSSVKYINNWEQAKALSVELEINFETRGKAGQDAMYEYMASACAGNRVRRSVGSSLSCINLDWDVIRDKTKTKIESLKEHGPISNKMSESPNKTVSEEKAKSYLEEFHQTALEHPELSELKTVTGTNPVFAGANYAAWAVNVAQVIDSSTADNLEKTTAALSILPGIGSVMGIADGAVHHNTEEIVAQSIALSSLMVAQAIPLVGELVDIGFAAYNFVESIINLFQVVHNSYNRPAYSPGHGTQPFLEASGGPEANIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPLGAGENLYFQGAGWSHPQFEKGAGWSHPQFEK

SEQ ID NO: 90 - protein, 134 aa, synthetic construct
  source 1..134: note = B8 sdAb
QAHVQLQQSGGGLVQPGGSLRLSCAASGSIFSIYAMGWYRQAPGKQRELVAAISSYGSTNYADSVKGRFTISRDNAKNTVYLQMNSLKPEDTAVYYCNADIATMTAVGGFDYWGQGTQVTVSSAHHSEDPTSQS

SEQ ID NO: 91 - protein, 630 aa, synthetic construct
  source 1..630: note = B8 sdAb propeptide fusion
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSGSGGGGSGAGENLYFQSGSNGGAGQSGAGEGGGGSGGGGSGGGGSQAHVQLQQSGGGLVQPGGSLRLSCAASGSIFSIYAMGWYRQAPGKQRELVAAISSYGSTNYADSVKGRFTISRDNAKNTVYLQMNSLKPEDTAVYYCNADIATMTAVGGFDYWGQGTQVTVSSAHHSEDPTSQSTSGGGGSGGGGSGGGGSGGGGSGTSGSTGGGGSGGGGSGAGDIPATYEFTDGKHYITNEPIPPKGAGENLYFQGHHHHHH

SEQ ID NO: 92 - protein, 137 aa, synthetic construct
  source 1..137: note = JSG-C1 sdAb
AGTSQVQLVESGGGLVQTGGSLRLSCAASGRTFRRNTMGWFRQAPGKVREFVAAISWSGDRTYCADSVKGRFTISRDNAKNTVDLLMNSLKPEDTAIYYCAADGTASVFNSYASADRNKYNYWGQGTQVTVSSGSTA

SEQ ID NO: 93 - protein, 633 aa, synthetic construct
  source 1..633: note = JSG-C1 sdAb propeptide fusion
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSGSGGGGSGAGENLYFQSGSNGGAGQSGAGEGGGGSGGGGSGGGGSAGTSQVQLVESGGGLVQTGGSLRLSCAASGRTFRRNTMGWFRQAPGKVREFVAAISWSGDRTYCADSVKGRFTISRDNAKNTVDLLMNSLKPEDTAIYYCAADGTASVFNSYASADRNKYNYWGQGTQVTVSSGSTATSGGGGSGGGGSGGGGSGGGGSGTSGSTGGGGSGGGGSGAGDIPATYEFTDGKHYITNEPIPPKGAGENLYFQGHHHHHH

SEQ ID NO: 94 - protein, 121 aa, synthetic construct
  source 1..121: note = 2B8 sdAb
QVQLAESGGGLVQAGGSLRLSCVVSGRTFSTSQMGWFRQPPGKERELVARISWRGKQHYADSVKGRFTISRDYAKNTVYLQMNGLKSEDTAVYYCAADRRRTYLGQQHDYWGQGTLVTVSS

SEQ ID NO: 95 - protein, 617 aa, synthetic construct
  source 1..617: note = 2B8 sdAb propeptide fusion
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSGSGGGGSGAGENLYFQSGSNGGAGQSGAGEGGGGSGGGGSGGGGSQVQLAESGGGLVQAGGSLRLSCVVSGRTFSTSQMGWFRQPPGKERELVARISWRGKQHYADSVKGRFTISRDYAKNTVYLQMNGLKSEDTAVYYCAADRRRTYLGQQHDYWGQGTLVTVSSTSGGGGSGGGGSGGGGSGGGGSGTSGSTGGGGSGGGGSGAGDIPATYEFTDGKHYITNEPIPPKGAGENLYFQGHHHHHH

SEQ ID NO: 96 - protein, 240 aa, synthetic construct
  source 1..240: note = anti-beta sheet scFv
GEVQLQQSVAELVRPGASVKLSCTASGFNIKNTYMHWVKQRPEQGLEWIGRIDPANGNTKYAPKFQGKATITADTSSNTAYLQLSSLTSEDTAIYYCARGSFYAMDYWGQGTSVTVSSGGGGSGGGGSGGGGSDVQITQSPSYLAASPGETITINCRASKSINKYLAWYQEKPGKTNKLLIYSGSTLQSGIPSRFSGSGSGTDFTLTISSLEPEDFAMYHCQQHNEYPWTFGGGTKLEIK

SEQ ID NO: 97 - protein, 695 aa, synthetic construct
  source 1..695: note = anti-beta sheet scFv propeptide fusion
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSGSGGGGSGAGGENLYFQGEVQLQQSVAELVRPGASVKLSCTASGFNIKNTYMHWVKQRPEQGLEWIGRIDPANGNTKYAPKFQGKATITADTSSNTAYLQLSSLTSEDTAIYYCARGSFYAMDYWGQGTSVTVSSGGGGSGGGGSGGGGSDVQITQSPSYLAASPGETITINCRASKSINKYLAWYQEKPGKTNKLLIYSGSTLQSGIPSRFSGSGSGTDFTLTISSLEPEDFAMYHCQQHNEYPWTFGGGTKLEIKGAGSGFANELGPRLMGKSGGGGSGSGDIPATYEFTDGKHYITNEPIPPKGSGENLYFQGHHHHHHHH

SEQ ID NO: 98 - protein, 243 aa, synthetic construct
  source 1..243: note = anti-phosphorylated tau scFv
EVQLVQSGAEVKKPGESLKISCKGSGYTFSNYWIEWVRQMPGKGLEWMGEILPGSDSIKYEKNFKGQVTISADKSISTAYLQWSSLKASDTAMYYCARRGNYVDDWGQGTLVTVSSGGGGSGGGGSGGGGSEIVLTQSPGTLSLSPGERATLSCRSSQSLVHSNQNTYLHWYQQKPGQAPRLLIYKVDNRFSGIPDRFSGSGSGTDFTLTISRLEPEDFAVYYCSQSTLVPLTFGGGTKVEIK

SEQ ID NO: 99 - protein, 698 aa, synthetic construct
  source 1..698: note = anti-phosphorylated tau scFv propeptide fusion
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSGSGGGGSGAGGENLYFQEVQLVQSGAEVKKPGESLKISCKGSGYTFSNYWIEWVRQMPGKGLEWMGEILPGSDSIKYEKNFKGQVTISADKSISTAYLQWSSLKASDTAMYYCARRGNYVDDWGQGTLVTVSSGGGGSGGGGSGGGGSEIVLTQSPGTLSLSPGERATLSCRSSQSLVHSNQNTYLHWYQQKPGQAPRLLIYKVDNRFSGIPDRFSGSGSGTDFTLTISRLEPEDFAVYYCSQSTLVPLTFGGGTKVEIKGAGSGFANELGPRLMGKSGGGGSGSGDIPATYEFTDGKHYITNEPIPPKGSGENLYFQGHHHHHHHH

SEQ ID NO: 100 - DNA, 1683 bp, Corynebacterium diphtheriae
  source 1..1683: mol_type = genomic DNA; note = diphtheria toxin
gtgagcagaaaactgtttgcgtcaatcttaataggggcgctactggggataggggccccaccttcagcccatgcaggcgctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatcagtaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctccggggcataaaacgcaaccatttcttcatgacgggtatgctgtcagttggaacactgttgaagattcgataatccgaactggttttcaaggggagagtgggcacgacataaaaattactgctgaaaataccccgcttccaatcgcgggtgtcctactaccgactattcctggaaagctggacgttaataagtccaagactcatatttccgtaaatggtcggaaaataaggatgcgttgcagagctatagacggtgatgtaactttttgtcgccctaaatctcctgtttatgttggtaatggtgtgcatgcgaatcttcacgtggcatttcacagaagcagctcggagaaaattcattctaatgaaatttcgtcggattccataggcgttcttgggtaccagaaaacagtagatcacaccaaggttaattctaagctatcgctattttttgaaatcaaaagctga

SEQ ID NO: 101 - DNA, 567 bp, synthetic construct
  source 1..567: mol_type = other DNA; note = DT-C domain
ggcgctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaat

SEQ ID NO: 102 - DNA, 552 bp, synthetic construct
  source 1..552: mol_type = other DNA; note = DT-T domain
tcagtaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgt

SEQ ID NO: 103 - DNA, 3891 bp, Clostridium botulinum
  source 1..3891: mol_type = genomic DNA; note = BoNT/A1
atgccatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataataacttctaaaactaaatcattagataaaggatacaataaggcattaaatgatttatgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcatcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa

SEQ ID NO: 104 - DNA, 1214 bp, synthetic construct
  source 1..1214: mol_type = other DNA; note = receptor binding domain
ctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcatcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactg

SEQ ID NO: 105 - DNA, 2766 bp, synthetic construct
  source 1..2766: mol_type = other DNA; note = DTnd
atgggtcaccatcatcaccatcatcaccaccaccaccaccacgacgtgtcaggtttcgctaacgagctgggacccaggctgatgggcaagggagctggtgagaacctgtacttccagggtaagctgggctcaattgagttcatcaaggttaacaaaggcggtggcggtagcggcggtggcggtagcggcggtggcggatccggtgctgacgacgtcgttgatagcagcaagagcttcgttatggaaaacttcagcagctaccacggcaccaaaccgggttacgtggacagcatccagaagggcatccagaagccgaaaagcggtacccagggcaactacgacgatgactgggaaggtttctacagcaccgataacaagtacgacgcggctggctacagcgttgataacgagaacccgctgagcggtaaagcgggtggcgtggttaaggtgacctacccaggcctgaccaaagtgctggctctgaaggttgacaacgcggaaaccatcaagaaagagctgggcctgagcctgaccgaaccgctgatggagcaagtgggtactgaggaattcatcagccgtttcggtgacggcgcgagccgtgtggttctgagcctgccgttcgcggaaggtagcagcagcgttaaatacatcaacaactgggagcaggcgaaggccttaagcgtggagctggaaatcaacttcgagacgcgtggcaaggctggccaggatgctatgtacgagtacatggcgagcgcatgcgctggcaaccgtgtgcgtcgtagcgttggtagcagcttgagctgcatcaacctggattgggacgttatccgtgacaagaccaaaaccaagatcgaaagcttgaaagagcacgggcccatcagcaacaagatgagcgagagcccgaacaagaccgtgagcgaggaaaaagctaagagctacctggaggagttccaccagaccgctctggagcacccggaactgagcgagctgaagaccgtgaccggtacgaacccggttttcgctggtgcgaactacgcggcttgggctgtgaacgttgcgcaggttatcgatagcggcaccgctgacaacctggagaaaaccaccgcggctctgagcatcctgccgggtatcggcagcgtgatgggcatcgctgatggtgcggttcaccacaacaccgaggaaatcgtggctcagagcatcgcgctgagcagcctgatggttgctcaggcgatcccgctggttggtgaactggttgacatcggtttcgcggcttacaacttcgtggagagcatcatcaacctgttccaggttgtacacaacagctacaaccgtccggcttacagcccgggtcacggcacccagccgttcctggaggcgagcggtggaccggaggctaacatcatcaacacctccatcctgaacctccgttacgagtctaaccacctcatcgacttgagcagatacgctagcaagatcaacatcggttccaaggtgaacttcgacccaatcgataagaaccagatccaactgttcaacctcgaatcctctaagatcgaagtgatcctgaagaacgctatcgtctacaactccatgtacgagaacttctctaccagcttctggatcaggattccgaaatacttcaactcaatctcgctcaacaacgagtacactatcatcaactgcatggaaaacaactcgggatggaaggtgtccctcaactacggcgagatcatctggactttgcaggacacacaagaaatcaagcagagggtcgtgttcaagtacagccaaatgatcaacatcagcgattacatcaaccgttggatcttcgtcacaatcaccaacaaccgcctgaacaactccaagatttacatcaacggtagactgatcgaccagaagccaatcagcaacctcggcaacatccacgcctcaaacaacatcatgttcaagttggacggctgtagggatacacacagatacatctggatcaaatacttcaacctgttcgacaaggagctcaacgagaaggaaatcaaggacctctacgataaccagtccaactctggtatcttgaaggacttctggggcgattacctgcaatacgacaagccctactacatgttgaacctgtacgaccctaacaagtacgttgatgtgaacaacgtcggtatcaggggctacatgtacctgaagggaccacgtggttctgttatgaccactaacatctacctcaacagctcattgtaccgtggcacaaagttcatcatcaagaagtacgcctccggaaacaaggacaacatcgtccgtaacaacgatcgcgtttacatcaacgttgtggtcaagaacaaggagtacagactggctaccaacgcttcgcaggctggagttgagaagatcctgtctgctctggaaatccctgacgtgggcaacctctcacaggttgtggtcatgaagtcgaagaacgatcaaggcatcactaacaagtgcaagatgaacttgcaggacaacaacggaaacgacatcggcttcatcggattccaccaattcaacaacatcgccaagttggtggccagcaactggtacaaccgtcagatcgagcgttcgtcccgcaccttaggatgctcgtgggagttcattccagtcgatgacggatggggagagagacctttgggcgcaggagagaacctgtacttccagggtgcaggatggtcccacccacaattcgagaagggtgcaggatggagtcacccacagttcgagaagtaa

SEQ ID NO: 106 - DNA, 1893 bp, synthetic construct
  source 1..1893: mol_type = other DNA; note = MBP-B8 propeptide fusion
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgggtagcggcggtggcggtagcggtgcgggcgagaatctgtacttccagtccggttcgaacggcggggccggccagtcgggcgccggcgaaggcggtggcggtagcggcggtggcggtagcggcggtggcggatcccaggcgcacgtgcaactgcagcagtctggaggaggcttggtgcagcctgggggttccctgcgcctgtcatgtgcagcctctggaagcatcttcagtatttacgctatgggctggtacaggcaggctcctggcaagcaacgtgaactggttgctgccatctccagctacggtagtaccaactacgctgattcggtcaagggcaggttcaccatctcccgcgacaatgccaagaataccgtctatttgcaaatgaactctctgaaacctgaggatacggccgtctactactgcaacgctgacattgctactatgaccgcggtaggcggattcgactactggggacagggaactcaggtgacggtctcttccgcgcaccactccgaggaccctacttctcaaagcactagtggtggcggtggcagcggtggcggtggcagcggtggcggtggcagcggcggaggtggcagtgggacgtccgggtcgacagggggtggcggtagcggtggcggtggcagcggtgcaggagatatcccggcgacctacgagttcaccgacggcaagcactacatcaccaacgaaccgatcccgccgaaaggtgcaggagagaatctgtacttccagggccaccaccaccaccaccactaa

SEQ ID NO: 107 - DNA, 1902 bp, synthetic construct
  source 1..1902: mol_type = other DNA; note = MBP- JSG-C1 propeptide fusion
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgggtagcggcggtggcggtagcggtgcgggcgagaatctgtacttccagtccggttcgaacggcggggccggccagtcgggcgccggcgaaggcggtggcggtagcggcggtggcggtagcggcggtggcggatccgcggggacgtcccaggtgcagctggttgagagcggtggcggtctggtgcagaccggcggtagcctgcgtctgagctgcgctgctagcggtcgtaccttccgtcgtaacaccatgggttggttccgtcaggctccgggtaaagtgcgtgaattcgttgcggctatcagctggagcggcgaccgtacctactgcgctgatagcgtgaagggtcgtttcaccatcagccgtgacaacgctaaaaacaccgttgatctgctgatgaacagcctgaagccggaggacaccgcgatctactactgcgcggctgatggtaccgctagcgttttcaacagctacgcgagcgctgaccgtaacaagtacaactactggggccagggtacccaggtgaccgttagcagcgggtcgacagccactagtggtggcggtggcagcggtggcggtggcagcggtggcggtggcagcggcggaggtggcagtgggacgtccgggtcgacagggggtggcggtagcggtggcggtggcagcggtgcaggagatatcccggcgacctacgagttcaccgacggcaagcactacatcaccaacgaaccgatcccgccgaaaggtgcaggagagaatctgtacttccagggccaccaccaccaccaccactaa

SEQ ID NO: 108 - protein, 12 aa, synthetic construct
  source 1..12: note = SnoopTag
KLGDIEFIKVNK

SEQ ID NO: 109 - protein, 133 aa, synthetic construct
  source 1..133: note = SnoopCatcher
MGSSHHHHHHSSGLVPRGSHMKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 110 - protein, 104 aa, synthetic construct
  source 1..104: note = SnoopLigase
VNKNDKKPLRGAVFSLQKQHPDYPDIYGAIDQNGTYQNVRTGEDGKLTFKNLSDGKYRLFENSEPPGYKPVQNKPIVAFQIVNGEVRDVTSIVPPGVPATYEFT

SEQ ID NO: 111 - protein, 483 aa, synthetic construct
  source 1..483: note = HalobtnSnoopLigase
MGSSHHHHHHSSGEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGGSGGGGASGENLYFQGGLNDIFEAQKIEWHEGGGGSGGGGSGGGGSVNKNDKKPLRGAVFSLQKQHPDYPDIYGAIDQNGTYQNVRTGEDGKLTFKNLSDGKYRLFENSEPPGYKPVQNKPIVAFQIVNGEVRDVTSIVPPGVPATYEFTGAGWSHPQFEKGAGWSHPQFEK

SEQ ID NO: 112 - protein, 157 aa, synthetic construct
  source 1..157: note = btnSnoopLigase
GGLNDIFEAQKIEWHEGGGGSGGGGSGGGGSVNKNDKKPLRGAVFSLQKQHPDYPDIYGAIDQNGTYQNVRTGEDGKLTFKNLSDGKYRLFENSEPPGYKPVQNKPIVAFQIVNGEVRDVTSIVPPGVPATYEFTGAGWSHPQFEKGAGWSHPQFEK

SEQ ID NO: 113 - DNA, 1452 bp, synthetic construct
  source 1..1452: mol_type = other DNA; note = HalobtnSnoopLigase
atgggtagcagccatcaccaccaccaccacagcagcggtgaaatcggtaccggcttcccgtttgacccgcactacgttgaggtgctgggtgaacgtatgcactatgtggacgttggtccgcgtgatggcaccccggtgctgttcctgcacggcaacccgaccagcagctacgtgtggcgtaacatcattccgcatgttgcgccgacccaccgttgcattgcgccggatctgattggtatgggcaagagcgacaaaccggatctgggttatttctttgacgatcacgtgcgtttcatggacgcgtttatcgaggcgctgggcctggaggaagtggttctggttattcacgattggggtagcgcgctgggctttcactgggcgaagcgtaacccggagcgtgttaaaggtatcgcgttcatggaatttatccgtccgattccgacctgggacgagtggccggaattcgcgcgtgaaaccttccaggcgtttcgtaccaccgacgtgggccgtaagctgatcatcgatcaaaacgttttcattgagggtaccctgccgatgggcgtggttcgtccgctgaccgaggtggaaatggaccactaccgtgagccgttcctgaacccggttgatcgtgaaccgctgtggcgttttccgaacgagctgccgatcgcgggtgaaccggcgaacattgtggcgctggttgaggaatatatggactggctgcaccagagcccggtgccgaaactgctgttctggggtaccccgggcgttctgatcccgccggcggaagcggcgcgtctggcgaagagcctgccgaactgcaaagcggtggatattggtccgggcctgaacctgctgcaagaggacaacccggatctgatcggtagcgaaattgcgcgttggctgagcaccctggagatcagcggtggtggaggcggaagcggcggtggcggagctagcggtgagaacctctacttccagggtggactgaacgatattttcgaagcgcaaaagatcgaatggcacgagggtggaggcggaagcggcggtggcggaagcggtggtggcggatccgtgaacaagaacgacaagaaaccgctgcgtggtgcggttttcagcctgcagaaacaacacccggactacccggatatctatggtgcgattgatcagaacggcacctaccaaaacgtgcgtaccggcgaggacggcaagctgaccttcaaaaacctgagcgatggcaagtaccgtctgtttgagaacagcgaaccgccgggctataagccggttcagaacaaaccgatcgtggcgttccaaattgttaacggtgaagtgcgtgacgttaccagcattgtgccgccgggcgttccggcgacctatgaatttaccggtgcgggttggagccacccgcagtttgaaaagggtgcgggctggagccacccgcaatttgagaaataa

SEQ ID NO: 114 - protein, 1072 aa, synthetic construct
  source 1..1072: note = B8DTnd Fusion Protein
  CROSSLNK 221..222: note = NK isopeptide bond
SGSNGGAGQSGAGEGGGGSGGGGSGGGGSQAHVQLQQSGGGLVQPGGSLRLSCAASGSIFSIYAMGWYRQAPGKQRELVAAISSYGSTNYADSVKGRFTISRDNAKNTVYLQMNSLKPEDTAVYYCNADIATMTAVGGFDYWGQGTQVTVSSAHHSEDPTSQSTSGGGGSGGGGSGGGGSGGGGSGTSGSTGGGGSGGGGSGAGDIPATYEFTDGKHYITNKVNKGGGGSGGGGSGGGGSGADDVVDSSKSFVMENFSSYHGTKPGYVDSIQKGIQKPKSGTQGNYDDDWEGFYSTDNKYDAAGYSVDNENPLSGKAGGVVKVTYPGLTKVLALKVDNAETIKKELGLSLTEPLMEQVGTEEFISRFGDGASRVVLSLPFAEGSSSVKYINNWEQAKALSVELEINFETRGKAGQDAMYEYMASACAGNRVRRSVGSSLSCINLDWDVIRDKTKTKIESLKEHGPISNKMSESPNKTVSEEKAKSYLEEFHQTALEHPELSELKTVTGTNPVFAGANYAAWAVNVAQVIDSGTADNLEKTTAALSILPGIGSVMGIADGAVHHNTEEIVAQSIALSSLMVAQAIPLVGELVDIGFAAYNFVESIINLFQVVHNSYNRPAYSPGHGTQPFLEASGGPEANIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPLGAGENLYFQ